SEQUENCE LISTING (98 sequences)

SEQ ID NO: 1 - DNA, 1219 bp, Pichia pastoris
tgaatggcctgactaacagaagaatatttgttttccaggaagttaatgtctcttggaactgattcaattagttctttgtggtttgcttgatcgtctttcaagctctctgcgatgtccatttgctgctgaatgtactgatcaagttgttctatttctttttgataggcatctggtagatccgtgactctcgtaagggatgttatctgtggttgagaagcattgttagggttgttgggtgctgcattgttgcccagtaaactattattgttattacttgaattgaaaagcccaccagcattactgttagtattgtttccaaatagactccctgtagttgtattagcgttcgtattattgctcccaaaaagacctccagtgttaccactcggattatttgagcctgagaaaccactttgtgcaggtttattggcatttccaaacaacccccctccagtttgggcattactatttgcagtattgctgcctccaaaaagaccacccgattgtgtgttatttgaattggttccaaatagccctcctgattgtgtgttaccagaatttcctccaaataatcctccctgttgagtgttagagttagtgttggtgttacctccaaagagtcctttcgattgagtattactagcatttcccccaaacaaccctcctgattgattgctgttgctagcagtattcccaccaaacaatccttgcgattgagtatttcccgtgttggtgttggcgccaaataagctacctgactgattcgtgttggtattaccagaattgtttccaaacataccgcctgtattggtactacctgaagcgttcgtactgccaaacaatcctccgctattgcctccagaatttgtactagcattatttccaaacaaacctcctgtgttattcgtattcgtagcaggctttgctccaaacaagcctccagtgttaccagaggagctacctccaaaagccccaacattgctacctgaggcgtttccaaacataccgcctgaattggcggggttcttgttggaatttccaaacatttgactttaaggttttaaagaacgttgttttgacaagggaaacaaaagttcccactaaatttttcgatgtaaggacttgggaggagcaactgcttacatgaactccctctaactttcctcataaaaatccttccaagctcgcgaggtcccttccaactaagtcgggtaagttt

SEQ ID NO: 2 - DNA, 1475 bp, Pichia pastoris
tgcccggacttctatccagcaaatttacggaacggtgttcaatcaagtgttgagcgctcaacctcagttgcagcctgtcagaggcttctcaaatccggtacctgaaacccctgtaaatggagtccaagcgaatgaacaacactctgattctacccctcaaaatcattctagggatgaaaaccaaggaagaggtcgtggtagaggcagaggaaatagaagaggaagaggtcgaggtagaggcaaaggaggacagtaaatcagaatgaaggtgtccctccgattgaatagaattgtgttgtaatattagtgtattgcgttaatggtactaataatcatcctgcattgtataactaagaactttcttctcctgccttagagcagtcgtcttgaaaactttttgctcccagccattagacctatcaattccgtcccatctataccctggcattatagagaaacgattgtccgggaaagaagatttgtatagtttacggcctgtctgtgagatgtaacggtctttttgcagcgacttgactttatctggagaaaatgctagcaacgggtcatctacatagcttggcggagctactcttttagtggccgtttgagtaacctcggggagattcatattgcgtaatttttcagatgcttctttggctttctgtttttcttgctgtgatttctgtctctgttgttctagcatttcctcatgagagaggacgtttcctgataagtctctataaatagtggcattctcgggttttgcaggttttgtgggtttttgtatcgacgacttgggtttcggcttcagtgatttatcccttttcttggacttggatttgccatatttggaatccaggtagtcctgtagtgacattcgtttcgatactaggtgcgatggttcattcgatacgatatataatgcttacataagctaatggtactgggaacatcacttatactatccgttcagatcaacaggagaattcattcatacaacatgccaaattcattgaagacccagtcatcattccatcagactgcccttggttaaaggtgataaggaattaattgaggtttatcggggtttaaagtgaggcgggcatcaagaaaaaaaaaaagagggcaggagcagtggaactttcaaaacaagaaagagataaatcttatctcgtgacccctatcttagcaaataacgtttacgtttgaaggtaatagattaagcaaccaattacctcatcctaacttacgagtaatatcccgtttcatctcatcatcaatgagggacttgattttataccgacattgttggctccccacattaaccctttaaagcaggaagatccaattccccgagggacaaacttgacaccctaactttcccggggttcacgaaatattcatgaacccccccccttgatacgaacatctgcgccgtatgcacccttctgggacataccgcctgaggccacctc

SEQ ID NO: 3 - DNA, 3029 bp, Saccharomyces cerevisiae
aggcctcgcaacaacctataattgagttaagtgcctttccaagctaaaaagtttgaggttataggggcttagcatccacacgtcacaatctcgggtatcgagtatagtatgtagaattacggcaggaggtttcccaatgaacaaaggacaggggcacggtgagctgtcgaaggtatccattttatcatgtttcgtttgtacaagcacgacatactaagacatttaccgtatgggagttgttgtcctagcgtagttctcgctcccccagcaaagctcaaaaaagtacgtcatttagaatagtttgtgagcaaattaccagtcggtatgctacgttagaaaggcccacagtattcttctaccaaaggcgtgcctttgttgaactcgatccattatgagggcttccattattccccgcatttttattactctgaacaggaataaaaagaaaaaacccagtttaggaaattatccgggggcgaagaaatacgcgtagcgttaatcgaccccacgtccagggtttttccatggaggtttctggaaaaactgacgaggaatgtgattataaatccctttatgtgatgtctaagacttttaaggtacgcccgatgtttgcctattaccatcatagagacgtttcttttcgaggaatgcttaaacgactttgtttgacaaaaatgttgcctaagggctctatagtaaaccatttggaagaaagatttgacgactttttttttttggatttcgatcctataatccttcctcctgaaaagaaacatataaatagatatgtattattcttcaaaacattctcttgttcttgtgctttttttttaccatatatcttacttttttttttctctcagagaaacaagcaaaacaaaaagcttttcttttcactaacgtatatgatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagaggttataaaacttattgtcttttttatttttttcaaaagccattctaaagggctttagctaacgagtgacgaatgtaaaactttatgatttcaaagaatacctccaaaccattgaaaatgtatttttatttttattttctcccgaccccagttacctggaatttgttctttatgtactttatataagtataattctcttaaaaatttttactactttgcaatagacatcattttttcacgtaataaacccacaatcgtaatgtagttgccttacactactaggatggacctttttgcctttatctgttttgttactgacacaatgaaaccgggtaaagtattagttatgtgaaaatttaaaagcattaagtagaagtataccatattgtaaaaaaaaaaagcgttgtcttctacgtaaaagtgttctcaaaaagaagtagtgagggaaatggataccaagctatctgtaacaggagctaaaaaatctcagggaaaagcttctggtttgggaaacggtcgac

SEQ ID NO: 4 - DNA, 898 bp, Pichia pastoris
atcggcctttgttgatgcaagttttacgtggatcatggactaaggagttttatttggaccaagttcatcgtcctagacattacggaaagggttctgctcctctttttggaaactttttggaacctctgagtatgacagcttggtggattgtacccatggtatggcttcctgtgaatttctattttttctacattggattcaccaatcaaaacaaattagtcgccatggctttttggcttttgggtctatttgtttggaccttcttggaatatgctttgcatagatttttgttccacttggactactatcttccagagaatcaaattgcatttaccattcatttcttattgcatgggatacaccactatttaccaatggataaatacagattggtgatgccacctacacttttcattgtactttgctacccaatcaagacgctcgtcttttctgttctaccatattacatggcttgttctggatttgcaggtggattcctgggctatatcatgtatgatgtcactcattacgttctgcatcactccaagctgcctcgttatttccaagagttgaagaaatatcatttggaacatcactacaagaattacgagttaggctttggtgtcacttccaaattctgggacaaagtctttgggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactcgaggggtaccaaaaaaggtttttggatgctgcagtggcttcgc

SEQ ID NO: 5 - DNA, 1060 bp, Pichia pastoris
ggtcttttcaacaaagctccattagtgagtcagctggctgaatcttatgcacaggccatcattaacagcaacctggagatagacgttgtatttggaccagcttataaaggtattcctttggctgctattaccgtgttgaagttgtacgagctcggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggtagtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcgggtggtgcgggctcaggttccctttcataaacagatttagtacttggatgcttaatagtgaatggcgaatgcaaaggaacaatttcgttcatctttaaccctttcactcggggtacacgttctggaatgtacccgccctgttgcaactcaggtggaccgggcaattcttgaactttctgtaacgttgttggatgttcaaccagaaattgtcctaccaactgtattagtttccttttggtcttatattgttcatcgagatacttcccactctccttgatagccactctcactcttcctggattaccaaaatcttgaggatgagtcttttcaggctccaggatgcaaggtatatccaagtacctgcaagcatctaatattgtctttgccagggggttctccacaccatactccttttggcgcatgc

SEQ ID NO: 6 - DNA, 957 bp, Pichia pastoris
tctagagggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactggaggggtaccaaaaaaggtttttggatgctgcagtggcttcgcaggccttgaagtttggaactttcaccttgaaaagtggaagacagtctccatacttctttaacatgggtcttttcaacaaagctccattagtgagtcagctggctgaatcttatgctcaggccatcattaacagcaacctggagatagacgttgtatttggaccagcttataaaggtattcctttggctgctattaccgtgttgaagttgtacgagctgggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggttgtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcggatcc

SEQ ID NO: 7 - DNA, 709 bp, Escherichia coli
cctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccag

SEQ ID NO: 8 - DNA, 2875 bp, Pichia pastoris
aaaaccttttttcctattcaaacacaaggcattgcttcaacacgtgtgcgtatccttaacacagatactccatacttctaataatgtgatagacgaatacaaagatgttcactctgtgttgtgtctacaagcatttcttattctgattggggatattctagttacagcactaaacaactggcgatacaaacttaaattaaataatccgaatctagaaaatgaacttttggatggtccgcctgttggttggataaatcaataccgattaaatggattctattccaatgagagagtaatccaagacactctgatgtcaataatcatttgcttgcaacaacaaacccgtcatctaatcaaagggtttgatgaggcttaccttcaattgcagataaactcattgctgtccactgctgtattatgtgagaatatgggtgatgaatctggtcttctccactcagctaacatggctgtttgggcaaaggtggtacaattatacggagatcaggcaatagtgaaattgttgaatatggctactggacgatgcttcaaggatgtacgtctagtaggagccgtgggaagattgctggcagaaccagttggcacgtcgcaacaatccccaagaaatgaaataagtgaaaacgtaacgtcaaagacagcaatggagtcaatattgataacaccactggcagagcggttcgtacgtcgttttggagccgatatgaggctcagcgtgctaacagcacgattgacaagaagactctcgagtgacagtaggttgagtaaagtattcgcttagattcccaaccttcgttttattctttcgtagacaaagaagctgcatgcgaacatagggacaacttttataaatccaattgtcaaaccaacgtaaaaccctctggcaccattttcaacatatatttgtgaagcagtacgcaatatcgataaatactcaccgttgtttgtaacagccccaacttgcatacgccttctaatgacctcaaatggataagccgcagcttgtgctaacataccagcagcaccgcccgcggtcagctgcgcccacacatataaaggcaatctacgatcatgggaggaattagttttgaccgtcaggtcttcaagagttttgaactcttcttcttgaactgtgtaaccttttaaatgacgggatctaaatacgtcatggatgagatcatgtgtgtaaaaactgactccagcatatggaatcattccaaagattgtaggagcgaacccacgataaaagtttcccaaccttgccaaagtgtctaatgctgtgacttgaaatctgggttcctcgttgaagaccctgcgtactatgcccaaaaactttcctccacgagccctattaacttctctatgagtttcaaatgccaaacggacacggattaggtccaatgggtaagtgaaaaacacagagcaaaccccagctaatgagccggccagtaaccgtcttggagctgtttcataagagtcattagggatcaataacgttctaatctgttcataacatacaaattttatggctgcatagggaaaaattctcaacagggtagccgaatgaccctgatatagacctgcgacaccatcatacccatagatctgcctgacagccttaaagagcccgctaaaagacccggaaaaccgagagaactctggattagcagtctgaaaaagaatcttcactctgtctagtggagcaattaatgtcttagcggcacttcctgctactccgccagctactcctgaatagatcacatactgcaaagactgcttgtcgatgaccttggggttatttagcttcaagggcaatttttgggacattttggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagccatggcgaaggcagatggcagtttgctctactataatcctcacaatccacccagaaggtattacttctacatggctatattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcg

SEQ ID NO: 9 - DNA, 997 bp, Pichia pastoris
aaagctagagtaaaatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaaccaaacaccaagaagcaaaaactaaccccatataaaaacatcctggtagataatgctggtaacccgctctccttccatattctgggctacttcacgaagtctgaccggtctcagttgatcaacatgatcctcgaaatgggtggcaagatcgttccagacctgcctcctctggtagatggagtgttgtttttgacaggggattacaagtctattgatgaagataccctaaagcaactgggggacgttccaatatacagagactccttcatctaccagtgttttgtgcacaagacatctcttcccattgacactttccgaattgacaagaacgtcgacttggctcaagatttgatcaatagggcccttcaagagtctgtggatcatgtcacttctgccagcacagctgcagctgctgctgttgttgtcgctaccaacggcctgtcttctaaaccagacgctcgtactagcaaaatacagttcactcccgaagaagatcgttttattcttgactttgttaggagaaatcctaaacgaagaaacacacatcaactgtacactgagctcgctcagcacatgaaaaaccatacgaatcattctatccgccacagatttcgtcgtaatctttccgctcaacttgattgggtttatgatatcgatccattgaccaaccaacctcgaaaagatgaaaacgggaactacatcaaggtacaaggccttcca

SEQ ID NO: 10 - DNA, 2159 bp, Kluyveromyces lactis
aaacgtaacgcctggcactctattttctcaaacttctgggacggaagagctaaatattgtgttgcttgaacaaacccaaaaaaacaaaaaaatgaacaaactaaaactacacctaaataaaccgtgtgtaaaacgtagtaccatattactagaaaagatcacaagtgtatcacacatgtgcatctcatattacatcttttatccaatccattctctctatcccgtctgttcctgtcagattctttttccataaaaagaagaagaccccgaatctcaccggtacaatgcaaaactgctgaaaaaaaaagaaagttcactggatacgggaacagtgccagtaggcttcaccacatggacaaaacaattgacgataaaataagcaggtgagcttctttttcaagtcacgatccctttatgtctcagaaacaatatatacaagctaaacccttttgaaccagttctctcttcatagttatgttcacataaattgcgggaacaagactccgctggctgtcaggtacacgttgtaacgttttcgtccgcccaattattagcacaacattggcaaaaagaaaaactgctcgttttctctacaggtaaattacaatttttttcagtaattttcgctgaaaaatttaaagggcaggaaaaaaagacgatctcgactttgcatagatgcaagaactgtggtcaaaacttgaaatagtaattttgctgtgcgtgaactaataaatatatatatatatatatatatatatttgtgtattttgtatatgtaattgtgcacgtcttggctattggatataagattttcgcgggttgatgacatagagcgtgtactactgtaatagttgtatattcaaaagctgctgcgtggagaaagactaaaatagataaaaagcacacattttgacttcggtaccgtcaacttagtgggacagtcttttatatttggtgtaagctcatttctggtactattcgaaacagaacagtgttttctgtattaccgtccaatcgtttgtcatgagttttgtattgattttgtcgttagtgttcggaggatgttgttccaatgtgattagtttcgagcacatggtgcaaggcagcaatataaatttgggaaatattgttacattcactcaattcgtgtctgtgacgctaattcagttgcccaatgctttggacttctctcactttccgtttaggttgcgacctagacacattcctcttaagatccatatgttagctgtgtttttgttctttaccagttcagtcgccaataacagtgtgtttaaatttgacatttccgttccgattcatattatcattagattttcaggtaccactttgacgatgataataggttgggctgtttgtaataagaggtactccaaacttcaggtgcaatctgccatcattatgacgcttggtgcgattgtcgcatcattataccgtgacaaagaattttcaatggacagtttaaagttgaatacggattcagtgggtatgacccaaaaatctatgtttggtatctttgttgtgctagtggccactgccttgatgtcattgttgtcgttgctcaacgaatggacgtataacaagtacgggaaacattggaaagaaactttgttctattcgcatttcttggctctaccgttgtttatgttggggtacacaaggctcagagacgaattcagagacctcttaatttcctcagactcaatggatattcctattgttaaattaccaattgctacgaaacttttcatgctaatagcaaataacgtgacccagttcatttgtatcaaaggtgttaacatgctagctagtaacacggatgctttgacactttctgtcgtgcttctagtgcgtaaatttgttagtcttttactcagtgtctacatctacaagaacgtcctatccgtgactgcatacctagggaccatcaccgtgttcctgggagctggtttgtattcatatggttcggtcaaaactgcactgcctcgctgaaacaatccacgtctgtatgatactcgtttcagaatttttttgattttctgccggatatggtttctcatctttacaatcgcattcttaattataccagaacgtaattcaatgatcccagtgactcgtaactcttatatgtcaatttaagc

SEQ ID NO: 11 - DNA, 870 bp, Pichia pastoris
ggccgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagcttcaacgacatggaggtcgactcctatgtcaccaacatctacgacaatgctccagtgctaggatgtacggatttgtcttatcatggattgttgaaagtcaccccaaagcatgacttagcttgcgatttggagttcataagagctcagattttggacattgacgtttactccgccataaaagacttagaagataaagccttgactgtaaaacaaaaggttgaaaaacactggtttacgttttatggtagttcagtctttctgcccgaacacgatgtgcattacctggttagacgagtcatcttttcggctgaaggaaaggcgaactctccagtaacatc

SEQ ID NO: 12 - DNA, 1733 bp, Pichia pastoris
ccatatgatgggtgtttgctcactcgtatggatcaaaattccatggtttcttctgtacaacttgtacacttatttggacttttctaacggtttttctggtgatttgagaagtccttattttggtgttcgcagcttatccgtgattgaaccatcagaaatactgcagctcgttatctagtttcagaatgtgttgtagaatacaatcaattctgagtctagtttgggtgggtcttggcgacgggaccgttatatgcatctatgcagtgttaaggtacatagaatgaaaatgtaggggttaatcgaaagcatcgttaatttcagtagaacgtagttctattccctacccaaataatttgccaagaatgcttcgtatccacatacgcagtggacgtagcaaatttcactttggactgtgacctcaagtcgttatcttctacttggacattgatggtcattacgtaatccacaaagaattggatagcctctcgttttatctagtgcacagcctaatagcacttaagtaagagcaatggacaaatttgcatagacattgagctagatacgtaactcagatcttgttcactcatggtgtactcgaagtactgctggaaccgttacctcttatcatttcgctactggctcgtgaaactactggatgaaaaaaaaaaaagagctgaaagcgagatcatcccattttgtcatcatacaaattcacgcttgcagttttgcttcgttaacaagacaagatgtctttatcaaagacccgttttttcttcttgaagaatacttccctgttgagcacatgcaaaccatatttatctcagatttcactcaacttgggtgcttccaagagaagtaaaattcttcccactgcatcaacttccaagaaacccgtagaccagtttctcttcagccaaaagaagttgctcgccgatcaccgcggtaacagaggagtcagaaggtttcacacccttccatcccgatttcaaagtcaaagtgctgcgttgaaccaaggttttcaggttgccaaagcccagtctgcaaaaactagttccaaatggcctattaattcccataaaagtgttggctacgtatgtatcggtacctccattctggtatttgctattgttgtcgttggtgggttgactagactgaccgaatccggtctttccataacggagtggaaacctatcactggttcggttcccccactgactgaggaagactggaagttggaatttgaaaaatacaaacaaagccctgagtttcaggaactaaattctcacataacattggaagagttcaagtttatattttccatggaatggggacatagattgttgggaagggtcatcggcctgtcgtttgttcttcccacgttttacttcattgcccgtcgaaagtgttccaaagatgttgcattgaaactgcttgcaatatgctctatgataggattccaaggtttcatcggctggtggatggtgtattccggattggacaaacagcaattggctgaacgtaactccaaaccaactgtgtctccatatcgcttaactacccatcttggaactgcatttgttatttactgttacatgatttacacagggcttcaagttttgaagaactataagatcatgaaacagcctgaagcgtatgttcaaattttcaagcaaattgcgtctccaaaattgaaaactttcaagagactctcttcagttctattaggcctggtg

SEQ ID NO: 13 - DNA, 981 bp, Mus musculus
atgtctgccaacctaaaatatctttccttgggaattttggtgtttcagactaccagtctggttctaacgatgcggtattctaggactttaaaagaggaggggcctcgttatctgtcttctacagcagtggttgtggctgaatttttgaagataatggcctgcatctttttagtctacaaagacagtaagtgtagtgtgagagcactgaatagagtactgcatgatgaaattcttaataagcccatggaaaccctgaagctcgctatcccgtcagggatatatactcttcagaacaacttactctatgtggcactgtcaaacctagatgcagccacttaccaggttacatatcagttgaaaatacttacaacagcattattttctgtgtctatgcttggtaaaaaattaggtgtgtaccagtggctctccctagtaattctgatggcaggagttgcttttgtacagtggccttcagattctcaagagctgaactctaaggacctttcaacaggctcacagtttgtaggcctcatggcagttctcacagcctgtttttcaagtggctttgctggagtttattttgagaaaatcttaaaagaaacaaaacagtcagtatggataaggaacattcaacttggtttctttggaagtatatttggattaatgggtgtatacgtttatgatggagaattggtctcaaagaatggattttttcagggatataatcaactgacgtggatagttgttgctctgcaggcacttggaggccttgtaatagctgctgtcatcaaatatgcagataacattttaaaaggatttgcgacctccttatccataatattgtcaacaataatatcttatttttggttgcaagattttgtgccaaccagtgtctttttccttggagccatccttgtaatagcagctactttcttgtatggttacgatcccaaacctgcaggaaatcccactaaagcatag

SEQ ID NO: 14 - DNA, 486 bp, Pichia pastoris
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 15 - DNA, 293 bp, Saccharomyces cerevisiae
acaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgccggctcttaag

SEQ ID NO: 16 - DNA, 1128 bp, Pichia pastoris
gatctggccattgtgaaacttgacactaaagacaaaactcttagagtttccaatcacttaggagacgatgtttcctacaacgagtacgatccctcattgatcatgagcaatttgtatgtgaaaaaagtcatcgaccttgacaccttggataaaagggctggaggaggtggaaccacctgtgcaggcggtctgaaagtgttcaagtacggatctactaccaaatatacatctggtaacctgaacggcgtcaggttagtatactggaacgaaggaaagttgcaaagctccaaatttgtggttcgatcctctaattactctcaaaagcttggaggaaacagcaacgccgaatcaattgacaacaatggtgtgggttttgcctcagctggagactcaggcgcatggattctttccaagctacaagatgttagggagtaccagtcattcactgaaaagctaggtgaagctacgatgagcattttcgatttccacggtcttaaacaggagacttctactacagggcttggggtagttggtatgattcattcttacgacggtgagttcaaacagtttggtttgttcactccaatgacatctattctacaaagacttcaacgagtgaccaatgtagaatggtgtgtagcgggttgcgaagatggggatgtggacactgaaggagaacacgaattgagtgatttggaacaactgcatatgcatagtgattccgactagtcaggcaagagagagccctcaaatttacctctctgcccctcctcactccttttggtacgcataattgcagtataaagaacttgctgccagccagtaatcttatttcatacgcagttctatatagcacataatcttgcttgtatgtatgaaatttaccgcgttttagttgaaattgtttatgttgtgtgccttgcatgaaatctctcgttagccctatccttacatttaactggtctcaaaacctctaccaattccattgctgtacaacaatatgaggcggcattactgtagggttggaaaaaaattgtcattccagctagagatcacacgacttcatcacgcttattgctcctcattgctaaatcatttactcttgacttcgacccagaaaagttcgcc

SEQ ID NO: 17 - DNA, 1231 bp, Pichia pastoris
gcatgtcaaacttgaacacaacgactagatagttgttttttctatataaaacgaaacgttatcatctttaataatcattgaggtttacccttatagttccgtattttcgtttccaaacttagtaatcttttggaaatatcatcaaagctggtgccaatcttcttgtttgaagtttcaaactgctccaccaagctacttagagactgttctaggtctgaagcaacttcgaacacagagacagctgccgccgattgttcttttttgtgtttttcttctggaagaggggcatcatcttgtatgtccaatgcccgtatcctttctgagttgtccgacacattgtccttcgaagagtttcctgacattgggcttcttctatccgtgtattaattttgggttaagttcctcgtttgcatagcagtggatacctcgatttttttggctcctatttacctgacataatattctactataatccaacttggacgcgtcatctatgataactaggctctcctttgttcaaaggggacgtcttcataatccactggcacgaagtaagtctgcaacgaggcggcttttgcaacagaacgatagtgtcgtttcgtacttggactatgctaaacaaaaggatctgtcaaacatttcaaccgtgtttcaaggcactctttacgaattatcgaccaagaccttcctagacgaacatttcaacatatccaggctactgcttcaaggtggtgcaaatgataaaggtatagatattagatgtgtttgggacctaaaacagttcttgcctgaagattcccttgagcaacaggcttcaatagccaagttagagaagcagtaccaaatcggtaacaaaagggggaagcatataaaacctttactattgcgacaaaatccatccttgaaagtaaagctgtttgttcaatgtaaagcatacgaaacgaaggaggtagatcctaagatggttagagaacttaacgggacatactccagctgcatcccatattacgatcgctggaagacttttttcatgtacgtatcgcccaccaacctttcaaagcaagctaggtatgattttgacagttctcacaatccattggttttcatgcaacttgaaaaaacccaactcaaacttcatggggatccatacaatgtaaatcattacgagagggcgaggttgaaaagtttccattgcaatcacgtcgcatcatggctactgaaaggccttaac

SEQ ID NO: 18 - DNA, 937 bp, Pichia pastoris
tcattctatatgttcaagaaaagggtagtgaaaggaaagaaaaggcatataggcgagggagagttagctagcatacaagataatgaaggatcaatagcggtagttaaagtgcacaagaaaagagcacctgttgaggctgatgataaagctccaattacattgccacagagaaacacagtaacagaaataggaggggatgcaccacgagaagagcattcagtgaacaactttgccaaattcataaccccaagcgctaataagccaatgtcaaagtcggctactaacattaatagtacaacaactatcgattttcaaccagatgtttgcaaggactacaaacagacaggttactgcggatatggtgacacttgtaagtttttgcacctgagggatgatttcaaacagggatggaaattagatagggagtgggaaaatgtccaaaagaagaagcataatactctcaaaggggttaaggagatccaaatgtttaatgaagatgagctcaaagatatcccgtttaaatgcattatatgcaaaggagattacaaatcacccgtgaaaacttcttgcaatcattatttttgcgaacaatgtttcctgcaacggtcaagaagaaaaccaaattgtattatatgtggcagagacactttaggagttgctttaccagcaaagaagttgtcccaatttctggctaagatacataataatgaaagtaataaagtttagtaattgcattgcgttgactattgattgcattgatgtcgtgtgatactttcaccgaaaaaaaacacgaagcgcaataggagcggttgcatattagtccccaaagctatttaattgtgcctgaaactgttttttaagctcatcaagcataattgtatgcattgcgacgtaaccaacgtttaggcgcagtttaatcatagcccactgctaagcc

SEQ ID NO: 19 - DNA, 1906 bp, Pichia pastoris
cggaggaatgcaaataataatctccttaattacccactgataagctcaagagacgcggtttgaaaacgatataatgaatcatttggattttataataaaccctgacagtttttccactgtattgttttaacactcattggaagctgtattgattctaagaagctagaaatcaatacggccatacaaaagatgacattgaataagcaccggcttttttgattagcatataccttaaagcatgcattcatggctacatagttgttaaagggcttcttccattatcagtataatgaattacataatcatgcacttatatttgcccatctctgttctctcactcttgcctgggtatattctatgaaattgcgtatagcgtgtctccagttgaaccccaagcttggcgagtttgaagagaatgctaaccttgcgtattccttgcttcaggaaacattcaaggagaaacaggtcaagaagccaaacattttgatccttcccgagttagcattgactggctacaattttcaaagccagcagcggatagagccttttttggaggaaacaaccaagggagctagtacccaatgggctcaaaaagtatccaagacgtgggattgctttactttaataggatacccagaaaaaagtttagagagccctccccgtatttacaacagtgcggtacttgtatcgcctcagggaaaagtaatgaacaactacagaaagtccttcttgtatgaagctgatgaacattggggatgttcggaatcttctgatgggtttcaaacagtagatttattaattgaaggaaagactgtaaagacatcatttggaatttgcatggatttgaatccttataaatttgaagctccattcacagacttcgagttcagtggccattgcttgaaaaccggtacaagactcattttgtgcccaatggcctggttgtcccctctatcgccttccattaaaaaggatcttagtgatatagagaaaagcagacttcaaaagttctaccttgaaaaaatagataccccggaatttgacgttaattacgaattgaaaaaagatgaagtattgcccacccgtatgaatgaaacgttggaaacaattgactttgagccttcaaaaccggactactctaatataaattattggatactaaggttttttccctttctgactcatgtctataaacgagatgtgctcaaagagaatgcagttgcagtcttatgcaaccgagttggcattgagagtgatgtcttgtacggaggatcaaccacgattctaaacttcaatggtaagttagcatcgacacaagaggagctggagttgtacgggcagactaatagtctcaaccccagtgtggaagtattgggggcccttggcatgggtcaacagggaattctagtacgagacattgaattaacataatatacaatatacaataaacacaaataaagaatacaagcctgacaaaaattcacaaattattgcctagacttgtcgttatcagcagcgacctttttccaatgctcaatttcacgatatgccttttctagctctgctttaagcttctcattggaattggctaactcgttgactgcttggtcagtgatgagtttctccaaggtccatttctcgatgttgttgttttcgttttcctttaatctcttgatataatcaacagccttctttaatatctgagccttgttcgagtcccctgttggcaacagagcggccagttcctttattccgtggtttatattttctcttctacgcctttctacttctttgtgattctctttacgcatcttatgccattcttcagaaccagtggctggcttaaccgaatagccagagcctgaagaagccgcactagaagaagcagtggcattgttgactatgg

SEQ ID NO: 20 - DNA, 1224 bp, Homo sapiens
tcagtcagtgctcttgatggtgacccagcaagtttgaccagagaagtgattagattggcccaagacgcagaggtggagttggagagacaacgtggactgctgcagcaaatcggagatgcattgtctagtcaaagaggtagggtgcctaccgcagctcctccagcacagcctagagtgcatgtgacccctgcaccagctgtgattcctatcttggtcatcgcctgtgacagatctactgttagaagatgtctggacaagctgttgcattacagaccatctgctgagttgttccctatcatcgttagtcaagactgtggtcacgaggagactgcccaagccatcgcctcctacggatctgctgtcactcacatcagacagcctgacctgtcatctattgctgtgccaccagaccacagaaagttccaaggttactacaagatcgctagacactacagatgggcattgggtcaagtcttcagacagtttagattccctgctgctgtggtggtggaggatgacttggaggtggctcctgacttctttgagtactttagagcaacctatccattgctgaaggcagacccatccctgtggtgtgtctctgcctggaatgacaacggtaaggagcaaatggtggacgcttctaggcctgagctgttgtacagaaccgacttctttcctggtctgggatggttgctgttggctgagttgtgggctgagttggagcctaagtggccaaaggcattctgggacgactggatgagaagacctgagcaaagacagggtagagcctgtatcagacctgagatctcaagaaccatgacctttggtagaaagggagtgtctcacggtcaattctttgaccaacacttgaagtttatcaagctgaaccagcaatttgtgcacttcacccaactggacctgtcttacttgcagagagaggcctatgacagagatttcctagctagagtctacggagctcctcaactgcaagtggagaaagtgaggaccaatgacagaaaggagttgggagaggtgagagtgcagtacactggtagggactcctttaaggctttcgctaaggctctgggtgtcatggatgaccttaagtctggagttcctagagctggttacagaggtattgtcacctttcaattcagaggtagaagagtccacttggctcctccacctacttgggagggttatgatccttcttggaattag

SEQ ID NO: 21 - DNA, 99 bp, Pichia pastoris
atgcccagaaaaatatttaactacttcattttgactgtattcatggcaattcttgctattgttttacaatggtctatagagaatggacatgggcgcgcc

SEQ ID NO: 22 - DNA, 435 bp, Pichia pastoris
gaagtaaagttggcgaaactttgggaacctttggttaaaactttgtaatttttgtcgctacccattaggcagaatctgcatcttgggagggggatgtggtggcgttctgagatgtacgcgaagaatgaagagccagtggtaacaacaggcctagagagatacgggcataatgggtataacctacaagttaagaatgtagcagccctggaaaccagattgaaacgaaaaacgaaatcatttaaactgtaggatgttttggctcattgtctggaaggctggctgtttattgccctgttctttgcatgggaataagctattatatccctcacataatcccagaaaatagattgaagcaacgcgaaatccttacgtatcgaagtagccttcttacacattcacgttgtacggataagaaaactactcaaacgaacaatc

SEQ ID NO: 23 - DNA, 404 bp, Pichia pastoris
aatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaacacaaacaccaagaagcaaaaactaaccccatataaaaacatcctggtagataatgctggtaacccgctctccttccatattctgggctacttcacgaagtctgaccggtctcagttgatcaacatgatcctcgaaatgg

SEQ ID NO: 24 - DNA, 1407 bp, Mus musculus
gagcccgctgacgccaccatccgtgagaagagggcaaagatcaaagagatgatgacccatgcttggaataattataaacgctatgcgtggggcttgaacgaactgaaacctatatcaaaagaaggccattcaagcagtttgtttggcaacatcaaaggagctacaatagtagatgccctggatacccttttcattatgggcatgaagactgaatttcaagaagctaaatcgtggattaaaaaatatttagattttaatgtgaatgctgaagtttctgtttttgaagtcaacatacgcttcgtcggtggactgctgtcagcctactatttgtccggagaggagatatttcgaaagaaagcagtggaacttggggtaaaattgctacctgcatttcatactccctctggaataccttgggcattgctgaatatgaaaagtgggatcgggcggaactggccctgggcctctggaggcagcagtatcctggccgaatttggaactctgcatttagagtttatgcacttgtcccacttatcaggagacccagtctttgccgaaaaggttatgaaaattcgaacagtgttgaacaaactggacaaaccagaaggcctttatcctaactatctgaaccccagtagtggacagtggggtcaacatcatgtgtcggttggaggacttggagacagcttttatgaatatttgcttaaggcgtggttaatgtctgacaagacagatctcgaagccaagaagatgtattttgatgctgttcaggccatcgagactcacttgatccgcaagtcaagtgggggactaacgtacatcgcagagtggaaggggggcctcctggaacacaagatgggccacctgacgtgctttgcaggaggcatgtttgcacttggggcagatggagctccggaagcccgggcccaacactaccttgaactcggagctgaaattgcccgcacttgtcatgaatcttataatcgtacatatgtgaagttgggaccggaagcgtttcgatttgatggcggtgtggaagctattgccacgaggcaaaatgaaaagtattacatcttacggcccgaggtcatcgagacatacatgtacatgtggcgactgactcacgaccccaagtacaggacctgggcctgggaagccgtggaggctctagaaagtcactgcagagtgaacggaggctactcaggcttacgggatgtttacattgcccgtgagagttatgacgatgtccagcaaagtttcttcctggcagagacactgaagtatttgtacttgatattttccgatgatgaccttcttccactagaacactggatcttcaacaccgaggctcatcctttccctatactccgtgaacagaagaaggaaattgatggcaaagagaaatga

SEQ ID NO: 25 - DNA, 318 bp, Saccharomyces cerevisiae
atgaacactatccacataataaaattaccgcttaactacgccaactacacctcaatgaaacaaaaaatctctaaatttttcaccaacttcatccttattgtgctgctttcttacattttacagttctcctataagcacaatttgcattccatgcttttcaattacgcgaaggacaattttctaacgaaaagagacaccatctcttcgccctacgtagttgatgaagacttacatcaaacaactttgtttggcaaccacggtacaaaaacatctgtacctagcgtagattccataaaagtgcatggcgtggggcgcgcc

SEQ ID NO: 26 - DNA, 1250 bp, Pichia pastoris
gagtcggccaagagatgataactgttactaagcttctccgtaattagtggtattttgtaacttttaccaataatcgtttatgaatacggatatttttcgaccttatccagtgccaaatcacgtaacttaatcatggtttaaatactccacttgaacgattcattattcagaaaaaagtcaggttggcagaaacacttgggcgctttgaagagtataagagtattaagcattaaacatctgaactttcaccgccccaatatactactctaggaaactcgaaaaattcctttccatgtgtcatcgcttccaacacactttgctgtatccttccaagtatgtccattgtgaacactgatctggacggaatcctacctttaatcgccaaaggaaaggttagagacatttatgcagtcgatgagaacaacttgctgttcgtcgcaactgaccgtatctccgcttacgatgtgattatgacaaacggtattcctgataagggaaagattttgactcagctctcagttttctggtttgattttttggcaccctacataaagaatcatttggttgcttctaatgacaaggaagtctttgctttactaccatcaaaactgtctgaagaaaaatacaaatctcaattagagggacgatccttgatagtaaaaaagcacagactgatacctttggaagccattgtcagaggttacatcactggaagtgcatggaaagagtacaagaactcaaaaactgtccatggagtcaaggttgaaaacgagaaccttcaagagagcgacgcctttccaactccgattttcacaccttcaacgaaagctgaacagggtgaacacgatgaaaacatctctattgaacaagctgctgagattgtaggtaaagacatttgtgagaaggtcgctgtcaaggcggtcgagttgtattctgctgcaaaaaacctcgcccttttgaaggggatcattattgctgatacgaaattcgaatttggactggacgaaaacaatgaattggtactagtagatgaagttttaactccagattcttctagattttggaatcaaaagacttaccaagtgggtaaatcgcaagagagttacgataagcagtttctcagagattggttgacggccaacggattgaatggcaaagagggcgtagccatggatgcagaaattgctatcaagagtaaagaaaagtatattgaagcttatgaagcaattactggcaagaaatgggcttga

SEQ ID NO: 27 - DNA, 376 bp, Pichia pastoris
atttacaattagtaatattaaggtggtaaaaacattcgtagaattgaaatgaattaatatagtatgacaatggttcatgtctataaatctccggcttcggtaccttctccccaattgaatacattgtcaaaatgaatggttgaactattaggttcgccagtttcgttattaagaaaactgttaaaatcaaattccatatcatcggttccagtgggaggaccagttccatcgccaaaatcctgtaagaatccattgtcagaacctgtaaagtcagtttgagatgaaatttttccggtctttgttgacttggaagcttcgttaaggttaggtgaaacagtttgatcaaccagcggctcccgttttcgtcgcttagtag

SEQ ID NO: 28 - DNA, 882 bp, Pichia pastoris
atgattagtaccctcctcgcctttttcagacatctgaaatttcccttattcttccaattccatataaaatcctatttaggtaattagtaaacaatgatcataaagtgaaatcattcaagtaaccattccgtttatcgttgatttaaaatcaataacgaatgaatgtcggtctgagtagtcaatttgttgccttggagctcattggcagggggtcttttggctcagtatggaaggttgaaaggaaaacagatggaaagtggttcgtcagaaaagaggtatcctacatgaagatgaatgccaaagagatatctcaagtgatagctgagttcagaattcttagtgagttaagccatcccaacattgtgaagtaccttcatcacgaacatatttctgagaataaaactgtcaatttatacatggaatactgtgatggtggagatctctccaagctgattcgaacacatagaaggaacaaagagtacatttcagaagaaaaaatatggagtatttttacgcaggttttattagcattgtatcgttgtcattatggaactgatttcacggcttcaaaggagtttgaatcgctcaataaaggtaatagacgaacccagaatccttcgtgggtagactcgacaagagttattattcacagggatataaaacccgacaacatctttctgatgaacaattcaaaccttgtcaaactgggagattttggattagcaaaaattctggaccaagaaaacgattttgccaaaacatacgtcggtacgccgtattacatgtctcctgaagtgctgttggaccaaccctactcaccattatgtgatatatggtctcttgggtgcgtcatgtatgagctatgtgcattgaggcctcctt

SEQ ID NO: 29 - DNA, 2100 bp, Saccharomyces cerevisiae
atgacagctcagttacaaagtgaaagtacttctaaaattgttttggttacaggtggtgctggatacattggttcacacactgtggtagagctaattgagaatggatatgactgtgttgttgctgataacctgtcgaattcaacttatgattctgtagccaggttagaggtcttgaccaagcatcacattcccttctatgaggttgatttgtgtgaccgaaaaggtctggaaaaggttttcaaagaatataaaattgattcggtaattcactttgctggtttaaaggctgtaggtgaatctacacaaatcccgctgagatactatcacaataacattttgggaactgtcgttttattagagttaatgcaacaatacaacgtttccaaatttgttttttcatcttctgctactgtctatggtgatgctacgagattcccaaatatgattcctatcccagaagaatgtcccttagggcctactaatccgtatggtcatacgaaatacgccattgagaatatcttgaatgatctttacaatagcgacaaaaaaagttggaagtttgctatcttgcgttattttaacccaattggcgcacatccctctggattaatcggagaagatccgctaggtataccaaacaatttgttgccatatatggctcaagtagctgttggtaggcgcgagaagctttacatcttcggagacgattatgattccagagatggtaccccgatcagggattatatccacgtagttgatctagcaaaaggtcatattgcagccctgcaatacctagaggcctacaatgaaaatgaaggtttgtgtcgtgagtggaacttgggttccggtaaaggttctacagtttttgaagtttatcatgcattctgcaaagcttctggtattgatcttccatacaaagttacgggcagaagagcaggtgatgttttgaacttgacggctaaaccagatagggccaaacgcgaactgaaatggcagaccgagttgcaggttgaagactcctgcaaggatttatggaaatggactactgagaatccttttggttaccagttaaggggtgtcgaggccagattttccgctgaagatatgcgttatgacgcaagatttgtgactattggtgccggcaccagatttcaagccacgtttgccaatttgggcgccagcattgttgacctgaaagtgaacggacaatcagttgttcttggctatgaaaatgaggaagggtatttgaatcctgatagtgcttatataggcgccacgatcggcaggtatgctaatcgtatttcgaagggtaagtttagtttatgcaacaaagactatcagttaaccgttaataacggcgttaatgcgaatcatagtagtatcggttctttccacagaaaaagatttttgggacccatcattcaaaatccttcaaaggatgtttttaccgccgagtacatgctgatagataatgagaaggacaccgaatttccaggtgatctattggtaaccatacagtatactgtgaacgttgcccaaaaaagtttggaaatggtatataaaggtaaattgactgctggtgaagcgacgccaataaatttaacaaatcatagttatttcaatctgaacaagccatatggagacactattgagggtacggagattatggtgcgttcaaaaaaatctgttgatgtcgacaaaaacatgattcctacgggtaatatcgtcgatagagaaattgctacctttaactctacaaagccaacggtcttaggccccaaaaatccccagtttgattgttgttttgtggtggatgaaaatgctaagccaagtcaaatcaatactctaaacaatgaattgacgcttattgtcaaggcttttcatcccgattccaatattacattagaagttttaagtacagagccaacttatcaattttataccggtgatttcttgtctgctggttacgaagcaagacaaggttttgcaattgagcctggtagatacattgatgctatcaatcaagagaactggaaagattgtgtaaccttgaaaaacggtgaaacttacgggtccaagattgtctacagattttcctga

SEQ ID NO: 30 - DNA, 1037 bp, Pichia pastoris
aaatgcgtacctcttctacgagattcaagcgaatgagaataatgtaatatgcaagatcagaaagaatgaaaggagttgaaaaaaaaaaccgttgcgttttgaccttgaatggggtggaggtttccattcaaagtaaagcctgtgtcttggtattttcggcggcacaagaaatcgtaattttcatcttctaaacgatgaagatcgcagcccaacctgtatgtagttaaccggtcggaattataagaaagattttcgatcaacaaaccctagcaaatagaaagcagggttacaactttaaaccgaagtcacaaacgataaaccactcagctcccacccaaattcattcccactagcagaaaggaattatttaatccctcaggaaacctcgatgattctcccgttcttccatgggcgggtatcgcaaaatgaggaatttttcaaatttctctattgtcaagactgtttattatctaagaaatagcccaatccgaagctcagttttgaaaaaatcacttccgcgtttcttttttacagcccgatgaatatccaaatttggaatatggattactctatcgggactgcagataatatgacaacaacgcagattacattttaggtaaggcataaacaccagccagaaatgaaacgcccactagccatggtcgaatagtccaatgaattcagatagctatggtctaaaagctgatgttttttattgggtaatggcgaagagtccagtacgacttccagcagagctgagatggccatttttgggggtattagtaactttttgagctcttttcacttcgatgaagtgtcccattcgggatataatcggatcgcgtcgttttctcgaaaatacagcttagcgtcgtccgcttgttgtaaaagcagcaccacattcctaatctcttatataaacaaaacaacccaaattatcagtgctgttttcccaccagatataagtttcttttctcttccgctttttgattttttatctctttcctttaaaaacttctttaccttaaagggcggcc

SEQ ID NO: 31 - DNA, 512 bp, Pichia pastoris
taagcttcacgatttgtgttccagtttatcccccctttatataccgttaaccctttccctgttgagctgactgttgttgtattaccgcaatttttccaagtttgccatgcttttcgtgttatttgaccgatgtcttttttcccaaatcaaactatatttgttaccatttaaaccaagttatcttttgtattaagagtctaagtttgttcccaggcttcatgtgagagtgataaccatccagactatgattcttgttttttattgggtttgtttgtgtgatacatctgagttgtgattcgtaaagtatgtcagtctatctagatttttaatagttaattggtaatcaatgacttgtttgttttaacttttaaattgtgggtcgtatccacgcgtttagtatagctgttcatggctgttagaggagggcgatgtttatatacagaggacaagaatgaggaggcggcgtgtatttttaaaatggagacgcgactcctgtacaccttatcggttgg

SEQ ID NO: 32 - DNA, 1068 bp, Homo sapiens
ggtagagatttgtctagattgccacagttggttggtgtttccactccattgcaaggaggttctaactctgctgctgctattggtcaatcttccggtgagttgagaactggtggagctagaccacctccaccattgggagcttcctctcaaccaagaccaggtggtgattcttctccagttgttgactctggtccaggtccagcttctaacttgacttccgttccagttccacacactactgctttgtccttgccagcttgtccagaagaatccccattgttggttggtccaatgttgatcgagttcaacatgccagttgacttggagttggttgctaagcagaacccaaacgttaagatgggtggtagatacgctccaagagactgtgtttccccacacaaagttgctatcatcatcccattcagaaacagacaggagcacttgaagtactggttgtactacttgcacccagttttgcaaagacagcagttggactacggtatctacgttatcaaccaggctggtgacactattttcaacagagctaagttgttgaatgttggtttccaggaggctttgaaggattacgactacacttgtttcgttttctccgacgttgacttgattccaatgaacgaccacaacgcttacagatgtttctcccagccaagacacatttctgttgctatggacaagttcggtttctccttgccatacgttcaatacttcggtggtgtttccgctttgtccaagcagcagttcttgactatcaacggtttcccaaacaattactggggatggggtggtgaagatgacgacatctttaacagattggttttcagaggaatgtccatctctagaccaaacgctgttgttggtagatgtagaatgatcagacactccagagacaagaagaacgagccaaacccacaaagattcgacagaatcgctcacactaaggaaactatgttgtccgacggattgaactccttgacttaccaggttttggacgttcagagatacccattgtacactcagatcactgttgacatcggtactccatcctag

SEQ ID NO: 33 - DNA, 183 bp, Saccharomyces cerevisiae
atggccctctttctcagtaagagactgttgagatttaccgtcattgcaggtgcggttattgttctcctcctaacattgaattccaacagtagaactcagcaatatattccgagttccatctccgctgcatttgattttacctcaggatctatatcccctgaacaacaagtcatcgggcgcgcc

SEQ ID NO: 34 - DNA, 1074 bp, Drosophila melanogaster
atgaatagcatacacatgaacgccaatacgctgaagtacatcagcctgctgacgctgaccctgcagaatgccatcctgggcctcagcatgcgctacgcccgcacccggccaggcgacatcttcctcagctccacggccgtactcatggcagagttcgccaaactgatcacgtgcctgttcctggtcttcaacgaggagggcaaggatgcccagaagtttgtacgctcgctgcacaagaccatcattgcgaatcccatggacacgctgaaggtgtgcgtcccctcgctggtctatatcgttcaaaacaatctgctgtacgtctctgcctcccatttggatgcggccacctaccaggtgacgtaccagctgaagattctcaccacggccatgttcgcggttgtcattctgcgccgcaagctgctgaacacgcagtggggtgcgctgctgctcctggtgatgggcatcgtcctggtgcagttggcccaaacggagggtccgacgagtggctcagccggtggtgccgcagctgcagccacggccgcctcctctggcggtgctcccgagcagaacaggatgctcggactgtgggccgcactgggcgcctgcttcctctccggattcgcgggcatctactttgagaagatcctcaagggtgccgagatctccgtgtggatgcggaatgtgcagttgagtctgctcagcattcccttcggcctgctcacctgtttcgttaacgacggcagtaggatcttcgaccagggattcttcaagggctacgatctgtttgtctggtacctggtcctgctgcaggccggcggtggattgatcgttgccgtggtggtcaagtacgcggataacattctcaagggcttcgccacctcgctggccatcatcatctcgtgcgtggcctccatatacatcttcgacttcaatctcacgctgcagttcagcttcggagctggcctggtcatcgcctccatatttctctacggctacgatccggccaggtcggcgccgaagccaactatgcatggtcctggcggcgatgaggagaagctgctgccgcgcgtctag

SEQ ID NO: 35 - DNA, 798 bp, Pichia pastoris
tggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagcc

SEQ ID NO: 36 - DNA, 302 bp, Pichia pastoris
aatatatacctcatttgttcaatttggtgtaaagagtgtggcggatagacttcttgtaaatcaggaaagctacaattccaattgctgcaaaaaataccaatgcccataaaccagtatgagcggtgccttcgacggattgcttactttccgaccctttgtcgtttgattcttctgcctttggtgagtcagtttgtttcgactttatatctgactcatcaacttcctttacggttgcgtttttaatcataattttagccgttggcttattatcccttgagttggtaggagttttgatgatgctg

SEQ ID NO: 37 - DNA, 461 bp, Pichia pastoris
taactggccctttgacgtttctgacaatagttctagaggagtcgtccaaaaactcaactctgacttgggtgacaccaccacgggatccggttcttccgaggaccttgatgaccttggctaatgtaactggagttttagtatccattttaagatgtgtgtttctgtaggttctgggttggaaaaaaattttagacaccagaagagaggagtgaactggtttgcgtgggtttagactgtgtaaggcactactctgtcgaagttttagataggggttacccgctccgatgcatgggaagcgattagcccggctgttgcccgtttggtttttgaagggtaattttcaatatctctgtttgagtcatcaatttcatattcaaagattcaaaaacaaaatctggtccaaggagcgcatttaggattatggagttggcgaatcacttgaacgatagactattatttgc

SEQ ID NO: 38 - DNA, 1841 bp, Pichia pastoris
gtgacattcttgtctttgagatcagtaattgtagagcatagatagaataatattcaagaccaacggcttctcttcggaagctccaagtagcttatagtgatgagtaccggcatatatttataggcttaaaatttcgagggttcactatattcgtttagtgggaagagttcctttcactcttgttatctatattgtcagcgtggactgtttataactgtaccaacttagtttctttcaactccaggttaagagacataaatgtcctttgatgctgacaataatcagtggaattcaaggaaggacaatcccgacctcaatctgttcattaatgaagagttcgaatcgtccttaaatcaagcgctagactcaattgtcaatgagaaccctttctttgaccaagaaactataaatagatcgaatgacaaagttggaaatgagtccattagcttacatgatattgagcaggcagaccaaaataaaccgtcctttgagagcgatattgatggttcggcgccgttgataagagacgacaaattgccaaagaaacaaagctgggggctgagcaattttttttcaagaagaaatagcatatgtttaccactacatgaaaatgattcaagtgttgttaagaccgaaagatctattgcagtgggaacaccccatcttcaatactgcttcaatggaatctccaatgccaagtacaatgcatttacctttttcccagtcatcctatacgagcaattcaaattttttttcaatttatactttactttagtggctctctctcaagcgataccgcaacttcgcattggatatctttcttcgtatgtcgtcccacttttgtttgtactcatagtgaccatgtcaaaagaggcgatggatgatattcaacgccgaagaagggatagagaacagaacaatgaaccatatgaggttctgtccagcccatcaccagttttgtccaaaaacttaaaatgtggtcacttggttcgattgcataagggaatgagagtgcccgcagatatggttcttgtccagtcaagcgaatccaccggagagtcatttatcaagacagatcagctggatggtgagactgattggaagcttcggattgtttctccagttacacaatcgttaccaatgactgaacttcaaaatgtcgccatcactgcaagcgcaccctcaaaatcaattcactcctttcttggaagattgacctacaatgggcaatcatatggtcttacgatagacaacacaatgtggtgtaatactgtattagcttctggttcagcaattggttgtataatttacacaggtaaagatactcgacaatcgatgaacacaactcagcccaaactgaaaacgggcttgttagaactggaaatcaatagtttgtccaagatcttatgtgtttgtgtgtttgcattatctgtcatcttagtgctattccaaggaatagctgatgattggtacgtcgatatcatgcggtttctcattctattctccactattatcccagtgtctctgagagttaaccttgatcttggaaagtcagtccatgctcatcaaatagaaactgatagctcaatacctgaaaccgttgttagaactagtacaataccggaagacctgggaagaattgaatacctattaagtgacaaaactggaactcttactcaaaatgatatggaaatgaaaaaactacacctaggaacagtctcttatgctggtgataccatggatattatttctgatcatgttaaaggtcttaataacgctaaaacatcgaggaaagatcttggtatgagaataagagatttggttacaactctggccatctg

SEQ ID NO: 39 - DNA, 3105 bp, Drosophila melanogaster
agagacgatccaattagacctccattgaaggttgctagatccccaagaccaggtcaatgtcaagatgttgttcaggacgtcccaaacgttgatgtccagatgttggagttgtacgatagaatgtccttcaaggacattgatggtggtgtttggaagcagggttggaacattaagtacgatccattgaagtacaacgctcatcacaagttgaaggtcttcgttgtcccacactcccacaacgatcctggttggattcagaccttcgaggaatactaccagcacgacaccaagcacatcttgtccaacgctttgagacatttgcacgacaacccagagatgaagttcatctgggctgaaatctcctacttcgctagattctaccacgatttgggtgagaacaagaagttgcagatgaagtccatcgtcaagaacggtcagttggaattcgtcactggtggatgggtcatgccagacgaggctaactcccactggagaaacgttttgttgcagttgaccgaaggtcaaacttggttgaagcaattcatgaacgtcactccaactgcttcctgggctatcgatccattcggacactctccaactatgccatacattttgcagaagtctggtttcaagaatatgttgatccagagaacccactactccgttaagaaggagttggctcaacagagacagttggagttcttgtggagacagatctgggacaacaaaggtgacactgctttgttcacccacatgatgccattctactcttacgacattcctcatacctgtggtccagatccaaaggtttgttgtcagttcgatttcaaaagaatgggttccttcggtttgtcttgtccatggaaggttccacctagaactatctctgatcaaaatgttgctgctagatccgatttgttggttgatcagtggaagaagaaggctgagttgtacagaaccaacgtcttgttgattccattgggtgacgacttcagattcaagcagaacaccgagtgggatgttcagagagtcaactacgaaagattgttcgaacacatcaactctcaggctcacttcaatgtccaggctcagttcggtactttgcaggaatacttcgatgctgttcaccaggctgaaagagctggacaagctgagttcccaaccttgtctggtgacttcttcacttacgctgatagatctgataactactggtctggttactacacttccagaccataccataagagaatggacagagtcttgatgcactacgttagagctgctgaaatgttgtccgcttggcactcctgggacggtatggctagaatcgaggaaagattggagcaggctagaagagagttgtccttgttccagcaccacgacggtattactggtactgctaaaactcacgttgtcgtcgactacgagcaaagaatgcaggaagctttgaaagcttgtcaaatggtcatgcaacagtctgtctacagattgttgactaagccatccatctactctccagacttctccttctcctacttcactttggacgactccagatggccaggttctggtgttgaggactctagaactaccatcatcttgggtgaggatatcttgccatccaagcatgttgtcatgcacaacaccttgccacactggagagagcagttggttgacttctacgtctcctctccattcgtttctgttaccgacttggctaacaatccagttgaggctcaggtttctccagtttggtcttggcaccacgacactttgactaagactatccacccacaaggttccaccaccaagtacagaatcatcttcaaggctagagttccaccaatgggtttggctacctacgttttgaccatctccgattccaagccagagcacacctcctacgcttccaatttgttgcttagaaagaacccaacttccttgccattgggtcaatacccagaggatgtcaagttcggtgatccaagagagatctccttgagagttggtaacggtccaaccttggctttctctgagcagggtttgttgaagtccattcagttgactcaggattctccacatgttccagttcacttcaagttcttgaagtacggtgttagatctcatggtgatagatctggtgcttacttgttcttgccaaatggtccagcttctccagtcgagttgggtcagccagttgtcttggtcactaagggtaaattggagtcttccgtttctgttggtttgccatctgtcgttcaccagaccatcatgagaggtggtgctccagagattagaaatttggtcgatattggttctttggacaacactgagatcgtcatgagattggagactcatatcgactctggtgatatcttctacactgatttgaatggattgcaattcatcaagaggagaagattggacaagttgccattgcaggctaactactacccaattccatctggtatgttcattgaggatgctaataccagattgactttgttgaccggtcaaccattgggtggatcttctttggcttctggtgagttggagattatgcaagatagaagattggcttctgatgatgaaagaggtttgggtcagggtgttttggacaacaagccagttttgcatatttacagattggtcttggagaaggttaacaactgtgtcagaccatctaagttgcatccagctggttacttgacttctgctgctcacaaagcttctcagtctttgttggatccattggacaagttcatcttcgctgaaaatgagtggatcggtgctcagggtcaattcggtggtgatcatccatctgctagagaggatttggatgtctctgtcatgagaagattgaccaagtcttctgctaaaacccagagagttggttacgttttgcacagaaccaatttgatgcaatgtggtactccagaggagcatactcagaagttggatgtctgtcacttgttgccaaatgttgctagatgtgagagaactaccttgactttcttgcagaatttggagcacttggatggtatggttgctccagaagtttgtccaatggaaaccgctgcttacgtctcttctcactcttcttga

SEQ ID NO: 40 - DNA, 108 bp, Saccharomyces cerevisiae
atgctgcttaccaaaaggttttcaaagctgttcaagctgacgttcatagttttgatattgtgcgggctgttcgtcattacaaacaaatacatggatgagaacacgtcg

SEQ ID NO: 41 - DNA, 1729 bp, Pichia pastoris
caagttgcgtccggtatacgtaacgtctcacgatgatcaaagataatacttaatcttcatggtctactgaataactcatttaaacaattgactaattgtacattatattgaacttatgcatcctattaacgtaatcttctggcttctctctcagactccatcagacacagaatatcgttctctctaactggtcctttgacgtttctgacaatagttctagaggagtcgtccaaaaactcaactctgacttgggtgacaccaccacgggatccggttcttccgaggaccttgatgaccttggctaatgtaactggagttttagtatccattttaagatgtgtgtttctgtaggttctgggttggaaaaaaattttagacaccagaagagaggagtgaactggtttgcgtgggtttagactgtgtaaggcactactctgtcgaagttttagataggggttacccgctccgatgcatgggaagcgattagcccggctgttgcccgtttggtttttgaagggtaattttcaatatctctgtttgagtcatcaatttcatattcaaagattcaaaaacaaaatctggtccaaggagcgcatttaggattatggagttggcgaatcacttgaacgatagactattatttgctgttcctaaagagggcagattgtatgagaaatgcgttgaattacttaggggatcagatattcagtttcgaagatccagtagattggatatagctttgtgcactaacctgcccctggcattggttttccttccagctgctgacattcccacgtttgtaggagagggtaaatgtgatttgggtataactggtattgaccaggttcaggaaagtgacgtagatgtcatacctttattagacttgaatttcggtaagtgcaagttgcagattcaagttcccgagaatggtgacttgaaagaacctaaacagctaattggtaaagaaattgtttcctcctttactagcttaaccaccaggtactttgaacaactggaaggagttaagcctggtgagccactaaagacaaaaatcaaatatgttggagggtctgttgaggcctcttgtgccctaggagttgccgatgctattgtggatcttgttgagagtggagaaaccatgaaagcggcagggctgatcgatattgaaactgttctttctacttccgcttacctgatctcttcgaagcatcctcaacacccagaactgatggatactatcaaggagagaattgaaggtgtactgactgctcagaagtatgtcttgtgtaattacaacgcacctagaggtaaccttcctcagctgctaaaactgactccaggcaagagagctgctaccgtttctccattagatgaagaagattgggtgggagtgtcctcgatggtagagaagaaagatgttggaagaatcatggacgaattaaagaaacaaggtgccagtgacattcttgtctttgagatcagtaattgtagagcatagatagaataatattcaagaccaacggcttctcttcggaagctccaagtagcttatagtgatgagtaccggcatatatttataggcttaaaatttcgagggttcactatattcgtttagtgggaagagttcctttcactcttgttatctatattgtcagcgtggactgtttataactgtaccaacttagtttctttcaactccaggttaagagacataaatgtcctttgatgc

SEQ ID NO: 42 - DNA, 1068 bp, Rattus
tccttggtttaccaattgaacttcgaccagatgttgagaaacgttgacaaggacggtacttggtctcctggtgagttggttttggttgttcaggttcacaacagaccagagtacttgagattgttgatcgactccttgagaaaggctcaaggtatcagagaggttttggttatcttctcccacgatttctggtctgctgagatcaactccttgatctcctccgttgacttctgtccagttttgcaggttttcttcccattctccatccaattgtacccatctgagttcccaggttctgatccaagagactgtccaagagacttgaagaagaacgctgctttgaagttgggttgtatcaacgctgaatacccagattctttcggtcactacagagaggctaagttctcccaaactaagcatcattggtggtggaagttgcactttgtttgggagagagttaaggttttgcaggactacactggattgatcttgttcttggaggaggatcattacttggctccagacttctaccacgttttcaagaagatgtggaagttgaagcaacaagagtgtccaggttgtgacgttttgtccttgggaacttacactactatcagatccttctacggtatcgctgacaaggttgacgttaagacttggaagtccactgaacacaacatgggattggctttgactagagatgcttaccagaagttgatcgagtgtactgacactttctgtacttacgacgactacaactgggactggactttgcagtacttgactttggcttgtttgccaaaagtttggaaggttttggttccacaggctccaagaattttccacgctggtgactgtggaatgcaccacaagaaaacttgtagaccatccactcagtccgctcaaattgagtccttgttgaacaacaacaagcagtacttgttcccagagactttggttatcggagagaagtttccaatggctgctatttccccaccaagaaagaatggtggatggggtgatattagagaccacgagttgtgtaaatcctacagaagattgcagtag

SEQ ID NO: 43 - DNA, 300 bp, Saccharomyces cerevisiae
atgctgcttaccaaaaggttttcaaagctgttcaagctgacgttcatagttttgatattgtgcgggctgttcgtcattacaaacaaatacatggatgagaacacgtcggtcaaggagtacaaggagtacttagacagatatgtccagagttactccaataagtattcatcttcctcagacgccgccagcgctgacgattcaaccccattgagggacaatgatgaggcaggcaatgaaaagttgaaaagcttctacaacaacgttttcaactttctaatggttgattcgcccgggcgcgcc

SEQ ID NO: 44 - DNA, 1373 bp, Pichia pastoris
gatctggccttccctgaatttttacgtccagctatacgatccgttgtgactgtatttcctgaaatgaagtttcaacctaaagttttggttgtacttgctccacctaccacggaaactaatatcgaaaccaatgaaaaagtagaactggaatcgtcaatcgaaattcgcaaccaagtggaacccaaagacttgaatctttctaaagtctattctagtgacactaatggcaacagaagatttgagctgacttttcaaatgaatctcaataatgcaatatcaacatcagacaatcaatgggctttgtctagtgacacaggatcaattatagtagtgtcttctgcaggaagaataacttccccgatcctagaagtcggggcatccgtctgtgtcttaagatcgtacaacgaacaccttttggcaataacttgtgaaggaacatgcttttcatggaatttaaagaagcaagaatgtgttctaaacagcatttcattagcacctatagtcaattcacacatgctagttaagaaagttggagatgcaaggaactattctattgtatctgccgaaggagacaacaatccgttaccccagattctagactgcgaactttccaaaaatggcgctccaattgtggctcttagcacgaaagacatctactcttattcaaagaaaatgaaatgctggatccatttgattgattcgaaatactttgaattgttgggtgctgacaatgcactgtttgagtgtgtggaagcgctagaaggtccaattggaatgctaattcatagattggtagatgagttcttccatgaaaacactgccggtaaaaaactcaaactttacaacaagcgagtactggaggacctttcaaattcacttgaagaactaggtgaaaatgcgtctcaattaagagagaaacttgacaaactctatggtgatgaggttgaggcttcttgacctcttctctctatctgcgtttctttttttttttttttttttttttttttcagttgagccagaccgcgctaaacgcataccaattgccaaatcaggcaattgtgagacagtggtaaaaaagatgcctgcaaagttagattcacacagtaagagagatcctactcataaatgaggcgcttatttagtagctagtgatagccactgcggttctgctttatgctatttgttgtatgccttactatctttgtttggctcctttttcttgacgttttccgttggagggactccctattctgagtcatgagccgcacagattatcgcccaaaattgacaaaatcttctggcgaaaaaagtataaaaggagaaaaaagctcacccttttccagcgtagaaagtatatatcagtcattgaagac

SEQ ID NO: 45 - DNA, 1470 bp, Pichia pastoris
gggactttaactcaagtaaaaggatagttgtacaattatatatacgaagaataaatcattacaaaaagtattcgtttctttgattcttaacaggattcattttctgggtgtcatcaggtacagcgctgaatatcttgaagttaacatcgagctcatcatcgacgttcatcacactagccacgtttccgcaacggtagcaataattaggagcggaccacacagtgacgacatctttctctttgaaatggtatctgaagccttccatgaccaattgatgggctctagcgatgagttgcaagttattaatgtggttgaactcacgtgctactcgagcaccgaataaccagccagctccacgaggagaaacagcccaactgtcgacttcatctgggtcagaccaaaccaagtcacaaaatcctccttcatgagggacctcttgcgctcggctgagaactctgatttgatctaacatgcgaatatcgggagagagaccaccatggatacataatattttaccatcaatgatggcactaagggttaaaaagtcgaacacctggcaacagtacttccagacagtggtggaaccatatttattgagacattcctcataaaatccataaacctgagtgatctgtctggattcatgatttccccttaccaatgtgatatgttgaggaaacttaatttttaaaatcatgagtaacgtgaacgtctccaacgagaaatagcctctatccacatagtctcctaggaagatatagttctgttttattccattagaggaggatccgggaaacccaccactaatcttgaaaagttccagtagatcgtgaaattggccgtgaatatctccgcatactgtcactggactctgcactggctgtatattggattcctccatcagcaaatccttcacccgttcgcaaagatgcttcatatcattttcacttaaagccttgcagcttttgacttcttcaaaccactgatctggtcctctttctggcatgattaaggtctataatatttctgagctgagatgtaaaaaaaaataataaaaatggggagtgaaaaagtgtgtagcttttaggagtttgggattgataccccaaaatgatctttatgagaattaaaaggtagatacgcttttaataagaacacctatctatagtactttgtggtcttgagtaattgagatgttcagcttctgaggtttgccgttattctgggatagtagtgcgcgaccaaacaacccgccaggcaaagtgtgttgtgctcgaagacgattgccagaagagtaagtccgtcctgcctcagatgttacacactttcttccctagacagtcgatgcatcatcggatttaaacctgaaactttgatgccatgatacgcctagtcacgtcgactgagattttagataagccccgatccctttagtacattcctgttatccatggatggaatggcctgata

SEQ ID NO: 46 - DNA, 1043 bp, Pichia pastoris
aagcttgttcaccgttgggacttttccgtggacaatgttgactactccaggagggattccagctttctctactagctcagcaataatcaatgcagccccaggcgcccgttctgatggcttgatgaccgttgtattgcctgtcactatagccaggggtagggtccataaaggaatcatagcagggaaattaaaagggcatattgatgcaatcactcccaatggctctcttgccattgaagtctccatatcagcactaacttccaagaaggaccccttcaagtctgacgtgatagagcacgcttgctctgccacctgtagtcctctcaaaacgtcaccttgtgcatcagcaaagactttaccttgctccaatactatgacggaggcaattctgtcaaaattctctctcagcaattcaaccaacttgaaagcaaattgctgtctcttgatgatggagacttttttccaagattgaaatgcaatgtgggacgactcaattgcttcttccagctcctcttcggttgattgaggaacttttgaaaccacaaaattggtcgttgggtcatgtacatcaaaccattctgtagatttagattcgacgaaagcgttgttgatgaaggaaaaggttggatacggtttgtcggtctctttggtatggccggtggggtatgcaattgcagtagaagataattggacagccattgttgaaggtagagaaaaggtcagggaacttgggggttatttataccattttaccccacaaataacaactgaaaagtacccattccatagtgagaggtaaccgacggaaaaagacgggcccatgttctgggaccaatagaactgtgtaatccattgggactaatcaacagacgattggcaatataatgaaatagttcgttgaaaagccacgtcagctgtcttttcattaactttggtcggacacaacattttctactgttgtatctgtcctactttgcttatcatctgccacagggcaagtggatttccttctcgcgcggctgggtgaaaacggttaacgtgaa

SEQ ID NO: 47 - DNA, 695 bp, Pichia pastoris
gccttgggggacttcaagtctttgctagaaactagatgaggtcaggccctcttatggttgtgtcccaattgggcaatttcactcacctaaaaagcatgacaattatttagcgaaataggtagtatattttccctcatctcccaagcagtttcgtttttgcatccatatctctcaaatgagcagctacgactcattagaaccagagtcaagtaggggtgagctcagtcatcagccttcgtttctaaaacgattgagttcttttgttgctacaggaagcgccctagggaactttcgcactttggaaatagattttgatgaccaagagcgggagttgatattagagaggctgtccaaagtacatgggatcaggccggccaaattgattggtgtgactaaaccattgtgtacttggacactctattacaaaagcgaagatgatttgaagtattacaagtcccgaagtgttagaggattctatcgagcccagaatgaaatcatcaaccgttatcagcagattgataaactcttggaaagcggtatcccattttcattattgaagaactacgataatgaagatgtgagagacggcgaccctctgaacgtagacgaagaaacaaatctacttttggggtacaatagagaaagtgaatcaagggaggtatttgtggccataatactcaactctatcattaatg

SEQ ID NO: 48 - DNA, 411 bp, Pichia pastoris
catatggtgagagccgttctgcacaactagatgttttcgagcttcgcattgtttcctgcagctcgactattgaattaagatttccggatatctccaatctcacaaaaacttatgttgaccacgtgctttcctgaggcgaggtgttttatatgcaagctgccaaaaatggaaaacgaatggccatttttcgcccaggcaaattattcgattactgctgtcataaagacagtgttgcaaggctcacatttttttttaggatccgagataaagtgaatacaggacagcttatctctatatcttgtaccattcgtgaatcttaagagttcggttagggggactctagttgagggttggcactcacgtatggctgggcgcagaaataaaattcaggcgcagcagcacttatcgatg

SEQ ID NO: 49 - DNA, 692 bp, Pichia pastoris
gaattcacagttataaataaaaacaaaaactcaaaaagtttgggctccacaaaataacttaatttaaatttttgtctaataaatgaatgtaattccaagattatgtgatgcaagcacagtatgcttcagccctatgcagctactaatgtcaatctcgcctgcgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagctt

SEQ ID NO: 50 - DNA, 546 bp, Pichia pastoris
gatatctccctggggacaatatgtgttgcaactgttcgttgttggtgccccagtcccccaaccggtactaatcggtctatgttcccgtaactcatattcggttagaactagaacaataagtgcatcattgttcaacattgtggttcaattgtcgaacattgctggtgcttatatctacagggaagacgataagcctttgtacaagagaggtaacagacagttaattggtatttctttgggagtcgttgccctctacgttgtctccaagacatactacattctgagaaacagatggaagactcaaaaatgggagaagcttagtgaagaagagaaagttgcctacttggacagagctgagaaggagaacctgggttctaagaggctggactttttgttcgagagttaaactgcataattttttctaagtaaatttcatagttatgaaatttctgcagcttagtgtttactgcatcgtttactgcatcaccctgtaaataatgtgagcttttttccttccattgcttggtatcttccttgctgctgttt

SEQ ID NO: 51 - DNA, 378 bp, Pichia pastoris
acaaaacagtcatgtacagaactaacgcctttaagatgcagaccactgaaaagaattgggtcccatttttcttgaaagacgaccaggaatctgtccattttgtttactcgttcaatcctctgagagtactcaactgcagtcttgataacggtgcatgtgatgttctatttgagttaccacatgattttggcatgtcttccgagctacgtggtgccactcctatgctcaatcttcctcaggcaatcccgatggcagacgacaaagaaatttgggtttcattcccaagaacgagaatatcagattgcgggtgttctgaaacaatgtacaggccaatgttaatgctttttgttagagaaggaacaaacttttttgctgagc

SEQ ID NO: 52 - DNA, 1494 bp, Trichoderma reesei
cgcgccggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacatttggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgcttaa

SEQ ID NO: 53 - DNA, 57 bp, Saccharomyces cerevisiae
atgagattcccatccatcttcactgctgttttgttcgctgcttcttctgctttggct

SEQ ID NO: 54 - protein, 19 aa, Saccharomyces cerevisiae
MRFPSIFTAVLFAASSALA

SEQ ID NO: 55 - DNA, 934 bp, Pichia pastoris
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 56 - DNA, 1792 bp, Pichia pastoris
taccaattgccaaatcaggcaattgtgagacagtggtaaaaaagatgcctgcaaagttagattcacacagtaagagagatcctactcataaatgaggcgcttatttagtagctagtgatagccactgcggttctgctttatgctatttgttgtatgccttactatctttgtttggctcctttttcttgacgttttccgttggagggactccctattctgagtcatgagccgcacagattatcgcccaaaattgacaaaatcttctggcgaaaaaagtataaaaggagaaaaaagctcacccttttccagcgtagaaagtatatatcagtcattgaagactattatttaaataacacaatgtctaaaggaaaagtttgtttggcctactccggtggtttggatacctccatcatcctagcttggttgttggagcagggatacgaagtcgttgcctttttagccaacattggtcaagaggaagactttgaggctgctagagagaaagctctgaagatcggtgctaccaagtttatcgtcagtgacgttaggaaggaatttgttgaggaagttttgttcccagcagtccaagttaacgctatctacgagaacgtctacttactgggtacctctttggccagaccagtcattgccaaggcccaaatagaggttgctgaacaagaaggttgttttgctgttgcccacggttgtaccggaaagggtaacgatcaggttagatttgagctttccttttatgctctgaagcctgacgttgtctgtatcgccccatggagagacccagaattcttcgaaagattcgctggtagaaatgacttgctgaattacgctgctgagaaggatattccagttgctcagactaaagccaagccatggtctactgatgagaacatggctcacatctccttcgaggctggtattctagaagatccaaacactactcctccaaaggacatgtggaagctcactgttgacccagaagatgcaccagacaagccagagttctttgacgtccactttgagaagggtaagccagttaaattagttctcgagaacaaaactgaggtcaccgatccggttgagatctttttgactgctaacgccattgctagaagaaacggtgttggtagaattgacattgtcgagaacagattcatcggaatcaagtccagaggttgttatgaaactccaggtttgactctactgagaaccactcacatcgacttggaaggtcttaccgttgaccgtgaagttagatcgatcagagacacttttgttaccccaacctactctaagttgttatacaacgggttgtactttaccccagaaggtgagtacgtcagaactatgattcagccttctcaaaacaccgtcaacggtgttgttagagccaaggcctacaaaggtaatgtgtataacctaggaagatactctgaaaccgagaaattgtacgatgctaccgaatcttccatggatgagttgaccggattccaccctcaagaagctggaggatttatcacaacacaagccatcagaatcaagaagtacggagaaagtgtcagagagaagggaaagtttttgggactttaactcaagtaaaaggatagttgtacaattatatatacgaagaataaatcattacaaaaagtattcgtttctttgattcttaacaggattcattttctgggtgtcatcaggtacagcgctgaatatcttgaagttaacatcgagctcatcatcgacgttcatcacactagccacgtttccgcaacggtag

SEQ ID NO: 57 - DNA, 1470 bp, Pichia pastoris; identical to SEQ ID NO: 45

SEQ ID NO: 58 - DNA, 1350 bp, Homo sapiens
gaggttcagttggttgaatctggaggaggattggttcaacctggtggttctttgagattgtcctgtgctgcttccggtttcaacatcaaggacacttacatccactgggttagacaagctccaggaaagggattggagtgggttgctagaatctacccaactaacggttacacaagatacgctgactccgttaagggaagattcactatctctgctgacacttccaagaacactgcttacttgcagatgaactccttgagagctgaggatactgctgtttactactgttccagatggggtggtgatggtttctacgctatggactactggggtcaaggaactttggttactgtttcctccgcttctactaagggaccatctgttttcccattggctccatcttctaagtctacttccggtggtactgctgctttgggatgtttggttaaagactacttcccagagccagttactgtttcttggaactccggtgctttgacttctggtgttcacactttcccagctgttttgcaatcttccggtttgtactctttgtcctccgttgttactgttccatcctcttccttgggtactcagacttacatctgtaacgttaaccacaagccatccaacactaaggttgacaagaaggttgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaagagcagtacaactccacttacagagttgtttccgttttgactgttttgcaccaggactggttgaacggtaaagaatacaagtgtaaggtttccaacaaggctttgccagctccaatcgaaaagactatctccaaggctaagggtcaaccaagagagccacaggtttacactttgccaccatccagagaagagatgactaagaaccaggtttccttgacttgtttggttaaaggattctacccatccgacattgctgttgagtgggaatctaacggtcaaccagagaacaactacaagactactccaccagttttggattctgatggttccttcttcttgtactccaagttgactgttgacaagtccagatggcaacagggtaacgttttctcctgttccgttatgcatgaggctttgcacaaccactacactcaaaagtccttgtctttgtcccctggttaa

SEQ ID NO: 59 - DNA, 645 bp, Homo sapiens
gacatccaaatgactcaatccccatcttctttgtctgcttccgttggtgacagagttactatcacttgtagagcttcccaggacgttaatactgctgttgcttggtatcaacagaagccaggaaaggctccaaagttgttgatctactccgcttccttcttgtactctggtgttccatccagattctctggttccagatccggtactgacttcactttgactatctcctccttgcaaccagaagatttcgctacttactactgtcagcagcactacactactccaccaactttcggacagggtactaaggttgagatcaagagaactgttgctgctccatccgttttcattttcccaccatccgacgaacagttgaagtctggtacagcttccgttgtttgtttgttgaacaacttctacccaagagaggctaaggttcagtggaaggttgacaacgctttgcaatccggtaactcccaagaatccgttactgagcaagactctaaggactccacttactccttgtcctccactttgactttgtccaaggctgattacgagaagcacaaggtttacgcttgtgaggttacacatcagggtttgtcctccccagttactaagtccttcaacagaggagagtgttaa

SEQ ID NO: 60 - DNA, 375 bp, Streptoalloteichus hindustanus
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 61 - DNA, 427 bp, Saccharomyces cerevisiae
gatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 62 - DNA, 1617 bp, Pichia pastoris
atgagtgtaagtgatagtcatcttgcaacagattattttggaacgcaactaacaaagcagatacacccttcagcagaatcctttctggatattgtgaagaatgatcgccaaagtcacagtcctgagacagttcctaatctttaccccatttacaagttcatccaatcagacttcttaacgcctcatctggcttatatcaagcttaccaacagttcagaaactcccagtccaagtttcttgcttgaaagtgcgaagaatggtgacaccgttgacaggtacacctttatgggacattcccccagaaaaataatcaagactgggcctttagagggtgctgaagttgaccccttggtgcttctggaaaaagaactgaagggcaccagacaagcgcaacttcctggtattcctcgtctaagtggtggtgccataggatacatctcgtacgattgtattaagtactttgaaccaaaaactgaaagaaaactgaaagatgttttgcaacttccggaagcagctttgatgttgttcgacacgatcgtggcttttgacaatgtttatcaaagattccaggtaattggaaacgtttctctatccgttgatgactcggacgaagctattcttgagaaatattataagacaagagaagaagtggaaaagatcagtaaagtggtatttgacaataaaactgttccctactatgaacagaaagatattattcaaggccaaacgttcacctctaatattggtcaggaagggtatgaaaaccatgttcgcaagctgaaagaacatattctgaaaggagacatcttccaagctgttccctctcaaagggtagccaggccgacctcattgcaccctttcaacatctatcgtcatttgagaactgtcaatccttctccatacatgttctatattgactatctagacttccaagttgttggtgcttcacctgaattactagttaaatccgacaacaacaacaaaatcatcacacatcctattgctggaactcttcccagaggtaaaactatcgaagaggacgacaattatgctaagcaattgaagtcgtctttgaaagacagggccgagcacgtcatgctggtagatttggccagaaatgatattaaccgtgtgtgtgagcccaccagtaccacggttgatcgtttattgactgtggagagattttctcatgtgatgcatcttgtgtcagaagtcagtggaacattgagaccaaacaagactcgcttcgatgctttcagatccattttcccagcaggaaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatga

SEQ ID NO: 63 - DNA, 414 bp, Pichia pastoris
ccggccatttaaatatgtgacgactgggtgatccgggttagtgagttgttctcccatctgtatatttttcatttacgatgaatacgaaatgagtattaagaaatcaggcgtagcaatatgggcagtgttcagtcctgtcatagatggcaagcactggcacatccttaataggttagagaaaatcattgaatcatttgggtggtgaaaaaaaattgatgtaaacaagccacccacgctgggagtcgaacccagaatcttttgattagaagtcaaacgcgttaaccattacgctacgcaggcatgtttcacgtccatttttgattgctttctatcataatctaaagatgtgaactcaattagttgcaatttgaccaattcttccattacaagtcgtgcttcctccgttgatgcaac

SEQ ID NO: 64 - DNA, 582 bp, Streptomyces noursei
atgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactg

SEQ ID NO: 65 - DNA, 388 bp, Ashbya gossypii
gatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaacc

SEQ ID NO: 66 - DNA, 247 bp, Ashbya gossypii
taatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaac

SEQ ID NO: 67 - DNA, 672 bp, Pichia pastoris
gcggaaacggcagtaaacaatggagcttcattagtgggtgttattatggtccctggccgggaacgaacggtgaaacaagaggttgcgagggaaatttcgcagatggtgcgggaaaagagaatttcaaagggctcaaaatacttggattccagacaactgaggaaagagtgggacgactgtcctctggaagactggtttgagtacaacgtgaaagaaataaacagcagtggtccatttttagttggagtttttcgtaatcaaagtatagatgaaatccagcaagctatccacactcatggtttggatttcgtccaactacatgggtctgaggattttgattcgtatatacgcaatatcccagttcctgtgattaccagatacacagataatgccgtcgatggtcttaccggagaagacctcgctataaatagggccctggtgctactggacagcgagcaaggaggtgaaggaaaaaccatcgattgggctcgtgcacaaaaatttggagaacgtagaggaaaatatttactagccggaggtttgacacctgataatgttgctcatgctcgatctcatactggctgtattggtgttgacgtctctggtggggtagaaacaaatgcctcaaaagatatggacaagatcacacaatttatcagaaacgctacataa

SEQ ID NO: 68 - DNA, 834 bp, Pichia pastoris
aagtcaattaaatacacgcttgaaaggacattacatagctttcgatttaagcagaaccagaaatgtagaaccacttgtcaatagattggtcaatcttagcaggagcggctgggctagcagttggaacagcagaggttgctgaaggtgagaaggatggagtggattgcaaagtggtgttggttaagtcaatctcaccagggctggttttgccaaaaatcaacttctcccaggcttcacggcattcttgaatgacctcttctgcatacttcttgttcttgcattcaccagagaaagcaaactggttctcaggttttccatcagggatcttgtaaattctgaaccattcgttggtagctctcaacaagcccggcatgtgcttttcaacatcctcgatgtcattgagcttaggagccaatgggtcgttgatgtcgatgacgatgaccttccagtcagtctctccctcatccaacaaagccataacaccgaggaccttgacttgcttgacctgtccagtgtaacctacggcttcaccaatttcgcaaacgtccaatggatcattgtcacccttggccttggtctctggatgagtgacgttagggtcttcccatgtctgagggaaggcaccgtagttgtgaatgtatccgtggtgagggaaacagttacgaacgaaacgaagttttcccttctttgtgtcctgaagaattgggttcagtttctcctccttggaaatctccaacttggcgttggtccaacgggggacttcaacaaccatgttgagaaccttcttggattcgtcagcataaagtgggatgtcgtggaaaggagatacgactt

SEQ ID NO: 69 - DNA, 29 bp, Pichia pastoris
gttaaatgactctaacaccttgcacttga

SEQ ID NO: 70 - DNA, 27 bp, Pichia pastoris
cctcccactggaaccgatgatatggaa

SEQ ID NO: 71 - DNA, 33 bp, Pichia pastoris
gatgcgaagttaagtgcgcagaaagtaatatca

SEQ ID NO: 72 - DNA, 24 bp, Pichia pastoris
ttgcaaaaaccagtgaggaatagc

SEQ ID NO: 73 - DNA, 26 bp, Pichia pastoris
gaatgctgaagaacgtcaaagaaact

SEQ ID NO: 74 - DNA, 25 bp, Pichia pastoris
tgagacttcagagctttccatacga

SEQ ID NO: 75 - DNA, 4356 bp, Pichia pastoris
atgggtattccaaagttttttcggtacatctctgagagatggccgatgatatctgagccaatagaagacagccaaattgccgagtttgataacctgtatctggacatgaactcaattcttcataattgtacacatagtaacgatggatcagttgatttaatgaaagaagaagagatgttcagtgctatttttgcttacattgaacatcttttcactttgatcaaacctggaaagacgtttttcatggccattgatggtgttgctcctagagcaaagatgaaccagcaacgatctagaagattcaggacggccattgaggcagaaaaaagtgtagagatagcccaaaagaatggactcattactagcaaagacgagaattttgacagtaactgtatcactcctggaacggagtttatggccaaggttaccactaacttgaagtttttcatccaccagaaaatctcttcagatgcaaaatggcagaaagtccaggttattctcagtggacatgaagtccccggagaaggagagcacaaaattatggactatattcgttttttgaaggctcaagaggggtatgatccgaatacgagacattgtatttacgggctggatgcagacttgatcatgttgggattggtcattcacgatcctcattttgccatcttgagagaagaggttgtgttcggaagaggatccaaggcctcgtcaacagatgtgtcagaacagcaattctaccttctacatctctccttgttacgtgaataccttgctcttgagttcaaagatttagaagaccagataaagtttgattatgacttagagagaatcttggacgattttatttttatcatgtatgttattggaaatgatttcttacccaacttacctgacttgcatatcaacaaaggtgccttccccagactcttagcaacgatcaaagaaaccatgatagattcggatggatatctccaagaaggaggtgtcatcaatatggaacgattcggtctgtggcttgaccacttgtcacaatttgagcttcaaaattttgagcaggtcgacgtggatgtagaatggtttaacaagcagctagagaacatatccctagatggtgagcgaaagagggaaagagctggaagaaagttgttgcttagacgtcaacaggaattaatttcaaaacttagaccatggattctcgagttttattcatcaagagacaatatatattctgctcatgatgatgactccttaattccaactttgcaattggacaccgaattgatggaagaagaaatcaagttacccttcataaagcagtttgcacttgatgttggtttctttattgtgcacagcaaatctcagaatacgtaccatgctaagatagacattgatggtataaatgtcaatgaatctgatgaagaatttgaagctcgtgtcttgtatatcaggaagaagatcaaagaatatgaaaactcaatttttgttgaagatgaaaacactttagaggaacaaaagaacatttatgataccaagttcgtcaactggaaaaacaaatattacaaagaaaagtttggatttactctttctgacacagaggacattttgaagttgacggagtcgtatattcagggtcttcaatgggttttgttctactattatcgaggagttccttcttggcaatggtactatccttattactatgctcctagaatctctgacatcaaattagggattaaggcttgcctggagtttgacaaaggtacaccgttcaagccatttgaacaattaatggctgtccttcctgcaagatcaaaacagttagttcctgcttgttatagaccgttgatgaccgatccaaactctcccattattgaattttaccctgatgaggttgaaattgacaagaatggaaagactgcctcttgggaggctgttgttaaaatcaactttgtcgatgaaaaacgactattagaggcactggaaccgtataatagtaagttgaatgctgaagaacgtcaaagaaactctttgggcacgaatatcgagtttagctatcatcctcaagtgaatcaagttcattcttccccaattccaacaatattcccagatatcactgaggatcactgctacgaaatagtggtcgaatttgacaagctgcactcttcaacttatgctaaacctatgaaaggtgccaaacaaggtataaatctattggctggattcccaactttgaagaccattcccttcacttcgcagctcatgttagcggaatgccatatttttaaccagccaacaaaatcggaatcgatgattctaagtacacagaatccgtttgaaggactcactgtagagcagtttgctgctcagaatttgggccaaacaatctatgtcaactggccttatttgaaagaggccaaagttgttgcagtttctgacggtttgaacgtttttgagcctggaaacaagtctatccgaacgactcgtatggaaagctctgaagtctcagaatttgccagtgacgttcgctatatcagtgagacgctattcaagagaaaaggtgttttactggtgaactacactgaggaagagttgcaaggaacacctgagcctcgtagatcccctcacaatgacgatgcgatccaaggaattgtgtttgtaaagaaagtgaatggtgttattcgcactagatcgggtgcctatgtgaagacatacagtgacaagattgagaaataccctattaatttgcttgttgacgacgtggttaacaaggatcgtcgattattagaaaaacctcctgtgccattagaagaagaattcccaaagggtactactctcataagtttgggagcttttgcttacggaactcctgctactgttgttgaccaccaaaatgacttaatgaccgtcagattcgtaaaccagccaattaagcatgaatttaactatggtgagattcaagcacaaagggaggcacacaccaatgtgtattatccctcctttaaagttgccaaaatcgttggaattacggcgctagccttgtccagaataacctcttcctatagaatagttaatggagctaagaaaactgttaacatcggattagatttgaagtttgaaggaagaaagttaaaggttctgggatacactaaaagaaacgagaaacattggtcatacagcgctttggctgtaaacttgctgcaacaatatcagaaacgtttcccatctgtttttaagataatttctctccgaaatgattcttcaattcctgaagccaaagaactgtttcctcaagtccccgctagccaagtggatgaaaaagtcaatgaacttgtggcttgggttaaagaacaaaagaaaagctttgttgttgcaacattggagtctgagtctttgaccaaggttagtatcggaaagattgaacaggaagtgattaagtttgtttcaaaaccacatgaacatattccccaaaagggtttgaaaggagttcctcgtgaggctactctggatatcagcaactcttctcaatttctttccaagcagacttttaatctaggagacagagtgatctacgtagaagattctggtaaggtacccaacttcagtaaaggtacagttattggggttcgaagtgtcggtacgaaagtgactttgaacgtattgtttgacttgccattgctctctggtaatacctttgatggaagacttgcaacccccaggggtgtcactgttgatagttcattggtattgaacttaacaaagcgtcaattgatttaccacgataggaaacccgctaagaaagaaggtaagcctggagttaagaagacatctcaggactccaaaaagcaaacagtgaagttgcagaacggttcaggtaaggccaaacaggcacaagatgctgaactgtcagtcactacgacacctgttccggttcctgccgctaacactgcaccagttactgcatctgtacaggctacctcggtggttactgctaccgttccaacttctaaaccttcaaacaactctgaggacgaacatgaattacttcgcttactgaaaggtaacaaggacagtagtgaatcgcaaggctctcaagagcctcttgcccggacttctatccagcaaatttacggaacggtgttcaatcaagtgttgagcgctcaacctcagttgcagcctgtcagaggcttctcaaatccggtacctgaaacccctgtaaatggagtccaagcgaatgaacaacactctgattctacccctcaaaatcattctagggatgaaaaccaaggaagaggtcgtggtagaggcagaggaaatagaagaggaagaggtcgaggtagaggcaaaggaggacagtaa

SEQ ID NO: 76 - protein, 1451 aa, Pichia pastoris
MGIPKFFRYISERWPMISEPIEDSQIAEFDNLYLDMNSILHNCTHSNDGSVDLMKEEEMFSAIFAYIEHLFTLIKPGKTFFMAIDGVAPRAKMNQQRSRRFRTAIEAEKSVEIAQKNGLITSKDENFDSNCITPGTEFMAKVTTNLKFFIHQKISSDAKWQKVQVILSGHEVPGEGEHKIMDYIRFLKAQEGYDPNTRHCIYGLDADLIMLGLVIHDPHFAILREEVVFGRGSKASSTDVSEQQFYLLHLSLLREYLALEFKDLEDQIKFDYDLERILDDFIFIMYVIGNDFLPNLPDLHINKGAFPRLLATIKETMIDSDGYLQEGGVINMERFGLWLDHLSQFELQNFEQVDVDVEWFNKQLENISLDGERKRERAGRKLLLRRQQELISKLRPWILEFYSSRDNIYSAHDDDSLIPTLQLDTELMEEEIKLPFIKQFALDVGFFIVHSKSQNTYHAKIDIDGINVNESDEEFEARVLYIRKKIKEYENSIFVEDENTLEEQKNIYDTKFVNWKNKYYKEKFGFTLSDTEDILKLTESYIQGLQWVLFYYYRGVPSWQWYYPYYYAPRISDIKLGIKACLEFDKGTPFKPFEQLMAVLPARSKQLVPACYRPLMTDPNSPIIEFYPDEVEIDKNGKTASWEAVVKINFVDEKRLLEALEPYNSKLNAEERQRNSLGTNIEFSYHPQVNQVHSSPIPTIFPDITEDHCYEIVVEFDKLHSSTYAKPMKGAKQGINLLAGFPTLKTIPFTSQLMLAECHIFNQPTKSESMILSTQNPFEGLTVEQFAAQNLGQTIYVNWPYLKEAKVVAVSDGLNVFEPGNKSIRTTRMESSEVSEFASDVRYISETLFKRKGVLLVNYTEEELQGTPEPRRSPHNDDAIQGIVFVKKVNGVIRTRSGAYVKTYSDKIEKYPINLLVDDVVNKDRRLLEKPPVPLEEEFPKGTTLISLGAFAYGTPATVVDHQNDLMTVRFVNQPIKHEFNYGEIQAQREAHTNVYYPSFKVAKIVGITALALSRITSSYRIVNGAKKTVNIGLDLKFEGRKLKVLGYTKRNEKHWSYSALAVNLLQQYQKRFPSVFKIISLRNDSSIPEAKELFPQVPASQVDEKVNELVAWVKEQKKSFVVATLESESLTKVSIGKIEQEVIKFVSKPHEHIPQKGLKGVPREATLDISNSSQFLSKQTFNLGDRVIYVEDSGKVPNFSKGTVIGVRSVGTKVTLNVLFDLPLLSGNTFDGRLATPRGVTVDSSLVLNLTKRQLIYHDRKPAKKEGKPGVKKTSQDSKKQTVKLQNGSGKAKQAQDAELSVTTTPVPVPAANTAPVTASVQATSVVTATVPTSKPSNNSEDEHELLRLLKGNKDSSESQGSQEPLARTSIQQIYGTVFNQVLSAQPQLQPVRGFSNPVPETPVNGVQANEQHSDSTPQNHSRDENQGRGRGRGRGNRRGRGRGRGKGGQ

SEQ ID NO: 77 - DNA, 1014 bp, Mus
atggctccagctagagaaaacgtttccttgttcttcaagttgtactgtttggctgttatgactttggttgctgctgcttacactgttgctttgagatacactagaactactgctgaggagttgtacttctccactactgctgtttgtatcactgaggttatcaagttgttgatctccgttggtttgttggctaaggagactggttctttgggaagattcaaggcttccttgtccgaaaacgttttgggttccccaaaggagttggctaagttgtctgttccatccttggtttacgctgttcagaacaacatggctttcttggctttgtctaacttggacgctgctgtttaccaagttacttaccagttgaagatcccatgtactgctttgtgtactgttttgatgttgaacagaacattgtccaagttgcagtggatctccgttttcatgttgtgtggtggtgttactttggttcagtggaagccagctcaagcttccaaagttgttgttgctcagaacccattgttgggtttcggtgctattgctatcgctgttttgtgttccggtttcgctggtgtttacttcgagaaggttttgaagtcctccgacacttctttgtgggttagaaacatccagatgtacttgtccggtatcgttgttactttggctggtacttacttgtctgacggtgctgagattcaagagaagggattcttctacggttacacttactatgtttggttcgttatcttcttggcttccgttggtggtttgtacacttccgttgttgttaagtacactgacaacatcatgaagggattctctgctgctgctgctattgttttgtccactatcgcttccgttttgttgttcggattgcagatcacattgtcctttgctttgggagctttgttggtttgtgtttccatctacttgtacggattgccaagacaagacactacttccattcagcaagaggctacttccaaggagagaatcatcggtgtttagtag

SEQ ID NO: 78 - DNA, 2172 bp, Homo sapiens
atggaaaagaacggtaacaacagaaagttgagagtttgtgttgctacttgtaacagagctgactactccaagttggctccaatcatgttcggtatcaagactgagccagagttcttcgagttggacgttgttgttttgggttcccacttgattgatgactacggtaacacttacagaatgatcgagcaggacgacttcgacatcaacactagattgcacactattgttagaggagaggacgaagctgctatggttgaatctgttggattggctttggttaagttgccagacgttttgaacagattgaagccagacatcatgattgttcacggtgacagattcgatgctttggctttggctacttccgctgctttgatgaacattagaatcttgcacatcgagggtggtgaagtttctggtactatcgacgactccatcagacacgctatcactaagttggctcactaccatgtttgttgtactagatccgctgagcaacacttgatttccatgtgtgaggaccacgacagaattttgttggctggttgtccatcttacgacaagttgttgtccgctaagaacaaggactacatgtccatcatcagaatgtggttgggtgacgacgttaagtctaaggactacatcgttgctttgcagcacccagttactactgacatcaagcactccatcaagatgttcgagttgactttggacgctttgatctccttcaacaagagaactttggttttgttcccaaacattgacgctggttccaaagagatggttagagttatgagaaagaagggtatcgaacaccacccaaacttcagagctgttaagcacgttccattcgaccaattcatccagttggttgctcatgctggttgtatgatcggtaactcctcctgtggtgttagagaagttggtgctttcggtactccagttatcaacttgggtactagacagatcggtagagagactggagaaaacgttttgcatgttagagatgctgacactcaggacaagattttgcaggctttgcacttgcaattcggaaagcagtacccatgttccaaaatctacggtgacggtaacgctgttccaagaatcttgaagtttttgaagtccatcgacttgcaagagccattgcagaagaagttctgtttcccaccagttaaggagaacatctcccaggacattgaccacatcttggagacattgtccgctttggctgttgatttgggtggaactaacttgagagttgctatcgtttccatgaagggagagatcgttaagaagtacactcagttcaacccaaagacttacgaggagagaatcaacttgatcttgcagatgtgtgttgaagctgctgctgaggctgttaagttgaactgtagaatcttgggtgttggtatctctactggtggtagagttaatccaagagagggtatcgttttgcactccactaagttgattcaggagtggaactccgttgatttgagaactccattgtccgacacattgcacttgccagtttgggttgacaacgacggtaattgtgctgctttggctgagagaaagttcggtcaaggaaagggattggagaacttcgttactttgatcactggtactggtattggtggtggtatcattcaccagcacgagttgattcacggttcttccttctgtgctgctgaattgggacacttggttgtttctttggacggtccagactgttcttgtggttcccacggttgtattgaagcttacgcatcaggaatggcattgcagagagaggctaagaagttgcacgacgaggacttgttgttggttgagggaatgtctgttccaaaggacgaggctgttggtgctttgcatttgatccaggctgctaagttgggtaatgctaaggctcagtccatcttgagaactgctggtactgctttgggattgggtgttgttaatatcttgcacactatgaacccatccttggttatcttgtccggtgttttggcttctcactacatccacatcgttaaggacgttatcagacagcaagctttgtcctccgttcaagacgttgatgttgttgtttccgacttggttgacccagctttgttgggtgctgcttccatggttttggactacactactagaagaatctactaatag

SEQ ID NO: 79 - DNA, 1854 bp, Pichia pastoris
cagttgagccagaccgcgctaaacgcataccaattgccaaatcaggcaattgtgagacagtggtaaaaaagatgcctgcaaagttagattcacacagtaagagagatcctactcataaatgaggcgcttatttagtagctagtgatagccactgcggttctgctttatgctatttgttgtatgccttactatctttgtttggctcctttttcttgacgttttccgttggagggactccctattctgagtcatgagccgcacagattatcgcccaaaattgacaaaatcttctggcgaaaaaagtataaaaggagaaaaaagctcacccttttccagcgtagaaagtatatatcagtcattgaagactattatttaaataacacaatgtctaaaggaaaagtttgtttggcctactccggtggtttggatacctccatcatcctagcttggttgttggagcagggatacgaagtcgttgcctttttagccaacattggtcaagaggaagactttgaggctgctagagagaaagctctgaagatcggtgctaccaagtttatcgtcagtgacgttaggaaggaatttgttgaggaagttttgttcccagcagtccaagttaacgctatctacgagaacgtctacttactgggtacctctttggccagaccagtcattgccaaggcccaaatagaggttgctgaacaagaaggttgttttgctgttgcccacggttgtaccggaaagggtaacgatcaggttagatttgagctttccttttatgctctgaagcctgacgttgtctgtatcgccccatggagagacccagaattcttcgaaagattcgctggtagaaatgacttgctgaattacgctgctgagaaggatattccagttgctcagactaaagccaagccatggtctactgatgagaacatggctcacatctccttcgaggctggtattctagaagatccaaacactactcctccaaaggacatgtggaagctcactgttgacccagaagatgcaccagacaagccagagttctttgacgtccactttgagaagggtaagccagttaaattagttctcgagaacaaaactgaggtcaccgatccggttgagatctttttgactgctaacgccattgctagaagaaacggtgttggtagaattgacattgtcgagaacagattcatcggaatcaagtccagaggttgttatgaaactccaggtttgactctactgagaaccactcacatcgacttggaaggtcttaccgttgaccgtgaagttagatcgatcagagacacttttgttaccccaacctactctaagttgttatacaacgggttgtactttaccccagaaggtgagtacgtcagaactatgattcagccttctcaaaacaccgtcaacggtgttgttagagccaaggcctacaaaggtaatgtgtataacctaggaagatactctgaaaccgagaaattgtacgatgctaccgaatcttccatggatgagttgaccggattccaccctcaagaagctggaggatttatcacaacacaagccatcagaatcaagaagtacggagaaagtgtcagagagaagggaaagtttttgggactttaactcaagtaaaaggatagttgtacaattatatatacgaagaataaatcattacaaaaagtattcgtttctttgattcttaacaggattcattttctgggtgtcatcaggtacagcgctgaatatcttgaagttaacatcgagctcatcatcgacgttcatcacactagccacgtttccgcaacggtagcaataattaggagcggaccacacagtgacgacatc

SEQ ID NO: 80 - DNA, 1308 bp, Homo sapiens
atggactctgttgaaaagggtgctgctacttctgtttccaacccaagaggtagaccatccagaggtagacctcctaagttgcagagaaactccagaggtggtcaaggtagaggtgttgaaaagccaccacacttggctgctttgatcttggctagaggaggttctaagggtatcccattgaagaacatcaagcacttggctggtgttccattgattggatgggttttgagagctgctttggactctggtgctttccaatctgtttgggtttccactgaccacgacgagattgagaacgttgctaagcaattcggtgctcaggttcacagaagatcctctgaggtttccaaggactcttctacttccttggacgctatcatcgagttcttgaactaccacaacgaggttgacatcgttggtaacatccaagctacttccccatgtttgcacccaactgacttgcaaaaagttgctgagatgatcagagaagagggttacgactccgttttctccgttgttagaaggcaccagttcagatggtccgagattcagaagggtgttagagaggttacagagccattgaacttgaacccagctaaaagaccaagaaggcaggattgggacggtgaattgtacgaaaacggttccttctacttcgctaagagacacttgatcgagatgggatacttgcaaggtggaaagatggcttactacgagatgagagctgaacactccgttgacatcgacgttgatatcgactggccaattgctgagcagagagttttgagatacggttacttcggaaaggagaagttgaaggagatcaagttgttggtttgtaacatcgacggttgtttgactaacggtcacatctacgtttctggtgaccagaaggagattatctcctacgacgttaaggacgctattggtatctccttgttgaagaagtccggtatcgaagttagattgatctccgagagagcttgttccaagcaaacattgtcctctttgaagttggactgtaagatggaggtttccgtttctgacaagttggctgttgttgacgaatggagaaaggagatgggtttgtgttggaaggaagttgcttacttgggtaacgaagtttctgacgaggagtgtttgaagagagttggtttgtctggtgctccagctgatgcttgttccactgctcaaaaggctgttggttacatctgtaagtgtaacggtggtagaggtgctattagagagttcgctgagcacatctgtttgttgatggagaaagttaataactcctgtcagaagtagtag

SEQ ID NO: 81 - DNA, 1080 bp, Homo sapiens
atgccattggaattggagttgtgtcctggtagatgggttggtggtcaacacccatgtttcatcatcgctgagatcggtcaaaaccaccaaggagacttggacgttgctaagagaatgatcagaatggctaaggaatgtggtgctgactgtgctaagttccagaagtccgagttggagttcaagttcaacagaaaggctttggaaagaccatacacttccaagcactcttggggaaagacttacggagaacacaagagacacttggagttctctcacgaccaatacagagagttgcagagatacgctgaggaagttggtatcttcttcactgcttctggaatggacgaaatggctgttgagttcttgcacgagttgaacgttccattcttcaaagttggttccggtgacactaacaacttcccatacttggaaaagactgctaagaaaggtagaccaatggttatctcctctggaatgcagtctatggacactatgaagcaggtttaccagatcgttaagccattgaacccaaacttttgtttcttgcagtgtacttccgcttacccattgcaaccagaggacgttaatttgagagttatctccgagtaccagaagttgttcccagacatcccaattggttactctggtcacgagactggtattgctatttccgttgctgctgttgctttgggtgctaaggttttggagagacacatcactttggacaagacttggaagggttctgatcactctgcttctttggaacctggtgagttggctgaacttgttagatcagttagattggttgagagagctttgggttccccaactaagcaattgttgccatgtgagatggcttgtaacgagaagttgggaaagtccgttgttgctaaggttaagatcccagagggtactatcttgactatggacatgttgactgttaaagttggagagccaaagggttacccaccagaggacatctttaacttggttggtaaaaaggttttggttactgttgaggaggacgacactattatggaggagttggttgacaaccacggaaagaagatcaagtcctag

SEQ ID NO: 82 - DNA, 1092 bp, Mus
gtttttcaaatgccaaagtcccaggagaaagttgctgttggtccagctccacaagctgttttctccaactccaagcaagatccaaaggagggtgttcaaatcttgtcctacccaagagttactgctaaggttaagccacaaccatccttgcaagtttgggacaaggactccacttactccaagttgaacccaagattgttgaagatttggagaaactacttgaacatgaacaagtacaaggtttcctacaagggtccaggtccaggtgttaagttctccgttgaggctttgagatgtcacttgagagaccacgttaacgtttccatgatcgaggctactgacttcccattcaacactactgaatgggagggatacttgccaaaggagaacttcagaactaaggctggtccatggcataagtgtgctgttgtttcttctgctggttccttgaagaactcccagttgggtagagaaattgacaaccacgacgctgttttgagattcaacggtgctccaactgacaacttccagcaggatgttggtactaagactactatcagattggttaactcccaattggttactactgagaagagattcttgaaggactccttgtacactgagggaatcttgattttgtgggacccatctgtttaccacgctgacattccacaatggtatcagaagccagactacaacttcttcgagacttacaagtcctacagaagattgcacccatcccagccattctacatcttgaagccacaaatgccatgggaattgtgggacatcatccaggaaatttccccagacttgatccaaccaaacccaccatcttctggaatgttgggtatcatcatcatgatgactttgtgtgaccaggttgacatctacgagttcttgccatccaagagaaagactgatgtttgttactaccaccagaagttcttcgactccgcttgtactatgggagcttaccacccattgttgttcgagaagaacatggttaagcacttgaacgaaggtactgacgaggacatctacttgttcggaaaggctactttgtccggtttcagaaacaacagatgttag

SEQ ID NO: 83 - DNA, 1302 bp, Pichia pastoris
actgggcctttagagggtgctgaagttgaccccttggtgcttctggaaaaagaactgaagggcaccagacaagcgcaacttcctggtattcctcgtctaagtggtggtgccataggatacatctcgtacgattgtattaagtactttgaaccaaaaactgaaagaaaactgaaagatgttttgcaacttccggaagcagctttgatgttgttcgacacgatcgtggcttttgacaatgtttatcaaagattccaggtaattggaaacgtttctctatccgttgatgactcggacgaagctattcttgagaaatattataagacaagagaagaagtggaaaagatcagtaaagtggtatttgacaataaaactgttccctactatgaacagaaagatattattcaaggccaaacgttcacctctaatattggtcaggaagggtatgaaaaccatgttcgcaagctgaaagaacatattctgaaaggagacatcttccaagctgttccctctcaaagggtagccaggccgacctcattgcaccctttcaacatctatcgtcatttgagaactgtcaatccttctccatacatgttctatattgactatctagacttccaagttgttggtgcttcacctgaattactagttaaatccgacaacaacaacaaaatcatcacacatcctattgctggaactcttcccagaggtaaaactatcgaagaggacgacaattatgctaagcaattgaagtcgtctttgaaagacagggccgagcacgtcatgctggtagatttggccagaaatgatattaaccgtgtgtgtgagcccaccagtaccacggttgatcgtttattgactgtggagagattttctcatgtgatgcatcttgtgtcagaagtcagtggaacattgagaccaaacaagactcgcttcgatgctttcagatccattttcccagcaggtaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatga

SEQ ID NO: 84 - DNA, 1085 bp, Pichia pastoris
acggaggacgtaagtaggaatttatgtaatcatgccaatacatctttagatttcttcctcttctttttaacgaaagacctccagttttgcactctcgactctctagtatcttcccatttctgttgctgcaacctcttgccttctgtttccttcaattgttcttctttcttctgttgcacttggccttcttcctccatctttcgttttttttcaagccttttcagcagttcttcttccaagagcagttctttgattttctctctccaatccaccaaaaaactggatgaattcaaccgggcatcatcaatgttccactttctttctcttatcaataatctacgtgcttcggcatacgaggaatccagttgctccctaatcgagtcatccacaaggttagcatgggcctttttcagggtgtcaaaagcatctggagctcgtttattcggagtcttgtctggatggatcagcaaagactttttgcggaaagtctttcttatatcttccggagaacaacctggtttcaaatccaagatggcatagctgtccaatttgaaagtggaaagaatcctgccaatttccttctctcgtgtcagctcgttctcctccttttgcaacaggtccacttcatctggcatttttctttatgttaactttaattattattaattataaagttgattatcgttatcaaaataatcatattcgagaaataatccgtccatgcaatatataaataagaattcataataatgtaatgataacagtacctctgatgacctttgatgaaccgcaattttctttccaatgacaagacatccctataatacaattatacagtttatatatcacaaataatcacctttttataagaaaaccgtcctctccgtaacagaacttattatccgcacgttatggttaacacactactaataccgatatagtgtatgaagtcgctacgagatagccatccaggaaacttaccaattcatcagcactttcatgatccgattgttggctttattctttgcgagacagatacttgccaatgaaataactgatcccacagatgagaatccggtgctcgt

SEQ ID NO: 85 - DNA, 747 bp, Pichia pastoris
ttgggggcctccaggacttgctgaaatttgctgactcatcttcgccatccaaggataatgagttagctaatgtgacagttaatgagtcgtcttgactaacggggaacatttcattatttatatccagagtcaatttgatagcagagtttgtggttgaaatacctatgattcgggagactttgttgtaacgaccattatccacagtttggaccgtgaaaatgtcatcgaagagagcagacgacatattatctattgtggtaagtgatagttggaagtccgactaaggcatgaaaatgagaagactgaaaatttaaagtttttgaaaacactaatcgggtaataacttggaaattacgtttacgtgcctttagctcttgtccttacccctgataatctatccatttcccgagagacaatgacatctcggacagctgagaacccgttcgatatagagcttcaagagaatctaagtccacgttcttccaattcgtccatattggaaaacattaatgagtatgctagaagacatcgcaatgattcgctttcccaagaatgtgataatgaagatgagaacgaaaatctcaattatactgataacttggccaagttttcaaagtctggagtatcaagaaagagctgtatgctaatatttggtatttgctttgttatctggctgtttctctttgccttgtatgcgagggacaatcgattttccaatttgaacgagtacgttccagattcaaacag

SEQ ID NO: 86 - DNA, 924 bp, Pichia pastoris
ctactgggaaccacgagacatcactgcagtagtttccaagtggatttcagatcactcatttgtgaatcctgacaaaactgcgatatgggggtggtcttacggtgggttcactacgcttaagacattggaatatgattctggagaggttttcaaatatggtatggctgttgctccagtaactaattggcttttgtatgactccatctacactgaaagatacatgaaccttccaaaggacaatgttgaaggctacagtgaacacagcgtcattaagaaggtttccaattttaagaatgtaaaccgattcttggtttgtcacgggactactgatgataacgtgcattttcagaacacactaaccttactggaccagttcaatattaatggtgttgtgaattacgatcttcaggtgtatcccgacagtgaacatagcattgcccatcacaacgcaaataaagtgatctacgagaggttattcaagtggttagagcgggcatttaacgatagatttttgtaacattccgtacttcatgccatactatatatcctgcaaggtttccctttcagacacaataattgctttgcaattttacataccaccaattggcaaaaataatctcttcagtaagttgaatgcttttcaagccagcaccgtgagaaattgctacagcgcgcattctaacatcactttaaaattccctcgccggtgctcactggagtttccaacccttagcttatcaaaatcgggtgataactctgagttttttttttcacttctattcctaaaccttcgcccaatgctaccacctccaatcaacatcccgaaatggatagaagagaatggacatctcttgcaacctccggttaataattactgtctccacagaggaggatttacggtaatgattgtaggtgggcctaatg

SEQ ID NO: 87 - DNA, 980 bp, Pichia pastoris
cacctgggcctgttgctgctggtactgctgttggaactgttggtattgttgctgatctaaggccgcctgttccacaccgtgtgtatcgaatgcttgggcaaaatcatcgcctgccggaggccccactaccgcttgttcctcctgctcttgtttgttttgctcattgatgatatcggcgtcaatgaattgatcctcaatcgtgtggtggtggtgtcgtgattcctcttctttcttgagtgccttatccatattcctatcttagtgtaccaataattttgttaaacacacgctgttgtttatgaaaagtcgtcaaaaggttaaaaattctacttggtgtgtgtcagagaaagtagtgcagacccccagtttgttgactagttgagaaggcggctcactattgcgcgaatagcatgagaaatttgcaaacatctggcaaagtggtcaatacctgccaacctgccaatcttcgcgacggaggctgttaagcgggttgggttcccaaagtgaatggatattacgggcaggaaaaacagccccttccacactagtctttgctactgacatcttccctctcatgtatcccgaacacaagtatcgggagtatcaacggagggtgcccttatggcagtactccctgttggtgattgtactgctatacgggtctcatttgcttatcagcaccatcaacttgatacactataaccacaaaaattatcatgcacacccagtcaatagtggtatcgttcttaatgagtttgctgatgacgattcattctctttgaatggcactctgaacttggagaactggagaaatggtaccttttcccctaaatttcattccattcagtggaccgaaataggtcaggaagatgaccagggatattacattctctcttccaattcctcttacatagtaaagtctttatccgacccagactttgaatctgttctattcaacgagtctacaatcacttacaacg

SEQ ID NO: 88 - DNA, 1117 bp, Pichia pastoris
ggcagcaaagccttacgttgatgagaatagactggccatttggggttggtcttatggaggttacatgacgctaaaggttttagaacaggataaaggtgaaacattcaaatatggaatgtctgttgcccctgtgacgaattggaaattctatgattctatctacacagaaagatacatgcacactcctcaggacaatccaaactattataattcgtcaatccatgagattgataatttgaagggagtgaagaggttcttgctaatgcacggaactggtgacgacaatgttcacttccaaaatacactcaaagttctagatttatttgatttacatggtcttgaaaactatgatatccacgtgttccctgatagtgatcacagtattagatatcacaacggtaatgttatagtgtatgataagctattccattggattaggcgtgcattcaaggctggcaaataaataggtgcaaaaatattattagactttttttttcgttcgcaagttattactgtgtaccataccgatccaatccgtattgtaattcatgttctagatccaaaatttgggactctaattcatgaggtctaggaagatgatcatctctatagttttcagcggggggctcgatttgcggttggtcaaagctaacatcaaaatgtttgtcaggttcagtgaatggtaactgctgctcttgaattggtcgtctgacaaattctctaagtgatagcacttcatctacaatcatttgcttcatcgtttctatatcgtccacgacctcaaacgagaaatcgaatttggaagaacagacgggctcatcgttaggatcatgccaaaccttgagatatggatgctctaaagcctcagtaactgtaattctgtgagtgggatctaccgtgagcattcgatccagtaagtctatcgcttcagggttggcaccgggaaataactggctgaatgggatcttgggcatgaatggcagggagcgaacataatcctgggcacgctctgatctgatagactgaagtgtctcttccgaaacagtacccagcgtactcaaaatcaagttcaattgatccacatagtctcttcctctaaaaatgggtcggccaccta

SEQ ID NO: 89 - DNA, 1666 bp, Escherichia coli
gatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcggacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagct

SEQ ID NO: 90 - DNA, 365 bp, Pichia pastoris
acgacggccaaattcatgatacacactctgtttcagctggtttggactaccctggagttggtcctgaattggctgcctggaaagcaaatggtagagcccaattttccgctgtaactgatgcccaagcattagagggattcaaaatcctgtctcaattggaagggatcattccagcactagagtctagtcatgcaatctacggcgcattgcaaattgcaaagactatgtcttcggaccagtccttagttattaatgtatctggaaggggtgataaggacgtccagagtgtagctgagattttacctaaattgggacctcaaattggatgggatttgcgtttcagcgaagacattactaaagagtga

SEQ ID NO: 91 - DNA, 613 bp, Pichia pastoris
tcgatagcacaatattcaacttgactgggtgttaagaactaagagctctgggaaactttgtatttattactaccaacacagtcaaattattggatgtgtttttttttccagtacatttcactgagcagtttgttatactcggtctttaatctccatatacatgcagattgtaatacagatctgaacagtttgattctgattgatcttgccaccaatattctatttttgtatcaagtaacagagtcaatgatcattggtaacgtaacggttttcgtgtatagtagttagagcccatcttgtaacctcatttcctcccatattaaagtatcagtgattcgctggaacgattaactaagaaaaaaaaaatatctgcacatactcatcagtctgtaaatctaagtcaaaactgctgtatccaatagaaatcgggatatacctggatgttttttccacataaacaaacgggagttcagcttacttatggtgttgatgcaattcagtatgatcctaccaataaaacgaaactttgggattttggctgtttgagggatcaaaagctgcacctttacaagattgacggatcgaccattagaccaaagcaaatggccaccaa

SEQ ID NO: 92 - protein, 21 aa, Saccharomyces cerevisiae
MKLKTVRSAVLSSLFASQVLG

SEQ ID NO: 93 - protein, 44 aa, Artificial sequence
  TA57 pro
QPIDDTESQTTSVNLMADDTESAFATQTNSGGLDVVGLISMAKR

SEQ ID NO: 94 - protein, 6 aa, Artificial sequence
  N-terminal spacer
EEGEPK

SEQ ID NO: 95 - protein, 30 aa, Artificial sequence
  Glycosylated insulin B chain P28N
FVNQHLCGSHLVEALYLVCGERGFFYTNKT

SEQ ID NO: 96 - protein, 21 aa, Homo sapiens
GIVEQCCTSICSLYQLENYCN

SEQ ID NO: 97 - protein, 125 aa, Artificial sequence
  Pre-proinsulin analogue Yps1ss+TA57 propeptide+N-terminal spacer+B chain P28N+C-peptide “AAK”+ insulin A chain
MKLKTVRSAVLSSLFASQVLGQPIDDTESQTTSVNLMADDTESAFATQTNSGGLDVVGLISMAKREEGEPKFVNQHLCGSHLVEALYLVCGERGFFYTNKTAAKGIVEQCCTSICSLYQLENYCN

SEQ ID NO: 98 - DNA, 378 bp, Artificial sequence
  DNA encoding pre-proinsulin analogue Yps1ss+TA57 propeptide+N-terminal spacer+B chain P28N+C-peptide “AAK”+ insulin A chain
atgaagttgaagactgttagatccgctgttttgtcttctttgtttgcttctcaagttttgggtcaaccaattgatgatactgaatctcaaactacttctgttaacttgatggctgatgatactgaatctgcttttgctactcaaactaactctggtggtttggatgttgttggtttgatttctatggctaagagagaagaaggtgaaccaaagtttgttaaccaacatttgtgtggttctcatttggttgaagctttgtacttggtttgtggtgaaagaggttttttttacactaacaagactgctgctaagggtattgttgaacaatgttgtacttctatttgttctttgtaccaattggaaaactactgtaactaa

(19) United States
(12) Patent Application Publication
(10) Pub. No.: US 2014/0287463 A1
(43) Pub. Date: Sep. 25, 2014

(54) ENGINEERED PICHIA STRAINS WITH IMPROVED FERMENTATION YIELD AND N-GLYCOSYLATION QUALITY

(71) Applicant: MERCK SHARP & DOHME CORP., Rahway, NJ (US)

(72) Inventors: Ming-Tang Chen, Lebanon, NH (US); Byung-Kwon Choi, Lebanon, NH (US)

(21) Appl. No.: 14/354,862

(22) PCT Filed: Oct. 24, 2012
(86) PCT No.: PCT/US12/61599

§ 371 Date: Apr. 28, 2014

Related U.S. Application Data
(60) Provisional application No. 61/553,801, filed on Oct. 31, 2011.

(51) Int. Cl. C12P 21/00; C07K 14/505; C07K 14/62; C12N 15/81; C07K 16/30

(52) U.S. Cl. CPC C12P 21/005; C12N 15/815; C07K 16/3015; C07K 14/62; C07K 14/505
USPC 435/69.4; 435/254.23; 435/69.1; 435/69.6

(57) ABSTRACT

The present invention relates to novel engineered Pichia strains with improved fermentation yields for expressing heterologous proteins with improved N-glycosylation quality, as well as to methods of generating such strains.

This application claims the benefit of U.S. provisional patent application No. 61/553,801; filed Oct. 31, 2011; which is herein incorporated by reference in its entirety.

### FIELD OF THE INVENTION

The present invention relates to novel engineered Pichia strains with improved fermentation yields for expressing heterologous proteins with improved N-glycosylation quality, as well as to methods of generating such strains.

### BACKGROUND OF THE INVENTION

The methylotrophic yeast Pichia pastoris is one of the most widely used expression hosts for genetic engineering. This ascomycetous single-celled budding yeast has been used for the heterologous expression of hundreds of proteins (Lin-Cereghino, Curr Opin Biotech, 2002; Macauley-Patrick, Yeast, 2005). Importantly, P. pastoris is a lower eukaryote which provides the further advantage of having basic machinery for protein folding and post-translational modifications.

As a protein expression system, P. pastoris provides the advantages of a microbial system with facile genetics, shorter cycle times and the capability of achieving high cell densities. Secreted protein productivities have routinely been reported in the multi-gram per liter ranges. Several promoter systems are available for expression of proteins, for example, the methanol-inducible AOX1 promoter. The AOX1 promoter is a desirable feature of the P. pastoris system because it is tightly regulated and highly induced upon exposure to methanol (Cregg, Biotechnology, 1993, 11:905-910). The native Aox1p can be expressed up to 30% of total cellular protein when cells are grown on methanol. A drawback to this system is that cultivation on methanol during large scale fermentation can be complicated.

Constitutive promoter systems have been developed using the GAPDH promoter and more recently the TEF promoter (Waterham, Gene 1997, 186: 37-44; Ahn, Appl Microb Biotech, 2007, 74:601-608). These promoters are not as strong as AOX1, but, in some instances have lead to yield higher levels of secreted product than expression by AOX1, probably due to cultivation on a more energetically rich carbon source such as glycerol or glucose. However, such alternative promoter systems can be unpredictable for heterologous protein production.

Engineered Pichia strains have been utilized as an alternative host system for producing recombinant glycoproteins with human-like glycosylation. However, the extensive genetic modifications have also caused fundamental changes in cell wall structures in many glycoengineered yeast strains, predisposing some glyco-engineered strains to cell lysis and reduced cell robustness during fermentation.

Certain glyco-engineered strains have substantial reductions in cell viability as well as a marked increase in intracellular protease leakage into the fermentation broth, resulting in a reduction in both recombinant product yield and quality. Current strategies for identifying robust glyco-engineered Pichia production strains rely heavily on screening a large number of clones using various platforms such as 96-deep-well plates, 5 ml mini-scale fermenters (Micro24), and 0.5 L-scale bioreactors (DasGip) to empirically identify clones that are compatible for large-scale (40 L and above) fermentation processes (Barnard et al. 2010). Despite the fact that high-throughput screening has been successfully used to identify several Pichia hosts capable of producing recombinant mAb with yields in excess of 1 g/L (anti-RSV and anti-Her2) (Potgieter et al. 2009; Zhang et al. 2011), these large-scale screening approach is very resource-intensive and time-consuming, and often only identify clones with incremental increases in cell-robustness.

Therefore, host strains that have improved robustness and the ability to produce high quality human-like proteins would be of value and interest to the field. Here, we present engineered Pichia host strains having a deletion, nonsense mutation, or other modification resulting in a truncation of a P. pastoris gene XRN1, which under bioprocess conditions produce both higher titer protein products that also exhibit improved N-glycosylation compared to protein produced produced in XRN1 naïve parental strains under similar production conditions. These strains are especially useful for heterologous gene expression and production of therapeutic proteins.

### SUMMARY OF THE INVENTION

The present invention relates to a modified Pichia sp. host cell wherein the host cell has been modified to reduce or eliminate expression of a functional gene product of a nucleic acid sequence encoding a polypeptide having the amino acid sequence set forth in SEQ ID NO:76. In additional embodiments, the modified host cell comprises a disruption or deletion in the nucleic acid sequence encoding a polypeptide having the amino acid sequence set forth in SEQ ID NO:76. In further embodiments, the host cell further comprises disruption or deletion of one or more of a functional gene product encoding an alpha-1,6-mannosyltransferase activity, mannosylphosphate transferase activity, and β-mannosyltransferase activity.

In yet additional embodiments, the invention further comprises one or more nucleic acid sequences of interest. In certain embodiments, the nucleic acid sequences of interest encode one or more glycosylation enzymes or oligosaccharyltransferases. In certain embodiments, the glycosylation enzymes or oligosaccharyltransferases are selected from the group consisting of glycosidases, mannosidases, phosphomannosidases, phosphatases, nucleotide sugar transporters, mannosyltransferases, the N-acetylglucosaminyltransferases, the UDP-N-acetylglucosamine transporters, the galactosyltransferases, the sialyltransferases, the protein mannosyltransferases, and the oligosaccharyltransferases STT3A, STT3B, STT3C and STT3D.

In yet additional embodiments, the nucleic acid sequences of interest encode one or more therapeutic proteins. In certain embodiments, the therapeutic proteins are selected from the group consisting of an immunoglobulin heavy chain variable domain (optionally wherein the variable domain is linked to an immunoglobulin heavy chain constant domain), an immunoglobulin light chain variable domain (optionally wherein the variable domain is linked to an immunoglobulin light chain constant domain), kringle domains of the human plasminogen, erythropoietin, cytokines, coagulation factors, soluble IgE receptor α-chain, IgG, IgG fragments, IgM, urokinase, chymase, urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1 antitrypsin, DNase II, insulin, Fc-fusions, and HSA-fusions.

The present invention further provides a Pichia sp. host cell comprising a disruption or deletion of the XRN1 gene in the genomic DNA of the host cell that encodes a protein having of a nucleic acid sequence encoding a polypeptide having the amino acid sequence set forth in SEQ ID NO:76. In yet additional embodiments, the host cell further comprises a nucleic acid sequence of interest.

In yet additional embodiments, the modified host cell of the present invention produces proteins with improved N-glycosylation compared with the XRN1 naïve parental host cell under similar culture conditions.

In yet additional embodiments, the invention relates to a method for producing glycoprotein compositions in Pichia sp. host cells, said method comprising growing the modified host cells described herein under inducing conditions.

In further embodiments, the host cell further comprises disruption or deletion of one or more of a functional gene product encoding an alpha-1,6-mannosyltransferase activity, mannosylphosphate transferase activity, β-mannosyltransferase activity, or a dolichol-P-Man dependent alpha(1-3) mannosyltransferase activity.

In yet additional embodiments, the invention further comprises one or more nucleic acid sequences of interest. In certain embodiments, the nucleic acid sequences of interest encode one or more glycosylation enzymes or oligosaccharyltransferases. In certain embodiments, the glycosylation enzymes or oligosaccharyltransferases are selected from the group consisting of glycosidases, mannosidases, phosphomannosidases, phosphatases, nucleotide sugar transporters, mannosyltransferases, the N-acetylglucosaminyltransferases, the UDP-N-acetylglucosamine transporters, the galactosyltransferases, the sialyltransferases, the protein mannosyltransferases, and the oligosaccharyltransferases STT3A, STT3B, STT3C and STT3D.

In yet additional embodiments, the nucleic acid sequences of interest encode one or more therapeutic proteins. In certain embodiments, the therapeutic proteins are selected from the group consisting of kringle domains of the human plasminogen, erythropoietin, cytokines, coagulation factors, soluble IgE receptor α-chain, IgG, IgG fragments, IgM, urokinase, chymase, urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1 antitrypsin, DNase II,α-feto proteins, insulin, Fc-fusions, and HSA-fusions.

In certain embodiments, the invention also provides host cells comprising a disruption, deletion or mutation of a nucleic acid sequence selected from the group consisting of the coding sequence of the P. pastoris XRN1 gene, a nucleic acid sequence that is a degenerate variant of the coding sequence of the P. pastoris XRN1 gene and related nucleic acid sequences and fragments, in which the host cells have a reduced activity of the polypeptide encoded by the nucleic acid sequence compared to a host cell without the disruption, deletion or mutation.

In addition, the invention provides methods for the genetic integration of a heterologous nucleic acid sequence into a host cell comprising a disruption or deletion of the P. pastoris XRN1 gene in the genomic DNA of the host cell. These methods comprise the step of introducing a sequence of interest into the host cell comprising a disrupted, deleted or mutated nucleic acid sequence derived from a sequence selected from the group consisting of the coding sequence of the P. pastoris XRN1 gene, a nucleic acid sequence that is a degenerate variant of the coding sequence of the P. pastoris XRN1 gene and related nucleic acid sequences and fragments.

### BRIEF DESCRIPTION OF THE DRAWINGS

FIG. 1A-H shows the genealogy of P. pastoris strain YGLY12501 (FIG. 1F), YGLY13992 (FIG. 1G), and strain YGLY14836 (FIG. 1H) beginning from wild-type strain NRRL-Y11430 (FIG. 1A).

FIGS. 2 A-C shows the genealogy of P. pastoris glycoinsulin producing strain YGLY21058 (FIG. 2A) beginning from glycoengineering strain YGLY7961.

FIG. 3 shows as map of plasmid pGLY7392. Plasmid pGLY7392 is an integration vector that targets the XRN1/KEM1 loci contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the XRN1 gene (PpXRN1-5′) and on the other side by a nucleic acid molecule comprising a nucleotide sequence from the 3′ region of the XRN1 gene (Pp XRN1-3′).

FIG. 4 shows a map of plasmid pGLY6. Plasmid pGLY6 is an integration vector that targets the URA5 locus and contains a nucleic acid molecule comprising the S. cerevisiae invertase gene or transcription unit (ScSUC2) flanked on one side by a nucleic acid molecule comprising a nucleotide sequence from the 5′ region of the P. pastoris URA5 gene (PpURA5-5′) and on the other side by a nucleic acid molecule comprising the a nucleotide sequence from the 3′ region of the P. pastoris URA5 gene (PpURA5-3′).

FIG. 5 shows a map of plasmid pGLY40. Plasmid pGLY40 is an integration vector that targets the OCH1 locus and contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the OCH1 gene (PpOCH1-5′) and on the other side by a nucleic acid molecule comprising a nucleotide sequence from the 3′ region of the OCH1 gene (PpOCH1-3′).

FIG. 6 shows a map of plasmid pGLY43a. Plasmid pGLY43a is an integration vector that targets the BMT2 locus and contains a nucleic acid molecule comprising the K. lactis UDP-N-acetylglucosamine (UDP-GlcNAc) transporter gene or transcription unit (KlGlcNAc Transp.) adjacent to a nucleic acid molecule comprising the P. pastoris URA5 gene or transcription unit (PpURA5) flanked by nucleic acid molecules comprising lacZ repeats (lacZ repeat). The adjacent genes are flanked on one side by a nucleic acid molecule comprising a nucleotide sequence from the 5′ region of the BMT2 gene (PpPBS2-5′) and on the other side by a nucleic acid molecule comprising a nucleotide sequence from the 3′ region of the BMT2 gene (PpPBS2-3′).

FIG. 7 shows a map of plasmid pGLY48. Plasmid pGLY48 is an integration vector that targets the MNN4L1 locus and contains an expression cassette comprising a nucleic acid molecule encoding the mouse homologue of the UDP-GlcNAc transporter (MmGlcNAc Transp.) open reading frame (ORF) operably linked at the 5′ end to a nucleic acid molecule comprising the P. pastoris GAPDH promoter (PpGAPDH Prom) and at the 3′ end to a nucleic acid molecule comprising the S. cerevisiae CYC termination sequence (ScCYC TT) adjacent to a nucleic acid molecule comprising the P. pastoris URA5 gene or transcription unit (PpURA5) flanked by lacZ repeats (lacZ repeat) and in which the expression cassettes together are flanked on one side by a nucleic acid molecule comprising a nucleotide sequence from the 5′ region of the P. pastoris MNN4L1 gene (PpMNN4L1-5′) and on the other side by a nucleic acid molecule comprising a nucleotide sequence from the 3′ region of the MNN4L1 gene (PpMNN4L1-3′).

FIG. 8 shows as map of plasmid pGLY45. Plasmid pGLY45 is an integration vector that targets the PNO1/MNN4 loci contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the PNO1 gene (PpPNO1-5′) and on the other side by a nucleic acid molecule comprising a nucleotide sequence from the 3′ region of the MNN4 gene (PpMNN4-3′).

FIG. 9 shows a map of plasmid pGLY1430. Plasmid pGLY1430 is a KINKO integration vector that targets the ADE1 locus without disrupting expression of the locus and contains in tandem four expression cassettes encoding (1) the human GlcNAc transferase I catalytic domain (codon optimized) fused at the N-terminus to P. pastoris SEC12 leader peptide (CO-NA10), (2) mouse homologue of the UDP-GlcNAc transporter (MmTr), (3) the mouse mannosidase IA catalytic domain (FB) fused at the N-terminus to S. cerevisiae SEC12 leader peptide (FB8), and (4) the P. pastoris URA5 gene or transcription unit (PpURA5) flanked by lacZ repeats (lacZ). All flanked by the 5′ region of the ADE1 gene and ORF (ADE1 5′ and ORF) and the 3′ region of the ADE1 gene (PpADE1-3′). PpPMA1 prom is the P. pastoris PMA1 promoter; PpPMA1 TT is the P. pastoris PMA1 termination sequence; SEC4 is the P. pastoris SEC4 promoter; OCH1 TT is the P. pastoris OCH1 termination sequence; ScCYC TT is the S. cerevisiae CYC termination sequence; PpOCH1 Prom is the P. pastoris OCH1 promoter; PpALG3 TT is the P. pastoris ALG3 termination sequence; and PpGAPDH is the P. pastoris GADPH promoter.

FIG. 10 shows a map of plasmid pGLY582. Plasmid pGLY582 is an integration vector that targets the HIS1 locus and contains in tandem four expression cassettes encoding (1) the S. cerevisiae UDP-glucose epimerase (ScGAL10), (2) the human galactosyltransferase I (hGalT) catalytic domain fused at the N-terminus to the S. cerevisiae KRE2-s leader peptide (33), (3) the P. pastoris URA5 gene or transcription unit (PpURA5) flanked by lacZ repeats (lacZ repeat), and (4) the D. melanogaster UDP-galactose transporter (DmUGT). All flanked by the 5′ region of the HIS1 gene (PpHIS1-5′) and the 3′ region of the HIS1 gene (PpHIS1-3′). PMA1 is the P. pastoris PMA1 promoter; PpPMA1 TT is the P. P. pastoris PMA1 termination sequence; GAPDH is the P. pastoris GADPH promoter and ScCYC TT is the S. cerevisiae CYC termination sequence; PpOCH1 Prom is the P. pastoris OCH1 promoter and PpALG12 TT is the P. pastoris ALG12 termination sequence.

FIG. 11 shows a map of plasmid pGLY167b. Plasmid pGLY167b is an integration vector that targets the ARG1 locus and contains in tandem three expression cassettes encoding (1) the D. melanogaster mannosidase II catalytic domain (codon optimized) fused at the N-terminus to S. cerevisiae MNN2 leader peptide (CO-KD53), (2) the P. pastoris HIS1 gene or transcription unit, and (3) the rat N-acetylglucosamine (GlcNAc) transferase II catalytic domain (codon optimized) fused at the N-terminus to S. cerevisiae MNN2 leader peptide (CO-TC54). All flanked by the 5′ region of the ARG1 gene (PpARG1-5′) and the 3′ region of the ARG1 gene (PpARG1-3′). PpPMA1 prom is the P. pastoris PMA1 promoter; PpPMA1 TT is the P. pastoris PMA1 termination sequence; PpGAPDH is the P. pastoris GADPH promoter; ScCYC TT is the S. cerevisiae CYC termination sequence; PpOCH1 Prom is the P. pastoris OCH1 promoter; and PpALG12 TT is the P. pastoris ALG12 termination sequence.

FIG. 12 shows a map of plasmid pGLY3411 (pSH1092). Plasmid pGLY3411 (pSH1092) is an integration vector that contains the expression cassette comprising the P. pastoris URA5 gene or transcription unit (PpURA5) flanked by lacZ repeats (lacZ repeat) flanked on one side with the 5′ nucleotide sequence of the P. pastoris BMT4 gene (PpPBS4 5′) and on the other side with the 3′ nucleotide sequence of the P. pastoris BMT4 gene (PpPBS4 3′).

FIG. 13 shows a map of plasmid pGLY3419 (pSH1110). Plasmid pGLY3430 (pSH1115) is an integration vector that contains an expression cassette comprising the P. pastoris URA5 gene or transcription unit (PpURA5) flanked by lacZ repeats (lacZ repeat) flanked on one side with the 5′ nucleotide sequence of the P. pastoris BMT1 gene (PBS1 5′) and on the other side with the 3′ nucleotide sequence of the P. pastoris BMT1 gene (PBS 1 3′)

FIG. 14 shows a map of plasmid pGLY3421 (pSH1106). Plasmid pGLY4472 (pSH1186) contains an expression cassette comprising the P. pastoris URA5 gene or transcription unit (PpURA5) flanked by lacZ repeats (lacZ repeat) flanked on one side with the 5′ nucleotide sequence of the P. pastoris BMT3 gene (PpPBS3 5′) and on the other side with the 3′ nucleotide sequence of the P. pastoris BMT3 gene (PpPBS3 3′).

FIG. 15 shows a map of plasmid pGLY3673. Plasmid pGLY3673 is a KINKO integration vector that targets the PRO1 locus without disrupting expression of the locus and contains expression cassettes encoding the T. reesei α-1,2-mannosidase catalytic domain fused at the N-terminus to S. cerevisiae aMATpre signal peptide (aMATTrMan) to target the chimeric protein to the secretory pathway and secretion from the cell.

FIG. 16 shows a map of pGLY5883 encoding the light and heavy chains of an anti-Her2 antibody. The plasmid is a roll-in vector that targets the TRP2 locus. The ORFs encoding the light and heavy chains are under the control of a P. pastoris AOX1 promoter and the S. cerevisiae CYC 3UTR transcription termination sequence. Selection of transformants uses zeocin resistance encoded by the zeocin resistance protein (Zeocin^{R}) ORF under the control of the P. pastoris TEF1 promoter and S. cerevisiae CYC termination sequence.

FIG. 17 shows a map of pGLY6833 encoding the light and heavy chains of an anti-Her2 antibody. The plasmid is a roll-in vector that targets the TRP2 locus. The ORFs encoding the light and heavy chains are under the control of a P. pastoris AOX1 promoter and the P. pastoris CIT1 3UTR transcription termination sequence. Selection of transformants uses zeocin resistance encoded by the zeocin resistance protein (Zeocin^{R}) ORF under the control of the P. pastoris TEF1 promoter and S. cerevisiae CYC termination sequence.

FIG. 18 shows a map of plasmid pGLY3714. Plasmid pGLY3714 is a KINKO integration vector that targets the TRP1 locus without disrupting expression of the locus and contains expression cassettes encoding the mouse mannosidase IB catalytic domain (GD) fused at the N-terminus to S. cerevisiae SEC12 leader peptide (GD9) to target the chimeric enzyme to the ER or Golgi. For selecting transformants, the plasmid comprises an expression cassette encoding the Nourseothricin resistance (NAT^{R}) ORF (originally from pAG25 from EROSCARF, Scientific Research and Development GmbH, Daimlerstrasse 13a, D-61352 Bad Homburg, Germany, See Goldstein et al., Yeast 15: 1541 (1999)); wherein the nucleic acid molecule encoding the ORF (SEQ ID NO:64) is operably linked to at the 5′ end to a nucleic acid molecule having the Ashbya gossypii TEF1 promoter sequence (SEQ ID NO:65) and at the 3′ end to a nucleic acid molecule that has the Ashbya gossypii TEF1 termination sequence (SEQ ID NO:66).

FIG. 19 shows a map of plasmid pGLY2456. Plasmid pGLY2456 is a KINKO integration vector that targets the TRP2 locus without disrupting expression of the locus and contains six expression cassettes encoding (1) the mouse CMP-sialic acid transporter codon optimized (CO mCMP-Sia Transp), (2) the human UDP-GlcNAc 2-epimerase/N-acetylmannosamine kinase codon optimized (CO hGNE), (3) the Pichia pastoris ARG1 gene or transcription unit, (4) the human CMP-sialic acid synthase codon optimized (CO hCMP-NANA S), (5) the human N-acetylneuraminate-9-phosphate synthase codon optimized (CO hSIAP S), and, (6) the mouse a-2,6-sialyltransferase catalytic domain codon optimized fused at the N-terminus to S. cerevisiae KRE2 leader peptide (comST6-33). All flanked by the 5′ region of the TRP2 gene and ORF (PpTRP2 5′) and the 3′ region of the TRP2 gene (PpTRP2-3′). PpPMA1 prom is the P. pastoris PMA1 promoter; PpPMA1 TT is the P. pastoris PMA1 termination sequence; CYC TT is the S. cerevisiae CYC termination sequence; PpTEF Prom is the P. pastoris TEF1 promoter; PpTEF TT is the P. pastoris TEF1 termination sequence; PpALG3 TT is the P. pastoris ALG3 termination sequence; and pGAP is the P. pastoris GAPDHpromoter.

FIG. 20 shows a map of plasmid pGLY5048 (pSH1275). Plasmid pGLY5048 (pSH1275) is an integration vector that targets the STE13 locus and contains expression cassettes encoding (1) the T. reesei α-1,2-mannosidase catalytic domain fused at the N-terminus to S. cerevisiae aMATpre signal peptide (aMATTrMan) to target the chimeric protein to the secretory pathway and secretion from the cell and (2) the P. pastoris URA5 gene or transcription unit.

FIG. 21 shows a map of plasmid pGLY5019 (pSH1246). Plasmid pGLY5019 (pSH1246) is an integration vector that targets the DAP2 locus and contains an expression cassette comprising a nucleic acid molecule encoding the Nourseothricin resistance (NAT^{R}) ORF operably linked to the Ashbya gossypii TEF1 promoter and A. gossypii TEF1 termination sequences flanked one side with the 5′ nucleotide sequence of the P. pastoris DAP2 gene and on the other side with the 3′ nucleotide sequence of the P. pastoris DAP2 gene.

FIG. 22 shows a map of plasmid pGLY5085 (pSH1312). Plasmid pGLY5085 (pSH1312) is a KINKO plasmid for introducing a second set of the genes involved in producing sialylated N-glycans into P. pastoris. The plasmid is similar to plasmid YGIN2456 except that the P. pastoris ARG1 gene has been replaced with an expression cassette encoding hygromycin resistance (HygR) and the plasmid targets the P. pastoris TRP5 locus. The six tandem cassettes are flanked on one side by a nucleic acid molecule comprising a nucleotide sequence from the 5′ region and ORF of the TRP5 gene ending at the stop codon followed by a P. pastoris ALG3 termination sequence and on the other side by a nucleic acid molecule comprising a nucleotide sequence from the 3′ region of the TRP5 gene.

FIG. 23 shows map of plasmid pGLY4362, which is a roll-in integration plasmid that targets the TRP2 or AOX1p loci, includes an expression cassette encoding an insulin precursor fusion protein comprising a Yps1ss peptide fused to a TA57 propeptide fused to an N-terminal spacer fused to the human insulin B-chain with a P28N substitution fused to a C-peptide consisting of the amino acid sequence AAK fused to the human insulin A-chain.

### DETAILED DESCRIPTION OF THE INVENTION

### Molecular Biology

In accordance with the present invention there may be employed conventional molecular biology, microbiology, and recombinant DNA techniques within the skill of the art. Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., James M. Cregg (Editor), Pichia Protocols (Methods in Molecular Biology), Humana Press (2010), Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999), Animal Cell Culture (R.I. Freshney, ed. (1986)); Immobilized Cells And Enzymes (IRL Press, (1986)); B. Perbal, A Practical Guide To Molecular Cloning (1984);

A “polynucleotide”, “nucleic acid” includes DNA and RNA in single stranded form, double-stranded form or otherwise.

A “polynucleotide sequence” or “nucleotide sequence” is a series of nucleotide bases (also called “nucleotides”) in a nucleic acid, such as DNA or RNA, and means a series of two or more nucleotides. Any polynucleotide comprising a nucleotide sequence set forth herein (e.g., promoters of the present invention) forms part of the present invention.

A “coding sequence” or a sequence “encoding” an expression product, such as an RNA or polypeptide is a nucleotide sequence (e.g., heterologous polynucleotide) that, when expressed, results in production of the product (e.g., a heterologous polypeptide such as an immunoglobulin heavy chain and/or light chain).

As used herein, the term “oligonucleotide” refers to a nucleic acid, generally of no more than about 100 nucleotides (e.g., 30, 40, 50, 60, 70, 80, or 90), that may be hybridizable to a polynucleotide molecule. Oligonucleotides can be labeled, e.g., by incorporation of ³²P-nucleotides, ³H-nucleotides, 14C-nucleotides, ³⁵S-nucleotides or nucleotides to which a label, such as biotin, has been covalently conjugated.

A “protein”, “peptide” or “polypeptide” (e.g., a heterologous polypeptide such as an immunoglobulin heavy chain and/or light chain) includes a contiguous string of two or more amino acids.

A “protein sequence”, “peptide sequence” or “polypeptide sequence” or “amino acid sequence” refers to a series of two or more amino acids in a protein, peptide or polypeptide.

The term “isolated polynucleotide” or “isolated polypeptide” includes a polynucleotide or polypeptide, respectively, which is partially or fully separated from other components that are normally found in cells or in recombinant DNA expression systems or any other contaminant. These components include, but are not limited to, cell membranes, cell walls, ribosomes, polymerases, serum components and extraneous genomic sequences. The scope of the present invention includes the isolated polynucleotides set forth herein, e.g., the promoters set forth herein; and methods related thereto, e.g., as discussed herein.

An isolated polynucleotide or polypeptide will, preferably, be an essentially homogeneous composition of molecules but may contain some heterogeneity.

“Amplification” of DNA as used includes the use of polymerase chain reaction (PCR) to increase the concentration of a particular DNA sequence within a mixture of DNA sequences. For a description of PCR see Saiki, et al., Science (1988) 239:487.

In general, a “promoter” or “promoter sequence” is a DNA regulatory region capable of binding an RNA polymerase in a cell (e.g., directly or through other promoter-bound proteins or substances) and initiating transcription of a coding sequence to which it operably links.

A coding sequence (e.g., of a heterologous polynucleotide, e.g., reporter gene or immunoglobulin heavy and/or light chain) is “operably linked to”, “under the control of”, “functionally associated with” or “operably associated with” a transcriptional and translational control sequence (e.g., a promoter of the present invention) when the sequence directs RNA polymerase mediated transcription of the coding sequence into RNA, preferably mRNA, which then may be RNA spliced (if it contains introns) and, optionally, translated into a protein encoded by the coding sequence.

The present invention includes vectors or cassettes which comprise modified XRN1 including nonsense mutations, truncations, deletions, knock-outs, or overexpression cassettes, including promoters optionally operably linked to a heterologous polynucleotide. The term “vector” includes a vehicle (e.g., a plasmid) by which a DNA or RNA sequence can be introduced into a host cell, so as to transform the host and, optionally, promote expression and/or replication of the introduced sequence. Suitable vectors for use herein include plasmids, integratable DNA fragments, and other vehicles that may facilitate introduction of the nucleic acids into the genome of a host cell (e.g., Pichia pastoris). Plasmids are the most commonly used form of vector but all other forms of vectors which serve a similar function and which are, or become, known in the art are suitable for use herein. See, e.g., Pouwels, et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass.

A polynucleotide (e.g., a heterologous polynucleotide, e.g., encoding an immunoglobulin heavy chain and/or light chain), operably linked to a promoter, may be expressed in an expression system. The term “expression system” means a host cell and compatible vector which, under suitable conditions, can express a protein or nucleic acid which is carried by the vector and introduced to the host cell. Common expression systems include fungal host cells (e.g., Pichia pastoris) and plasmid vectors, insect host cells and Baculovirus vectors, and mammalian host cells and vectors.

The term methanol-induction refers to increasing expression of a polynucleotide (e.g., a heterologous polynucleotide) operably linked to a methanol-inducible promoter in a host cell of the present invention by exposing the host cells to methanol.

The term methanol-repression refers to decreasing expression of a polynucleotide (e.g., a heterologous polynucleotide) operably linked to a methanol-repressible promoter in a host cell of the present invention by exposing the host cells to methanol.

The following references regarding the BLAST algorithm are herein incorporated by reference: BLAST ALGORITHMS: Altschul, S. F., et al., J. Mol. Biol. (1990) 215:403-410; Gish, W., et al., Nature Genet. (1993) 3:266-272; Madden, T. L., et al., Meth. Enzymol. (1996) 266:131-141; Altschul, S. F., et al., Nucleic Acids Res. (1997) 25:3389-3402; Zhang, J., et al., Genome Res. (1997) 7:649-656; Wootton, J. C., et al., Comput. Chem. (1993) 17:149-163; Hancock, J. M., et al., Comput. Appl. Biosci. (1994) 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M.O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M.O. Dayhoff (ed.), pp. 353-358, Natl. Biomed. Res. Found., Washington, D.C.; Altschul, S.F., J. Mol. Biol. (1991) 219:555-565; States, D. J., et al., Methods (1991) 3:66-70; Henikoff, S., et al., Proc. Natl. Acad. Sci. USA (1992)89:10915-10919; Altschul, S. F., et al., J. Mol. Evol. (1993) 36:290-300; ALIGNMENT STATISTICS: Karlin, S., et al., Proc. Natl. Acad. Sci. USA (1990) 87:2264-2268; Karlin, S., et al., Proc. Natl. Acad. Sci. USA (1993) 90:5873-5877; Dembo, A., et al., Ann. Prob. (1994) 22:2022-2039; and Altschul, S.F. “Evaluating the statistical significance of multiple distinct local alignments.” in Theoretical and Computational Methods in Genome Research (S. Suhai, ed.), (1997) pp. 1-14, Plenum, N.Y.

### Host Cells

The present invention encompasses any isolated Pichia sp. host cell (e.g., such as Pichia pastoris) comprising a modified, truncated, or deleted form of the XRN1 gene, including host cells comprising a promoter e.g., operably linked to a polynucleotide encoding a heterologous polypeptide (e.g., a reporter or immunoglobulin heavy and/or light chain; e.g., optionally, wherein the immunoglobulin heavy chain or light chain is linked to an immunoglobulin constand domain) as well as methods of use thereof, e.g., methods for expressing the heterologous polypeptide in the host cell. Host cells of the present invention, may be also genetically engineered so as to express particular glycosylation patterns on polypeptides that are expressed in such cells. Host cells of the present invention are discussed in detail herein. Any engineered Pichia host cell comprising a modified, truncated, or deleted form of the XRN1 gene forms part of the present invention. In an embodiment of the invention, the host cell is selected from the group consisting of any Pichia cell, such as Pichia pastoris, Pichia angusta (Hansenula polymorpha), Pichia flnlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia.

As used herein, the terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. Predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)).

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.” “PNGase”, or “glycanase” or “glucosidase” refer to peptide N-glycosidase F (EC 3.2.2.18).

Thus, the present invention includes isolated Pichia host cells comprising a modified, truncated, or deleted form of the XRN1 gene, optionally further comprising an expression construct (e.g., a promoter operably linked to a heterologous polynucleotide encoding a heterologous polypeptide) and further comprising a deletion of one or more of the genes encoding PMTs, and/or, e.g., wherein the host cell can be cultivated in a medium that includes one or more Pmtp inhibitors. Pmtp inhibitors include but are not limited to a benzylidene thiazolidinedione. Examples of benzylidene thiazolidinediones are 5-[[3,4bis(phenylmethoxy)phenyl]methylene]-4-oxo-2-thioxo-3-thiazolidineacetic Acid; 5-[[3-(1-25 Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)] phenyl]methylene]-4-oxo-2-thioxo3-thiazolidineacetic acid.

In an embodiment of the invention, a Pichia host cell (e.g., Pichia pastoris) comprising a modified, truncated, or deleted form of the XRN1 gene is, in an embodiment of the invention, genetically engineered to include a nucleic acid that encodes an alpha-1,2-mannosidase that has a signal peptide that directs it for secretion. For example, in an embodiment of the invention, the host cell is engineered to express an exogenous alpha-1,2-mannosidase enzyme having an optimal pH between 5.1 and 8.0, preferably between 5.9 and 7.5. In an embodiment of the invention, the exogenous enzyme is targeted to the endoplasmic reticulum or Golgi apparatus of the host cell, where it trims N-glycans such as Man₈GlcNAc₂ to yield ManₛGlcNAc₂. See U.S. Pat. No. 7,029,872.

Pichia host cells (e.g., Pichia pastoris) comprising a modified, truncated, or deleted form of the XRN1 gene are, in an embodiment of the invention, genetically engineered to eliminate glycoproteins having alpha-mannosidase-resistant N-glycans by deleting or disrupting one or more of the beta-mannosyltransferasegenes (e.g., BMT1, BMT2, BMT3, and BMT4)(See, U.S. Pat. No. 7,465,577) or abrogating translation of RNAs encoding one or more of the beta-mannosyltransferasesusinginterfering RNA, antisense RNA, or the like. The scope of the present invention includes such an engineered Pichia host cell (e.g., Pichia pastoris) comprising an expression cassette (e.g., a promoter operably linked to a heterologous polynucleotide encoding a heterologous polypeptide).

Engineered host cells (e.g., Pichia pastoris) of the present invention also include those that are genetically engineered to eliminate glycoproteins having phosphomannose residues, e.g., by deleting or disrupting one or both of the phosphomannosyl transferase genes PNO1 and MNN4B (See for example, U.S. Pat. Nos. 7,198,921 and 7,259,007), which can include deleting or disrupting the MNN4A gene or abrogating translation of RNAs encoding one or more of the phosphomannosyltransferases using interfering RNA, antisense RNA, or the like. In an embodiment of the invention, an engineered Pichia host cell has been genetically modified to produce glycoproteins that have predominantly an N-glycan selected from the group consisting of complex N-glycans, hybrid N-glycans, and high mannose N-glycans wherein complex N-glycans are, in an embodiment of the invention, selected from the group consisting of Man₃GlcNAc₂, GlcNAC₍₁₋₄₎Man₃GlcNAc₂, NANA₍₁₋₄₎GlcNAc₍₁₋₄₎Man₃GlcNAc₂, and NANA₍₁₋₄₎Gal₍₁₋₄₎Man₃GlcNAc₂; hybrid N-glycans are, in an embodiment of the invention, selected from the group consisting of Man₅GlcNAc₂, GlcNAcMan₅GlcNAc₂, GalGlcNAcMan₅GlcNAc₂, and NANAGalGlcNAcMan₅GlcNAc₂; and high mannose N-glycans are, in an embodiment of the invention, selected from the group consisting of Man₆GlcNAc₂, Man₇GlcNAc₂, Man₉cNAc₂, and Man₉GlcNAc₂. The scope of the present invention includes such engineered Pichia host cells (e.g., Pichia pastoris) comprising g a modified, truncated, or deleted form of the XRN1 gene.

Additionally, engineered Pichia host cells (e.g., Pichia pastoris) of the present invention also include those that are genetically engineered to include a nucleic acid that encodes the Leishmania sp. single-subunit oligosaccharyltransferase STT3A protein, STT3B protein, STT3C protein, STT3D protein, or combinations thereof such as those described in WO2011/06389. Additionally, engineered host cells (e.g., Pichia pastoris) of the present invention also include those that are genetically engineered to eliminate nucleic acids encoding Dolichol-P-Man dependent alpha(1-3) mannosyltransferase, e.g., Alg3, such as described in US Patent Publication No. US2005/0170452. The scope of the present invention includes any such engineered Pichia host cells (e.g., Pichia pastoris) further comprising a modified, truncated, deleted form of the XRN1 gene.

As used herein, the term “essentially free of” as it relates to lack of a particular sugar residue, such as fucose, or galactose or the like, on a glycoprotein, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent.

As used herein, a glycoprotein composition “lacks” or “is lacking” a particular sugar residue, such as fucose or galactose, when no detectable amount of such sugar residue is present on the N-glycan structures. For example, in an embodiment of the present invention, glycoprotein compositions produced by host cells of the invention will “lack fucose,” because the cells do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

CHARACTERIZATION OF PICHIA PASTORISXRN1

The Pichia pastoris gene XRN1 (SEQ ID NO:75, GenBank Accession No.: 002492616.1, amino acid sequence: SEQ ID NO:76) (is homologous to Kem1 in yeast Saccharomyces cerevisiae), part of a family of evolutionarily conserved cytoplasmic 5′ to 3′ exoribonucleases. XRN1 is a member of a large family of conserved exonucleases, although little is known about the catalytic mechanism of its members. Capped RNA is resistant to Xrn1, and Xrn1 strongly prefers mRNA with a 5′ monophosphate as substrate over RNA with a 5′ hydroxyl end. Eukaryotic cells also contain a related exonuclease, Rat1, which is localized to the nucleus and seems to carry out the relevant 5′ to 3′ degradation and processing reactions in the nucleus.

To broadly improve protein quality produced by engineered host strains, several mutant XRN1 knock-out strains were produced from a series of Pichia host strains. While non-mutagenized glyco-engineered parental strains typically produce heterologous proteins with a variety of N-glycosylation patterns, the engineered Pichia host strains with XRN1 deletions produced heterologous protein products with decreased proteolytic degradation as well as desired glycosylation patterns. These engineered Pichia host strains produced glycoproteins with predominant complex N-glycans typically seen of the therapeutic proteins produced from mammalian cells (shown in Tables 7-11).

Such mutations in XRN1 when engineered into any Pichia host strain would serve to increase fermentation robustness, improve recombinant protein yield, and reduce protein product proteolytic degradation. The mRNA stabilization in the engineered Pichia XRN1 knockouts described herein provides useful strains and methods to improve protein fermentation titer and protein glycosylation quality simultaneously. Inhibition of global mRNA turnover by XRN1 knockout increases mRNA abundance of both target protein and corresponding glycosyltransferases. This leads to a yeast host strain with high protein productivity and enhanced complex N-glycan profile. Moreover, mutation of XRN1 may affect translation initiation to prevent stress-induced translation regulation and further improve the titer in these engineered Pichia host strains.

### EXAMPLE 1

### XRN1 Knock-Out Plasmids

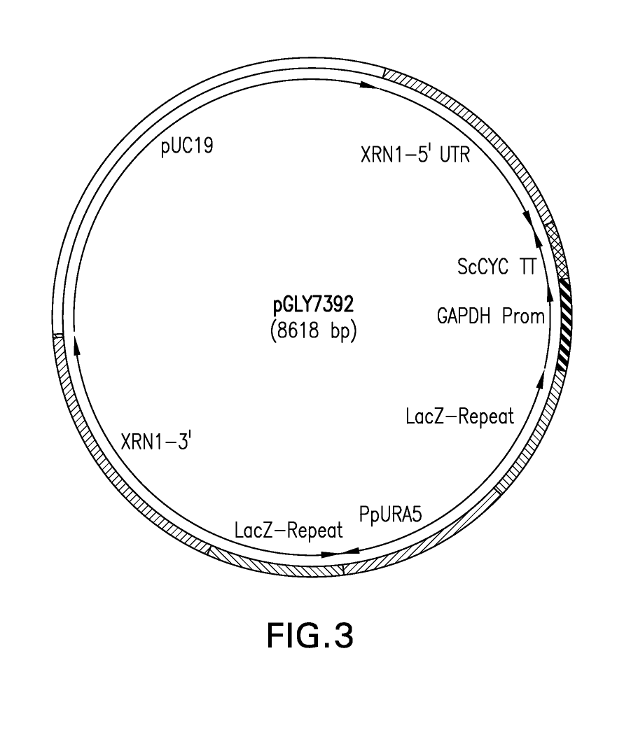

Plasmid pGLY7392 (FIG. 3) is an integration vector that targets the XRN1/KEM1 loci and contains a nucleic acid molecule comprising the P. pastoris URA5 gene or transcription unit flanked by nucleic acid molecules comprising lacZ repeats which in turn is flanked on one side by a nucleic acid molecule comprising a nucleotide sequence from the 5′ region of the XRN1 gene (SEQ ID NO:1) and on the other side by a nucleic acid molecule comprising a nucleotide sequence from the 3′ region of the XRN1 gene (SEQ ID NO: 2).

Plasmid pGLY7392 was linearized with SfiI and the linearized plasmid was transformed into a number of P. pastoris strains in which the URA5 gene flanked by the lacZ repeats has been inserted into the XRN1/KEM1 loci by double-crossover homologous recombination to generate the XRN1 knock-out strains as shown in the following examples.

### EXAMPLE 2

### Engineered Pichia pastoris Strains with Humanized Glycosylation Pathway for Producing Recombinant Human Antibodies

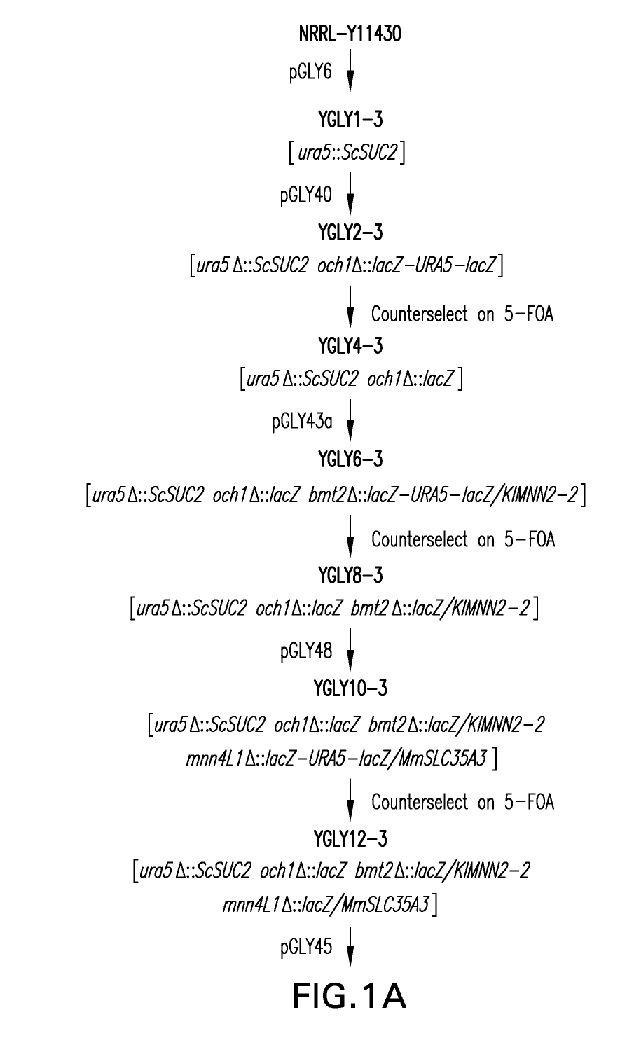

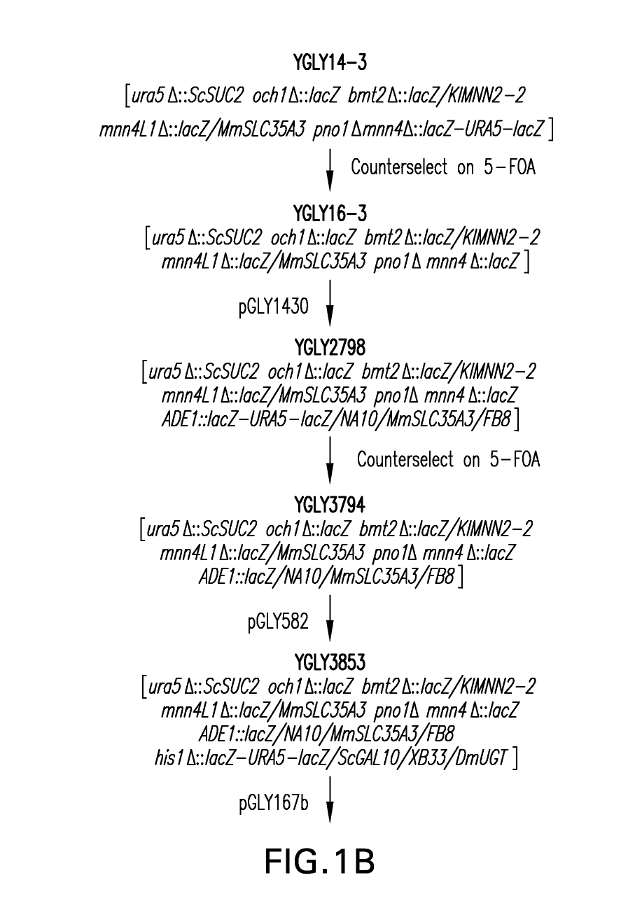

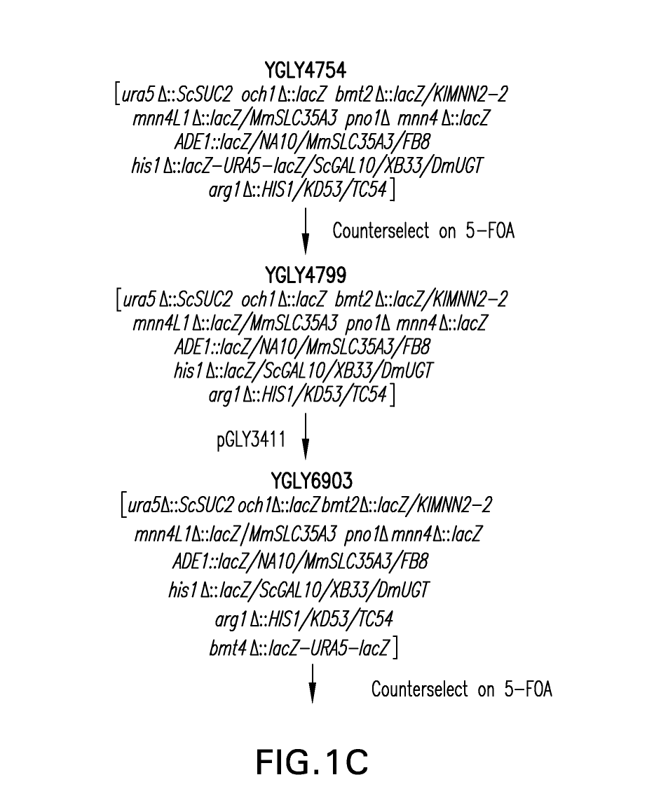

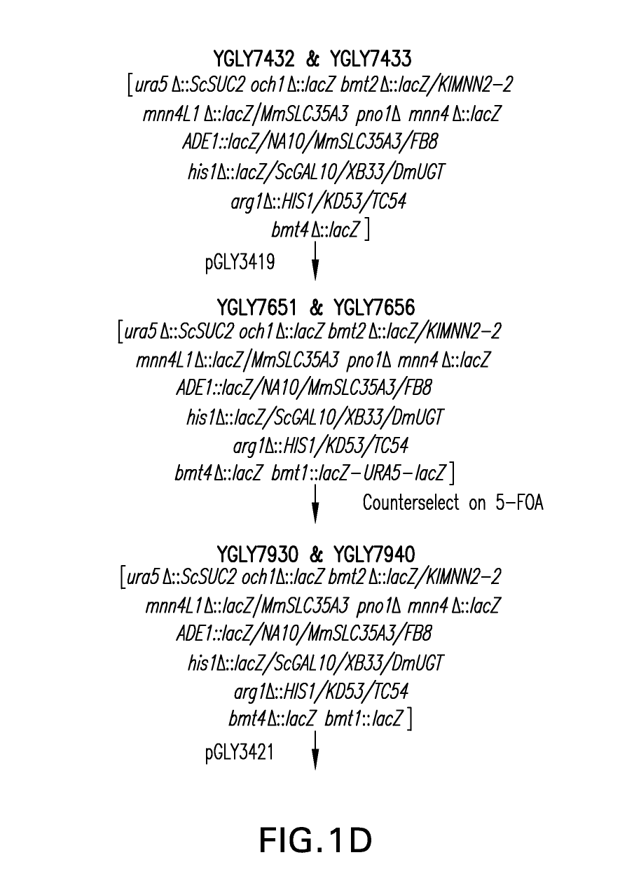

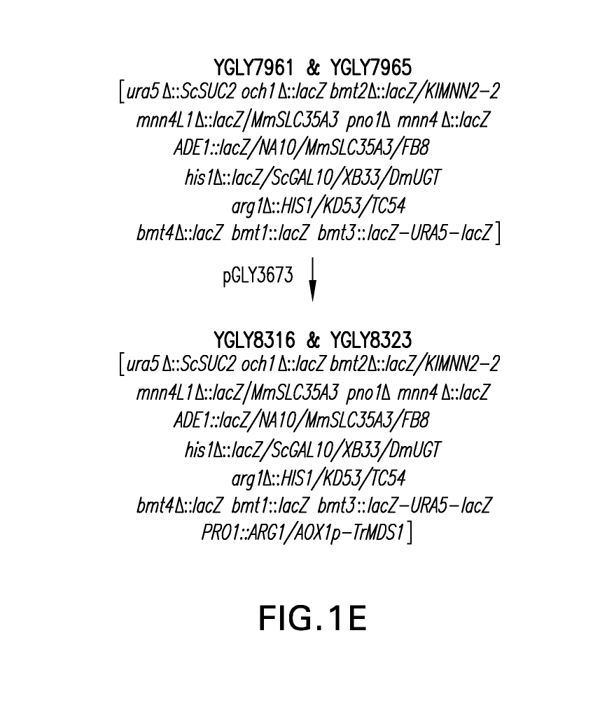

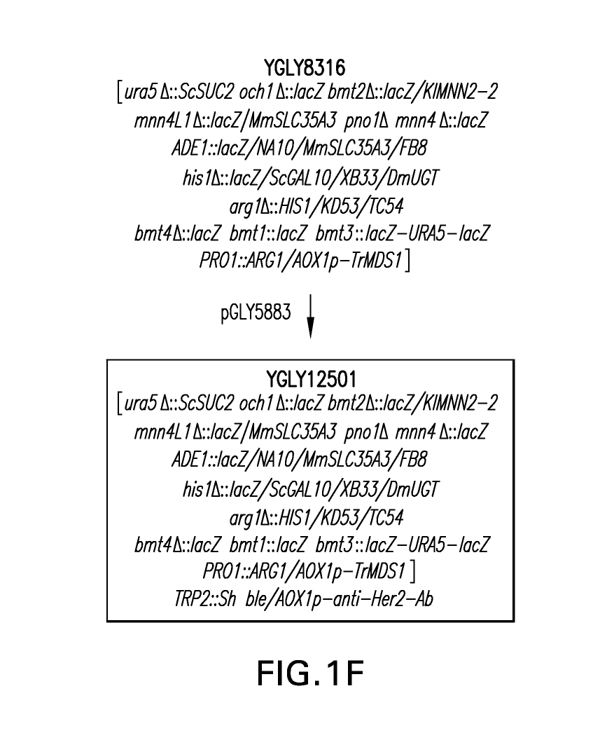

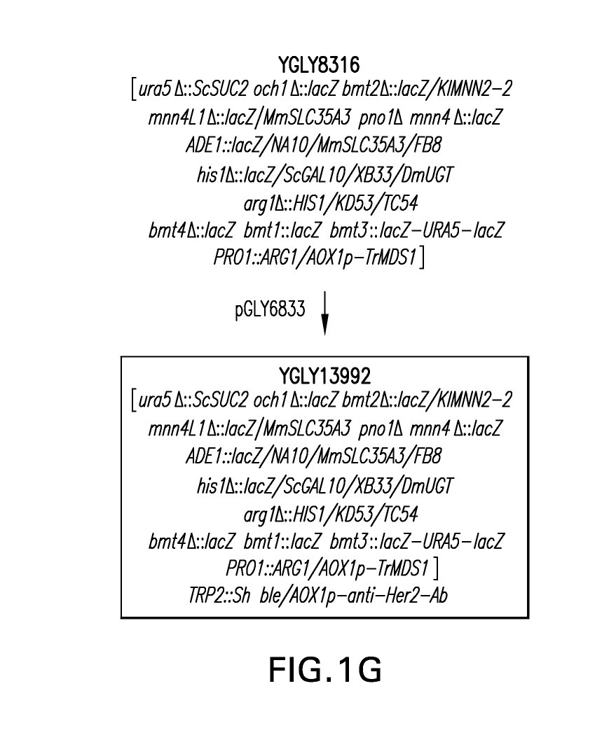

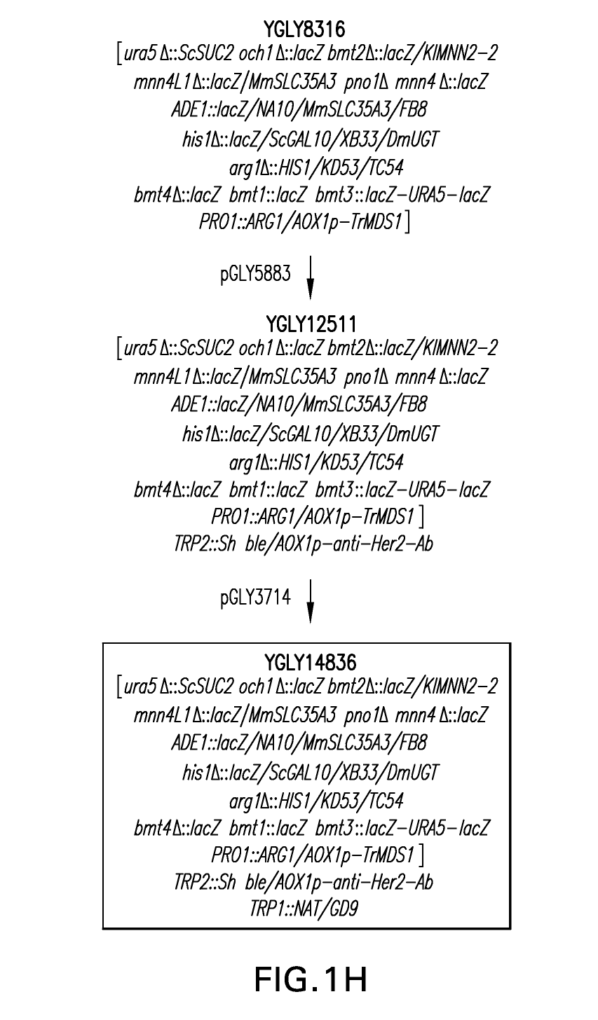

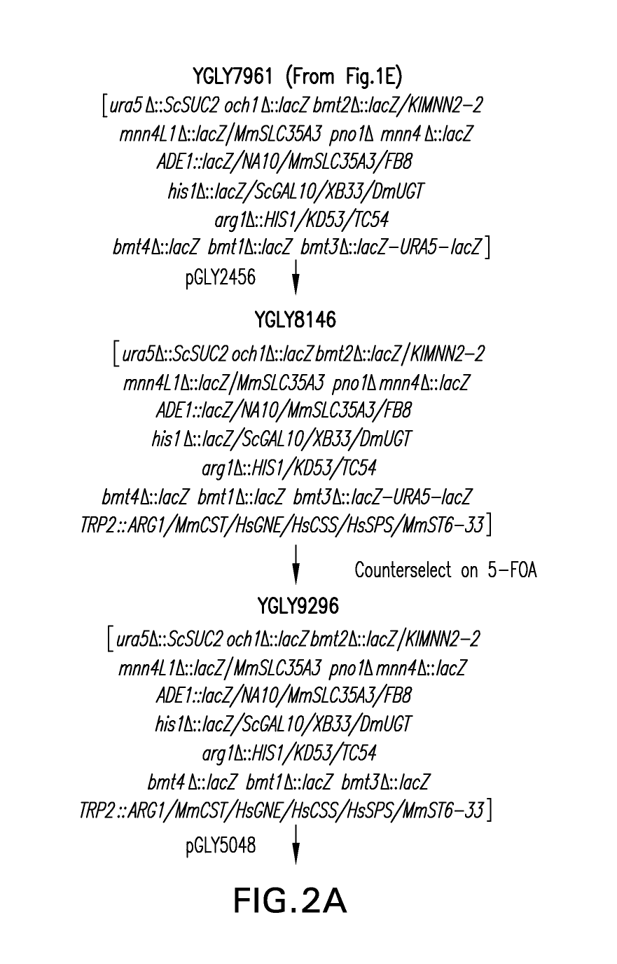

Genetically engineered Pichia pastoris strain YGLY12501, YGLY13992, and YGLY14836 are strains that produce recombinant human anti-Her2 antibodies. Construction of the strains is illustrated schematically in FIGS. 1A-1H. Briefly, the strains were constructed as follows.

The strain YGLY8316 was constructed from wild-type Pichia pastoris strain NRRL-Y 11430 using methods described earlier (See for example, U.S. Pat. No. 7,449,308; U.S. Pat. No. 7,479,389; U.S. Published Application No. 20090124000; Published PCT Application No. WO2009085135; Nett and Gerngross, Yeast 20:1279 (2003); Choi et al., Proc. Natl. Acad. Sci. USA 100:5022 (2003); Hamilton et al., Science 301:1244 (2003)). All plasmids were made in a pUC 19 plasmid using standard molecular biology procedures. For nucleotide sequences that were optimized for expression in P. pastoris, the native nucleotide sequences were analyzed by the GENEOPTIMIZER software (GeneArt, Regensburg, Germany) and the results used to generate nucleotide sequences in which the codons were optimized for P. pastoris expression. Yeast strains were transformed by electroporation (using standard techniques as recommended by BioRad, Hercules, Calif.).

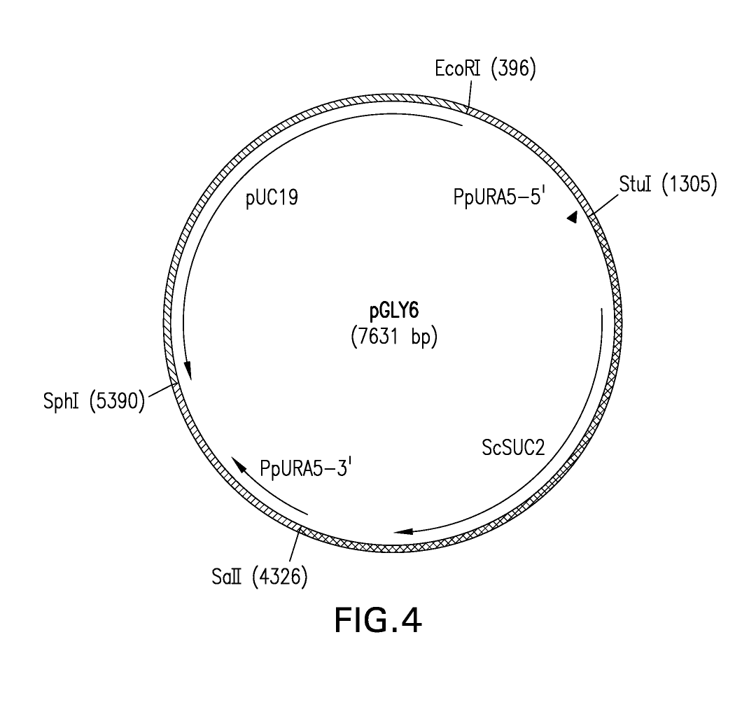

Plasmid pGLY6 (FIG. 4) is an integration vector that targets the URA5 locus. It contains a nucleic acid molecule comprising the S. cerevisiae invertase gene or transcription unit (ScSUC2; SEQ ID NO:3) flanked on one side by a nucleic acid molecule comprising a nucleotide sequence from the 5′ region of the P. pastoris URA5 gene (SEQ ID NO:4) and on the other side by a nucleic acid molecule comprising the nucleotide sequence from the 3′ region of the P. pastoris URA5 gene (SEQ ID NO:5). Plasmid pGLY6 was linearized and the linearized plasmid transformed into wild-type strain NRRL-Y 11430 to produce a number of strains in which the ScSUC2 gene was inserted into the URA5 locus by double-crossover homologous recombination. Strain YGLY1-3 was selected from the strains produced and is auxotrophic for uracil.

Plasmid pGLY40 (FIG. 5) is an integration vector that targets the OCH1 locus and contains a nucleic acid molecule comprising the P. pastoris URA5 gene or transcription unit (SEQ ID NO:6) flanked by nucleic acid molecules comprising lacZ repeats (SEQ ID NO:7) which in turn is flanked on one side by a nucleic acid molecule comprising a nucleotide sequence from the 5′ region of the OCH1 gene (SEQ ID NO:8) and on the other side by a nucleic acid molecule comprising a nucleotide sequence from the 3′ region of the OCH1 gene (SEQ ID NO:9). Plasmid pGLY40 was linearized with SfiI and the linearized plasmid transformed into strain YGLY1-3 to produce a number of strains in which the URA5 gene flanked by the lacZ repeats has been inserted into the OCH1 locus by double-crossover homologous recombination. Strain YGLY2-3 was selected from the strains produced and is prototrophic for URA5. Strain YGLY2-3 was counterselected in the presence of 5-fluoroorotic acid (5-FOA) to produce a number of strains in which the URA5 gene has been lost and only the lacZ repeats remain in the OCH1 locus. This renders the strain auxotrophic for uracil. Strain YGLY4-3 was selected.

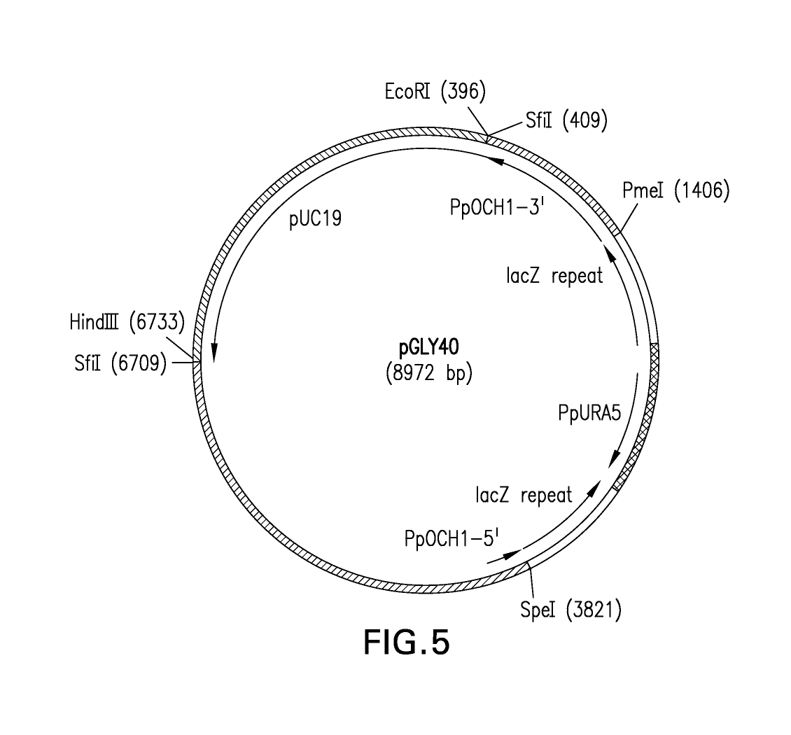

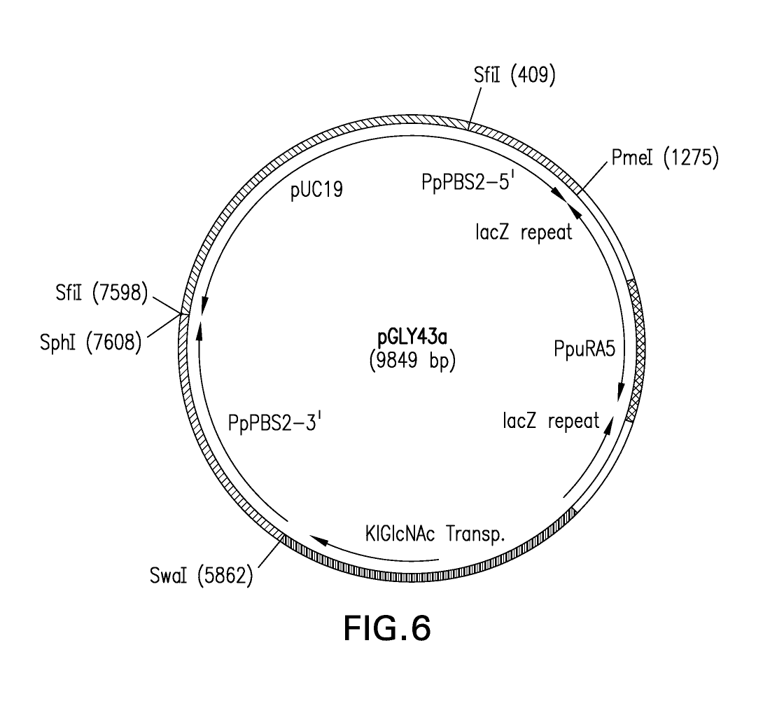

Plasmid pGLY43a (FIG. 6) is an integration vector that targets the BMT2 locus and contains a nucleic acid molecule comprising the K. lactis UDP-N-acetylglucosamine (UDP-GlcNAc) transporter gene or transcription unit (KlMNN2-2, SEQ ID NO:10) adjacent to a nucleic acid molecule comprising the P. pastoris URA5 gene or transcription unit flanked by nucleic acid molecules comprising lacZ repeats. The adjacent genes are flanked on one side by a nucleic acid molecule comprising a nucleotide sequence from the 5′ region of the BMT2 gene (SEQ ID NO:11) and on the other side by a nucleic acid molecule comprising a nucleotide sequence from the 3′ region of the BMT2 gene (SEQ ID NO:12). Plasmid pGLY43a was linearized with SfiI and the linearized plasmid transformed into strain YGLY4-3 to produce to produce a number of strains in which the KlMNN2-2 gene and URA5 gene flanked by the lacZ repeats has been inserted into the BMT2 locus by double-crossover homologous recombination. The BMT2 gene was described in Mille et al., J. Biol. Chem. 283: 9724-9736 (2008) and U.S. Pat. No. 7,465,557. Strain YGLY6-3 was selected from the strains produced and is prototrophic for uracil. Strain YGLY6-3 was counterselected in the presence of 5-FOA to produce strains in which the URA5 gene has been lost and only the lacZ repeats remain. This renders the strain auxotrophic for uracil. Strain YGLY8-3 was selected.

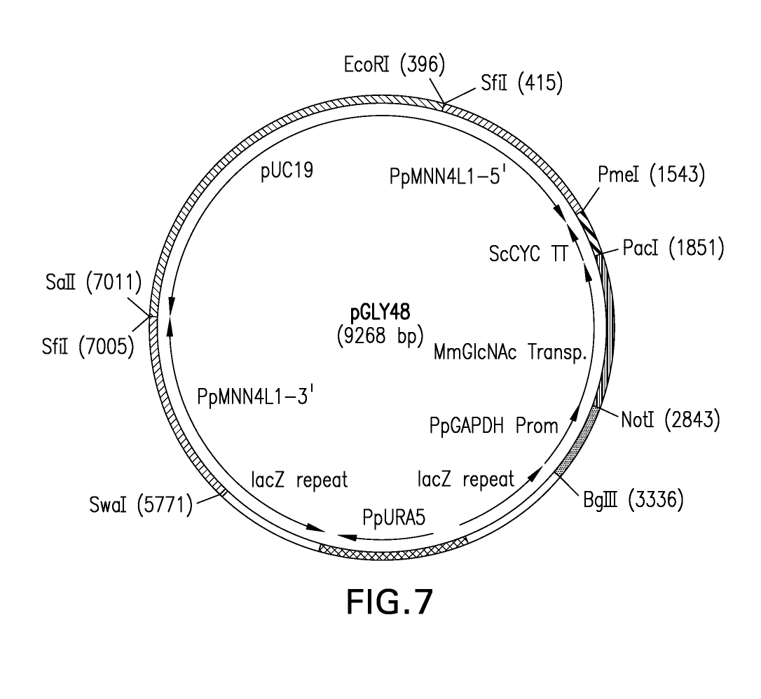

Plasmid pGLY48 (FIG. 7) is an integration vector that targets the MNN4L1 locus and contains an expression cassette comprising a nucleic acid molecule encoding the mouse homologue of the UDP-GlcNAc transporter (SEQ ID NO:13) open reading frame (ORF) operably linked at the 5′ end to a nucleic acid molecule comprising the P. pastoris GAPDH promoter (SEQ ID NO:14) and at the 3′ end to a nucleic acid molecule comprising the S. cerevisiae CYC termination sequences (SEQ ID NO:15) adjacent to a nucleic acid molecule comprising the P. pastoris URA5 gene flanked by lacZ repeats and in which the expression cassettes together are flanked on one side by a nucleic acid molecule comprising a nucleotide sequence from the 5′ region of the P. pastoris MNN4L1 gene (SEQ ID NO:16) and on the other side by a nucleic acid molecule comprising a nucleotide sequence from the 3′ region of the MNN4L1 gene (SEQ ID NO:17). Plasmid pGLY48 was linearized with SfiI and the linearized plasmid transformed into strain YGLY8-3 to produce a number of strains in which the expression cassette encoding the mouse UDP-GlcNAc transporter and the URA5 gene have been inserted into the MNN4L1 locus by double-crossover homologous recombination. The MNN4L1 gene (also referred to as MNN4B) has been disclosed in U.S. Pat. No. 7,259,007. Strain YGLY10-3 was selected from the strains produced and then counterselected in the presence of 5-FOA to produce a number of strains in which the URA5 gene has been lost and only the lacZ repeats remain. Strain YGLY12-3 was selected.

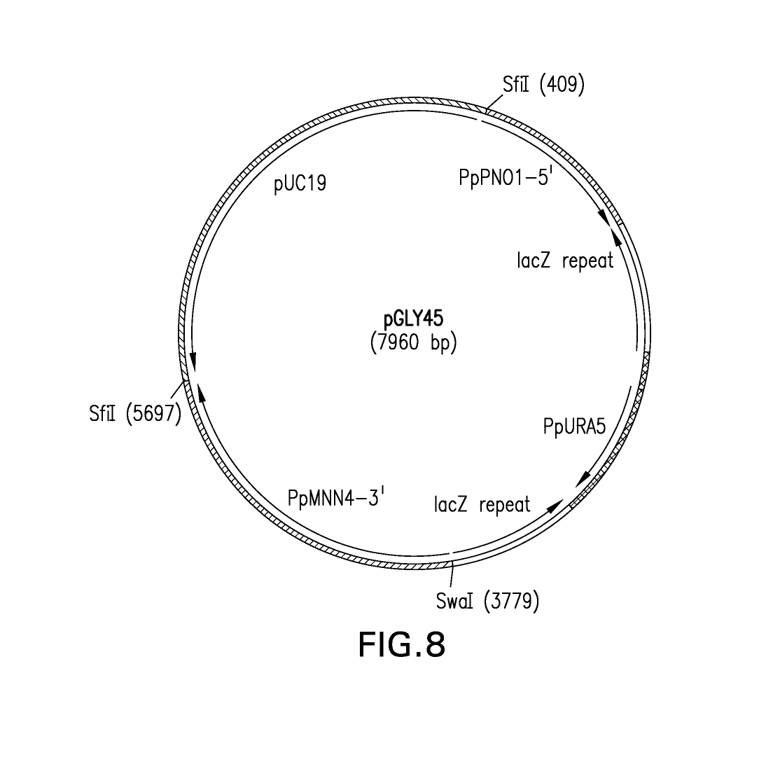

Plasmid pGLY45 (FIG. 8) is an integration vector that targets the PNO1/MNN4 loci and contains a nucleic acid molecule comprising the P. pastoris URA5 gene or transcription unit flanked by nucleic acid molecules comprising lacZ repeats which in turn is flanked on one side by a nucleic acid molecule comprising a nucleotide sequence from the 5′ region of the PNO1 gene (SEQ ID NO:18) and on the other side by a nucleic acid molecule comprising a nucleotide sequence from the 3′ region of the MNN4 gene (SEQ ID NO:19). Plasmid pGLY45 was linearized with SfiI and the linearized plasmid transformed into strain YGLY12-3 to produce a number of strains in which the URA5 gene flanked by the lacZ repeats has been inserted into the PNO1/MNN4 loci by double-crossover homologous recombination. The PNO1 gene has been disclosed in U.S. Pat. No. 7,198,921 and the MNN4 gene (also referred to as MNN4B) has been disclosed in U.S. Pat. No. 7,259,007. Strain YGLY14-3 was selected from the strains produced and then counterselected in the presence of 5-FOA to produce a number of strains in which the URA5 gene has been lost and only the lacZ repeats remain. Strain YGLY16-3 was selected.

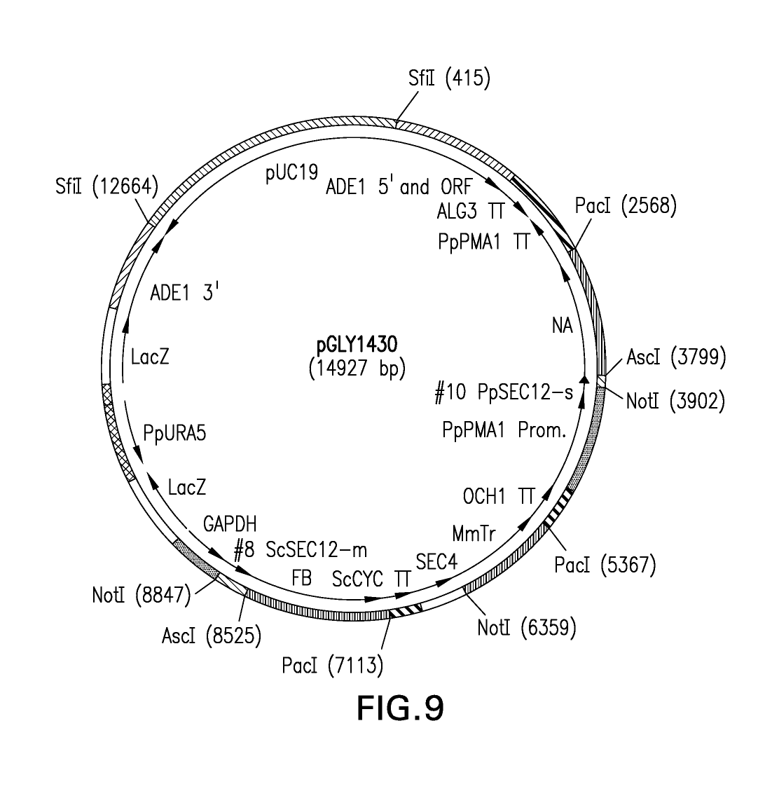

Plasmid pGLY1430 (FIG. 9) is a KINKO integration vector that targets the ADE1 locus without disrupting expression of the locus and contains in tandem four expression cassettes encoding (1) the human GlcNAc transferase I catalytic domain (NA) fused at the N-terminus to P. pastoris SEC12 leader peptide (10) to target the chimeric enzyme to the ER or Golgi, (2) mouse homologue of the UDP-GlcNAc transporter (MmTr), (3) the mouse mannosidase IA catalytic domain (FB) fused at the N-terminus to S. cerevisiae SEC12 leader peptide (8) to target the chimeric enzyme to the ER or Golgi, and (4) the P. pastoris URA5 gene or transcription unit. KINKO (Knock-In with little or No Knock-Out) integration vectors enable insertion of heterologous DNA into a targeted locus without disrupting expression of the gene at the targeted locus and have been described in U.S. Published Application No. 20090124000. The expression cassette encoding the NA10 comprises a nucleic acid molecule encoding the human GlcNAc transferase I catalytic domain codon-optimized for expression in P. pastoris (SEQ ID NO:20) fused at the 5′ end to a nucleic acid molecule encoding the SEC12 leader 10 (SEQ ID NO:21), which is operably linked at the 5′ end to a nucleic acid molecule comprising the P. pastoris PMA1 promoter and at the 3′ end to a nucleic acid molecule comprising the P. pastoris PMA1 transcription termination sequence. The expression cassette encoding MmTr comprises a nucleic acid molecule encoding the mouse homologue of the UDP-GlcNAc transporter ORF operably linked at the 5′ end to a nucleic acid molecule comprising the P. P. pastoris SEC4 promoter (SEQ ID NO:22) and at the 3′ end to a nucleic acid molecule comprising the P. pastoris OCH1 termination sequences (SEQ ID NO:23). The expression cassette encoding the FB8 comprises a nucleic acid molecule encoding the mouse mannosidase IA catalytic domain (SEQ ID NO:24) fused at the 5′ end to a nucleic acid molecule encoding the SEC12-m leader 8 (SEQ ID NO:25), which is operably linked at the 5′ end to a nucleic acid molecule comprising the P. pastoris GADPH promoter and at the 3′ end to a nucleic acid molecule comprising the S. cerevisiae CYC transcription termination sequence. The URA5 expression cassette comprises a nucleic acid molecule comprising the P. pastoris URA5 gene or transcription unit flanked by nucleic acid molecules comprising lacZ repeats. The four tandem cassettes are flanked on one side by a nucleic acid molecule comprising a nucleotide sequence from the 5′ region and complete ORF of the ADEJ gene (SEQ ID NO:26) followed by a P. pastoris ALG3 termination sequence (SEQ ID NO:27) and on the other side by a nucleic acid molecule comprising a nucleotide sequence from the 3′ region of the ADE1 gene (SEQ ID NO:28). Plasmid pGLY1430 was linearized with SfiI and the linearized plasmid transformed into strain YGLY16-3 to produce a number of strains in which the four tandem expression cassette have been inserted into the ADE1 locus immediately following the ADE1 ORF by double-crossover homologous recombination. The strain YGLY2798 was selected from the strains produced and is auxotrophic for arginine and now prototrophic for uridine, histidine, and adenine. The strain was then counterselected in the presence of 5-FOA to produce a number of strains now auxotrophic for uridine. Strain YGLY3794 was selected and is capable of making glycoproteins that have predominantly galactose terminated N-glycans.

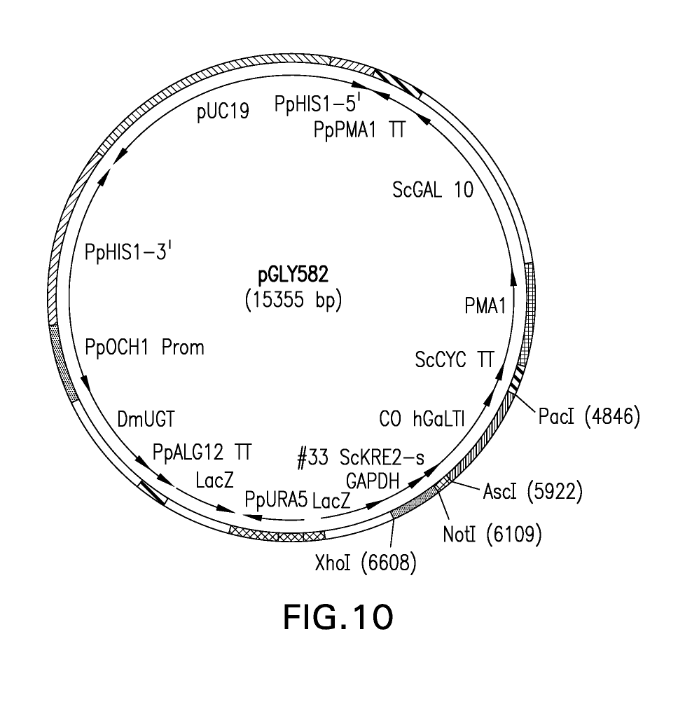

Plasmid pGLY582 (FIG. 10) is an integration vector that targets the HIS1 locus and contains in tandem four expression cassettes encoding (1) the S. cerevisiae UDP-glucose epimerase (ScGAL10), (2) the human galactosyltransferase I (hGalT) catalytic domain fused at the N-terminus to the S. cerevisiae KRE2-s leader peptide (33) to target the chimeric enzyme to the ER or Golgi, (3) the P. pastoris URA5 gene or transcription unit flanked by lacZ repeats, and (4) the D. melanogaster UDP-galactose transporter (DmUGT). The expression cassette encoding the ScGAL10 comprises a nucleic acid molecule encoding the ScGAL10 ORF (SEQ ID NO:29) operably linked at the 5′ end to a nucleic acid molecule comprising the P. pastoris PMA1 promoter (SEQ ID NO:30) and operably linked at the 3′ end to a nucleic acid molecule comprising the P. pastoris PMA1 transcription termination sequence (SEQ ID NO:31). The expression cassette encoding the chimeric galactosyltransferase I comprises a nucleic acid molecule encoding the hGalT catalytic domain codon optimized for expression in P. pastoris (SEQ ID NO:32) fused at the 5′ end to a nucleic acid molecule encoding the KRE2-s leader 33 (SEQ ID NO:33), which is operably linked at the 5′ end to a nucleic acid molecule comprising the P. pastoris GAPDH promoter and at the 3′ end to a nucleic acid molecule comprising the S. cerevisiae CYC transcription termination sequence. The URA5 expression cassette comprises a nucleic acid molecule comprising the P. pastoris URA5 gene or transcription unit flanked by nucleic acid molecules comprising lacZ repeats. The expression cassette encoding the DmUGT comprises a nucleic acid molecule encoding the DmUGT ORF (SEQ ID NO:34) operably linked at the 5′ end to a nucleic acid molecule comprising the P. pastoris OCH1 promoter (SEQ ID NO:35) and operably linked at the 3′ end to a nucleic acid molecule comprising the P. pastoris ALG12 transcription termination sequence (SEQ ID NO:36). The four tandem cassettes are flanked on one side by a nucleic acid molecule comprising a nucleotide sequence from the 5′ region of the HIS1 gene (SEQ ID NO:37) and on the other side by a nucleic acid molecule comprising a nucleotide sequence from the 3′ region of the HIS1 gene (SEQ ID NO:38). Plasmid pGLY582 was linearized and the linearized plasmid transformed into strain YGLY3794 to produce a number of strains in which the four tandem expression cassette have been inserted into the HIS1 locus by homologous recombination. Strain YGLY3853 was selected and is auxotrophic for histidine and prototrophic for uridine.

Plasmid pGLY167b (FIG. 11) is an integration vector that targets the ARG1 locus and contains in tandem three expression cassettes encoding (1) the D. melanogaster mannosidase II catalytic domain (KD) fused at the N-terminus to S. cerevisiae MNN2 leader peptide (53) to target the chimeric enzyme to the ER or Golgi, (2) the P. pastoris HIS1 gene or transcription unit, and (3) the rat N-acetylglucosamine (GlcNAc) transferase II catalytic domain (TC) fused at the N-terminus to S. cerevisiae MNN2 leader peptide (54) to target the chimeric enzyme to the ER or Golgi. The expression cassette encoding the KD53 comprises a nucleic acid molecule encoding the D. melanogaster mannosidase II catalytic domain codon-optimized for expression in P. pastoris (SEQ ID NO:39) fused at the 5′ end to a nucleic acid molecule encoding the MNN2 leader 53 (SEQ ID NO:40), which is operably linked at the 5′ end to a nucleic acid molecule comprising the P. pastoris GAPDH promoter and at the 3′ end to a nucleic acid molecule comprising the S. cerevisiae CYC transcription termination sequence. The HIS1 expression cassette comprises a nucleic acid molecule comprising the P. pastoris HIS1 gene or transcription unit (SEQ ID NO:41). The expression cassette encoding the TC54 comprises a nucleic acid molecule encoding the rat GlcNAc transferase II catalytic domain codon-optimized for expression in P. pastoris (SEQ ID NO:42) fused at the 5′ end to a nucleic acid molecule encoding the MNN2 leader 54 (SEQ ID NO:43), which is operably linked at the 5′ end to a nucleic acid molecule comprising the P. pastoris PMA1 promoter and at the 3′ end to a nucleic acid molecule comprising the P. pastoris PAM1 transcription termination sequence. The three tandem cassettes are flanked on one side by a nucleic acid molecule comprising a nucleotide sequence from the 5′ region of the ARG1 gene (SEQ ID NO:44) and on the other side by a nucleic acid molecule comprising a nucleotide sequence from the 3′ region of the ARG1 gene (SEQ ID NO:45). Plasmid pGLY167b was linearized with SfiI and the linearized plasmid transformed into strain YGLY3853 to produce a number of strains (in which the three tandem expression cassette have been inserted into the ARG1 locus by double-crossover homologous recombination. The strain YGLY4754 was selected from the strains produced and is auxotrophic for arginine and prototrophic for uridine and histidine. The strain was then counterselected in the presence of 5-FOA to produce a number of strains now auxotrophic for uridine. Strain YGLY4799 was selected.

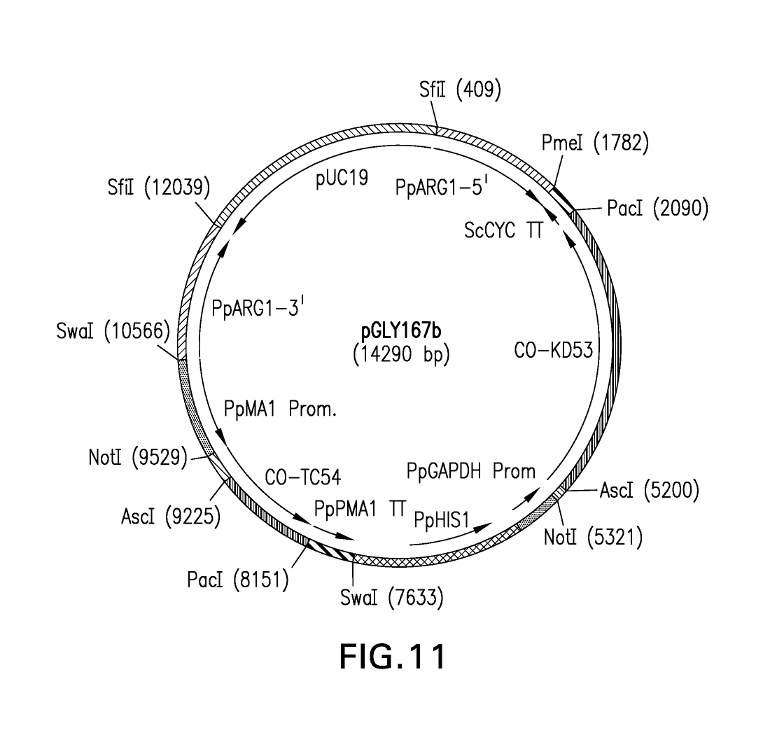

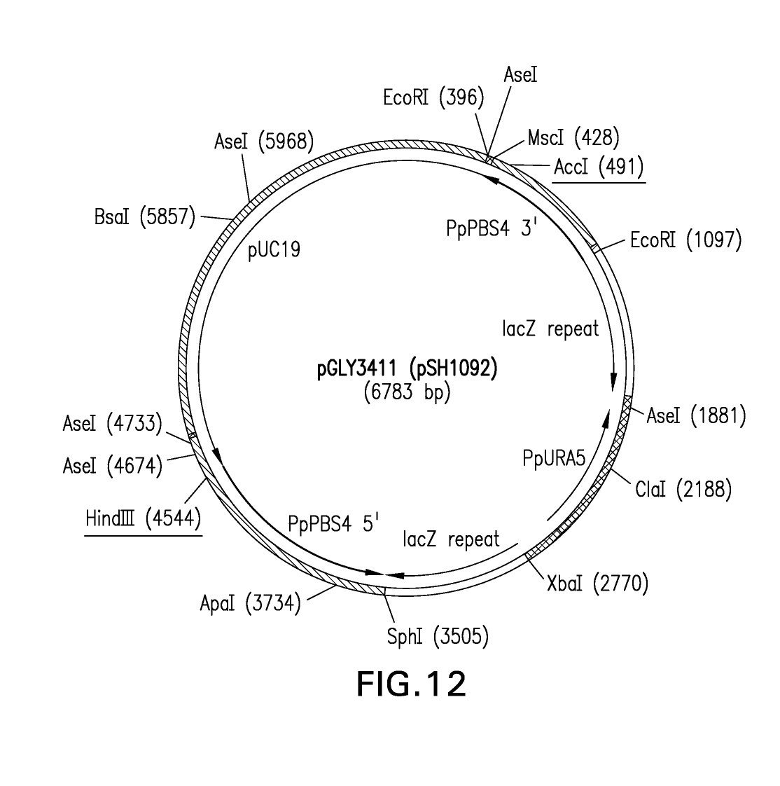

Plasmid pGLY3411 (FIG. 12) is an integration vector that contains the expression cassette comprising the P. pastoris URA5 gene flanked by lacZ repeats flanked on one side with the 5′ nucleotide sequence of the P. pastoris BMT4 gene (SEQ ID NO:46) and on the other side with the 3′ nucleotide sequence of the P. pastoris BMT4 gene (SEQ ID NO:47). Plasmid pGLY3411 was linearized and the linearized plasmid transformed into YGLY4799 to produce a number of strains in which the URA5 expression cassette has been inserted into the BMT4 locus by double-crossover homologous recombination. Strain YGLY6903 was selected from the strains produced and is prototrophic for uracil, adenine, histidine, proline, arginine, and tryptophan. The strain was then counterselected in the presence of 5-FOA to produce a number of strains now auxotrophic for uridine. Strains YGLY7432 and YGLY7433 were selected.

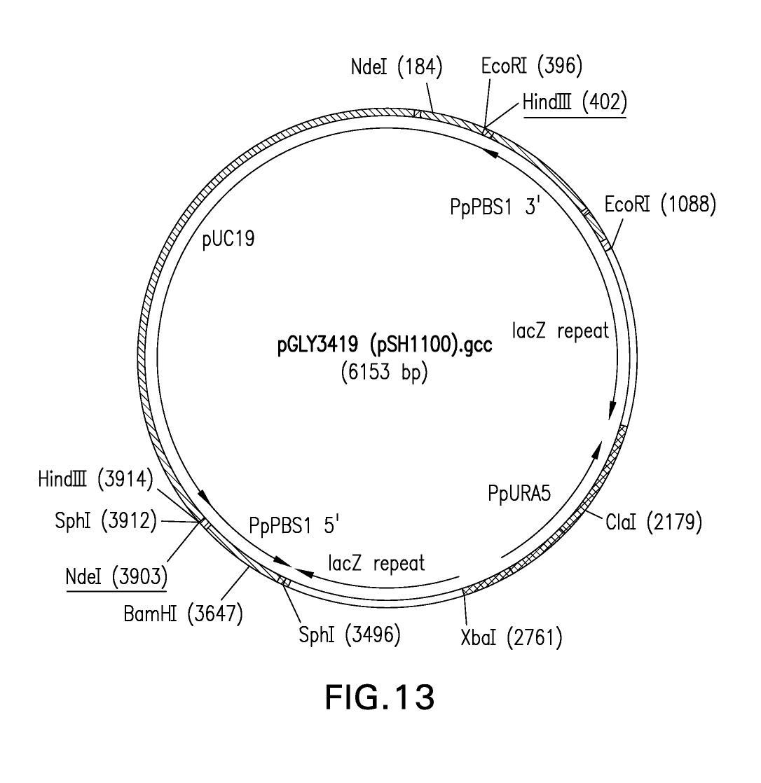

Plasmid pGLY3419 (FIG. 13) is an integration vector that contains an expression cassette comprising the P. pastoris URA5 gene flanked by lacZ repeats flanked on one side with the 5′ nucleotide sequence of the P. pastoris BMT1 gene (SEQ ID NO:48) and on the other side with the 3′ nucleotide sequence of the P. pastoris BMT1 gene (SEQ ID NO:49). Plasmid pGLY3419 was linearized and the linearized plasmid transformed into strain YGLY7432 and YGLY7433 to produce a number of strains in which the URA5 expression cassette has been inserted into the BMT1 locus by double-crossover homologous recombination. The strains YGLY7656 and YGLY7651 were selected from the strains produced and are prototrophic for uracil, adenine, histidine, proline, arginine, and tryptophan. The strains were then counterselected in the presence of 5-FOA to produce a number of strains now auxotrophic for uridine. Strains YGLY7930 and YGLY7940 were selected.

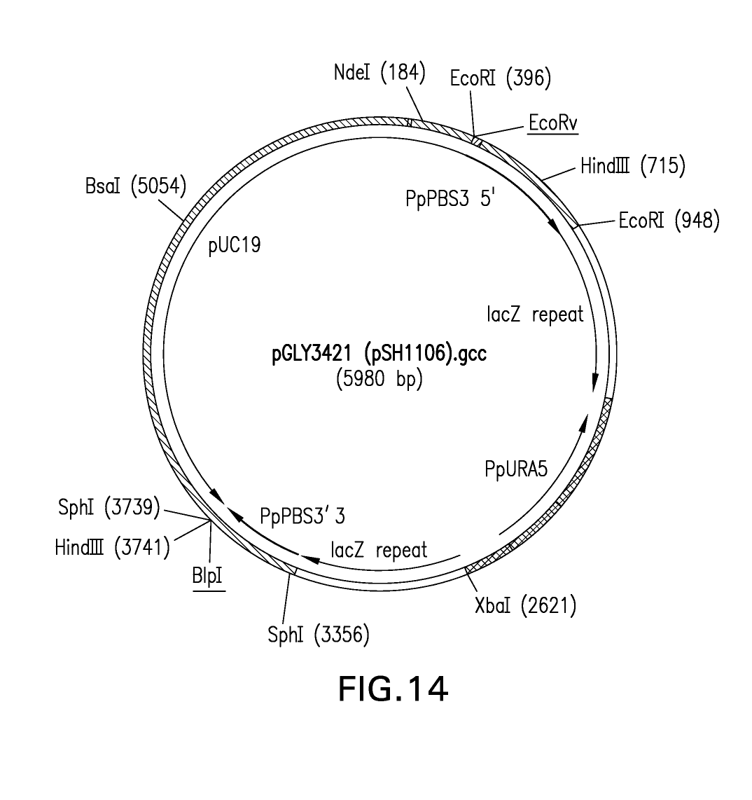

Plasmid pGLY3421 (FIG. 14) is an integration vector that contains an expression cassette comprising the P. pastoris URA5 gene flanked by lacZ repeats flanked on one side with the 5′ nucleotide sequence of the P. pastoris BMT3 gene (SEQ ID NO:50) and on the other side with the 3′ nucleotide sequence of the P. pastoris BMT3 gene (SEQ ID NO:51). Plasmid pGLY3419 was linearized and the linearized plasmid transformed into strain YGLY7930 and YGLY7940 to produce a number of strains in which the URA5 expression cassette has been inserted into the BMT1 locus by double-crossover homologous recombination. The strains YGLY7965 and YGLY7961 were selected from the strains produced and are prototrophic for uracil, adenine, histidine, proline, arginine, and tryptophan.

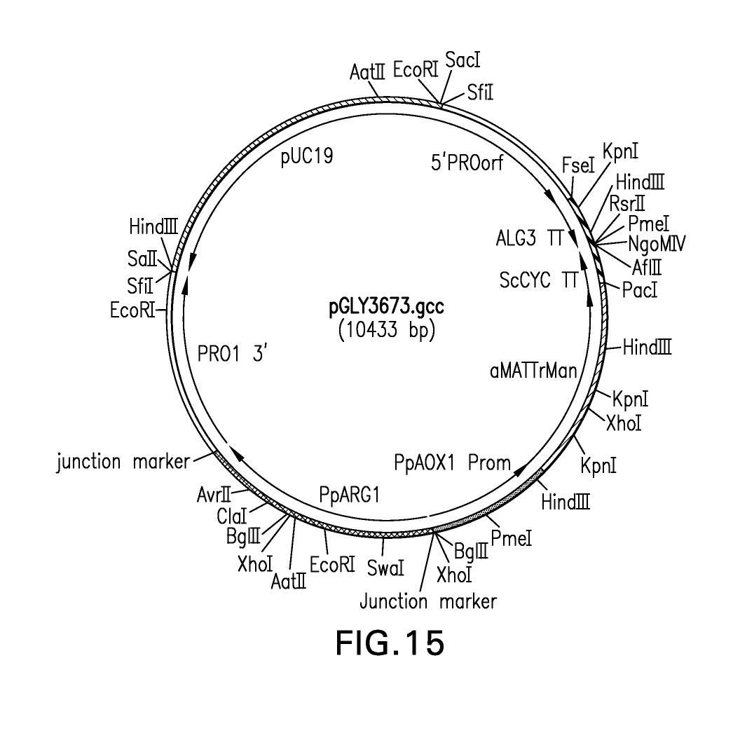

Plasmid pGLY3673 (FIG. 15) is a KINKO integration vector that targets the PRO1 locus without disrupting expression of the locus and contains expression cassettes encoding the T. reesei α-1,2-mannosidase catalytic domain fused at the N-terminus to S. cerevisiae aMATpre signal peptide (aMATTrMan) to target the chimeric protein to the secretory pathway and secretion from the cell. The expression cassette encoding the aMATTrMan comprises a nucleic acid molecule encoding the T. reesei catalytic domain (SEQ ID NO:52) fused at the 5′ end to a nucleic acid molecule encoding the S. cerevisiae aMATpre signal peptide (SEQ ID NO:53, 54), which is operably linked at the 5′ end to a nucleic acid molecule comprising the P. pastoris AOX1 promoter (SEQ ID NO:55) and at the 3′ end to a nucleic acid molecule comprising the S. cerevisiae CYC transcription termination sequence (SEQ ID NO:15). The cassette is flanked on one side by a nucleic acid molecule comprising a nucleotide sequence from the 5′ region and complete ORF of the ARG1 gene (SEQ ID NO:56) followed by a P. pastoris ALG3 termination sequence and on the other side by a nucleic acid molecule comprising a nucleotide sequence from the 3′ region of the ARG1 gene (SEQ ID NO:57). Plasmid pGLY3673 was linearized and the linearized plasmid transformed into strains YGLY7965 and YGLY7961 to produce a number of strains in which the URA5 expression cassette has been inserted into the BMT1 locus by double-crossover homologous recombination. The strains YGLY78316 and YGLY8323 were selected from the strains produced and are prototrophic for uracil, adenine, histidine, proline, arginine, and tryptophan.

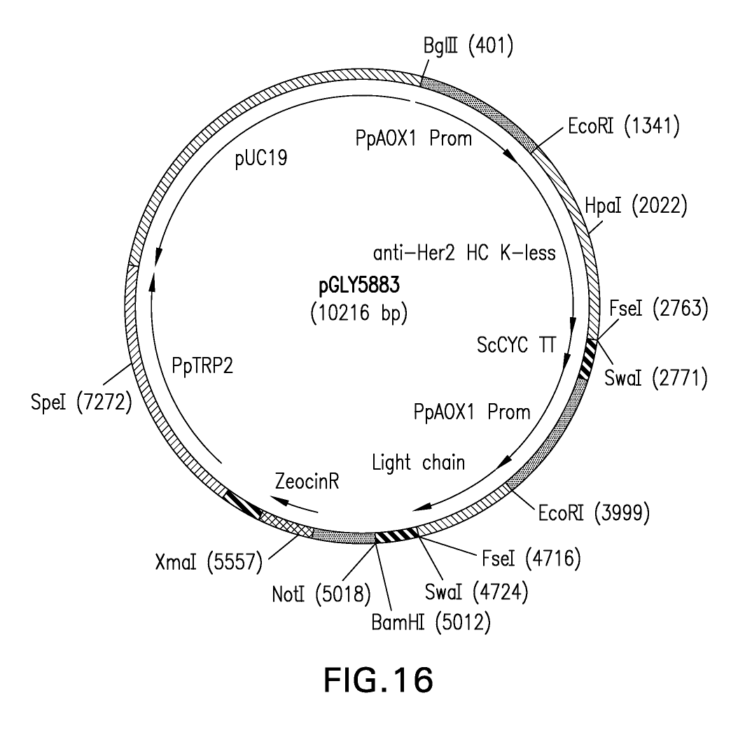

Plasmid p GLY5883 (FIG. 16) is a roll-in integration plasmid encoding the light and heavy chains of an anti-Her2 antibody that targets the TRP2 locus in P. pastoris. The expression cassette encoding the anti-Her2 heavy chain comprises a nucleic acid molecule encoding the heavy chain ORF codon-optimized for effective expression in P. pastoris (SEQ ID NO:58) operably linked at the 5′ end to a nucleic acid molecule encoding the Saccharomyces cerevisiae mating factor pre-signal sequence (SEQ ID NO:53) which in turn is fused at its N-terminus to a nucleic acid molecule that has the inducible P. pastoris AOX1 promoter sequence (SEQ ID NO:55) and at the 3′ end to a nucleic acid molecule that has the S. cerevisiae CYC transcription termination sequence (SEQ ID NO:15). The expression cassette encoding the anti-Her2 light chain comprises a nucleic acid molecule encoding the light chain ORF codon-optimized for effective expression in P. pastoris (SEQ ID NO:59) operably linked at the 5′ end to a nucleic acid molecule encoding the Saccharomyces cerevisiae mating factor pre-signal sequence (SEQ ID NO:53) which in turn is fused at its N-terminus to a nucleic acid molecule that has the inducible P. pastoris AOX1 promoter sequence (SEQ ID NO:55) and at the 3′ end to a nucleic acid molecule that has the S. cerevisiae CYC transcription termination sequence (SEQ ID NO:15). For selecting transformants, the plasmid comprises an expression cassette encoding the Zeocin ORF in which the nucleic acid molecule encoding the ORF (SEQ ID NO:60) is operably linked at the 5′ end to a nucleic acid molecule having the S. cerevisiae TEF promoter sequence (SEQ ID NO:61) and at the 3′ end to a nucleic acid molecule having the S. cerevisiae CYC transcription termination sequence (SEQ ID NO:15). The plasmid further includes a nucleic acid molecule for targeting the TRP2 locus (SEQ ID NO:62).

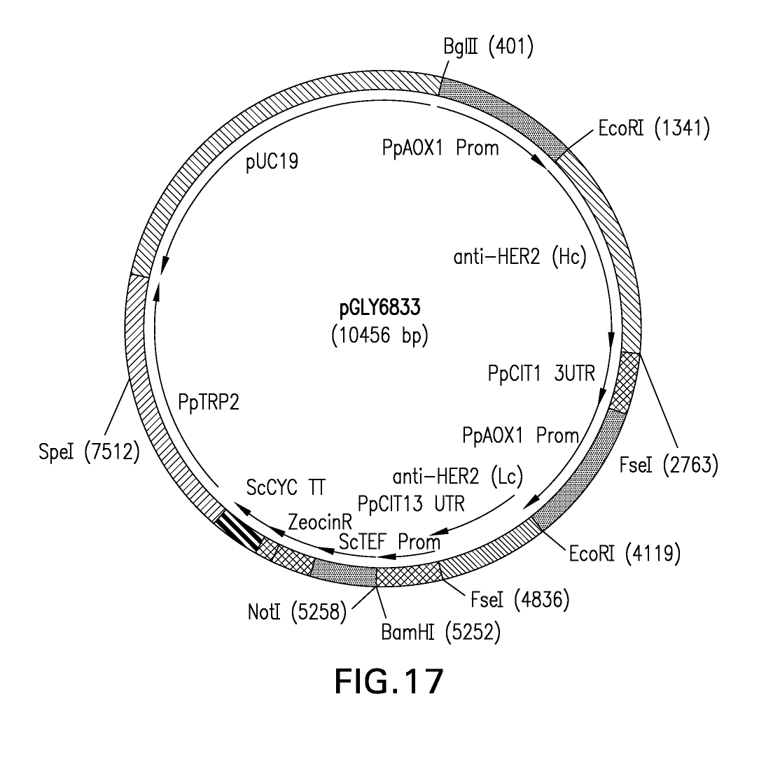

Plasmid p GLY6833 (FIG. 17) is a roll-in integration plasmid encoding the light and heavy chains of an anti-Her2 antibody that targets the TRP2 locus in P. pastoris. The expression cassette encoding the anti-Her2 heavy chain comprises a nucleic acid molecule encoding the heavy chain ORF codon-optimized for effective expression in P. pastoris (SEQ ID NO:58) operably linked at the 5′ end to a nucleic acid molecule encoding the Saccharomyces cerevisiae mating factor pre-signal sequence (SEQ ID NO:53) which in turn is fused at its N-terminus to a nucleic acid molecule that has the inducible P. pastoris AOX1 promoter sequence (SEQ ID NO:55) and at the 3′ end to a nucleic acid molecule that has the P. pastoris CIT1 transcription termination sequence (SEQ ID NO:63). The expression cassette encoding the anti-Her2 light chain comprises a nucleic acid molecule encoding the light chain ORF codon-optimized for effective expression in P. pastoris (SEQ ID NO:59) operably linked at the 5′ end to a nucleic acid molecule encoding the Saccharomyces cerevisiae mating factor pre-signal sequence (SEQ ID NO:53) which in turn is fused at its N-terminus to a nucleic acid molecule that has the inducible P. pastoris AOX1 promoter sequence (SEQ ID NO:55) and at the 3′ end to a nucleic acid molecule that has the P. pastoris CIT1 transcription termination sequence (SEQ ID NO:63). For selecting transformants, the plasmid comprises an expression cassette encoding the Zeocin ORF in which the nucleic acid molecule encoding the ORF (SEQ ID NO:60) is operably linked at the 5′ end to a nucleic acid molecule having the S. cerevisiae TEF promoter sequence (SEQ ID NO:61) and at the 3′ end to a nucleic acid molecule having the S. cerevisiae CYC transcription termination sequence (SEQ ID NO:15). The plasmid further includes a nucleic acid molecule for targeting the TRP2 locus (SEQ ID NO:62).

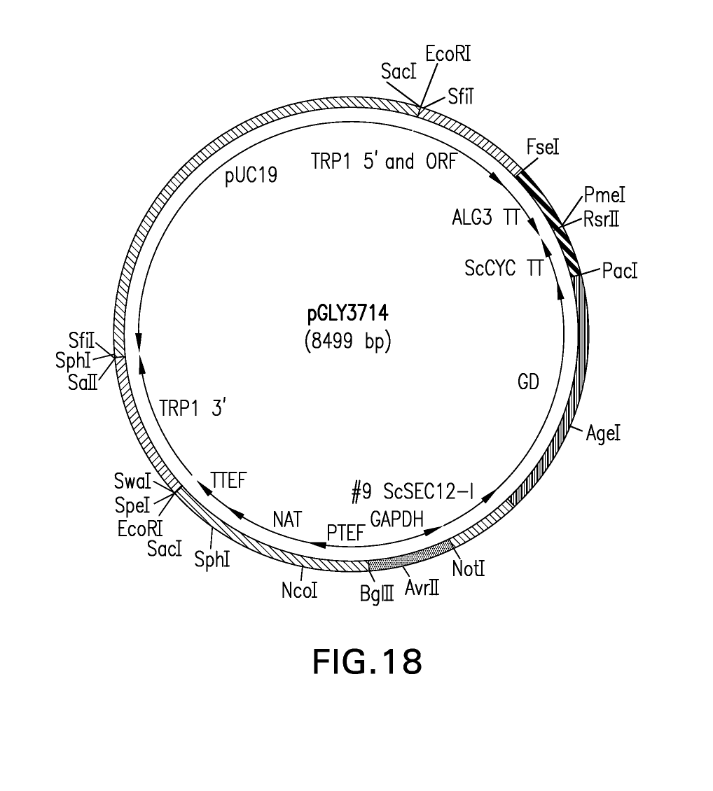

Plasmid pGLY3714 (FIG. 18) is a KINKO integration vector that targets the TRP1 locus without disrupting expression of the locus and contains expression cassettes encoding the mouse mannosidase IB catalytic domain (GD) fused at the N-terminus to S. cerevisiae SEC12 leader peptide (GD9) to target the chimeric enzyme to the ER or Golgi. For selecting transformants, the plasmid comprises an expression cassette encoding the Nourseothricin resistance (NAT^{R}) ORF (originally from pAG25 from EROSCARF, Scientific Research and Development GmbH, Daimlerstrasse 13a, D-61352 Bad Homburg, Germany, See Goldstein et al., Yeast 15: 1541 (1999)); wherein the nucleic acid molecule encoding the ORF (SEQ ID NO:64) is operably linked to at the 5′ end to a nucleic acid molecule having the Ashbya gossypii TEF1 promoter sequence (SEQ ID NO:65) and at the 3′ end to a nucleic acid molecule that has the Ashbya gossypii TEF1 termination sequence (SEQ ID NO:66). The two expression cassettes are flanked on one side by a nucleic acid molecule comprising a nucleotide sequence from the 5′ region of the ORF encoding Trp1p ending at the stop codon (SEQ ID NO:67 linked to a nucleic acid molecule having the P. pastoris ALG3 termination sequence (SEQ ID NO:27) and on the other side by a nucleic acid molecule comprising a nucleotide sequence from the 3′ region of the TRP1 gene (SEQ ID NO:68). Plasmid pGLY3714 was constructed by cloning the DNA fragment encoding the GD9 ORF flanked by a NotI site at the 5′ end and a Pad site at the 3′ end into plasmid pGLY597. An expression cassette comprising a nucleic acid molecule encoding the Nourseothricin resistance ORF (NAT) operably linked to the Ashbya gossypii TEF1 promoter (PTEF) and Ashbya gossypii TEF1 termination sequence (TTEF).

### EXAMPLE 3

### Engineered Pichia pastoris Host Strains Expressing Heterologous Proteins

Strain YGLY12501 was generated by transforming pGLY5883, which encodes the anti-Her2 antibody, into YGLY8316. The strain YGLY12501 was selected from the strains produced. In this strain, the expression cassettes encoding the anti-Her2 heavy and light chains are targeted to the Pichia pastoris TRP2 locus (PpTRP2). This strain contains the wild-type XRN1 sequence.

Strain YGLY13992 was generated by transforming pGLY6833, which encodes the anti-Her2 antibody, into YGLY8316. The strain YGLY13992 was selected from the strains produced. In this strain, the expression cassettes encoding the anti-Her2 heavy and light chains are targeted to the Pichia pastoris TRP2 locus (PpTRP2). This strain contains the wild-type XRN1 sequence.

Strain YGLY12511 was generated by transforming pGLY5883, which encodes the anti-Her2 antibody, into YGLY8316. The strain YGLY12511 was selected from the strains produced. Strain YGLY14836 was generated by transforming pGLY3714, which encodes the GD9, into YGLY12511. The strain YGLY14836 was selected from the strains produced. In this strain, the expression cassettes encoding the anti-Her2 heavy and light chains are targeted to the Pichia pastoris TRP2 locus (PpTRP2). This strain contains the wild-type XRN1 sequence.

Transformation of the appropriate strains disclosed herein with pGLY7392 XRN1 knock-out plasmid vector was performed essentially as follows. Appropriate Pichia pastoris strains were grown in 50 mL YPD media (yeast extract (1%), peptone (2%), and dextrose (2%)) overnight to an OD of about 0.2 to 6. After incubation on ice for 30 minutes, cells were pelleted by centrifugation at 2500-3000 rpm for five minutes. Media was removed and the cells washed three times with ice cold sterile 1 M sorbitol before resuspension in 0.5 mL ice cold sterile 1 M sorbitol. Ten μL linearized DNA (5-20 μg) and 100 μL cell suspension was combined in an electroporation cuvette and incubated for 5 minutes on ice. Electroporation was in a Bio-Rad GenePulser Xcell following the preset Pichia pastoris protocol (2 kV, 25 μF, 200 0), immediately followed by the addition of 1 mL YPDS recovery media (YPD media plus 1 M sorbitol). The transformed cells were allowed to recover for four hours to overnight at room temperature (24° C.) before plating the cells on selective media.

Strains YGLY13992, YGLY12501 and YGLY14836 were each then transformed with pGLY7392 as described above to produce the strains described in Example 4.

### EXAMPLE 4

Engineered Pichia pastoris Xrn1Δ Strains for Improved Fermentation Yield and N-Glycosylation Quality

The XRN1 knock-out integration plasmid pGLY7392 was linearized with SfiI and the linearized plasmid was transformed into each of the Pichia pastoris strains YGLY12501, YGLY13992, and YGLY14836 to produce respective Δxrn1 strains (i.e., xrn1 deletion strains) used in the following examples. Transformations were performed essentially as described in Example 3.

The genomic integration of pGLY7392 at the XRN1 locus was confirmed by cPCR using the primers, PpXRN1-5′ out/UP (5′-GTTAAATGACTCTAACACCTTGCACTTGA-3′; SEQ ID NO:69) and PpALG3TT/LP (5′-CCTCCCACTGGAACCGATGATATGGAA-3′; SEQ ID NO:70) or PpTEFTT/UP (5′-GATGCGAAGTTAAGTGCGCAGAAAGTAATATCA-3′; SEQ ID NO:71) and PpXRN1-3′ out/LP (5′-TTGCAAAAACCAGTGAGGAATAGC-3; SEQ ID NO:72). Loss of genomic XRN1 sequences was confirmed using cPCR primers, PpXRN1/iUP (5′-GAATGCTGAAGAACGTCAAAGAAACT-3′ (SEQ ID NO:73) and PpXRN1/iLP (5′-TGAGACTTCAGAGCTTTCCATACGA-3′ (SEQ ID NO:74). The PCR conditions were one cycle of 95° C. for two minutes, 35 cycles of 95° C. for 20 seconds, 52° C. for 20 seconds, and 72° C. for one minute; followed by one cycle of 72° C. for 10 minutes.

The strains were cultivated in either a DasGip 1 Liter or Micro24 5 mL fermentor to produce the antibodies for titer and protein N-glycosylation analyses.

Cell growth conditions of the transformed strains for antibody production in the Micro24 5 mL fermentor were generally as follows. Protein expression for the transformed yeast strain seed cultures were prepared by adding Pichia pastoris cells from YSD plates to each well of a Whatman 24-well Uniplate (10 ml, natural polypropylene) containing 3.5 ml of 4% BMGY medium buffered to pH 6.0 with potassium phosphate buffer. The seed cultures were grown for approximately 65-72 hours in a temperature controlled shaker at 24° C. and 650 rpm agitation. 1.0 mL of the 24 well plate grown seed culture and 4.0 ml of 4% BMGY medium was then used to inoculate each well of a Micro24 plate (Type:REG2). 30 μl of Antifoam 204 (1:25 dilution, Sigma Aldrich) was added to each well. The Micro24 was operated in Microaerobicl mode and the fermentations were controlled at 200% dissolved oxygen, pH at 6.5, temperature at 24° C. and agitation at 800 rpm. The induction phase was initiated upon observance of a DO spike after the growth phase by adding bolus shots of methanol feed solution (100% [w/w] methanol, 5 mg/l biotin and 12.5 ml/l PTM1 salts).

Cell growth conditions of the transformed strains for.antibody production in the DasGip fermentor were generally as follows. Protein expression for the transformed yeast strains was carried out in shake flasks at 24° C. with buffered glycerol-complex medium (BMGY) consisting of 1% yeast extract, 2% peptone, 100 mM potassium phosphate buffer pH 6.0, 1.34% yeast nitrogen base, 4×10⁻⁵% biotin, and 4% glycerol. The induction medium for protein expression was buffered methanol-complex medium (BMMY) consisting of 1% methanol instead of glycerol in BMGY. Pmt inhibitor Pmti-3 (5-[[3-(1-Phenyl-2-hydroxy)ethoxy)-4-(2-phenylethoxy)]phenyl]methylene]-4-oxo-2-thioxo-3-thiazolidineacetic Acid) (See Published International Application No. WO 2007061631) in methanol was added to the growth medium to a final concentration of 18.3 μM at the time the induction medium was added. Cells were harvested and centrifuged at 2,000 rpm for five minutes.

DasGip Fermentor Screening Protocol followed the parameters shown in Table 2.

TABLE 2
DasGip Fermentor Parameters

| Parameter | Set-point | Actuated Element |
|---|---|---|
| pH | 6.5 ± 0.1 | 30% NH₄OH |
| Temperature | 24 ± 0.1 | Cooling Water & Heating Blanket |
| Dissolved O2 | n/a | Initial impeller speed of 550 rpm is |
| | | ramped to 1200 rpm over first 10 hr, then |
| | | fixed at 1200 rpm for remainder of run |

At time of about 18 hours post-inoculation, DasGip vessels containing 350 mL media A (See Table 3 below) plus 4% glycerol were inoculated with strain of interest. A small dose (0.3 mL of 0.2 mg/mL in 100% methanol) of Pmti-3 was added with inoculum. At time about 20 hour, a bolus of 17 mL 50% glycerol solution (Glycerol Fed-Batch Feed, See Table 4 below) plus a larger dose (0.3 mL of 4 mg/mL) of Pmti-3 was added per vessel. At about 26 hours, when the glycerol was consumed, as indicated by a positive spike in the dissolved oxygen (DO) concentration, a methanol feed (See Table 5 below) was initiated at 0.7 mL/hr continuously. At the same time, another dose of Pmti-3 (0.3 mL of 4 mg/mL stock) was added per vessel. At time about 48 hours, another dose (0.3 mL of 4 mg/mL) of Pmti-3 was added per vessel. Cultures were harvested and processed at about 60 hours post-inoculation.

TABLE 3
Composition of Media A

| | | |
|---|---|---|
| Soytone L-1 | 20 | g/L |
| Yeast Extract | 10 | g/L |
| KH₂PO4 | 11.9 | g/L |
| K₂HPO₄ | 2.3 | g/L |
| Sorbitol | 18.2 | g/L |
| Glycerol | 40 | g/L |
| Antifoam Sigma 204 | 8 | drops/L |
| 10X YNB w/Ammonium Sulfate | 100 | mL/L |
| w/o Amino Acids (134 g/L) | | |
| 250X Biotin (0.4 g/L) | 10 | mL/L |
| 500X Chloramphenicol (50 g/L) | 2 | mL/L |
| 500X Kanamycin (50 g/L) | 2 | mL/L |

TABLE 4
Glycerol Fed-Batch Feed

| | | |
|---|---|---|
| Glycerol | 50 | % m/m |
| PTM1 Salts (see Table IV-E below) | 12.5 | mL/L |
| 250X Biotin (0.4 g/L) | 12.5 | mL/L |

TABLE 5
Methanol Feed

| | | |
|---|---|---|
| Methanol | 100 | % m/m |
| PTM1 Salts (See Table 6) | 12.5 | mL/L |
| 250X Biotin (0.4 g/L) | 12.5 | mL/L |

TABLE 6
PTM1 Salts

| | | |
|---|---|---|
| CuSO₄—5H₂O | 6 | g/L |
| NaI | 80 | mg/L |
| MnSO₄—7H₂O | 3 | g/L |
| NaMoO₄—2H₂O | 200 | mg/L |
| H₃BO₃ | 20 | mg/L |
| CoCl₂—6H₂O | 500 | mg/L |
| ZnCl₂ | 20 | g/L |
| FeSO₄—7H₂O | 65 | g/L |
| Biotin | 200 | mg/L |
| H₂SO₄ (98%) | 5 | mL/L |

The quality of N-glycan composition of the anti-Her2 antibodies was determined as follows. The antibodies were recovered from the cell culture medium and purified by protein A column chromatography. The N-glycans from protein A-purified antibodies were analyzed with 2AB labeling. The high performance liquid chromatography (HPLC) system used consisted of an Agilent 1200 equipped with autoinjector, a column-heating compartment and a UV detector detecting at 210 and 280 nm. All LC-MS experiments performed with this system were running at 1 mL/min. The flow rate was not split for MS detection. Mass spectrometric analysis was carried out in positive ion mode on Accurate-Mass Q-TOF LC/MS 6520 (Agilent technology). The temperature of dual ESI source was set at 350° C. The nitrogen gas flow rates were set at 13 L/h for the cone and 3501/h and nebulizer was set at 45 psig with 4500 volt applied to the capillary. Reference mass of 922.009 was prepared from HP-0921 according to API-TOF reference mass solution kit for mass calibration and the protein mass measurements. The data for ion spectrum range from 300-3000 m/z were acquired and processed using Agilent Masshunter.

Sample preparation was as follows. An intact antibody sample (50 μg) was prepared 50 μL 25 mM NH₄HCO₃, pH 7.8. For deglycosylated antibody, a 50 μL aliquot of intact antibody sample was treated with PNGase F (10 units) for 18 hours at 37° C. Reduced antibody was prepared by adding 1 M DTT to a final concentration of 10 mM to an aliquot of either intact antibody or deglycosylated antibody and incubated for 30 min at 37° C.

Three micrograms of intact or deglycosylated antibody sample was loaded onto a Poroshell 300SB-C3 column (2.1 mm×75 mm, 5 μm) (Agilent Technologies) maintained at 70°C. The protein was first rinsed on the cartridge for 1 minute with 90% solvent A (0.1% HCOOH), 5% solvent B (90% Acetonitrile in 0.1% HCOOH). Elution was then performed using a gradient of 5-100% of B over 26 minutes followed by a three-minute regeneration at 100% B and by a final equilibration period of 10 minute at 5% B.

For reduced antibody, a three microgram sample was loaded onto a Poroshell 300SB-C3 column (2.1 mm×75 mm, 5 μm) (Agilent Technologies) maintained at 40° C. The protein was first rinsed on the cartridge for three minutes with 90% solvent A, 5% solvent B. Elution was then performed using a gradient of 5-80% of B over 20 minutes followed by a seven-minute regeneration at 80% B and by a final equilibration period of 10 minutes at 5% B.

### EXAMPLE 5

### Production of Pichia pastoris Strains for Glycolnsulin Production

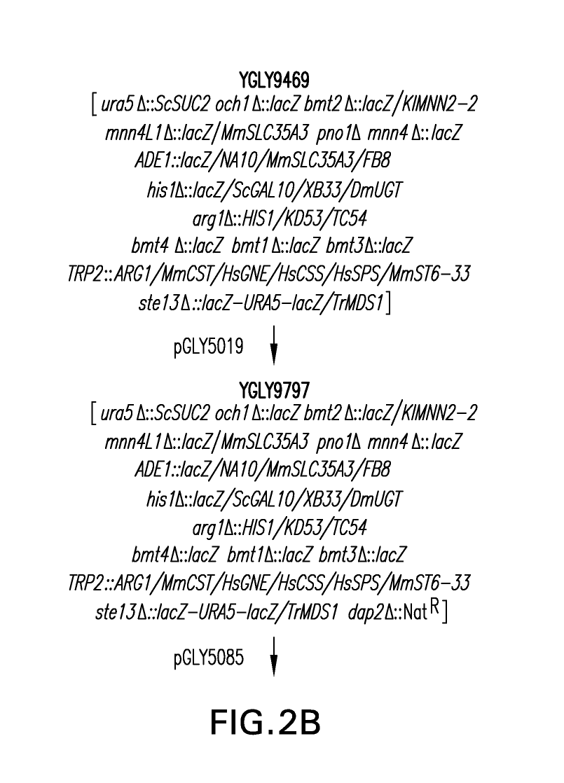

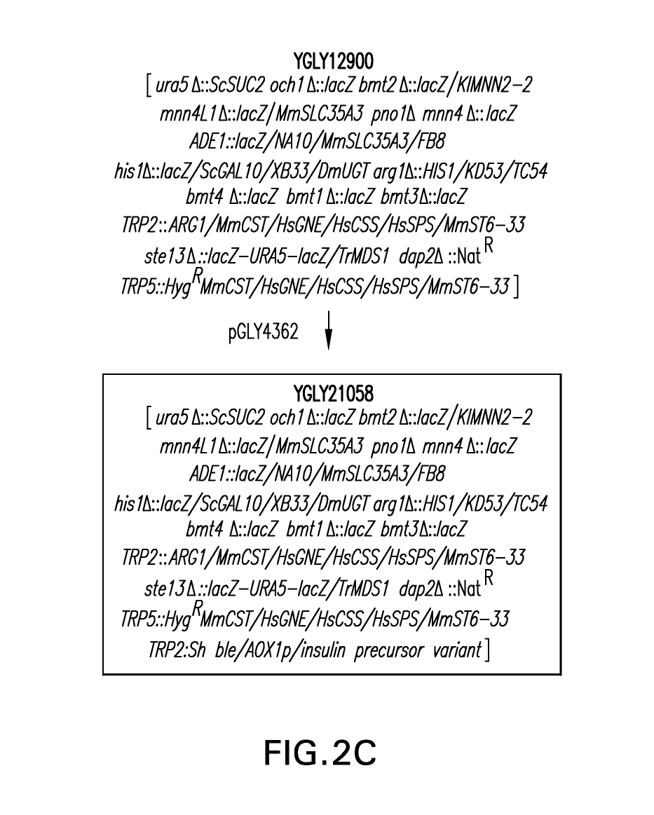

This example describes construction of strain YGLY21058. Genetically engineered Pichia pastoris strain YGLY21058 produces recombinant human glycoinsulin molecules. The strain produces glycoproteins having sialylated N-glycans and expressing the insulin analogue comprising an N-glycosylation site on the B-chain at position 28 encoded by the expression cassette in plasmid pGLY4362. Construction of the strains is illustrated schematically in FIGS. 2A-2D. Briefly, the strain YGLY21058 was constructed from glycoengineered Pichia pastoris strain YGLY7961 from Example 1 using methods described as follows:

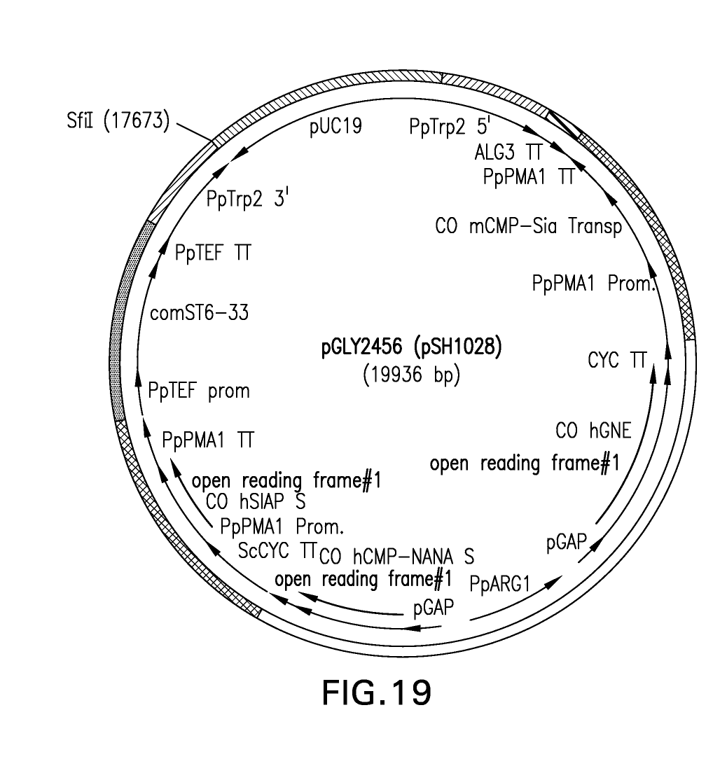

FIG. 19 shows as map of plasmid pGLY2456. Plasmid pGLY2456 is a KINKO integration vector that targets the TRP2 locus without disrupting expression of the locus and contains six expression cassettes encoding (1) the mouse CMP-sialic acid transporter (mCMP-Sia Transp), (2) the human UDP-GlcNAc 2-epimerase/N-acetylmannosamine kinase (hGNE), (3) the Pichia pastoris ARG1 gene or transcription unit, (4) the human CMP-sialic acid synthase (hCSS), (5) the human N-acetylneuraminate-9-phosphate synthase (hSPS), (6) the mouse α-2,6-sialyltransferase catalytic domain (mST6) fused at the N-terminus to S. cerevisiae KRE2 leader peptide (33) to target the chimeric enzyme to the ER or Golgi, and the P. pastoris ARG1 gene or transcription unit. The expression cassette encoding the mouse CMP-sialic acid transporter comprises a nucleic acid molecule encoding the mCMP Sia Transp ORF codon optimized for expression in P. pastoris (SEQ ID NO: 77), which is operably linked at the 5′ end to a nucleic acid molecule comprising the P. pastoris PMA1 promoter and at the 3′ end to a nucleic acid molecule comprising the P. pastoris PMA1 transcription termination sequence. The expression cassette encoding the human UDP-GlcNAc 2-epimerase/N-acetylmannosamine kinase comprises a nucleic acid molecule encoding the hGNE ORF codon optimized for expression in P. pastoris (SEQ ID NO: 78), which is operably linked at the 5′ end to a nucleic acid molecule comprising the P. pastoris GAPDH promoter and at the 3′ end to a nucleic acid molecule comprising the S. cerevisiae CYC transcription termination sequence. The expression cassette encoding the P. pastoris ARG1 gene comprises (SEQ ID NO: 79). The expression cassette encoding the human CMP-sialic acid synthase comprises a nucleic acid molecule encoding the hCSS ORF codon optimized for expression in P. pastoris (SEQ ID NO: 80), which is operably linked at the 5′ end to a nucleic acid molecule comprising the P. pastoris GAPDH promoter and at the 3′ end to a nucleic acid molecule comprising the S. cerevisiae CYC transcription termination sequence. The expression cassette encoding the human N-acetylneuraminate-9-phosphate synthase comprises a nucleic acid molecule encoding the hSIAP S ORF codon optimized for expression in P. pastoris (SEQ ID NO: 81), which is operably linked at the 5′ end to a nucleic acid molecule comprising the P. pastoris PMA1 promoter and at the 3′ end to a nucleic acid molecule comprising the P. pastoris PMA1 transcription termination sequence. The expression cassette encoding the chimeric mouse α-2,6-sialyltransferase comprises a nucleic acid molecule encoding the mST6 catalytic domain codon optimized for expression in P. pastoris (SEQ ID NO:82) fused at the 5′ end to a nucleic acid molecule encoding the S. cerevisiae KRE2 signal peptide, which is operably linked at the 5′ end to a nucleic acid molecule comprising the P. pastoris TEF promoter and at the 3′ end to a nucleic acid molecule comprising the P. pastoris TEF transcription termination sequence. The six tandem cassettes are flanked on one side by a nucleic acid molecule comprising a nucleotide sequence from the 5′ region and ORF of the TRP2 gene ending at the stop codon (SEQ ID NO: 83) followed by a P. pastoris ALG3 termination sequence and on the other side by a nucleic acid molecule comprising a nucleotide sequence from the 3′ region of the TRP2 gene (SEQ ID NO: 84). Plasmid pGLY2456 was linearized with SfiI and the linearized plasmid transformed into strain YGLY7961 to produce a number of strains in which the six expression cassette have been inserted into the TRP2 locus immediately following the TRP2 ORF by double-crossover homologous recombination. The strain YGLY8146 was selected from the strains produced. The strain was then counterselected in the presence of 5-FOA to produce a number of strains now auxotrophic for uridine. Strain YGLY9296 was selected.

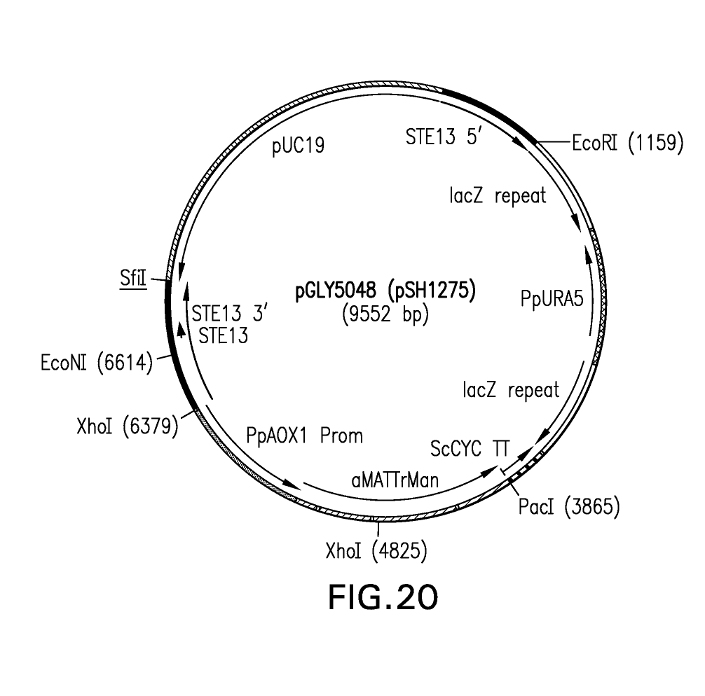

FIG. 20 shows as map of plasmid pGLY5048. Plasmid pGLY5048 is an integration vector that targets the STE13 locus and contains expression cassettes encoding (1) the T. reesei α-1,2-mannosidase catalytic domain fused at the N-terminus to S. cerevisiae αMATpre signal peptide (aMATTrMan) to target the chimeric protein to the secretory pathway and secretion from the cell and (2) the P. pastoris URA5 gene or transcription unit. The expression cassette encoding the aMATTrMan comprises a nucleic acid molecule encoding the T. reesei catalytic domain (SEQ ID NO:52) fused at the 5′ end to a nucleic acid molecule encoding the S. cerevisiae aMATpre signal peptide (SEQ ID NO:53 encoding amino acid sequence SEQ ID NO:54), which is operably linked at the 5′ end to a nucleic acid molecule comprising the P. pastoris AOX1 promoter and at the 3′ end to a nucleic acid molecule comprising the S. cerevisiae CYC transcription termination sequence. The URA5 expression cassette comprises a nucleic acid molecule comprising the P. pastoris URA5 gene or transcription unit flanked by nucleic acid molecules comprising lacZ repeats. The two tandem cassettes are flanked on one side by a nucleic acid molecule comprising a nucleotide sequence from the 5′ region of the STE13 gene (SEQ ID NO: 85) and on the other side by a nucleic acid molecule comprising a nucleotide sequence from the 3′ region of the STE13 gene (SEQ ID NO: 86). Plasmid pGLY5048 was linearized with SfiI and the linearized plasmid transformed into strain YGLY9296 to produce a number of strains. The strains YGLY9469 was selected from the strains produced. The strain is capable of producing glycoproteins that have single-mannose β-glycosylation (See Published U.S. Application No. 20090170159).

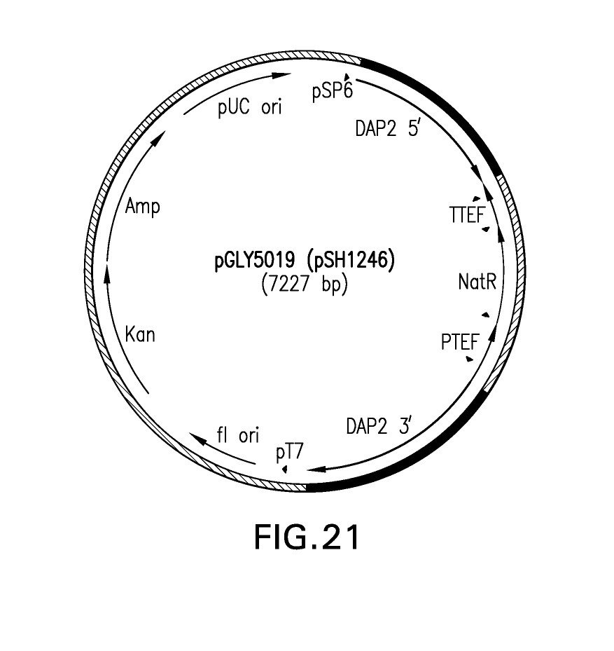

FIG. 21 shows as map of plasmid pGLY5019. Plasmid pGLY5019 is an integration vector that targets the DAP2 locus and contains an expression cassette comprising a nucleic acid molecule encoding the Nourseothricin resistance (NATR) expression cassette (originally from pAG25 from EROSCARF, Scientific Research and Development GmbH, Daimlerstrasse 13a, D-61352 Bad Homburg, Germany, See Goldstein et al., Yeast 15: 1541 (1999)). The NAT^{R} expression cassette (SEQ ID NO:64) is operably regulated to the Ashbya gossypii TEF1 promoter (SEQ ID NO:65) and A. gossypii TEF1 termination sequence (SEQ ID NO:66) flanked one side with the 5′ nucleotide sequence of the P. pastoris DAP2 gene (SEQ ID NO:87) and on the other side with the 3′ nucleotide sequence of the P. pastoris DAP2 gene (SEQ ID NO:88). Plasmid pGLY5019 was linearized and the linearized plasmid transformed into strain YGLY9469 to produce a number of strains in which the NATR expression cassette has been inserted into the DAP2 locus by double-crossover homologous recombination. The strain YGLY9797 was selected from the strains produced.

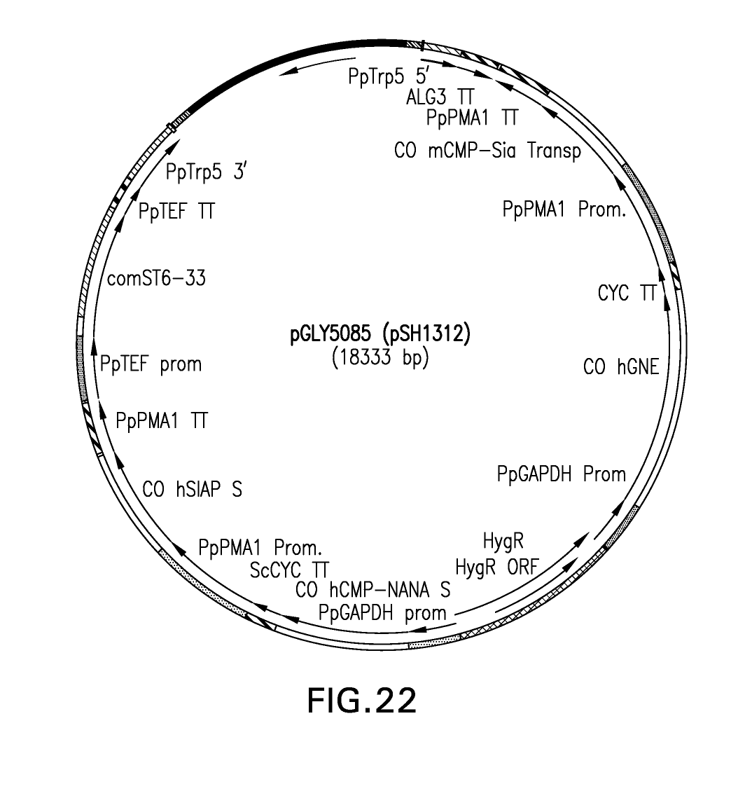

FIG. 22 shows as map of plasmid pGLY5085. Plasmid pGLY5085 is a KINKO plasmid for introducing a second set of the genes involved in producing sialylated N-glycans into P. pastoris. The plasmid is similar to plasmid YGLY2456 except that the P. pastoris ARGJ gene has been replaced with an expression cassette encoding hygromycin resistance (HygR) and the plasmid targets the P. pastoris TRP5 locus. The HYG^{R} resistance cassette is SEQ ID NO:89. The HYG^{R} expression cassette (SEQ ID NO:89) is operably regulated to the Ashbya gossypii TEF1 promoter and A. gossypii TEF1 termination sequences (See Goldstein et al., Yeast 15: 1541 (1999)). The six tandem cassettes are flanked on one side by a nucleic acid molecule comprising a nucleotide sequence from the 5′ region and ORF of the TRP5 gene ending at the stop codon (SEQ ID NO:90) followed by a P. pastoris ALG3 termination sequence and on the other side by a nucleic acid molecule comprising a nucleotide sequence from the 3′ region of the TRP5 gene (SEQ ID NO:91). Plasmid pGLY5085 was transformed into strain YGLY9797 to produce a number of strains of which strain YGLY12900 is selected.

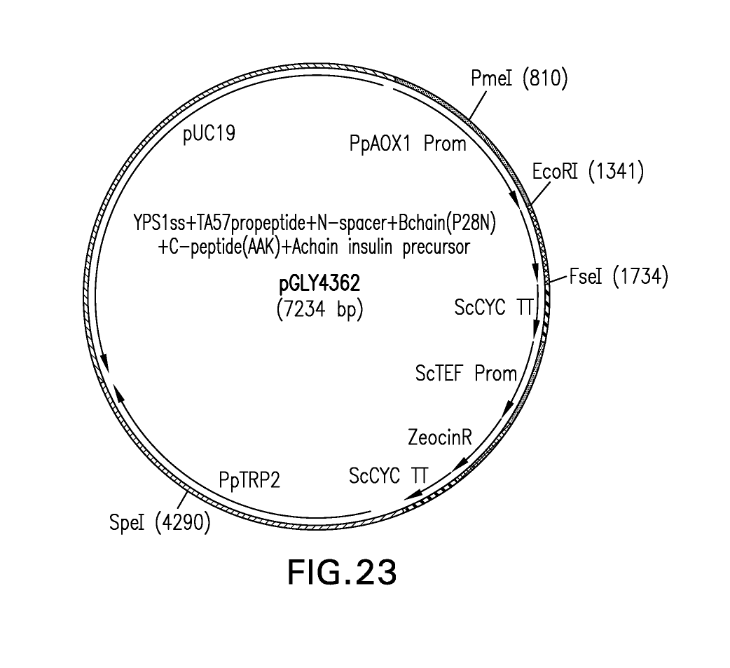

FIG. 23 shows as map of plasmid pGLY4362.Plamsid pGLY4362 is a roll-in integration plasmid that targets the TRP2 locus or AOX1 locus and includes an expression cassette encoding a pre-proinsulin analogue precursor comprising a Yps1ss peptide (SEQ ID NO:92) fused to a TA57 propeptide (SEQ ID NO:93) fused to an N-terminal spacer (SEQ ID NO:94) fused to the human insulin B-chain with a P28N substitution (SEQ ID NO:95) fused to a C-peptide consisting of the amino acid sequence AAK fused to the human insulin insulin A-chain (SEQ ID NO:96). The pre-proinsulin analogue precursor has the amino acid sequence shown in SEQ ID NO:97 and is encoded by the nucleotide sequence shown in SEQ ID NO:98. The expression cassette comprises a nucleic acid molecule encoding the fusion protein (SEQ ID NO:98) operably linked at the 5′ end to a nucleic acid molecule that has the inducible P. pastoris AOX1 promoter sequence (SEQ ID NO:55) and at the 3′ end to a nucleic acid molecule that has the Saccharomyces cerevisiae CYC transcription termination sequence (SEQ ID NO:15). For selecting transformants, the plasmid comprises an expression cassette encoding the Zeocin ORF in which the nucleic acid molecule encoding the ORF (SEQ ID NO:60) is operably linked at the 5′ end to a nucleic acid molecule having the S. cerevisiae TEF promoter sequence (SEQ ID NO:61) and at the 3′ end to a nucleic acid molecule having the S. cerevisiae CYC transcription termination sequence (SEQ ID NO:15). The plasmid further includes a nucleic acid molecule for targeting the TRP2 locus.

Strain YGLY12900 was transformed with plasmid pGLY4362, which is an expression plasmid that in Pichia pastoris enables expression of a glycosylated insulin analogue precursor molecule comprising the Yps1ss domain fused to the TA57 propeptide domain fused to an N-terminal spacer fused to the human insulin B-chain having a P28N substitution fused to a C-peptide having the amino acid sequence AAK fused to the human insulin A-chain, to produce a number of strains of which strain YGLY21058 was selected. The strain is capable of producing an N-glycosylated insulin analogue precursor comprising an N-terminal spacer fused to the human insulin B-chain having a P28N substitution fused to a C-peptide having the amino acid sequence AAK fused to the human insulin A-chain. The analysis of the N-glycosylated insulin analogue precursor expression from this engineered Pichia pastoris XRN1 knock-out strain is shown in Table 11.

### EXAMPLE 6

### Production of Pichia pastoris Strains for Human Erythropoietin (EPO) Production

This example describes construction of strain YGLY7117. Genetically engineered Pichia pastoris strain YGLY7117 produces recombinant human erythropoietin molecules. The strain produces glycoproteins having sialylated N-glycans. The strain YGLY7117 was constructed from wild-type Pichia pastoris strain NRRL-Y 11430 described earlier (“Add Reference here: J. Biotechnol. 2012 January; 157(1):198-206. Nett et al. “Optimization of erythropoietin production with controlled glycosylation-PEGylated erythropoietin produced in glycoengineered Pichia pastoris”). The analysis of the N-glycosylated insulin analogue precursor expression from this engineered Pichia pastoris XRN1 knock-out strain is shown in Table 7.

### Summary: XRN1 Knock-Out Mutants are Resistant to Stress-Induced Translational Inhibition

Analysis of xrn1Δ Pichia mutants illustrate that mRNA degradation enzymes are involved in regulating general translation repression in response to a variety of nutritional and environmental stresses. In two well-characterized examples, global protein synthesis is rapidly inhibited upon glucose deprivation or severe amino acid starvation. In general, the stress-induced translation inhibition is a rapid response mediated by a well-described pathway involving Gcn2 protein kinase and its subsequent phosphorylation of translation initiation factor eIF2. mRNA degradation enzymes have not been described to be involved in this Gcn2 protein phosphorylation process. However, Saccharomyces mutants effecting 5′ to 3′ mRNA decay such as Δdcp1 and Δxrn1 are generally resistant to this stress-induced translation repression. Thus, it has been surprisingly found that Δxrn1 Pichia strains of the present invention continue to translate at a rate typical of that seen with glucose-containing medium, even in glucose deprivation or amino acid starvation conditions. Because current high cell-density fermentors usually operate at oxygen-limited or carbon-source limited processes, it is likely that part of the yield improvement result of Δxrn1 Pichia cells can be attributed to this Δxrn1 translation derepression during fermentation process.

Tables 7-11 summarize yield improvement and N-Glycan quality improvement results with engineered Pichia xrn1 knockout host cells expressing exemplary heterologous proteins, in this case three different therapeutic proteins, as described in Examples 2-5.

TABLE 7
yGLY7117 human EPO XRN1 Knockout Yield Improvement

| Strain ID | yGLY7117 |
|---|---|
| Protein ID | Human EPO |
| Fermentation Platform | Micro-24 5 mL Reactor |
| Genotype | Average Titer (μg/L) |
| XRN1-wt (n = 3) | 32.5 |
| Δxrn1 (n = 4) | 58.1 |

TABLE 8
yGLY12501 Herceptin mAb XRN1 Knockout Yield Improvement

| Strain ID | yGLY12501 |
|---|---|
| Protein ID | Herceptin mAb |
| Fermentation Platform | Micro-24 5 mL Reactor |
| Genotype | Average Titer (mg/L) |
| XRN1-wt (n = 4) | 504 |
| Δxrn1 (n = 6) | 600 |

TABLE 9
yGLY13992 Herceptin mAb XRN1 Knockout N-Glycan Quality and Yield Improvement

| | |
|---|---|
| Strain ID | yGLY13992 |
| Protein ID | Herceptin mAb |
| Fermentation Platform | DasGip 1 Liter Reactor |

| | Man5 | G0 | G1 | G2 | Complex | WCW | Supernatant | Broth | Induction |
|---|---|---|---|---|---|---|---|---|---|
| Genotype | % | % | % | % | % | (g/L) | Titer (mg/L) | Titer (mg/L) | Hours |
| XRN1-wt | 15.0 | 65.9 | 15.5 | 1.2 | 85.0 | 319 | 1112 | 757.2 | 105.6 |
| (n = 2) | | | | | | | | | |
| Δxrn1 | 10.5 | 55.5 | 23.8 | 4.7 | 89.5 | 234 | 1061 | 812.7 | 89.4 |
| (n = 5) | | | | | | | | | |

TABLE 10
yGLY14836 Herceptin mAb XRN1 Knockout N-Glycan Quality Improvement

| | |
|---|---|
| Strain ID | yGLY14836 |
| Protein ID | Herceptin mAb |
| Fermentation Platform | DasGip 1 Liter Reactor |

| | Man5 | G0 | G1 | G2 | Complex | WCW | Supernatant | Broth | Induction |
|---|---|---|---|---|---|---|---|---|---|
| Genotype | % | % | % | % | % | (g/L) | Titer (mg/L) | Titer (mg/L) | Hours |
| XRN1-wt | 23.0 | 42.7 | 16.3 | 4.7 | 77.0 | 285 | 1525 | 1090 | 103.0 |
| (n = 3) | | | | | | | | | |
| Δxrn1 | 12.6 | 55.2 | 18.7 | 5.1 | 87.4 | 203 | 1067 | 850 | 94.3 |
| (n = 3) | | | | | | | | | |

TABLE 11
yGLY21080 P28N Glyco-Insulin XRN1 Knockout N-Glycan Quality and Yield Improvement

| | |
|---|---|
| Strain ID | yGLY21080 |
| Protein ID | P28N Glyco-Insulin |
| Fermentation Platform | DasGip 1 Liter Reactor |

| | Man5 | G0 | A1 | A2 | Complex | WCW | Supernatant | Broth | Induction |
|---|---|---|---|---|---|---|---|---|---|
| Genotype | % | % | % | % | % | (g/L) | Titer (mg/L) | Titer (mg/L) | Hours |
| XRN1-wt | 17.4 | 0 | 21.3 | 54.1 | 82.6 | 329 | 53 | 35.5 | 86.9 |
| (n = 2) | | | | | | | | | |
| Δxrn1 | 8.3 | 7.8 | 48.4 | 32.0 | 91.7 | 388 | 123 | 75.2 | 80.7 |
| (n = 4) | | | | | | | | | |

In summary, the mRNA stabilization technique presented herein provides a powerful and flexible method to improve protein fermentation titer and protein glycosylation quality simultaneously. Inhibition of global mRNA turnover by XRN1 knockout increases mRNA abundance of both target protein and corresponding glycosyltransferases. Moreover, mutation of XRN 1 may affect translation initiation to prevent stress-induced translation regulation and further improve the titer.

### GLOSSARY

ScSUC2 S. cerevisiae Invertase
OCH1 Alpha-1,6-mannosyltransferase
K1MNN2-2: K. lactis UDP-GlcNAc transporter
BMT1: Beta-mannose-transfer (beta-mannose elimination)
BMT2: Beta-mannose-transfer (beta-mannose elimination)
BMT3: Beta-mannose-transfer (beta-mannose elimination)
BMT4: Beta-mannose-transfer (beta-mannose elimination)
MNN4L1: MNN4-like 1 (charge elimination)
MmSLC35A3 Mouse homologue of UDP-GlcNAc transporter
PNO1: Phosphomannosylation of N-glycans (charge elimination)
MNN4: Mannosyltransferase (charge elimination)
ScGAL10 UDP-glucose 4-epimerase
XB33 Truncated HsGalT1 fused to ScKRE2 leader
DmUGT UDP-Galactose transporter
KD53 Truncated DmMNSII fused to ScMNN2 leader
TC54 Truncated RnGNTII fused to ScMNN2 leader
NA10 Truncated HsGNTI fused to PpSEC12 leader
FB8: Truncated MmMNS1A fused to ScSEC12 leader
TrMDS1: Secreted T. reseei MNS 1
Sh ble: Zeocin resistance marker
Nat: Streptomyces noursei nourseothricin acetyltransferase
GD9: Truncated MmMNS1B fused to ScSEC12 leader
MmCST Mouse CMP-sialic acid transporter
HsGNE Human UDP-GlcNAc 2-epimerase/N-acetylmannosamine kinase
HsCSS Human CMP-sialic acid synthase
HsSPS Human N-acetylneuraminate-9-phosphate synthase
MmST6-33 Truncated Mouse alpha-2,6-sailyl transferase fused to ScKRE2 leader
TrMDS 1: Secreted T. reseei MNS 1
STE13 Golgi dipeptidyl aminopeptidase
DAP2 Vacuolar dipeptidyl aminopeptidase
NatR Nourseothricin resistance marker
HygR Hygromycin resistance marker
TRP2 Tryptophan biosynthesis
Sh ble: Zeocin resistance marker
Insulin precursor variant: YPS1ss+TA57propeptide+N-spacer+Bchain(P28N)+C-peptide(AAK)+Achain insulin precursor

TABLE 12
BRIEF DESCRIPTION OF THE SEQUENCES

| SEQ | | |
|---|---|---|
| ID NO: | Description | Sequence |
| 1 | Pichia pastoris | TGAATGGCCTGACTAACAGAAGAATATTTGTTTTCCA |
| | Sequence of the | GGAAGTTAATGTCTCTTGGAACTGATTCAATTAGTTCT |
| | 5′-Region used | TTGTGGTTTGCTTGATCGTCTTTCAAGCTCTCTGCGAT |
| | for knock out of | GTCCATTTGCTGCTGAATGTACTGATCAAGTTGTTCTA |
| | PpXRN1: | TTTCTTTTTGATAGGCATCTGGTAGATCCGTGACTCTC |
| | | GTAAGGGATGTTATCTGTGGTTGAGAAGCATTGTTAG |
| | | GGTTGTTGGGTGCTGCATTGTTGCCCAGTAAACTATTA |
| | | TTGTTATTACTTGAATTGAAAAGCCCACCAGCATTACT |
| | | GTTAGTATTGTTTCCAAATAGACTCCCTGTAGTTGTAT |
| | | TAGCGTTCGTATTATTGCTCCCAAAAAGACCTCCAGTG |
| | | TTACCACTCGGATTATTTGAGCCTGAGAAACCACTTTG |
| | | TGCAGGTTTATTGGCATTTCCAAACAACCCCCCTCCAG |
| | | TTTGGGCATTACTATTTGCAGTATTGCTGCCTCCAAAA |
| | | AGACCACCCGATTGTGTGTTATTTGAATTGGTTCCAAA |
| | | TAGCCCTCCTGATTGTGTGTTACCAGAATTTCCTCCAA |
| | | ATAATCCTCCCTGTTGAGTGTTAGAGTTAGTGTTGGTG |
| | | TTACCTCCAAAGAGTCCTTTCGATTGAGTATTACTAGC |
| | | ATTTCCCCCAAACAACCCTCCTGATTGATTGCTGTTGC |
| | | TAGCAGTATTCCCACCAAACAATCCTTGCGATTGAGT |
| | | ATTTCCCGTGTTGGTGTTGGCGCCAAATAAGCTACCTG |
| | | ACTGATTCGTGTTGGTATTACCAGAATTGTTTCCAAAC |
| | | ATACCGCCTGTATTGGTACTACCTGAAGCGTTCGTACT |
| | | GCCAAACAATCCTCCGCTATTGCCTCCAGAATTTGTAC |
| | | TAGCATTATTTCCAAACAAACCTCCTGTGTTATTCGTA |
| | | TTCGTAGCAGGCTTTGCTCCAAACAAGCCTCCAGTGTT |
| | | ACCAGAGGAGCTACCTCCAAAAGCCCCAACATTGCTA |
| | | CCTGAGGCGTTTCCAAACATACCGCCTGAATTGGCGG |
| | | GGTTCTTGTTGGAATTTCCAAACATTTGACTTTAAGGT |
| | | TTTAAAGAACGTTGTTTTGACAAGGGAAACAAAAGTT |
| | | CCCACTAAATTTTTCGATGTAAGGACTTGGGAGGAGC |
| | | AACTGCTTACATGAACTCCCTCTAACTTTCCTCATAAA |
| | | AATCCTTCCAAGCTCGCGAGGTCCCTTCCAACTAAGTC |
| | | GGGTAAGTTT |
| 2 | Pichia pastoris | TGCCCGGACTTCTATCCAGCAAATTTACGGAACGGTG |
| | Sequence of the | TTCAATCAAGTGTTGAGCGCTCAACCTCAGTTGCAGCC |
| | 3′-Region used | TGTCAGAGGCTTCTCAAATCCGGTACCTGAAACCCCT |
| | for knock out of | GTAAATGGAGTCCAAGCGAATGAACAACACTCTGATT |
| | PpXRN1: | CTACCCCTCAAAATCATTCTAGGGATGAAAACCAAGG |
| | | AAGAGGTCGTGGTAGAGGCAGAGGAAATAGAAGAGG |
| | | AAGAGGTCGAGGTAGAGGCAAAGGAGGACAGTAAAT |
| | | CAGAATGAAGGTGTCCCTCCGATTGAATAGAATTGTG |
| | | TTGTAATATTAGTGTATTGCGTTAATGGTACTAATAAT |
| | | CATCCTGCATTGTATAACTAAGAACTTTCTTCTCCTGC |
| | | CTTAGAGCAGTCGTCTTGAAAACTTTTTGCTCCCAGCC |
| | | ATTAGACCTATCAATTCCGTCCCATCTATACCCTGGCA |
| | | TTATAGAGAAACGATTGTCCGGGAAAGAAGATTTGTA |
| | | TAGTTTACGGCCTGTCTGTGAGATGTAACGGTCTTTTT |
| | | GCAGCGACTTGACTTTATCTGGAGAAAATGCTAGCAA |
| | | CGGGTCATCTACATAGCTTGGCGGAGCTACTCTTTTAG |
| | | TGGCCGTTTGAGTAACCTCGGGGAGATTCATATTGCGT |
| | | AATTTTTCAGATGCTTCTTTGGCTTTCTGTTTTTCTTGC |
| | | TGTGATTTCTGTCTCTGTTGTTCTAGCATTTCCTCATGA |
| | | GAGAGGACGTTTCCTGATAAGTCTCTATAAATAGTGG |
| | | CATTCTCGGGTTTTGCAGGTTTTGTGGGTTTTTGTATC |
| | | GACGACTTGGGTTTCGGCTTCAGTGATTTATCCCTTTT |
| | | CTTGGACTTGGATTTGCCATATTTGGAATCCAGGTAGT |
| | | CCTGTAGTGACATTCGTTTCGATACTAGGTGCGATGGT |
| | | TCATTCGATACGATATATAATGCTTACATAAGCTAATG |
| | | GTACTGGGAACATCACTTATACTATCCGTTCAGATCAA |
| | | CAGGAGAATTCATTCATACAACATGCCAAATTCATTG |
| | | AAGACCCAGTCATCATTCCATCAGACTGCCCTTGGTTA |
| | | AAGGTGATAAGGAATTAATTGAGGTTTATCGGGGTTT |
| | | AAAGTGAGGCGGGCATCAAGAAAAAAAAAAAGAGGG |
| | | CAGGAGCAGTGGAACTTTCAAAACAAGAAAGAGATA |
| | | AATCTTATCTCGTGACCCCTATCTTAGCAAATAACGTT |
| | | TACGTTTGAAGGTAATAGATTAAGCAACCAATTACCT |
| | | CATCCTAACTTACGAGTAATATCCCGTTTCATCTCATC |
| | | ATCAATGAGGGACTTGATTTTATACCGACATTGTTGGC |
| | | TCCCCACATTAACCCTTTAAAGCAGGAAGATCCAATT |
| | | CCCCGAGGGACAAACTTGACACCCTAACTTTCCCGGG |
| | | GTTCACGAAATATTCATGAACCCCCCCCCTTGATACGA |
| | | ACATCTGCGCCGTATGCACCCTTCTGGGACATACCGCC |
| | | TGAGGCCACCTC |
| 3 | S. cerevisiae | AGGCCTCGCAACAACCTATAATTGAGTTAAGTGCCTTT |
| | invertase gene | CCAAGCTAAAAAGTTTGAGGTTATAGGGGCTTAGCAT |
| | (ScSUC2) ORF | CCACACGTCACAATCTCGGGTATCGAGTATAGTATGT |
| | underlined | AGAATTACGGCAGGAGGTTTCCCAATGAACAAAGGAC |
| | (909-2507 bp)— | AGGGGCACGGTGAGCTGTCGAAGGTATCCATTTTATC |
| | | ATGTTTCGTTTGTACAAGCACGACATACTAAGACATTT |
| | | ACCGTATGGGAGTTGTTGTCCTAGCGTAGTTCTCGCTC |
| | | CCCCAGCAAAGCTCAAAAAAGTACGTCATTTAGAATA |
| | | GTTTGTGAGCAAATTACCAGTCGGTATGCTACGTTAG |
| | | AAAGGCCCACAGTATTCTTCTACCAAAGGCGTGCCTTT |
| | | GTTGAACTCGATCCATTATGAGGGCTTCCATTATTCCC |
| | | CGCATTTTTATTACTCTGAACAGGAATAAAAAGAAAA |
| | | AACCCAGTTTAGGAAATTATCCGGGGGCGAAGAAATA |
| | | CGCGTAGCGTTAATCGACCCCACGTCCAGGGTTTTTCC |
| | | ATGGAGGTTTCTGGAAAAACTGACGAGGAATGTGATT |
| | | ATAAATCCCTTTATGTGATGTCTAAGACTTTTAAGGTA |
| | | CGCCCGATGTTTGCCTATTACCATCATAGAGACGTTTC |
| | | TTTTCGAGGAATGCTTAAACGACTTTGTTTGACAAAAA |
| | | TGTTGCCTAAGGGCTCTATAGTAAACCATTTGGAAGA |
| | | AAGATTTGACGACTTTTTTTTTTTGGATTTCGATCCTAT |
| | | AATCCTTCCTCCTGAAAAGAAACATATAAATAGATAT |
| | | GTATTATTCTTCAAAACATTCTCTTGTTCTTGTGCTTTT |
| | | TTTTTACCATATATCTTACTTTTTTTTTTCTCTCAGAGA |
| | | AACAAGCAAAACAAAAAGCTTTTCTTTTCACTAACGT |
| | | ATATGATGCTTTTGCAAGCTTTCCTTTTCCTTTTGGCTG |
| | | GTTTTGCAGCCAAAATATCTGCATCAATGACAAACGA |
| | | AACTAGCGATAGACCTTTGGTCCACTTCACACCCAAC |
| | | AAGGGCTGGATGAATGACCCAAATGGGTTGTGGTACG |
| | | ATGAAAAAGATGCCAAATGGCATCTGTACTTTCAATA |
| | | CAACCCAAATGACACCGTATGGGGTACGCCATTGTTT |
| | | TGGGGCCATGCTACTTCCGATGATTTGACTAATTGGGA |
| | | AGATCAACCCATTGCTATCGCTCCCAAGCGTAACGAT |
| | | TCAGGTGCTTTCTCTGGCTCCATGGTGGTTGATTACAA |
| | | CAACACGAGTGGGTTTTTCAATGATACTATTGATCCAA |
| | | GACAAAGATGCGTTGCGATTTGGACTTATAACACTCC |
| | | TGAAAGTGAAGAGCAATACATTAGCTATTCTCTTGAT |
| | | GGTGGTTACACTTTTACTGAATACCAAAAGAACCCTG |
| | | TTTTAGCTGCCAACTCCACTCAATTCAGAGATCCAAAG |
| | | GTGTTCTGGTATGAACCTTCTCAAAAATGGATTATGAC |
| | | GGCTGCCAAATCACAAGACTACAAAATTGAAATTTAC |
| | | TCCTCTGATGACTTGAAGTCCTGGAAGCTAGAATCTGC |
| | | ATTTGCCAATGAAGGTTTCTTAGGCTACCAATACGAAT |
| | | GTCCAGGTTTGATTGAAGTCCCAACTGAGCAAGATCC |
| | | TTCCAAATCTTATTGGGTCATGTTTATTTCTATCAACC |
| | | CAGGTGCACCTGCTGGCGGTTCCTTCAACCAATATTTT |
| | | GTTGGATCCTTCAATGGTACTCATTTTGAAGCGTTTGA |
| | | CAATCAATCTAGAGTGGTAGATTTTGGTAAGGACTAC |
| | | TATGCCTTGCAAACTTTCTTCAACACTGACCCAACCTA |
| | | CGGTTCAGCATTAGGTATTGCCTGGGCTTCAAACTGG |
| | | GAGTACAGTGCCTTTGTCCCAACTAACCCATGGAGAT |
| | | CATCCATGTCTTTGGTCCGCAAGTTTTCTTTGAACACT |
| | | GAATATCAAGCTAATCCAGAGACTGAATTGATCAATT |
| | | TGAAAGCCGAACCAATATTGAACATTAGTAATGCTGG |
| | | TCCCTGGTCTCGTTTTGCTACTAACACAACTCTAACTA |
| | | AGGCCAATTCTTACAATGTCGATTTGAGCAACTCGACT |
| | | GGTACCCTAGAGTTTGAGTTGGTTTACGCTGTTAACAC |
| | | CACACAAACCATATCCAAATCCGTCTTTGCCGACTTAT |
| | | CACTTTGGTTCAAGGGTTTAGAAGATCCTGAAGAATA |
| | | TTTGAGAATGGGTTTTGAAGTCAGTGCTTCTTCCTTCT |
| | | TTTTGGACCGTGGTAACTCTAAGGTCAAGTTTGTCAAG |
| | | GAGAACCCATATTTCACAAACAGAATGTCTGTCAACA |
| | | ACCAACCATTCAAGTCTGAGAACGACCTAAGTTACTA |
| | | TAAAGTGTACGGCCTACTGGATCAAAACATCTTGGAA |
| | | TTGTACTTCAACGATGGAGATGTGGTTTCTACAAATAC |
| | | CTACTTCATGACCACCGGTAACGCTCTAGGATCTGTGA |
| | | ACATGACCACTGGTGTCGATAATTTGTTCTACATTGAC |
| | | AAGTTCCAAGTAAGGGAAGTAAAATAGAGGTTATAA |
| | | AACTTATTGTCTTTTTTATTTTTTTCAAAAGCCATTCTA |
| | | AAGGGCTTTAGCTAACGAGTGACGAATGTAAAACTTT |
| | | ATGATTTCAAAGAATACCTCCAAACCATTGAAAATGT |
| | | ATTTTTATTTTTATTTTCTCCCGACCCCAGTTACCTGGA |
| | | ATTTGTTCTTTATGTACTTTATATAAGTATAATTCTCTT |
| | | AAAAATTTTTACTACTTTGCAATAGACATCATTTTTTC |
| | | ACGTAATAAACCCACAATCGTAATGTAGTTGCCTTAC |
| | | ACTACTAGGATGGACCTTTTTGCCTTTATCTGTTTTGTT |
| | | ACTGACACAATGAAACCGGGTAAAGTATTAGTTATGT |
| | | GAAAATTTAAAAGCATTAAGTAGAAGTATACCATATT |
| | | GTAAAAAAAAAAAGCGTTGTCTTCTACGTAAAAGTGT |
| | | TCTCAAAAAGAAGTAGTGAGGGAAATGGATACCAAGC |
| | | TATCTGTAACAGGAGCTAAAAAATCTCAGGGAAAAGC |
| | | TTCTGGTTTGGGAAACGGTCGAC |
| 4 | Pichia pastoris | ATCGGCCTTTGTTGATGCAAGTTTTACGTGGATCATGG |
| | Sequence of the | ACTAAGGAGTTTTATTTGGACCAAGTTCATCGTCCTAG |
| | 5′-Region used | ACATTACGGAAAGGGTTCTGCTCCTCTTTTTGGAAACT |
| | for knock out of | TTTTGGAACCTCTGAGTATGACAGCTTGGTGGATTGTA |
| | PpURA5: | CCCATGGTATGGCTTCCTGTGAATTTCTATTTTTTCTAC |
| | | ATTGGATTCACCAATCAAAACAAATTAGTCGCCATGG |
| | | CTTTTTGGCTTTTGGGTCTATTTGTTTGGACCTTCTTGG |
| | | AATATGCTTTGCATAGATTTTTGTTCCACTTGGACTAC |
| | | TATCTTCCAGAGAATCAAATTGCATTTACCATTCATTT |
| | | CTTATTGCATGGGATACACCACTATTTACCAATGGATA |
| | | AATACAGATTGGTGATGCCACCTACACTTTTCATTGTA |
| | | CTTTGCTACCCAATCAAGACGCTCGTCTTTTCTGTTCT |
| | | ACCATATTACATGGCTTGTTCTGGATTTGCAGGTGGAT |
| | | TCCTGGGCTATATCATGTATGATGTCACTCATTACGTT |
| | | CTGCATCACTCCAAGCTGCCTCGTTATTTCCAAGAGTT |
| | | GAAGAAATATCATTTGGAACATCACTACAAGAATTAC |
| | | GAGTTAGGCTTTGGTGTCACTTCCAAATTCTGGGACAA |
| | | AGTCTTTGGGACTTATCTGGGTCCAGACGATGTGTATC |
| | | AAAAGACAAATTAGAGTATTTATAAAGTTATGTAAGC |
| | | AAATAGGGGCTAATAGGGAAAGAAAAATTTTGGTTCT |
| | | TTATCAGAGCTGGCTCGCGCGCAGTGTTTTTCGTGCTC |
| | | CTTTGTAATAGTCATTTTTGACTACTGTTCAGATTGAA |
| | | ATCACATTGAAGATGTCACTCGAGGGGTACCAAAAAA |
| | | GGTTTTTGGATGCTGCAGTGGCTTCGC |
| 5 | Pichia pastoris | GGTCTTTTCAACAAAGCTCCATTAGTGAGTCAGCTGGC |
| | Sequence of the | TGAATCTTATGCACAGGCCATCATTAACAGCAACCTG |
| | 3′-Region used | GAGATAGACGTTGTATTTGGACCAGCTTATAAAGGTA |
| | for knock out of | TTCCTTTGGCTGCTATTACCGTGTTGAAGTTGTACGAG |
| | PpURA5: | CTCGGCGGCAAAAAATACGAAAATGTCGGATATGCGT |
| | | TCAATAGAAAAGAAAAGAAAGACCACGGAGAAGGTG |
| | | GAAGCATCGTTGGAGAAAGTCTAAAGAATAAAAGAGT |
| | | ACTGATTATCGATGATGTGATGACTGCAGGTACTGCT |
| | | ATCAACGAAGCATTTGCTATAATTGGAGCTGAAGGTG |
| | | GGAGAGTTGAAGGTAGTATTATTGCCCTAGATAGAAT |
| | | GGAGACTACAGGAGATGACTCAAATACCAGTGCTACC |
| | | CAGGCTGTTAGTCAGAGATATGGTACCCCTGTCTTGA |
| | | GTATAGTGACATTGGACCATATTGTGGCCCATTTGGGC |
| | | GAAACTTTCACAGCAGACGAGAAATCTCAAATGGAAA |
| | | CGTATAGAAAAAAGTATTTGCCCAAATAAGTATGAAT |
| | | CTGCTTCGAATGAATGAATTAATCCAATTATCTTCTCA |
| | | CCATTATTTTCTTCTGTTTCGGAGCTTTGGGCACGGCG |
| | | GCGGGTGGTGCGGGCTCAGGTTCCCTTTCATAAACAG |
| | | ATTTAGTACTTGGATGCTTAATAGTGAATGGCGAATGC |
| | | AAAGGAACAATTTCGTTCATCTTTAACCCTTTCACTCG |
| | | GGGTACACGTTCTGGAATGTACCCGCCCTGTTGCAACT |
| | | CAGGTGGACCGGGCAATTCTTGAACTTTCTGTAACGTT |
| | | GTTGGATGTTCAACCAGAAATTGTCCTACCAACTGTAT |
| | | TAGTTTCCTTTTGGTCTTATATTGTTCATCGAGATACTT |
| | | CCCACTCTCCTTGATAGCCACTCTCACTCTTCCTGGAT |
| | | TACCAAAATCTTGAGGATGAGTCTTTTCAGGCTCCAG |
| | | GATGCAAGGTATATCCAAGTACCTGCAAGCATCTAAT |
| | | ATTGTCTTTGCCAGGGGGTTCTCCACACCATACTCCTT |
| | | TTGGCGCATGC |
| 6 | Pichia pastoris | TCTAGAGGGACTTATCTGGGTCCAGACGATGTGTATC |
| | Sequence of the | AAAAGACAAATTAGAGTATTTATAAAGTTATGTAAGC |
| | PpURA5 | AAATAGGGGCTAATAGGGAAAGAAAAATTTTGGTTCT |
| | auxotrophic | TTATCAGAGCTGGCTCGCGCGCAGTGTTTTTCGTGCTC |
| | marker: | CTTTGTAATAGTCATTTTTGACTACTGTTCAGATTGAA |
| | | ATCACATTGAAGATGTCACTGGAGGGGTACCAAAAAA |
| | | GGTTTTTGGATGCTGCAGTGGCTTCGCAGGCCTTGAAG |
| | | TTTGGAACTTTCACCTTGAAAAGTGGAAGACAGTCTC |
| | | CATACTTCTTTAACATGGGTCTTTTCAACAAAGCTCCA |
| | | TTAGTGAGTCAGCTGGCTGAATCTTATGCTCAGGCCAT |
| | | CATTAACAGCAACCTGGAGATAGACGTTGTATTTGGA |
| | | CCAGCTTATAAAGGTATTCCTTTGGCTGCTATTACCGT |
| | | GTTGAAGTTGTACGAGCTGGGCGGCAAAAAATACGAA |
| | | AATGTCGGATATGCGTTCAATAGAAAAGAAAAGAAAG |
| | | ACCACGGAGAAGGTGGAAGCATCGTTGGAGAAAGTCT |
| | | AAAGAATAAAAGAGTACTGATTATCGATGATGTGATG |
| | | ACTGCAGGTACTGCTATCAACGAAGCATTTGCTATAA |
| | | TTGGAGCTGAAGGTGGGAGAGTTGAAGGTTGTATTAT |
| | | TGCCCTAGATAGAATGGAGACTACAGGAGATGACTCA |
| | | AATACCAGTGCTACCCAGGCTGTTAGTCAGAGATATG |
| | | GTACCCCTGTCTTGAGTATAGTGACATTGGACCATATT |
| | | GTGGCCCATTTGGGCGAAACTTTCACAGCAGACGAGA |
| | | AATCTCAAATGGAAACGTATAGAAAAAAGTATTTGCC |
| | | CAAATAAGTATGAATCTGCTTCGAATGAATGAATTAA |
| | | TCCAATTATCTTCTCACCATTATTTTCTTCTGTTTCGGA |
| | | GCTTTGGGCACGGCGGCGGATCC |
| 7 | Escherichia coli | CCTGCACTGGATGGTGGCGCTGGATGGTAAGCCGCTG |
| | Sequence of the | GCAAGCGGTGAAGTGCCTCTGGATGTCGCTCCACAAG |
| | part of the E. coli | GTAAACAGTTGATTGAACTGCCTGAACTACCGCAGCC |
| | lacZ gene | GGAGAGCGCCGGGCAACTCTGGCTCACAGTACGCGTA |
| | that was used to | GTGCAACCGAACGCGACCGCATGGTCAGAAGCCGGGC |
| | construct the | ACATCAGCGCCTGGCAGCAGTGGCGTCTGGCGGAAAA |
| | PpURA5 blaster | CCTCAGTGTGACGCTCCCCGCCGCGTCCCACGCCATCC |
| | (recyclable | CGCATCTGACCACCAGCGAAATGGATTTTTGCATCGA |
| | auxotrophic | GCTGGGTAATAAGCGTTGGCAATTTAACCGCCAGTCA |
| | marker) | GGCTTTCTTTCACAGATGTGGATTGGCGATAAAAAAC |
| | | AACTGCTGACGCCGCTGCGCGATCAGTTCACCCGTGC |
| | | ACCGCTGGATAACGACATTGGCGTAAGTGAAGCGACC |
| | | CGCATTGACCCTAACGCCTGGGTCGAACGCTGGAAGG |
| | | CGGCGGGCCATTACCAGGCCGAAGCAGCGTTGTTGCA |
| | | GTGCACGGCAGATACACTTGCTGATGCGGTGCTGATT |
| | | ACGACCGCTCACGCGTGGCAGCATCAGGGGAAAACCT |
| | | TATTTATCAGCCGGAAAACCTACCGGATTGATGGTAG |
| | | TGGTCAAATGGCGATTACCGTTGATGTTGAAGTGGCG |
| | | AGCGATACACCGCATCCGGCGCGGATTGGCCTGAACT |
| | | GCCAG |
| 8 | Pichia pastoris | AAAACCTTTTTTCCTATTCAAACACAAGGCATTGCTTC |
| | Sequence of the | AACACGTGTGCGTATCCTTAACACAGATACTCCATACT |
| | 5′-Region used | TCTAATAATGTGATAGACGAATACAAAGATGTTCACT |
| | for knock out of | CTGTGTTGTGTCTACAAGCATTTCTTATTCTGATTGGG |
| | PpOCH 1: | GATATTCTAGTTACAGCACTAAACAACTGGCGATACA |
| | | AACTTAAATTAAATAATCCGAATCTAGAAAATGAACT |
| | | TTTGGATGGTCCGCCTGTTGGTTGGATAAATCAATACC |
| | | GATTAAATGGATTCTATTCCAATGAGAGAGTAATCCA |
| | | AGACACTCTGATGTCAATAATCATTTGCTTGCAACAAC |
| | | AAACCCGTCATCTAATCAAAGGGTTTGATGAGGCTTA |
| | | CCTTCAATTGCAGATAAACTCATTGCTGTCCACTGCTG |
| | | TATTATGTGAGAATATGGGTGATGAATCTGGTCTTCTC |
| | | CACTCAGCTAACATGGCTGTTTGGGCAAAGGTGGTAC |
| | | AATTATACGGAGATCAGGCAATAGTGAAATTGTTGAA |
| | | TATGGCTACTGGACGATGCTTCAAGGATGTACGTCTA |
| | | GTAGGAGCCGTGGGAAGATTGCTGGCAGAACCAGTTG |
| | | GCACGTCGCAACAATCCCCAAGAAATGAAATAAGTGA |
| | | AAACGTAACGTCAAAGACAGCAATGGAGTCAATATTG |
| | | ATAACACCACTGGCAGAGCGGTTCGTACGTCGTTTTG |
| | | GAGCCGATATGAGGCTCAGCGTGCTAACAGCACGATT |
| | | GACAAGAAGACTCTCGAGTGACAGTAGGTTGAGTAAA |
| | | GTATTCGCTTAGATTCCCAACCTTCGTTTTATTCTTTCG |
| | | TAGACAAAGAAGCTGCATGCGAACATAGGGACAACTT |
| | | TTATAAATCCAATTGTCAAACCAACGTAAAACCCTCT |
| | | GGCACCATTTTCAACATATATTTGTGAAGCAGTACGC |
| | | AATATCGATAAATACTCACCGTTGTTTGTAACAGCCCC |
| | | AACTTGCATACGCCTTCTAATGACCTCAAATGGATAA |
| | | GCCGCAGCTTGTGCTAACATACCAGCAGCACCGCCCG |
| | | CGGTCAGCTGCGCCCACACATATAAAGGCAATCTACG |
| | | ATCATGGGAGGAATTAGTTTTGACCGTCAGGTCTTCA |
| | | AGAGTTTTGAACTCTTCTTCTTGAACTGTGTAACCTTT |
| | | TAAATGACGGGATCTAAATACGTCATGGATGAGATCA |
| | | TGTGTGTAAAAACTGACTCCAGCATATGGAATCATTC |
| | | CAAAGATTGTAGGAGCGAACCCACGATAAAAGTTTCC |
| | | CAACCTTGCCAAAGTGTCTAATGCTGTGACTTGAAATC |
| | | TGGGTTCCTCGTTGAAGACCCTGCGTACTATGCCCAAA |
| | | AACTTTCCTCCACGAGCCCTATTAACTTCTCTATGAGT |
| | | TTCAAATGCCAAACGGACACGGATTAGGTCCAATGGG |
| | | TAAGTGAAAAACACAGAGCAAACCCCAGCTAATGAG |
| | | CCGGCCAGTAACCGTCTTGGAGCTGTTTCATAAGAGT |
| | | CATTAGGGATCAATAACGTTCTAATCTGTTCATAACAT |
| | | ACAAATTTTATGGCTGCATAGGGAAAAATTCTCAACA |
| | | GGGTAGCCGAATGACCCTGATATAGACCTGCGACACC |
| | | ATCATACCCATAGATCTGCCTGACAGCCTTAAAGAGC |
| | | CCGCTAAAAGACCCGGAAAACCGAGAGAACTCTGGAT |
| | | TAGCAGTCTGAAAAAGAATCTTCACTCTGTCTAGTGG |
| | | AGCAATTAATGTCTTAGCGGCACTTCCTGCTACTCCGC |
| | | CAGCTACTCCTGAATAGATCACATACTGCAAAGACTG |
| | | CTTGTCGATGACCTTGGGGTTATTTAGCTTCAAGGGCA |
| | | ATTTTTGGGACATTTTGGACACAGGAGACTCAGAAAC |
| | | AGACACAGAGCGTTCTGAGTCCTGGTGCTCCTGACGT |
| | | AGGCCTAGAACAGGAATTATTGGCTTTATTTGTTTGTC |
| | | CATTTCATAGGCTTGGGGTAATAGATAGATGACAGAG |
| | | AAATAGAGAAGACCTAATATTTTTTGTTCATGGCAAAT |
| | | CGCGGGTTCGCGGTCGGGTCACACACGGAGAAGTAAT |
| | | GAGAAGAGCTGGTAATCTGGGGTAAAAGGGTTCAAAA |
| | | GAAGGTCGCCTGGTAGGGATGCAATACAAGGTTGTCT |
| | | TGGAGTTTACATTGACCAGATGATTTGGCTTTTTCTCT |
| | | GTTCAATTCACATTTTTCAGCGAGAATCGGATTGACGG |
| | | AGAAATGGCGGGGTGTGGGGTGGATAGATGGCAGAA |
| | | ATGCTCGCAATCACCGCGAAAGAAAGACTTTATGGAA |
| | | TAGAACTACTGGGTGGTGTAAGGATTACATAGCTAGT |
| | | CCAATGGAGTCCGTTGGAAAGGTAAGAAGAAGCTAAA |
| | | ACCGGCTAAGTAACTAGGGAAGAATGATCAGACTTTG |
| | | ATTTGATGAGGTCTGAAAATACTCTGCTGCTTTTTCAG |
| | | TTGCTTTTTCCCTGCAACCTATCATTTTCCTTTTCATAA |
| | | GCCTGCCTTTTCTGTTTTCACTTATATGAGTTCCGCCG |
| | | AGACTTCCCCAAATTCTCTCCTGGAACATTCTCTATCG |
| | | CTCTGCTTCCAAGTTGCGCCCCCTGGCACTGCCTAGTA |
| | | ATATTACCACGCGACTTATATTCAGTTCCACAATTTCC |
| | | AGTGTTCGTAGCAAATATCATCAGCCATGGCGAAGGC |
| | | AGATGGCAGTTTGCTCTACTATAATCCTCACAATCCAC |
| | | CCAGAAGGTATTACTTCTACATGGCTATATTCGCCGTT |
| | | TCTGTCATTTGCGTTTTGTACGGACCCTCACAACAATT |
| | | ATCATCTCCAAAAATAGACTATGATCCATTGACGCTCC |
| | | GATCACTTGATTTGAAGACTTTGGAAGCTCCTTCACAG |
| | | TTGAGTCCAGGCACCGTAGAAGATAATCTTCG |
| 9 | Pichia pastoris | AAAGCTAGAGTAAAATAGATATAGCGAGATTAGAGA |
| | Sequence of the | ATGAATACCTTCTTCTAAGCGATCGTCCGTCATCATAG |
| | 3′-Region used | AATATCATGGACTGTATAGTTTTTTTTTTGTACATATA |
| | for knock out of | ATGATTAAACGGTCATCCAACATCTCGTTGACAGATCT |
| | PpOCH1: | CTCAGTACGCGAAATCCCTGACTATCAAAGCAAGAAC |
| | | CGATGAAGAAAAAAACAACAGTAACCCAAACACCAC |
| | | AACAAACACTTTATCTTCTCCCCCCCAACACCAATCAT |
| | | CAAAGAGATGTCGGAACCAAACACCAAGAAGCAAAA |
| | | ACTAACCCCATATAAAAACATCCTGGTAGATAATGCT |
| | | GGTAACCCGCTCTCCTTCCATATTCTGGGCTACTTCAC |
| | | GAAGTCTGACCGGTCTCAGTTGATCAACATGATCCTC |
| | | GAAATGGGTGGCAAGATCGTTCCAGACCTGCCTCCTC |
| | | TGGTAGATGGAGTGTTGTTTTTGACAGGGGATTACAA |
| | | GTCTATTGATGAAGATACCCTAAAGCAACTGGGGGAC |
| | | GTTCCAATATACAGAGACTCCTTCATCTACCAGTGTTT |
| | | TGTGCACAAGACATCTCTTCCCATTGACACTTTCCGAA |
| | | TTGACAAGAACGTCGACTTGGCTCAAGATTTGATCAA |
| | | TAGGGCCCTTCAAGAGTCTGTGGATCATGTCACTTCTG |
| | | CCAGCACAGCTGCAGCTGCTGCTGTTGTTGTCGCTACC |
| | | AACGGCCTGTCTTCTAAACCAGACGCTCGTACTAGCA |
| | | AAATACAGTTCACTCCCGAAGAAGATCGTTTTATTCTT |
| | | GACTTTGTTAGGAGAAATCCTAAACGAAGAAACACAC |
| | | ATCAACTGTACACTGAGCTCGCTCAGCACATGAAAAA |
| | | CCATACGAATCATTCTATCCGCCACAGATTTCGTCGTA |
| | | ATCTTTCCGCTCAACTTGATTGGGTTTATGATATCGAT |
| | | CCATTGACCAACCAACCTCGAAAAGATGAAAACGGGA |
| | | ACTACATCAAGGTACAAGGCCTTCCA |
| 10 | Kluyveromyces | AAACGTAACGCCTGGCACTCTATTTTCTCAAACTTCTG |
| | lactis | GGACGGAAGAGCTAAATATTGTGTTGCTTGAACAAAC |
| | K. lactis UDP- | CCAAAAAAACAAAAAAATGAACAAACTAAAACTACA |
| | GlcNAc | CCTAAATAAACCGTGTGTAAAACGTAGTACCATATTA |
| | transporter gene | CTAGAAAAGATCACAAGTGTATCACACATGTGCATCT |
| | (KIMNN2-2) | CATATTACATCTTTTATCCAATCCATTCTCTCTATCCCG |
| | ORF underlined | TCTGTTCCTGTCAGATTCTTTTTCCATAAAAAGAAGAA |
| | | GACCCCGAATCTCACCGGTACAATGCAAAACTGCTGA |
| | | AAAAAAAAGAAAGTTCACTGGATACGGGAACAGTGC |
| | | CAGTAGGCTTCACCACATGGACAAAACAATTGACGAT |
| | | AAAATAAGCAGGTGAGCTTCTTTTTCAAGTCACGATC |
| | | CCTTTATGTCTCAGAAACAATATATACAAGCTAAACC |
| | | CTTTTGAACCAGTTCTCTCTTCATAGTTATGTTCACAT |
| | | AAATTGCGGGAACAAGACTCCGCTGGCTGTCAGGTAC |
| | | ACGTTGTAACGTTTTCGTCCGCCCAATTATTAGCACAA |
| | | CATTGGCAAAAAGAAAAACTGCTCGTTTTCTCTACAG |
| | | GTAAATTACAATTTTTTTCAGTAATTTTCGCTGAAAAA |
| | | TTTAAAGGGCAGGAAAAAAAGACGATCTCGACTTTGC |
| | | ATAGATGCAAGAACTGTGGTCAAAACTTGAAATAGTA |
| | | ATTTTGCTGTGCGTGAACTAATAAATATATATATATAT |
| | | ATATATATATATTTGTGTATTTTGTATATGTAATTGTGC |
| | | ACGTCTTGGCTATTGGATATAAGATTTTCGCGGGTTGA |
| | | TGACATAGAGCGTGTACTACTGTAATAGTTGTATATTC |
| | | AAAAGCTGCTGCGTGGAGAAAGACTAAAATAGATAA |
| | | AAAGCACACATTTTGACTTCGGTACCGTCAACTTAGTG |
| | | GGACAGTCTTTTATATTTGGTGTAAGCTCATTTCTGGT |
| | | ACTATTCGAAACAGAACAGTGTTTTCTGTATTACCGTC |
| | | CAATCGTTTGTCATGAGTTTTGTATTGATTTTGTCGTT |
| | | AGTGTTCGGAGGATGTTGTTCCAATGTGATTAGTTTCG |
| | | AGCACATGGTGCAAGGCAGCAATATAAATTTGGGAAA |
| | | TATTGTTACATTCACTCAATTCGTGTCTGTGACGCTAA |
| | | TTCAGTTGCCCAATGCTTTGGACTTCTCTCACTTTCCGT |
| | | TTAGGTTGCGACCTAGACACATTCCTCTTAAGATCCAT |
| | | ATGTTAGCTGTGTTTTTGTTCTTTACCAGTTCAGTCGCC |
| | | AATAACAGTGTGTTTAAATTTGACATTTCCGTTCCGAT |
| | | TCATATTATCATTAGATTTTCAGGTACCACTTTGACGA |
| | | TGATAATAGGTTGGGCTGTTTGTAATAAGAGGTACTCC |
| | | AAACTTCAGGTGCAATCTGCCATCATTATGACGCTTGG |
| | | TGCGATTGTCGCATCATTATACCGTGACAAAGAATTTT |
| | | CAATGGACAGTTTAAAGTTGAATACGGATTCAGTGGG |
| | | TATGACCCAAAAATCTATGTTTGGTATCTTTGTTGTGC |
| | | TAGTGGCCACTGCCTTGATGTCATTGTTGTCGTTGCTC |
| | | AACGAATGGACGTATAACAAGTACGGGAAACATTGGA |
| | | AAGAAACTTTGTTCTATTCGCATTTCTTGGCTCTACCG |
| | | TTGTTTATGTTGGGGTACACAAGGCTCAGAGACGAAT |
| | | TCAGAGACCTCTTAATTTCCTCAGACTCAATGGATATT |
| | | CCTATTGTTAAATTACCAATTGCTACGAAACTTTTCAT |
| | | GCTAATAGCAAATAACGTGACCCAGTTCATTTGTATC |
| | | AAAGGTGTTAACATGCTAGCTAGTAACACGGATGCTT |
| | | TGACACTTTCTGTCGTGCTTCTAGTGCGTAAATTTGTT |
| | | AGTCTTTTACTCAGTGTCTACATCTACAAGAACGTCCT |
| | | ATCCGTGACTGCATACCTAGGGACCATCACCGTGTTCC |
| | | TGGGAGCTGGTTTGTATTCATATGGTTCGGTCAAAACT |
| | | GCACTGCCTCGCTGAAACAATCCACGTCTGTATGATA |
| | | CTCGTTTCAGAATTTTTTTGATTTTCTGCCGGATATGGT |
| | | TTCTCATCTTTACAATCGCATTCTTAATTATACCAGAA |
| | | CGTAATTCAATGATCCCAGTGACTCGTAACTCTTATAT |
| | | GTCAATTTAAGC |
| 11 | Pichia pastoris | GGCCGAGCGGGCCTAGATTTTCACTACAAATTTCAAA |
| | Sequence of the | ACTACGCGGATTTATTGTCTCAGAGAGCAATTTGGCAT |
| | 5′-Region used | TTCTGAGCGTAGCAGGAGGCTTCATAAGATTGTATAG |
| | for knock out of | GACCGTACCAACAAATTGCCGAGGCACAACACGGTAT |
| | PpBMT2: | GCTGTGCACTTATGTGGCTACTTCCCTACAACGGAATG |
| | | AAACCTTCCTCTTTCCGCTTAAACGAGAAAGTGTGTCG |
| | | CAATTGAATGCAGGTGCCTGTGCGCCTTGGTGTATTGT |
| | | TTTTGAGGGCCCAATTTATCAGGCGCCTTTTTTCTTGG |
| | | TTGTTTTCCCTTAGCCTCAAGCAAGGTTGGTCTATTTC |
| | | ATCTCCGCTTCTATACCGTGCCTGATACTGTTGGATGA |
| | | GAACACGACTCAACTTCCTGCTGCTCTGTATTGCCAGT |
| | | GTTTTGTCTGTGATTTGGATCGGAGTCCTCCTTACTTG |
| | | GAATGATAATAATCTTGGCGGAATCTCCCTAAACGGA |
| | | GGCAAGGATTCTGCCTATGATGATCTGCTATCATTGGG |
| | | AAGCTTCAACGACATGGAGGTCGACTCCTATGTCACC |
| | | AACATCTACGACAATGCTCCAGTGCTAGGATGTACGG |
| | | ATTTGTCTTATCATGGATTGTTGAAAGTCACCCCAAAG |
| | | CATGACTTAGCTTGCGATTTGGAGTTCATAAGAGCTCA |
| | | GATTTTGGACATTGACGTTTACTCCGCCATAAAAGACT |
| | | TAGAAGATAAAGCCTTGACTGTAAAACAAAAGGTTGA |
| | | AAAACACTGGTTTACGTTTTATGGTAGTTCAGTCTTTC |
| | | TGCCCGAACACGATGTGCATTACCTGGTTAGACGAGT |
| | | CATCTTTTCGGCTGAAGGAAAGGCGAACTCTCCAGTA |
| | | ACATC |
| 12 | Pichia pastoris | CCATATGATGGGTGTTTGCTCACTCGTATGGATCAAAA |
| | Sequence of the | TTCCATGGTTTCTTCTGTACAACTTGTACACTTATTTGG |
| | 3′-Region used | ACTTTTCTAACGGTTTTTCTGGTGATTTGAGAAGTCCT |
| | for knock out of | TATTTTGGTGTTCGCAGCTTATCCGTGATTGAACCATC |
| | PpBMT2: | AGAAATACTGCAGCTCGTTATCTAGTTTCAGAATGTGT |
| | | TGTAGAATACAATCAATTCTGAGTCTAGTTTGGGTGGG |
| | | TCTTGGCGACGGGACCGTTATATGCATCTATGCAGTGT |
| | | TAAGGTACATAGAATGAAAATGTAGGGGTTAATCGAA |
| | | AGCATCGTTAATTTCAGTAGAACGTAGTTCTATTCCCT |
| | | ACCCAAATAATTTGCCAAGAATGCTTCGTATCCACAT |
| | | ACGCAGTGGACGTAGCAAATTTCACTTTGGACTGTGA |
| | | CCTCAAGTCGTTATCTTCTACTTGGACATTGATGGTCA |
| | | TTACGTAATCCACAAAGAATTGGATAGCCTCTCGTTTT |
| | | ATCTAGTGCACAGCCTAATAGCACTTAAGTAAGAGCA |
| | | ATGGACAAATTTGCATAGACATTGAGCTAGATACGTA |
| | | ACTCAGATCTTGTTCACTCATGGTGTACTCGAAGTACT |
| | | GCTGGAACCGTTACCTCTTATCATTTCGCTACTGGCTC |
| | | GTGAAACTACTGGATGAAAAAAAAAAAAGAGCTGAA |
| | | AGCGAGATCATCCCATTTTGTCATCATACAAATTCACG |
| | | CTTGCAGTTTTGCTTCGTTAACAAGACAAGATGTCTTT |
| | | ATCAAAGACCCGTTTTTTCTTCTTGAAGAATACTTCCC |
| | | TGTTGAGCACATGCAAACCATATTTATCTCAGATTTCA |
| | | CTCAACTTGGGTGCTTCCAAGAGAAGTAAAATTCTTCC |
| | | CACTGCATCAACTTCCAAGAAACCCGTAGACCAGTTT |
| | | CTCTTCAGCCAAAAGAAGTTGCTCGCCGATCACCGCG |
| | | GTAACAGAGGAGTCAGAAGGTTTCACACCCTTCCATC |
| | | CCGATTTCAAAGTCAAAGTGCTGCGTTGAACCAAGGT |
| | | TTTCAGGTTGCCAAAGCCCAGTCTGCAAAAACTAGTT |
| | | CCAAATGGCCTATTAATTCCCATAAAAGTGTTGGCTAC |
| | | GTATGTATCGGTACCTCCATTCTGGTATTTGCTATTGT |
| | | TGTCGTTGGTGGGTTGACTAGACTGACCGAATCCGGT |
| | | CTTTCCATAACGGAGTGGAAACCTATCACTGGTTCGGT |
| | | TCCCCCACTGACTGAGGAAGACTGGAAGTTGGAATTT |
| | | GAAAAATACAAACAAAGCCCTGAGTTTCAGGAACTAA |
| | | ATTCTCACATAACATTGGAAGAGTTCAAGTTTATATTT |
| | | TCCATGGAATGGGGACATAGATTGTTGGGAAGGGTCA |
| | | TCGGCCTGTCGTTTGTTCTTCCCACGTTTTACTTCATTG |
| | | CCCGTCGAAAGTGTTCCAAAGATGTTGCATTGAAACT |
| | | GCTTGCAATATGCTCTATGATAGGATTCCAAGGTTTCA |
| | | TCGGCTGGTGGATGGTGTATTCCGGATTGGACAAACA |
| | | GCAATTGGCTGAACGTAACTCCAAACCAACTGTGTCT |
| | | CCATATCGCTTAACTACCCATCTTGGAACTGCATTTGT |
| | | TATTTACTGTTACATGATTTACACAGGGCTTCAAGTTT |
| | | TGAAGAACTATAAGATCATGAAACAGCCTGAAGCGTA |
| | | TGTTCAAATTTTCAAGCAAATTGCGTCTCCAAAATTGA |
| | | AAACTTTCAAGAGACTCTCTTCAGTTCTATTAGGCCTG |
| | | GTG |
| 13 | Mus musculus | ATGTCTGCCAACCTAAAATATCTTTCCTTGGGAATTTT |
| | DNA encodes | GGTGTTTCAGACTACCAGTCTGGTTCTAACGATGCGGT |
| | MmSLC35A3 | ATTCTAGGACTTTAAAAGAGGAGGGGCCTCGTTATCT |
| | UDP-GlcNAc | GTCTTCTACAGCAGTGGTTGTGGCTGAATTTTTGAAGA |
| | transporter | TAATGGCCTGCATCTTTTTAGTCTACAAAGACAGTAAG |
| | | TGTAGTGTGAGAGCACTGAATAGAGTACTGCATGATG |
| | | AAATTCTTAATAAGCCCATGGAAACCCTGAAGCTCGC |
| | | TATCCCGTCAGGGATATATACTCTTCAGAACAACTTAC |
| | | TCTATGTGGCACTGTCAAACCTAGATGCAGCCACTTAC |
| | | CAGGTTACATATCAGTTGAAAATACTTACAACAGCAT |
| | | TATTTTCTGTGTCTATGCTTGGTAAAAAATTAGGTGTG |
| | | TACCAGTGGCTCTCCCTAGTAATTCTGATGGCAGGAGT |
| | | TGCTTTTGTACAGTGGCCTTCAGATTCTCAAGAGCTGA |
| | | ACTCTAAGGACCTTTCAACAGGCTCACAGTTTGTAGG |
| | | CCTCATGGCAGTTCTCACAGCCTGTTTTTCAAGTGGCT |
| | | TTGCTGGAGTTTATTTTGAGAAAATCTTAAAAGAAAC |
| | | AAAACAGTCAGTATGGATAAGGAACATTCAACTTGGT |
| | | TTCTTTGGAAGTATATTTGGATTAATGGGTGTATACGT |
| | | TTATGATGGAGAATTGGTCTCAAAGAATGGATTTTTTC |
| | | AGGGATATAATCAACTGACGTGGATAGTTGTTGCTCT |
| | | GCAGGCACTTGGAGGCCTTGTAATAGCTGCTGTCATC |
| | | AAATATGCAGATAACATTTTAAAAGGATTTGCGACCT |
| | | CCTTATCCATAATATTGTCAACAATAATATCTTATTTT |
| | | TGGTTGCAAGATTTTGTGCCAACCAGTGTCTTTTTCCT |
| | | TGGAGCCATCCTTGTAATAGCAGCTACTTTCTTGTATG |
| | | GTTACGATCCCAAACCTGCAGGAAATCCCACTAAAGC |
| | | ATAG |
| 14 | Pichia pastoris | TTTTTGTAGAAATGTCTTGGTGTCCTCGTCCAATCAGG |
| | PpGAPDH | TAGCCATCTCTGAAATATCTGGCTCCGTTGCAACTCCG |
| | promoter | AACGACCTGCTGGCAACGTAAAATTCTCCGGGGTAAA |
| | | ACTTAAATGTGGAGTAATGGAACCAGAAACGTCTCTT |
| | | CCCTTCTCTCTCCTTCCACCGCCCGTTACCGTCCCTAG |
| | | GAAATTTTACTCTGCTGGAGAGCTTCTTCTACGGCCCC |
| | | CTTGCAGCAATGCTCTTCCCAGCATTACGTTGCGGGTA |
| | | AAACGGAGGTCGTGTACCCGACCTAGCAGCCCAGGGA |
| | | TGGAAAAGTCCCGGCCGTCGCTGGCAATAATAGCGGG |
| | | CGGACGCATGTCATGAGATTATTGGAAACCACCAGAA |
| | | TCGAATATAAAAGGCGAACACCTTTCCCAATTTTGGTT |
| | | TCTCCTGACCCAAAGACTTTAAATTTAATTTATTTGTC |
| | | CCTATTTCAATCAATTGAACAACTATCAAAACACA |
| 15 | Saccharomyces | ACAGGCCCCTTTTCCTTTGTCGATATCATGTAATTAGT |
| | cerevisiae | TATGTCACGCTTACATTCACGCCCTCCTCCCACATCCG |
| | ScCYC TT | CTCTAACCGAAAAGGAAGGAGTTAGACAACCTGAAGT |
| | | CTAGGTCCCTATTTATTTTTTTTAATAGTTATGTTAGTA |
| | | TTAAGAACGTTATTTATATTTCAAATTTTTCTTTTTTTT |
| | | CTGTACAAACGCGTGTACGCATGTAACATTATACTGA |
| | | AAACCTTGCTTGAGAAGGTTTTGGGACGCTCGAAGGC |
| | | TTTAATTTGCAAGCTGCCGGCTCTTAAG |
| 16 | Pichia pastoris | GATCTGGCCATTGTGAAACTTGACACTAAAGACAAAA |
| | Sequence of the | CTCTTAGAGTTTCCAATCACTTAGGAGACGATGTTTCC |
| | 5′-Region used | TACAACGAGTACGATCCCTCATTGATCATGAGCAATTT |
| | for knock out of | GTATGTGAAAAAAGTCATCGACCTTGACACCTTGGAT |
| | PpMNN4L1: | AAAAGGGCTGGAGGAGGTGGAACCACCTGTGCAGGC |
| | | GGTCTGAAAGTGTTCAAGTACGGATCTACTACCAAAT |
| | | ATACATCTGGTAACCTGAACGGCGTCAGGTTAGTATA |
| | | CTGGAACGAAGGAAAGTTGCAAAGCTCCAAATTTGTG |
| | | GTTCGATCCTCTAATTACTCTCAAAAGCTTGGAGGAA |
| | | ACAGCAACGCCGAATCAATTGACAACAATGGTGTGGG |
| | | TTTTGCCTCAGCTGGAGACTCAGGCGCATGGATTCTTT |
| | | CCAAGCTACAAGATGTTAGGGAGTACCAGTCATTCAC |
| | | TGAAAAGCTAGGTGAAGCTACGATGAGCATTTTCGAT |
| | | TTCCACGGTCTTAAACAGGAGACTTCTACTACAGGGC |
| | | TTGGGGTAGTTGGTATGATTCATTCTTACGACGGTGAG |
| | | TTCAAACAGTTTGGTTTGTTCACTCCAATGACATCTAT |
| | | TCTACAAAGACTTCAACGAGTGACCAATGTAGAATGG |
| | | TGTGTAGCGGGTTGCGAAGATGGGGATGTGGACACTG |
| | | AAGGAGAACACGAATTGAGTGATTTGGAACAACTGCA |
| | | TATGCATAGTGATTCCGACTAGTCAGGCAAGAGAGAG |
| | | CCCTCAAATTTACCTCTCTGCCCCTCCTCACTCCTTTTG |
| | | GTACGCATAATTGCAGTATAAAGAACTTGCTGCCAGC |
| | | CAGTAATCTTATTTCATACGCAGTTCTATATAGCACAT |
| | | AATCTTGCTTGTATGTATGAAATTTACCGCGTTTTAGT |
| | | TGAAATTGTTTATGTTGTGTGCCTTGCATGAAATCTCT |
| | | CGTTAGCCCTATCCTTACATTTAACTGGTCTCAAAACC |
| | | TCTACCAATTCCATTGCTGTACAACAATATGAGGCGG |
| | | CATTACTGTAGGGTTGGAAAAAAATTGTCATTCCAGC |
| | | TAGAGATCACACGACTTCATCACGCTTATTGCTCCTCA |
| | | TTGCTAAATCATTTACTCTTGACTTCGACCCAGAAAAG |
| | | TTCGCC |
| 17 | Pichia pastoris | GCATGTCAAACTTGAACACAACGACTAGATAGTTGTT |
| | Sequence of the | TTTTCTATATAAAACGAAACGTTATCATCTTTAATAAT |
| | 3′-Region used | CATTGAGGTTTACCCTTATAGTTCCGTATTTTCGTTTCC |
| | for knock out of | AAACTTAGTAATCTTTTGGAAATATCATCAAAGCTGGT |
| | PpMNN4L1: | GCCAATCTTCTTGTTTGAAGTTTCAAACTGCTCCACCA |
| | | AGCTACTTAGAGACTGTTCTAGGTCTGAAGCAACTTC |
| | | GAACACAGAGACAGCTGCCGCCGATTGTTCTTTTTTGT |
| | | GTTTTTCTTCTGGAAGAGGGGCATCATCTTGTATGTCC |
| | | AATGCCCGTATCCTTTCTGAGTTGTCCGACACATTGTC |
| | | CTTCGAAGAGTTTCCTGACATTGGGCTTCTTCTATCCG |
| | | TGTATTAATTTTGGGTTAAGTTCCTCGTTTGCATAGCA |
| | | GTGGATACCTCGATTTTTTTGGCTCCTATTTACCTGAC |
| | | ATAATATTCTACTATAATCCAACTTGGACGCGTCATCT |
| | | ATGATAACTAGGCTCTCCTTTGTTCAAAGGGGACGTCT |
| | | TCATAATCCACTGGCACGAAGTAAGTCTGCAACGAGG |
| | | CGGCTTTTGCAACAGAACGATAGTGTCGTTTCGTACTT |
| | | GGACTATGCTAAACAAAAGGATCTGTCAAACATTTCA |
| | | ACCGTGTTTCAAGGCACTCTTTACGAATTATCGACCAA |
| | | GACCTTCCTAGACGAACATTTCAACATATCCAGGCTA |
| | | CTGCTTCAAGGTGGTGCAAATGATAAAGGTATAGATA |
| | | TTAGATGTGTTTGGGACCTAAAACAGTTCTTGCCTGAA |
| | | GATTCCCTTGAGCAACAGGCTTCAATAGCCAAGTTAG |
| | | AGAAGCAGTACCAAATCGGTAACAAAAGGGGGAAGC |
| | | ATATAAAACCTTTACTATTGCGACAAAATCCATCCTTG |
| | | AAAGTAAAGCTGTTTGTTCAATGTAAAGCATACGAAA |
| | | CGAAGGAGGTAGATCCTAAGATGGTTAGAGAACTTAA |
| | | CGGGACATACTCCAGCTGCATCCCATATTACGATCGCT |
| | | GGAAGACTTTTTTCATGTACGTATCGCCCACCAACCTT |
| | | TCAAAGCAAGCTAGGTATGATTTTGACAGTTCTCACA |
| | | ATCCATTGGTTTTCATGCAACTTGAAAAAACCCAACTC |
| | | AAACTTCATGGGGATCCATACAATGTAAATCATTACG |
| | | AGAGGGCGAGGTTGAAAAGTTTCCATTGCAATCACGT |
| | | CGCATCATGGCTACTGAAAGGCCTTAAC |
| 18 | Pichia pastoris | TCATTCTATATGTTCAAGAAAAGGGTAGTGAAAGGAA |
| | Sequence of the | AGAAAAGGCATATAGGCGAGGGAGAGTTAGCTAGCA |
| | 5′-Region used | TACAAGATAATGAAGGATCAATAGCGGTAGTTAAAGT |
| | for knock out of | GCACAAGAAAAGAGCACCTGTTGAGGCTGATGATAAA |
| | PpPNO1 and | GCTCCAATTACATTGCCACAGAGAAACACAGTAACAG |
| | PpMNN4: | AAATAGGAGGGGATGCACCACGAGAAGAGCATTCAG |
| | | TGAACAACTTTGCCAAATTCATAACCCCAAGCGCTAA |
| | | TAAGCCAATGTCAAAGTCGGCTACTAACATTAATAGT |
| | | ACAACAACTATCGATTTTCAACCAGATGTTTGCAAGG |
| | | ACTACAAACAGACAGGTTACTGCGGATATGGTGACAC |
| | | TTGTAAGTTTTTGCACCTGAGGGATGATTTCAAACAGG |
| | | GATGGAAATTAGATAGGGAGTGGGAAAATGTCCAAA |
| | | AGAAGAAGCATAATACTCTCAAAGGGGTTAAGGAGAT |
| | | CCAAATGTTTAATGAAGATGAGCTCAAAGATATCCCG |
| | | TTTAAATGCATTATATGCAAAGGAGATTACAAATCAC |
| | | CCGTGAAAACTTCTTGCAATCATTATTTTTGCGAACAA |
| | | TGTTTCCTGCAACGGTCAAGAAGAAAACCAAATTGTA |
| | | TTATATGTGGCAGAGACACTTTAGGAGTTGCTTTACCA |
| | | GCAAAGAAGTTGTCCCAATTTCTGGCTAAGATACATA |
| | | ATAATGAAAGTAATAAAGTTTAGTAATTGCATTGCGTT |
| | | GACTATTGATTGCATTGATGTCGTGTGATACTTTCACC |
| | | GAAAAAAAACACGAAGCGCAATAGGAGCGGTTGCAT |
| | | ATTAGTCCCCAAAGCTATTTAATTGTGCCTGAAACTGT |
| | | TTTTTAAGCTCATCAAGCATAATTGTATGCATTGCGAC |
| | | GTAACCAACGTTTAGGCGCAGTTTAATCATAGCCCAC |
| | | TGCTAAGCC |
| 19 | Pichia pastoris | CGGAGGAATGCAAATAATAATCTCCTTAATTACCCAC |
| | Sequence of the | TGATAAGCTCAAGAGACGCGGTTTGAAAACGATATAA |
| | 3′-Region used | TGAATCATTTGGATTTTATAATAAACCCTGACAGTTTT |
| | for knock out of | TCCACTGTATTGTTTTAACACTCATTGGAAGCTGTATT |
| | PpPNO1 and | GATTCTAAGAAGCTAGAAATCAATACGGCCATACAAA |
| | PpMNN4: | AGATGACATTGAATAAGCACCGGCTTTTTTGATTAGC |
| | | ATATACCTTAAAGCATGCATTCATGGCTACATAGTTGT |
| | | TAAAGGGCTTCTTCCATTATCAGTATAATGAATTACAT |
| | | AATCATGCACTTATATTTGCCCATCTCTGTTCTCTCACT |
| | | CTTGCCTGGGTATATTCTATGAAATTGCGTATAGCGTG |
| | | TCTCCAGTTGAACCCCAAGCTTGGCGAGTTTGAAGAG |
| | | AATGCTAACCTTGCGTATTCCTTGCTTCAGGAAACATT |
| | | CAAGGAGAAACAGGTCAAGAAGCCAAACATTTTGATC |
| | | CTTCCCGAGTTAGCATTGACTGGCTACAATTTTCAAAG |
| | | CCAGCAGCGGATAGAGCCTTTTTTGGAGGAAACAACC |
| | | AAGGGAGCTAGTACCCAATGGGCTCAAAAAGTATCCA |
| | | AGACGTGGGATTGCTTTACTTTAATAGGATACCCAGA |
| | | AAAAAGTTTAGAGAGCCCTCCCCGTATTTACAACAGT |
| | | GCGGTACTTGTATCGCCTCAGGGAAAAGTAATGAACA |
| | | ACTACAGAAAGTCCTTCTTGTATGAAGCTGATGAACA |
| | | TTGGGGATGTTCGGAATCTTCTGATGGGTTTCAAACAG |
| | | TAGATTTATTAATTGAAGGAAAGACTGTAAAGACATC |
| | | ATTTGGAATTTGCATGGATTTGAATCCTTATAAATTTG |
| | | AAGCTCCATTCACAGACTTCGAGTTCAGTGGCCATTGC |
| | | TTGAAAACCGGTACAAGACTCATTTTGTGCCCAATGG |
| | | CCTGGTTGTCCCCTCTATCGCCTTCCATTAAAAAGGAT |
| | | CTTAGTGATATAGAGAAAAGCAGACTTCAAAAGTTCT |
| | | ACCTTGAAAAAATAGATACCCCGGAATTTGACGTTAA |
| | | TTACGAATTGAAAAAAGATGAAGTATTGCCCACCCGT |
| | | ATGAATGAAACGTTGGAAACAATTGACTTTGAGCCTT |
| | | CAAAACCGGACTACTCTAATATAAATTATTGGATACT |
| | | AAGGTTTTTTCCCTTTCTGACTCATGTCTATAAACGAG |
| | | ATGTGCTCAAAGAGAATGCAGTTGCAGTCTTATGCAA |
| | | CCGAGTTGGCATTGAGAGTGATGTCTTGTACGGAGGA |
| | | TCAACCACGATTCTAAACTTCAATGGTAAGTTAGCATC |
| | | GACACAAGAGGAGCTGGAGTTGTACGGGCAGACTAAT |
| | | AGTCTCAACCCCAGTGTGGAAGTATTGGGGGCCCTTG |
| | | GCATGGGTCAACAGGGAATTCTAGTACGAGACATTGA |
| | | ATTAACATAATATACAATATACAATAAACACAAATAA |
| | | AGAATACAAGCCTGACAAAAATTCACAAATTATTGCC |
| | | TAGACTTGTCGTTATCAGCAGCGACCTTTTTCCAATGC |
| | | TCAATTTCACGATATGCCTTTTCTAGCTCTGCTTTAAG |
| | | CTTCTCATTGGAATTGGCTAACTCGTTGACTGCTTGGT |
| | | CAGTGATGAGTTTCTCCAAGGTCCATTTCTCGATGTTG |
| | | TTGTTTTCGTTTTCCTTTAATCTCTTGATATAATCAACA |
| | | GCCTTCTTTAATATCTGAGCCTTGTTCGAGTCCCCTGT |
| | | TGGCAACAGAGCGGCCAGTTCCTTTATTCCGTGGTTTA |
| | | TATTTTCTCTTCTACGCCTTTCTACTTCTTTGTGATTCT |
| | | CTTTACGCATCTTATGCCATTCTTCAGAACCAGTGGCT |
| | | GGCTTAACCGAATAGCCAGAGCCTGAAGAAGCCGCAC |
| | | TAGAAGAAGCAGTGGCATTGTTGACTATGG |
| 20 | human | TCAGTCAGTGCTCTTGATGGTGACCCAGCAAGTTTGAC |
| | DNA encodes | CAGAGAAGTGATTAGATTGGCCCAAGACGCAGAGGTG |
| | human GnTI | GAGTTGGAGAGACAACGTGGACTGCTGCAGCAAATCG |
| | catalytic domain | GAGATGCATTGTCTAGTCAAAGAGGTAGGGTGCCTAC |
| | (NA) | CGCAGCTCCTCCAGCACAGCCTAGAGTGCATGTGACC |
| | Codon- | CCTGCACCAGCTGTGATTCCTATCTTGGTCATCGCCTG |
| | optimized | TGACAGATCTACTGTTAGAAGATGTCTGGACAAGCTG |
| | | TTGCATTACAGACCATCTGCTGAGTTGTTCCCTATCAT |
| | | CGTTAGTCAAGACTGTGGTCACGAGGAGACTGCCCAA |
| | | GCCATCGCCTCCTACGGATCTGCTGTCACTCACATCAG |
| | | ACAGCCTGACCTGTCATCTATTGCTGTGCCACCAGACC |
| | | ACAGAAAGTTCCAAGGTTACTACAAGATCGCTAGACA |
| | | CTACAGATGGGCATTGGGTCAAGTCTTCAGACAGTTT |
| | | AGATTCCCTGCTGCTGTGGTGGTGGAGGATGACTTGG |
| | | AGGTGGCTCCTGACTTCTTTGAGTACTTTAGAGCAACC |
| | | TATCCATTGCTGAAGGCAGACCCATCCCTGTGGTGTGT |
| | | CTCTGCCTGGAATGACAACGGTAAGGAGCAAATGGTG |
| | | GACGCTTCTAGGCCTGAGCTGTTGTACAGAACCGACT |
| | | TCTTTCCTGGTCTGGGATGGTTGCTGTTGGCTGAGTTG |
| | | TGGGCTGAGTTGGAGCCTAAGTGGCCAAAGGCATTCT |
| | | GGGACGACTGGATGAGAAGACCTGAGCAAAGACAGG |
| | | GTAGAGCCTGTATCAGACCTGAGATCTCAAGAACCAT |
| | | GACCTTTGGTAGAAAGGGAGTGTCTCACGGTCAATTC |
| | | TTTGACCAACACTTGAAGTTTATCAAGCTGAACCAGC |
| | | AATTTGTGCACTTCACCCAACTGGACCTGTCTTACTTG |
| | | CAGAGAGAGGCCTATGACAGAGATTTCCTAGCTAGAG |
| | | TCTACGGAGCTCCTCAACTGCAAGTGGAGAAAGTGAG |
| | | GACCAATGACAGAAAGGAGTTGGGAGAGGTGAGAGT |
| | | GCAGTACACTGGTAGGGACTCCTTTAAGGCTTTCGCTA |
| | | AGGCTCTGGGTGTCATGGATGACCTTAAGTCTGGAGT |
| | | TCCTAGAGCTGGTTACAGAGGTATTGTCACCTTTCAAT |
| | | TCAGAGGTAGAAGAGTCCACTTGGCTCCTCCACCTAC |
| | | TTGGGAGGGTTATGATCCTTCTTGGAATTAG |
| 21 | Pichia pastoris | ATGCCCAGAAAAATATTTAACTACTTCATTTTGACTGT |
| | DNA encodes | ATTCATGGCAATTCTTGCTATTGTTTTACAATGGTCTA |
| | Pp SEC12 (10) | TAGAGAATGGACATGGGCGCGCC |
| | The last 9 | |
| | nucleotides are | |
| | the linker | |
| | containing the | |
| | AscI restriction | |
| | site used for | |
| | fusion to | |
| | proteins of | |
| | interest. | |
| 22 | Pichia pastoris | GAAGTAAAGTTGGCGAAACTTTGGGAACCTTTGGTTA |
| | Sequence of the | AAACTTTGTAATTTTTGTCGCTACCCATTAGGCAGAAT |
| | PpSEC4 | CTGCATCTTGGGAGGGGGATGTGGTGGCGTTCTGAGA |
| | promoter: | TGTACGCGAAGAATGAAGAGCCAGTGGTAACAACAG |
| | | GCCTAGAGAGATACGGGCATAATGGGTATAACCTACA |
| | | AGTTAAGAATGTAGCAGCCCTGGAAACCAGATTGAAA |
| | | CGAAAAACGAAATCATTTAAACTGTAGGATGTTTTGG |
| | | CTCATTGTCTGGAAGGCTGGCTGTTTATTGCCCTGTTC |
| | | TTTGCATGGGAATAAGCTATTATATCCCTCACATAATC |
| | | CCAGAAAATAGATTGAAGCAACGCGAAATCCTTACGT |
| | | ATCGAAGTAGCCTTCTTACACATTCACGTTGTACGGAT |
| | | AAGAAAACTACTCAAACGAACAATC |
| 23 | Pichia pastoris | AATAGATATAGCGAGATTAGAGAATGAATACCTTCTT |
| | Sequence of the | CTAAGCGATCGTCCGTCATCATAGAATATCATGGACT |
| | PpOCH1 | GTATAGTTTTTTTTTTGTACATATAATGATTAAACGGT |
| | terminator: | CATCCAACATCTCGTTGACAGATCTCTCAGTACGCGA |
| | | AATCCCTGACTATCAAAGCAAGAACCGATGAAGAAAA |
| | | AAACAACAGTAACCCAAACACCACAACAAACACTTTA |
| | | TCTTCTCCCCCCCAACACCAATCATCAAAGAGATGTCG |
| | | GAACACAAACACCAAGAAGCAAAAACTAACCCCATA |
| | | TAAAAACATCCTGGTAGATAATGCTGGTAACCCGCTC |
| | | TCCTTCCATATTCTGGGCTACTTCACGAAGTCTGACCG |
| | | GTCTCAGTTGATCAACATGATCCTCGAAATGG |
| 24 | Mus musculus | GAGCCCGCTGACGCCACCATCCGTGAGAAGAGGGCAA |
| | DNA encodes | AGATCAAAGAGATGATGACCCATGCTTGGAATAATTA |
| | Mm ManI | TAAACGCTATGCGTGGGGCTTGAACGAACTGAAACCT |
| | catalytic domain | ATATCAAAAGAAGGCCATTCAAGCAGTTTGTTTGGCA |
| | (FB) | ACATCAAAGGAGCTACAATAGTAGATGCCCTGGATAC |
| | | CCTTTTCATTATGGGCATGAAGACTGAATTTCAAGAA |
| | | GCTAAATCGTGGATTAAAAAATATTTAGATTTTAATGT |
| | | GAATGCTGAAGTTTCTGTTTTTGAAGTCAACATACGCT |
| | | TCGTCGGTGGACTGCTGTCAGCCTACTATTTGTCCGGA |
| | | GAGGAGATATTTCGAAAGAAAGCAGTGGAACTTGGGG |
| | | TAAAATTGCTACCTGCATTTCATACTCCCTCTGGAATA |
| | | CCTTGGGCATTGCTGAATATGAAAAGTGGGATCGGGC |
| | | GGAACTGGCCCTGGGCCTCTGGAGGCAGCAGTATCCT |
| | | GGCCGAATTTGGAACTCTGCATTTAGAGTTTATGCACT |
| | | TGTCCCACTTATCAGGAGACCCAGTCTTTGCCGAAAA |
| | | GGTTATGAAAATTCGAACAGTGTTGAACAAACTGGAC |
| | | AAACCAGAAGGCCTTTATCCTAACTATCTGAACCCCA |
| | | GTAGTGGACAGTGGGGTCAACATCATGTGTCGGTTGG |
| | | AGGACTTGGAGACAGCTTTTATGAATATTTGCTTAAGG |
| | | CGTGGTTAATGTCTGACAAGACAGATCTCGAAGCCAA |
| | | GAAGATGTATTTTGATGCTGTTCAGGCCATCGAGACTC |
| | | ACTTGATCCGCAAGTCAAGTGGGGGACTAACGTACAT |
| | | CGCAGAGTGGAAGGGGGGCCTCCTGGAACACAAGAT |
| | | GGGCCACCTGACGTGCTTTGCAGGAGGCATGTTTGCA |
| | | CTTGGGGCAGATGGAGCTCCGGAAGCCCGGGCCCAAC |
| | | ACTACCTTGAACTCGGAGCTGAAATTGCCCGCACTTGT |
| | | CATGAATCTTATAATCGTACATATGTGAAGTTGGGAC |
| | | CGGAAGCGTTTCGATTTGATGGCGGTGTGGAAGCTAT |
| | | TGCCACGAGGCAAAATGAAAAGTATTACATCTTACGG |
| | | CCCGAGGTCATCGAGACATACATGTACATGTGGCGAC |
| | | TGACTCACGACCCCAAGTACAGGACCTGGGCCTGGGA |
| | | AGCCGTGGAGGCTCTAGAAAGTCACTGCAGAGTGAAC |
| | | GGAGGCTACTCAGGCTTACGGGATGTTTACATTGCCC |
| | | GTGAGAGTTATGACGATGTCCAGCAAAGTTTCTTCCTG |
| | | GCAGAGACACTGAAGTATTTGTACTTGATATTTTCCGA |
| | | TGATGACCTTCTTCCACTAGAACACTGGATCTTCAACA |
| | | CCGAGGCTCATCCTTTCCCTATACTCCGTGAACAGAAG |
| | | AAGGAAATTGATGGCAAAGAGAAATGA |
| 25 | Saccharomyces | ATGAACACTATCCACATAATAAAATTACCGCTTAACT |
| | cerevisiae | ACGCCAACTACACCTCAATGAAACAAAAAATCTCTAA |
| | DNA encodes | ATTTTTCACCAACTTCATCCTTATTGTGCTGCTTTCTTA |
| | ScSEC12 (8) | CATTTTACAGTTCTCCTATAAGCACAATTTGCATTCCA |
| | The last 9 | TGCTTTTCAATTACGCGAAGGACAATTTTCTAACGAAA |
| | nucleotides are | AGAGACACCATCTCTTCGCCCTACGTAGTTGATGAAG |
| | the linker | ACTTACATCAAACAACTTTGTTTGGCAACCACGGTAC |
| | containing the | AAAAACATCTGTACCTAGCGTAGATTCCATAAAAGTG |
| | AscI restriction | CATGGCGTGGGGCGCGCC |
| | site used for | |
| | fusion to | |
| | proteins of | |
| | interest | |
| 26 | Pichia pastoris | GAGTCGGCCAAGAGATGATAACTGTTACTAAGCTTCT |
| | Sequence of the | CCGTAATTAGTGGTATTTTGTAACTTTTACCAATAATC |
| | 5′-region that | GTTTATGAATACGGATATTTTTCGACCTTATCCAGTGC |
| | was used to | CAAATCACGTAACTTAATCATGGTTTAAATACTCCACT |
| | knock into the | TGAACGATTCATTATTCAGAAAAAAGTCAGGTTGGCA |
| | PpADE1 locus: | GAAACACTTGGGCGCTTTGAAGAGTATAAGAGTATTA |
| | | AGCATTAAACATCTGAACTTTCACCGCCCCAATATACT |
| | | ACTCTAGGAAACTCGAAAAATTCCTTTCCATGTGTCAT |
| | | CGCTTCCAACACACTTTGCTGTATCCTTCCAAGTATGT |
| | | CCATTGTGAACACTGATCTGGACGGAATCCTACCTTTA |
| | | ATCGCCAAAGGAAAGGTTAGAGACATTTATGCAGTCG |
| | | ATGAGAACAACTTGCTGTTCGTCGCAACTGACCGTAT |
| | | CTCCGCTTACGATGTGATTATGACAAACGGTATTCCTG |
| | | ATAAGGGAAAGATTTTGACTCAGCTCTCAGTTTTCTGG |
| | | TTTGATTTTTTGGCACCCTACATAAAGAATCATTTGGT |
| | | TGCTTCTAATGACAAGGAAGTCTTTGCTTTACTACCAT |
| | | CAAAACTGTCTGAAGAAAAaTACAAATCTCAATTAGA |
| | | GGGACGATCCTTGATAGTAAAAAAGCACAGACTGATA |
| | | CCTTTGGAAGCCATTGTCAGAGGTTACATCACTGGAA |
| | | GTGCATGGAAAGAGTACAAGAACTCAAAAACTGTCCA |
| | | TGGAGTCAAGGTTGAAAACGAGAACCTTCAAGAGAGC |
| | | GACGCCTTTCCAACTCCGATTTTCACACCTTCAACGAA |
| | | AGCTGAACAGGGTGAACACGATGAAAACATCTCTATT |
| | | GAACAAGCTGCTGAGATTGTAGGTAAAGACATTTGTG |
| | | AGAAGGTCGCTGTCAAGGCGGTCGAGTTGTATTCTGC |
| | | TGCAAAAAACCTCGCCCTTTTGAAGGGGATCATTATT |
| | | GCTGATACGAAATTCGAATTTGGACTGGACGAAAACA |
| | | ATGAATTGGTACTAGTAGATGAAGTTTTAACTCCAGAT |
| | | TCTTCTAGATTTTGGAATCAAAAGACTTACCAAGTGG |
| | | GTAAATCGCAAGAGAGTTACGATAAGCAGTTTCTCAG |
| | | AGATTGGTTGACGGCCAACGGATTGAATGGCAAAGAG |
| | | GGCGTAGCCATGGATGCAGAAATTGCTATCAAGAGTA |
| | | AAGAAAAGTATATTGAAGCTTATGAAGCAATTACTGG |
| | | CAAGAAATGGGCTTGA |
| 27 | Pichia pastoris | ATTTACAATTAGTAATATTAAGGTGGTAAAAACATTC |
| | PpALG3 TT | GTAGAATTGAAATGAATTAATATAGTATGACAATGGT |
| | | TCATGTCTATAAATCTCCGGCTTCGGTACCTTCTCCCC |
| | | AATTGAATACATTGTCAAAATGAATGGTTGAACTATT |
| | | AGGTTCGCCAGTTTCGTTATTAAGAAAACTGTTAAAAT |
| | | CAAATTCCATATCATCGGTTCCAGTGGGAGGACCAGT |
| | | TCCATCGCCAAAATCCTGTAAGAATCCATTGTCAGAA |
| | | CCTGTAAAGTCAGTTTGAGATGAAATTTTTCCGGTCTT |
| | | TGTTGACTTGGAAGCTTCGTTAAGGTTAGGTGAAACA |
| | | GTTTGATCAACCAGCGGCTCCCGTTTTCGTCGCTTAGT |
| | | AG |
| 28 | Pichia pastoris | ATGATTAGTACCCTCCTCGCCTTTTTCAGACATCTGAA |
| | Sequence of the | ATTTCCCTTATTCTTCCAATTCCATATAAAATCCTATTT |
| | 3′-region that | AGGTAATTAGTAAACAATGATCATAAAGTGAAATCAT |
| | was used to | TCAAGTAACCATTCCGTTTATCGTTGATTTAAAATCAA |
| | knock into the | TAACGAATGAATGTCGGTCTGAGTAGTCAATTTGTTGC |
| | PpADE1 locus: | CTTGGAGCTCATTGGCAGGGGGTCTTTTGGCTCAGTAT |
| | | GGAAGGTTGAAAGGAAAACAGATGGAAAGTGGTTCG |
| | | TCAGAAAAGAGGTATCCTACATGAAGATGAATGCCAA |
| | | AGAGATATCTCAAGTGATAGCTGAGTTCAGAATTCTT |
| | | AGTGAGTTAAGCCATCCCAACATTGTGAAGTACCTTC |
| | | ATCACGAACATATTTCTGAGAATAAAACTGTCAATTT |
| | | ATACATGGAATACTGTGATGGTGGAGATCTCTCCAAG |
| | | CTGATTCGAACACATAGAAGGAACAAAGAGTACATTT |
| | | CAGAAGAAAAAATATGGAGTATTTTTACGCAGGTTTT |
| | | ATTAGCATTGTATCGTTGTCATTATGGAACTGATTTCA |
| | | CGGCTTCAAAGGAGTTTGAATCGCTCAATAAAGGTAA |
| | | TAGACGAACCCAGAATCCTTCGTGGGTAGACTCGACA |
| | | AGAGTTATTATTCACAGGGATATAAAACCCGACAACA |
| | | TCTTTCTGATGAACAATTCAAACCTTGTCAAACTGGGA |
| | | GATTTTGGATTAGCAAAAATTCTGGACCAAGAAAACG |
| | | ATTTTGCCAAAACATACGTCGGTACGCCGTATTACATG |
| | | TCTCCTGAAGTGCTGTTGGACCAACCCTACTCACCATT |
| | | ATGTGATATATGGTCTCTTGGGTGCGTCATGTATGAGC |
| | | TATGTGCATTGAGGCCTCCTT |
| 29 | Saccharomyces | ATGACAGCTCAGTTACAAAGTGAAAGTACTTCTAAAA |
| | cerevisiae | TTGTTTTGGTTACAGGTGGTGCTGGATACATTGGTTCA |
| | DNA encodes | CACACTGTGGTAGAGCTAATTGAGAATGGATATGACT |
| | ScGAL10 | GTGTTGTTGCTGATAACCTGTCGAATTCAACTTATGAT |
| | | TCTGTAGCCAGGTTAGAGGTCTTGACCAAGCATCACA |
| | | TTCCCTTCTATGAGGTTGATTTGTGTGACCGAAAAGGT |
| | | CTGGAAAAGGTTTTCAAAGAATATAAAATTGATTCGG |
| | | TAATTCACTTTGCTGGTTTAAAGGCTGTAGGTGAATCT |
| | | ACACAAATCCCGCTGAGATACTATCACAATAACATTT |
| | | TGGGAACTGTCGTTTTATTAGAGTTAATGCAACAATAC |
| | | AACGTTTCCAAATTTGTTTTTTCATCTTCTGCTACTGTC |
| | | TATGGTGATGCTACGAGATTCCCAAATATGATTCCTAT |
| | | CCCAGAAGAATGTCCCTTAGGGCCTACTAATCCGTAT |
| | | GGTCATACGAAATACGCCATTGAGAATATCTTGAATG |
| | | ATCTTTACAATAGCGACAAAAAAAGTTGGAAGTTTGC |
| | | TATCTTGCGTTATTTTAACCCAATTGGCGCACATCCCT |
| | | CTGGATTAATCGGAGAAGATCCGCTAGGTATACCAAA |
| | | CAATTTGTTGCCATATATGGCTCAAGTAGCTGTTGGTA |
| | | GGCGCGAGAAGCTTTACATCTTCGGAGACGATTATGA |
| | | TTCCAGAGATGGTACCCCGATCAGGGATTATATCCAC |
| | | GTAGTTGATCTAGCAAAAGGTCATATTGCAGCCCTGC |
| | | AATACCTAGAGGCCTACAATGAAAATGAAGGTTTGTG |
| | | TCGTGAGTGGAACTTGGGTTCCGGTAAAGGTTCTACA |
| | | GTTTTTGAAGTTTATCATGCATTCTGCAAAGCTTCTGG |
| | | TATTGATCTTCCATACAAAGTTACGGGCAGAAGAGCA |
| | | GGTGATGTTTTGAACTTGACGGCTAAACCAGATAGGG |
| | | CCAAACGCGAACTGAAATGGCAGACCGAGTTGCAGGT |
| | | TGAAGACTCCTGCAAGGATTTATGGAAATGGACTACT |
| | | GAGAATCCTTTTGGTTACCAGTTAAGGGGTGTCGAGG |
| | | CCAGATTTTCCGCTGAAGATATGCGTTATGACGCAAG |
| | | ATTTGTGACTATTGGTGCCGGCACCAGATTTCAAGCCA |
| | | CGTTTGCCAATTTGGGCGCCAGCATTGTTGACCTGAAA |
| | | GTGAACGGACAATCAGTTGTTCTTGGCTATGAAAATG |
| | | AGGAAGGGTATTTGAATCCTGATAGTGCTTATATAGG |
| | | CGCCACGATCGGCAGGTATGCTAATCGTATTTCGAAG |
| | | GGTAAGTTTAGTTTATGCAACAAAGACTATCAGTTAA |
| | | CCGTTAATAACGGCGTTAATGCGAATCATAGTAGTAT |
| | | CGGTTCTTTCCACAGAAAAAGATTTTTGGGACCCATCA |
| | | TTCAAAATCCTTCAAAGGATGTTTTTACCGCCGAGTAC |
| | | ATGCTGATAGATAATGAGAAGGACACCGAATTTCCAG |
| | | GTGATCTATTGGTAACCATACAGTATACTGTGAACGTT |
| | | GCCCAAAAAAGTTTGGAAATGGTATATAAAGGTAAAT |
| | | TGACTGCTGGTGAAGCGACGCCAATAAATTTAACAAA |
| | | TCATAGTTATTTCAATCTGAACAAGCCATATGGAGAC |
| | | ACTATTGAGGGTACGGAGATTATGGTGCGTTCAAAAA |
| | | AATCTGTTGATGTCGACAAAAACATGATTCCTACGGG |
| | | TAATATCGTCGATAGAGAAATTGCTACCTTTAACTCTA |
| | | CAAAGCCAACGGTCTTAGGCCCCAAAAATCCCCAGTT |
| | | TGATTGTTGTTTTGTGGTGGATGAAAATGCTAAGCCAA |
| | | GTCAAATCAATACTCTAAACAATGAATTGACGCTTATT |
| | | GTCAAGGCTTTTCATCCCGATTCCAATATTACATTAGA |
| | | AGTTTTAAGTACAGAGCCAACTTATCAATTTTATACCG |
| | | GTGATTTCTTGTCTGCTGGTTACGAAGCAAGACAAGG |
| | | TTTTGCAATTGAGCCTGGTAGATACATTGATGCTATCA |
| | | ATCAAGAGAACTGGAAAGATTGTGTAACCTTGAAAAA |
| | | CGGTGAAACTTACGGGTCCAAGATTGTCTACAGATTTT |
| | | CCTGA |
| 30 | Pichia pastoris | AAATGCGTACCTCTTCTACGAGATTCAAGCGAATGAG |
| | Sequence of the | AATAATGTAATATGCAAGATCAGAAAGAATGAAAGG |
| | PpPMA1 | AGTTGAAAAAAAAAACCGTTGCGTTTTGACCTTGAAT |
| | promoter: | GGGGTGGAGGTTTCCATTCAAAGTAAAGCCTGTGTCT |
| | | TGGTATTTTCGGCGGCACAAGAAATCGTAATTTTCATC |
| | | TTCTAAACGATGAAGATCGCAGCCCAACCTGTATGTA |
| | | GTTAACCGGTCGGAATTATAAGAAAGATTTTCGATCA |
| | | ACAAACCCTAGCAAATAGAAAGCAGGGTTACAACTTT |
| | | AAACCGAAGTCACAAACGATAAACCACTCAGCTCCCA |
| | | CCCAAATTCATTCCCACTAGCAGAAAGGAATTATTTA |
| | | ATCCCTCAGGAAACCTCGATGATTCTCCCGTTCTTCCA |
| | | TGGGCGGGTATCGCAAAATGAGGAATTTTTCAAATTT |
| | | CTCTATTGTCAAGACTGTTTATTATCTAAGAAATAGCC |
| | | CAATCCGAAGCTCAGTTTTGAAAAAATCACTTCCGCG |
| | | TTTCTTTTTTACAGCCCGATGAATATCCAAATTTGGAA |
| | | TATGGATTACTCTATCGGGACTGCAGATAATATGACA |
| | | ACAACGCAGATTACATTTTAGGTAAGGCATAAACACC |
| | | AGCCAGAAATGAAACGCCCACTAGCCATGGTCGAATA |
| | | GTCCAATGAATTCAGATAGCTATGGTCTAAAAGCTGA |
| | | TGTTTTTTATTGGGTAATGGCGAAGAGTCCAGTACGAC |
| | | TTCCAGCAGAGCTGAGATGGCCATTTTTGGGGGTATT |
| | | AGTAACTTTTTGAGCTCTTTTCACTTCGATGAAGTGTC |
| | | CCATTCGGGATATAATCGGATCGCGTCGTTTTCTCGAA |
| | | AATACAGCTTAGCGTCGTCCGCTTGTTGTAAAAGCAG |
| | | CACCACATTCCTAATCTCTTATATAAACAAAACAACCC |
| | | AAATTATCAGTGCTGTTTTCCCACCAGATATAAGTTTC |
| | | TTTTCTCTTCCGCTTTTTGATTTTTTATCTCTTTCCTTTA |
| | | AAAACTTCTTTACCTTAAAGGGCGGCC |
| 31 | Pichia pastoris | TAAGCTTCACGATTTGTGTTCCAGTTTATCCCCCCTTT |
| | Sequence of the | ATATACCGTTAACCCTTTCCCTGTTGAGCTGACTGTTG |
| | PpPMA1 | TTGTATTACCGCAATTTTTCCAAGTTTGCCATGCTTTTC |
| | terminator: | GTGTTATTTGACCGATGTCTTTTTTCCCAAATCAAACT |
| | | ATATTTGTTACCATTTAAACCAAGTTATCTTTTGTATT |
| | | AAGAGTCTAAGTTTGTTCCCAGGCTTCATGTGAGAGT |
| | | GATAACCATCCAGACTATGATTCTTGTTTTTTATTGGG |
| | | TTTGTTTGTGTGATACATCTGAGTTGTGATTCGTAAAG |
| | | TATGTCAGTCTATCTAGATTTTTAATAGTTAATTGGTA |
| | | ATCAATGACTTGTTTGTTTTAACTTTTAAATTGTGGGT |
| | | CGTATCCACGCGTTTAGTATAGCTGTTCATGGCTGTTA |
| | | GAGGAGGGCGATGTTTATATACAGAGGACAAGAATGA |
| | | GGAGGCGGCGTGTATTTTTAAAATGGAGACGCGACTC |
| | | CTGTACACCTTATCGGTTGG |
| 32 | human | GGTAGAGATTTGTCTAGATTGCCACAGTTGGTTGGTGT |
| | hGalT codon | TTCCACTCCATTGCAAGGAGGTTCTAACTCTGCTGCTG |
| | optimized (XB) | CTATTGGTCAATCTTCCGGTGAGTTGAGAACTGGTGG |
| | | AGCTAGACCACCTCCACCATTGGGAGCTTCCTCTCAAC |
| | | CAAGACCAGGTGGTGATTCTTCTCCAGTTGTTGACTCT |
| | | GGTCCAGGTCCAGCTTCTAACTTGACTTCCGTTCCAGT |
| | | TCCACACACTACTGCTTTGTCCTTGCCAGCTTGTCCAG |
| | | AAGAATCCCCATTGTTGGTTGGTCCAATGTTGATCGAG |
| | | TTCAACATGCCAGTTGACTTGGAGTTGGTTGCTAAGCA |
| | | GAACCCAAACGTTAAGATGGGTGGTAGATACGCTCCA |
| | | AGAGACTGTGTTTCCCCACACAAAGTTGCTATCATCAT |
| | | CCCATTCAGAAACAGACAGGAGCACTTGAAGTACTGG |
| | | TTGTACTACTTGCACCCAGTTTTGCAAAGACAGCAGTT |
| | | GGACTACGGTATCTACGTTATCAACCAGGCTGGTGAC |
| | | ACTATTTTCAACAGAGCTAAGTTGTTGAATGTTGGTTT |
| | | CCAGGAGGCTTTGAAGGATTACGACTACACTTGTTTC |
| | | GTTTTCTCCGACGTTGACTTGATTCCAATGAACGACCA |
| | | CAACGCTTACAGATGTTTCTCCCAGCCAAGACACATTT |
| | | CTGTTGCTATGGACAAGTTCGGTTTCTCCTTGCCATAC |
| | | GTTCAATACTTCGGTGGTGTTTCCGCTTTGTCCAAGCA |
| | | GCAGTTCTTGACTATCAACGGTTTCCCAAACAATTACT |
| | | GGGGATGGGGTGGTGAAGATGACGACATCTTTAACAG |
| | | ATTGGTTTTCAGAGGAATGTCCATCTCTAGACCAAAC |
| | | GCTGTTGTTGGTAGATGTAGAATGATCAGACACTCCA |
| | | GAGACAAGAAGAACGAGCCAAACCCACAAAGATTCG |
| | | ACAGAATCGCTCACACTAAGGAAACTATGTTGTCCGA |
| | | CGGATTGAACTCCTTGACTTACCAGGTTTTGGACGTTC |
| | | AGAGATACCCATTGTACACTCAGATCACTGTTGACAT |
| | | CGGTACTCCATCCTAG |
| 33 | Saccharomyces | ATGGCCCTCTTTCTCAGTAAGAGACTGTTGAGATTTAC |
| | cerevisiae | CGTCATTGCAGGTGCGGTTATTGTTCTCCTCCTAACAT |
| | DNA encodes | TGAATTCCAACAGTAGAACTCAGCAATATATTCCGAG |
| | ScMnt1 (Kre2) | TTCCATCTCCGCTGCATTTGATTTTACCTCAGGATCTA |
| | (33) | TATCCCCTGAACAACAAGTCATCGGGCGCGCC |
| 34 | Drosophila | ATGAATAGCATACACATGAACGCCAATACGCTGAAGT |
| | melanogaster | ACATCAGCCTGCTGACGCTGACCCTGCAGAATGCCAT |
| | DNA encodes | CCTGGGCCTCAGCATGCGCTACGCCCGCACCCGGCCA |
| | DmUGT | GGCGACATCTTCCTCAGCTCCACGGCCGTACTCATGGC |
| | | AGAGTTCGCCAAACTGATCACGTGCCTGTTCCTGGTCT |
| | | TCAACGAGGAGGGCAAGGATGCCCAGAAGTTTGTACG |
| | | CTCGCTGCACAAGACCATCATTGCGAATCCCATGGAC |
| | | ACGCTGAAGGTGTGCGTCCCCTCGCTGGTCTATATCGT |
| | | TCAAAACAATCTGCTGTACGTCTCTGCCTCCCATTTGG |
| | | ATGCGGCCACCTACCAGGTGACGTACCAGCTGAAGAT |
| | | TCTCACCACGGCCATGTTCGCGGTTGTCATTCTGCGCC |
| | | GCAAGCTGCTGAACACGCAGTGGGGTGCGCTGCTGCT |
| | | CCTGGTGATGGGCATCGTCCTGGTGCAGTTGGCCCAA |
| | | ACGGAGGGTCCGACGAGTGGCTCAGCCGGTGGTGCCG |
| | | CAGCTGCAGCCACGGCCGCCTCCTCTGGCGGTGCTCC |
| | | CGAGCAGAACAGGATGCTCGGACTGTGGGCCGCACTG |
| | | GGCGCCTGCTTCCTCTCCGGATTCGCGGGCATCTACTT |
| | | TGAGAAGATCCTCAAGGGTGCCGAGATCTCCGTGTGG |
| | | ATGCGGAATGTGCAGTTGAGTCTGCTCAGCATTCCCTT |
| | | CGGCCTGCTCACCTGTTTCGTTAACGACGGCAGTAGG |
| | | ATCTTCGACCAGGGATTCTTCAAGGGCTACGATCTGTT |
| | | TGTCTGGTACCTGGTCCTGCTGCAGGCCGGCGGTGGA |
| | | TTGATCGTTGCCGTGGTGGTCAAGTACGCGGATAACA |
| | | TTCTCAAGGGCTTCGCCACCTCGCTGGCCATCATCATC |
| | | TCGTGCGTGGCCTCCATATACATCTTCGACTTCAATCT |
| | | CACGCTGCAGTTCAGCTTCGGAGCTGGCCTGGTCATC |
| | | GCCTCCATATTTCTCTACGGCTACGATCCGGCCAGGTC |
| | | GGCGCCGAAGCCAACTATGCATGGTCCTGGCGGCGAT |
| | | GAGGAGAAGCTGCTGCCGCGCGTCTAG |
| 35 | Pichia pastoris | TGGACACAGGAGACTCAGAAACAGACACAGAGCGTT |
| | Sequence of the | CTGAGTCCTGGTGCTCCTGACGTAGGCCTAGAACAGG |
| | PpOCH1 | AATTATTGGCTTTATTTGTTTGTCCATTTCATAGGCTTG |
| | promoter: | GGGTAATAGATAGATGACAGAGAAATAGAGAAGACC |
| | | TAATATTTTTTGTTCATGGCAAATCGCGGGTTCGCGGT |
| | | CGGGTCACACACGGAGAAGTAATGAGAAGAGCTGGT |
| | | AATCTGGGGTAAAAGGGTTCAAAAGAAGGTCGCCTGG |
| | | TAGGGATGCAATACAAGGTTGTCTTGGAGTTTACATTG |
| | | ACCAGATGATTTGGCTTTTTCTCTGTTCAATTCACATTT |
| | | TTCAGCGAGAATCGGATTGACGGAGAAATGGCGGGGT |
| | | GTGGGGTGGATAGATGGCAGAAATGCTCGCAATCACC |
| | | GCGAAAGAAAGACTTTATGGAATAGAACTACTGGGTG |
| | | GTGTAAGGATTACATAGCTAGTCCAATGGAGTCCGTT |
| | | GGAAAGGTAAGAAGAAGCTAAAACCGGCTAAGTAAC |
| | | TAGGGAAGAATGATCAGACTTTGATTTGATGAGGTCT |
| | | GAAAATACTCTGCTGCTTTTTCAGTTGCTTTTTCCCTGC |
| | | AACCTATCATTTTCCTTTTCATAAGCCTGCCTTTTCTGT |
| | | TTTCACTTATATGAGTTCCGCCGAGACTTCCCCAAATT |
| | | CTCTCCTGGAACATTCTCTATCGCTCTCCTTCCAAGTT |
| | | GCGCCCCCTGGCACTGCCTAGTAATATTACCACGCGA |
| | | CTTATATTCAGTTCCACAATTTCCAGTGTTCGTAGCAA |
| | | ATATCATCAGCC |
| 36 | Pichia pastoris | AATATATACCTCATTTGTTCAATTTGGTGTAAAGAGTG |
| | Sequence of the | TGGCGGATAGACTTCTTGTAAATCAGGAAAGCTACAA |
| | PpALG12 | TTCCAATTGCTGCAAAAAATACCAATGCCCATAAACC |
| | terminator: | AGTATGAGCGGTGCCTTCGACGGATTGCTTACTTTCCG |
| | | ACCCTTTGTCGTTTGATTCTTCTGCCTTTGGTGAGTCA |
| | | GTTTGTTTCGACTTTATATCTGACTCATCAACTTCCTTT |
| | | ACGGTTGCGTTTTTAATCATAATTTTAGCCGTTGGCTT |
| | | ATTATCCCTTGAGTTGGTAGGAGTTTTGATGATGCTG |
| 37 | Pichia pastoris | |
| | Sequence of the | TAACTGGCCCTTTGACGTTTCTGACAATAGTTCTAGAG |
| | 5′-Region used | GAGTCGTCCAAAAACTCAACTCTGACTTGGGTGACAC |
| | for knock out of | CACCACGGGATCCGGTTCTTCCGAGGACCTTGATGAC |
| | PpHIS1: | CTTGGCTAATGTAACTGGAGTTTTAGTATCCATTTTAA |
| | | GATGTGTGTTTCTGTAGGTTCTGGGTTGGAAAAAAATT |
| | | TTAGACACCAGAAGAGAGGAGTGAACTGGTTTGCGTG |
| | | GGTTTAGACTGTGTAAGGCACTACTCTGTCGAAGTTTT |
| | | AGATAGGGGTTACCCGCTCCGATGCATGGGAAGCGAT |
| | | TAGCCCGGCTGTTGCCCGTTTGGTTTTTGAAGGGTAAT |
| | | TTTCAATATCTCTGTTTGAGTCATCAATTTCATATTCA |
| | | AAGATTCAAAAACAAAATCTGGTCCAAGGAGCGCATT |
| | | TAGGATTATGGAGTTGGCGAATCACTTGAACGATAGA |
| | | CTATTATTTGC |
| 38 | Pichia pastoris | GTGACATTCTTGTCTTTGAGATCAGTAATTGTAGAGCA |
| | Sequence of the | TAGATAGAATAATATTCAAGACCAACGGCTTCTCTTC |
| | 3′-Region used | GGAAGCTCCAAGTAGCTTATAGTGATGAGTACCGGCA |
| | for knock out of | TATATTTATAGGCTTAAAATTTCGAGGGTTCACTATAT |
| | PpHIS1: | TCGTTTAGTGGGAAGAGTTCCTTTCACTCTTGTTATCT |
| | | ATATTGTCAGCGTGGACTGTTTATAACTGTACCAACTT |
| | | AGTTTCTTTCAACTCCAGGTTAAGAGACATAAATGTCC |
| | | TTTGATGCTGACAATAATCAGTGGAATTCAAGGAAGG |
| | | ACAATCCCGACCTCAATCTGTTCATTAATGAAGAGTTC |
| | | GAATCGTCCTTAAATCAAGCGCTAGACTCAATTGTCA |
| | | ATGAGAACCCTTTCTTTGACCAAGAAACTATAAATAG |
| | | ATCGAATGACAAAGTTGGAAATGAGTCCATTAGCTTA |
| | | CATGATATTGAGCAGGCAGACCAAAATAAACCGTCCT |
| | | TTGAGAGCGATATTGATGGTTCGGCGCCGTTGATAAG |
| | | AGACGACAAATTGCCAAAGAAACAAAGCTGGGGGCT |
| | | GAGCAATTTTTTTTCAAGAAGAAATAGCATATGTTTAC |
| | | CACTACATGAAAATGATTCAAGTGTTGTTAAGACCGA |
| | | AAGATCTATTGCAGTGGGAACACCCCATCTTCAATAC |
| | | TGCTTCAATGGAATCTCCAATGCCAAGTACAATGCATT |
| | | TACCTTTTTCCCAGTCATCCTATACGAGCAATTCAAAT |
| | | TTTTTTTCAATTTATACTTTACTTTAGTGGCTCTCTCTC |
| | | AAGCGATACCGCAACTTCGCATTGGATATCTTTCTTCG |
| | | TATGTCGTCCCACTTTTGTTTGTACTCATAGTGACCAT |
| | | GTCAAAAGAGGCGATGGATGATATTCAACGCCGAAGA |
| | | AGGGATAGAGAACAGAACAATGAACCATATGAGGTTC |
| | | TGTCCAGCCCATCACCAGTTTTGTCCAAAAACTTAAAA |
| | | TGTGGTCACTTGGTTCGATTGCATAAGGGAATGAGAG |
| | | TGCCCGCAGATATGGTTCTTGTCCAGTCAAGCGAATCC |
| | | ACCGGAGAGTCATTTATCAAGACAGATCAGCTGGATG |
| | | GTGAGACTGATTGGAAGCTTCGGATTGTTTCTCCAGTT |
| | | ACACAATCGTTACCAATGACTGAACTTCAAAATGTCG |
| | | CCATCACTGCAAGCGCACCCTCAAAATCAATTCACTC |
| | | CTTTCTTGGAAGATTGACCTACAATGGGCAATCATATG |
| | | GTCTTACGATAGACAACACAATGTGGTGTAATACTGT |
| | | ATTAGCTTCTGGTTCAGCAATTGGTTGTATAATTTACA |
| | | CAGGTAAAGATACTCGACAATCGATGAACACAACTCA |
| | | GCCCAAACTGAAAACGGGCTTGTTAGAACTGGAAATC |
| | | AATAGTTTGTCCAAGATCTTATGTGTTTGTGTGTTTGC |
| | | ATTATCTGTCATCTTAGTGCTATTCCAAGGAATAGCTG |
| | | ATGATTGGTACGTCGATATCATGCGGTTTCTCATTCTA |
| | | TTCTCCACTATTATCCCAGTGTCTCTGAGAGTTAACCT |
| | | TGATCTTGGAAAGTCAGTCCATGCTCATCAAATAGAA |
| | | ACTGATAGCTCAATACCTGAAACCGTTGTTAGAACTA |
| | | GTACAATACCGGAAGACCTGGGAAGAATTGAATACCT |
| | | ATTAAGTGACAAAACTGGAACTCTTACTCAAAATGAT |
| | | ATGGAAATGAAAAAACTACACCTAGGAACAGTCTCTT |
| | | ATGCTGGTGATACCATGGATATTATTTCTGATCATGTT |
| | | AAAGGTCTTAATAACGCTAAAACATCGAGGAAAGATC |
| | | TTGGTATGAGAATAAGAGATTTGGTTACAACTCTGGC |
| | | CATCTG |
| 39 | Drosophila | AGAGACGATCCAATTAGACCTCCATTGAAGGTTGCTA |
| | melanogaster | GATCCCCAAGACCAGGTCAATGTCAAGATGTTGTTCA |
| | DNA encodes | GGACGTCCCAAACGTTGATGTCCAGATGTTGGAGTTG |
| | Drosophila | TACGATAGAATGTCCTTCAAGGACATTGATGGTGGTG |
| | melanogaster | TTTGGAAGCAGGGTTGGAACATTAAGTACGATCCATT |
| | Mann codon- | GAAGTACAACGCTCATCACAAGTTGAAGGTCTTCGTT |
| | optimized (KD) | GTCCCACACTCCCACAACGATCCTGGTTGGATTCAGA |
| | | CCTTCGAGGAATACTACCAGCACGACACCAAGCACAT |
| | | CTTGTCCAACGCTTTGAGACATTTGCACGACAACCCA |
| | | GAGATGAAGTTCATCTGGGCTGAAATCTCCTACTTCGC |
| | | TAGATTCTACCACGATTTGGGTGAGAACAAGAAGTTG |
| | | CAGATGAAGTCCATCGTCAAGAACGGTCAGTTGGAAT |
| | | TCGTCACTGGTGGATGGGTCATGCCAGACGAGGCTAA |
| | | CTCCCACTGGAGAAACGTTTTGTTGCAGTTGACCGAA |
| | | GGTCAAACTTGGTTGAAGCAATTCATGAACGTCACTC |
| | | CAACTGCTTCCTGGGCTATCGATCCATTCGGACACTCT |
| | | CCAACTATGCCATACATTTTGCAGAAGTCTGGTTTCAA |
| | | GAATATGTTGATCCAGAGAACCCACTACTCCGTTAAG |
| | | AAGGAGTTGGCTCAACAGAGACAGTTGGAGTTCTTGT |
| | | GGAGACAGATCTGGGACAACAAAGGTGACACTGCTTT |
| | | GTTCACCCACATGATGCCATTCTACTCTTACGACATTC |
| | | CTCATACCTGTGGTCCAGATCCAAAGGTTTGTTGTCAG |
| | | TTCGATTTCAAAAGAATGGGTTCCTTCGGTTTGTCTTG |
| | | TCCATGGAAGGTTCCACCTAGAACTATCTCTGATCAA |
| | | AATGTTGCTGCTAGATCCGATTTGTTGGTTGATCAGTG |
| | | GAAGAAGAAGGCTGAGTTGTACAGAACCAACGTCTTG |
| | | TTGATTCCATTGGGTGACGACTTCAGATTCAAGCAGA |
| | | ACACCGAGTGGGATGTTCAGAGAGTCAACTACGAAAG |
| | | ATTGTTCGAACACATCAACTCTCAGGCTCACTTCAATG |
| | | TCCAGGCTCAGTTCGGTACTTTGCAGGAATACTTCGAT |
| | | GCTGTTCACCAGGCTGAAAGAGCTGGACAAGCTGAGT |
| | | TCCCAACCTTGTCTGGTGACTTCTTCACTTACGCTGAT |
| | | AGATCTGATAACTACTGGTCTGGTTACTACACTTCCAG |
| | | ACCATACCATAAGAGAATGGACAGAGTCTTGATGCAC |
| | | TACGTTAGAGCTGCTGAAATGTTGTCCGCTTGGCACTC |
| | | CTGGGACGGTATGGCTAGAATCGAGGAAAGATTGGAG |
| | | CAGGCTAGAAGAGAGTTGTCCTTGTTCCAGCACCACG |
| | | ACGGTATTACTGGTACTGCTAAAACTCACGTTGTCGTC |
| | | GACTACGAGCAAAGAATGCAGGAAGCTTTGAAAGCTT |
| | | GTCAAATGGTCATGCAACAGTCTGTCTACAGATTGTTG |
| | | ACTAAGCCATCCATCTACTCTCCAGACTTCTCCTTCTC |
| | | CTACTTCACTTTGGACGACTCCAGATGGCCAGGTTCTG |
| | | GTGTTGAGGACTCTAGAACTACCATCATCTTGGGTGA |
| | | GGATATCTTGCCATCCAAGCATGTTGTCATGCACAAC |
| | | ACCTTGCCACACTGGAGAGAGCAGTTGGTTGACTTCT |
| | | ACGTCTCCTCTCCATTCGTTTCTGTTACCGACTTGGCT |
| | | AACAATCCAGTTGAGGCTCAGGTTTCTCCAGTTTGGTC |
| | | TTGGCACCACGACACTTTGACTAAGACTATCCACCCA |
| | | CAAGGTTCCACCACCAAGTACAGAATCATCTTCAAGG |
| | | CTAGAGTTCCACCAATGGGTTTGGCTACCTACGTTTTG |
| | | ACCATCTCCGATTCCAAGCCAGAGCACACCTCCTACG |
| | | CTTCCAATTTGTTGCTTAGAAAGAACCCAACTTCCTTG |
| | | CCATTGGGTCAATACCCAGAGGATGTCAAGTTCGGTG |
| | | ATCCAAGAGAGATCTCCTTGAGAGTTGGTAACGGTCC |
| | | AACCTTGGCTTTCTCTGAGCAGGGTTTGTTGAAGTCCA |
| | | TTCAGTTGACTCAGGATTCTCCACATGTTCCAGTTCAC |
| | | TTCAAGTTCTTGAAGTACGGTGTTAGATCTCATGGTGA |
| | | TAGATCTGGTGCTTACTTGTTCTTGCCAAATGGTCCAG |
| | | CTTCTCCAGTCGAGTTGGGTCAGCCAGTTGTCTTGGTC |
| | | ACTAAGGGTAAATTGGAGTCTTCCGTTTCTGTTGGTTT |
| | | GCCATCTGTCGTTCACCAGACCATCATGAGAGGTGGT |
| | | GCTCCAGAGATTAGAAATTTGGTCGATATTGGTTCTTT |
| | | GGACAACACTGAGATCGTCATGAGATTGGAGACTCAT |
| | | ATCGACTCTGGTGATATCTTCTACACTGATTTGAATGG |
| | | ATTGCAATTCATCAAGAGGAGAAGATTGGACAAGTTG |
| | | CCATTGCAGGCTAACTACTACCCAATTCCATCTGGTAT |
| | | GTTCATTGAGGATGCTAATACCAGATTGACTTTGTTGA |
| | | CCGGTCAACCATTGGGTGGATCTTCTTTGGCTTCTGGT |
| | | GAGTTGGAGATTATGCAAGATAGAAGATTGGCTTCTG |
| | | ATGATGAAAGAGGTTTGGGTCAGGGTGTTTTGGACAA |
| | | CAAGCCAGTTTTGCATATTTACAGATTGGTCTTGGAGA |
| | | AGGTTAACAACTGTGTCAGACCATCTAAGTTGCATCC |
| | | AGCTGGTTACTTGACTTCTGCTGCTCACAAAGCTTCTC |
| | | AGTCTTTGTTGGATCCATTGGACAAGTTCATCTTCGCT |
| | | GAAAATGAGTGGATCGGTGCTCAGGGTCAATTCGGTG |
| | | GTGATCATCCATCTGCTAGAGAGGATTTGGATGTCTCT |
| | | GTCATGAGAAGATTGACCAAGTCTTCTGCTAAAACCC |
| | | AGAGAGTTGGTTACGTTTTGCACAGAACCAATTTGAT |
| | | GCAATGTGGTACTCCAGAGGAGCATACTCAGAAGTTG |
| | | GATGTCTGTCACTTGTTGCCAAATGTTGCTAGATGTGA |
| | | GAGAACTACCTTGACTTTCTTGCAGAATTTGGAGCACT |
| | | TGGATGGTATGGTTGCTCCAGAAGTTTGTCCAATGGA |
| | | AACCGCTGCTTACGTCTCTTCTCACTCTTCTTGA |
| 40 | Saccharomyces | ATGCTGCTTACCAAAAGGTTTTCAAAGCTGTTCAAGCT |
| | cerevisiae | GACGTTCATAGTTTTGATATTGTGCGGGCTGTTCGTCA |
| | DNA encodes | TTACAAACAAATACATGGATGAGAACACGTCG |
| | ScMnn2 leader | |
| | (53) | |
| 41 | Pichia pastoris | CAAGTTGCGTCCGGTATACGTAACGTCTCACGATGAT |
| | Sequence of the | CAAAGATAATACTTAATCTTCATGGTCTACTGAATAAC |
| | PpHIS1 | TCATTTAAACAATTGACTAATTGTACATTATATTGAAC |
| | auxotrophic | TTATGCATCCTATTAACGTAATCTTCTGGCTTCTCTCTC |
| | marker: | AGACTCCATCAGACACAGAATATCGTTCTCTCTAACTG |
| | | GTCCTTTGACGTTTCTGACAATAGTTCTAGAGGAGTCG |
| | | TCCAAAAACTCAACTCTGACTTGGGTGACACCACCAC |
| | | GGGATCCGGTTCTTCCGAGGACCTTGATGACCTTGGCT |
| | | AATGTAACTGGAGTTTTAGTATCCATTTTAAGATGTGT |
| | | GTTTCTGTAGGTTCTGGGTTGGAAAAAAATTTTAGACA |
| | | CCAGAAGAGAGGAGTGAACTGGTTTGCGTGGGTTTAG |
| | | ACTGTGTAAGGCACTACTCTGTCGAAGTTTTAGATAG |
| | | GGGTTACCCGCTCCGATGCATGGGAAGCGATTAGCCC |
| | | GGCTGTTGCCCGTTTGGTTTTTGAAGGGTAATTTTCAA |
| | | TATCTCTGTTTGAGTCATCAATTTCATATTCAAAGATT |
| | | CAAAAACAAAATCTGGTCCAAGGAGCGCATTTAGGAT |
| | | TATGGAGTTGGCGAATCACTTGAACGATAGACTATTA |
| | | TTTGCTGTTCCTAAAGAGGGCAGATTGTATGAGAAAT |
| | | GCGTTGAATTACTTAGGGGATCAGATATTCAGTTTCGA |
| | | AGATCCAGTAGATTGGATATAGCTTTGTGCACTAACCT |
| | | GCCCCTGGCATTGGTTTTCCTTCCAGCTGCTGACATTC |
| | | CCACGTTTGTAGGAGAGGGTAAATGTGATTTGGGTAT |
| | | AACTGGTATTGACCAGGTTCAGGAAAGTGACGTAGAT |
| | | GTCATACCTTTATTAGACTTGAATTTCGGTAAGTGCAA |
| | | GTTGCAGATTCAAGTTCCCGAGAATGGTGACTTGAAA |
| | | GAACCTAAACAGCTAATTGGTAAAGAAATTGTTTCCT |
| | | CCTTTACTAGCTTAACCACCAGGTACTTTGAACAACTG |
| | | GAAGGAGTTAAGCCTGGTGAGCCACTAAAGACAAAA |
| | | ATCAAATATGTTGGAGGGTCTGTTGAGGCCTCTTGTGC |
| | | CCTAGGAGTTGCCGATGCTATTGTGGATCTTGTTGAGA |
| | | GTGGAGAAACCATGAAAGCGGCAGGGCTGATCGATAT |
| | | TGAAACTGTTCTTTCTACTTCCGCTTACCTGATCTCTTC |
| | | GAAGCATCCTCAACACCCAGAACTGATGGATACTATC |
| | | AAGGAGAGAATTGAAGGTGTACTGACTGCTCAGAAGT |
| | | ATGTCTTGTGTAATTACAACGCACCTAGAGGTAACCTT |
| | | CCTCAGCTGCTAAAACTGACTCCAGGCAAGAGAGCTG |
| | | CTACCGTTTCTCCATTAGATGAAGAAGATTGGGTGGG |
| | | AGTGTCCTCGATGGTAGAGAAGAAAGATGTTGGAAGA |
| | | ATCATGGACGAATTAAAGAAACAAGGTGCCAGTGACA |
| | | TTCTTGTCTTTGAGATCAGTAATTGTAGAGCATAGATA |
| | | GAATAATATTCAAGACCAACGGCTTCTCTTCGGAAGC |
| | | TCCAAGTAGCTTATAGTGATGAGTACCGGCATATATTT |
| | | ATAGGCTTAAAATTTCGAGGGTTCACTATATTCGTTTA |
| | | GTGGGAAGAGTTCCTTTCACTCTTGTTATCTATATTGT |
| | | CAGCGTGGACTGTTTATAACTGTACCAACTTAGTTTCT |
| | | TTCAACTCCAGGTTAAGAGACATAAATGTCCTTTGATGC |
| 42 | DNA encodes | TCCTTGGTTTACCAATTGAACTTCGACCAGATGTTGAG |
| | Rat GnT II | AAACGTTGACAAGGACGGTACTTGGTCTCCTGGTGAG |
| | (TC) | TTGGTTTTGGTTGTTCAGGTTCACAACAGACCAGAGTA |
| | Codon- | CTTGAGATTGTTGATCGACTCCTTGAGAAAGGCTCAA |
| | optimized | GGTATCAGAGAGGTTTTGGTTATCTTCTCCCACGATTT |
| | | CTGGTCTGCTGAGATCAACTCCTTGATCTCCTCCGTTG |
| | | ACTTCTGTCCAGTTTTGCAGGTTTTCTTCCCATTCTCCA |
| | | TCCAATTGTACCCATCTGAGTTCCCAGGTTCTGATCCA |
| | | AGAGACTGTCCAAGAGACTTGAAGAAGAACGCTGCTT |
| | | TGAAGTTGGGTTGTATCAACGCTGAATACCCAGATTCT |
| | | TTCGGTCACTACAGAGAGGCTAAGTTCTCCCAAACTA |
| | | AGCATCATTGGTGGTGGAAGTTGCACTTTGTTTGGGAG |
| | | AGAGTTAAGGTTTTGCAGGACTACACTGGATTGATCTT |
| | | GTTCTTGGAGGAGGATCATTACTTGGCTCCAGACTTCT |
| | | ACCACGTTTTCAAGAAGATGTGGAAGTTGAAGCAACA |
| | | AGAGTGTCCAGGTTGTGACGTTTTGTCCTTGGGAACTT |
| | | ACACTACTATCAGATCCTTCTACGGTATCGCTGACAAG |
| | | GTTGACGTTAAGACTTGGAAGTCCACTGAACACAACA |
| | | TGGGATTGGCTTTGACTAGAGATGCTTACCAGAAGTT |
| | | GATCGAGTGTACTGACACTTTCTGTACTTACGACGACT |
| | | ACAACTGGGACTGGACTTTGCAGTACTTGACTTTGGCT |
| | | TGTTTGCCAAAAGTTTGGAAGGTTTTGGTTCCACAGGC |
| | | TCCAAGAATTTTCCACGCTGGTGACTGTGGAATGCAC |
| | | CACAAGAAAACTTGTAGACCATCCACTCAGTCCGCTC |
| | | AAATTGAGTCCTTGTTGAACAACAACAAGCAGTACTT |
| | | GTTCCCAGAGACTTTGGTTATCGGAGAGAAGTTTCCA |
| | | ATGGCTGCTATTTCCCCACCAAGAAAGAATGGTGGAT |
| | | GGGGTGATATTAGAGACCACGAGTTGTGTAAATCCTA |
| | | CAGAAGATTGCAGTAG |
| 43 | Saccharomyces | ATGCTGCTTACCAAAAGGTTTTCAAAGCTGTTCAAGCT |
| | cerevisiae | GACGTTCATAGTTTTGATATTGTGCGGGCTGTTCGTCA |
| | DNA encodes | TTACAAACAAATACATGGATGAGAACACGTCGGTCAA |
| | ScMnn2 leader | GGAGTACAAGGAGTACTTAGACAGATATGTCCAGAGT |
| | (54) | TACTCCAATAAGTATTCATCTTCCTCAGACGCCGCCAG |
| | The last 9 | CGCTGACGATTCAACCCCATTGAGGGACAATGATGAG |
| | nucleotides are | GCAGGCAATGAAAAGTTGAAAAGCTTCTACAACAACG |
| | the linker | TTTTCAACTTTCTAATGGTTGATTCGCCCGGGCGCGCC |
| | containing the | |
| | AscI restriction | |
| | site) | |
| 44 | Pichia pastoris | GATCTGGCCTTCCCTGAATTTTTACGTCCAGCTATACG |
| | Sequence of the | ATCCGTTGTGACTGTATTTCCTGAAATGAAGTTTCAAC |
| | 5′-Region used | CTAAAGTTTTGGTTGTACTTGCTCCACCTACCACGGAA |
| | for knock out of | ACTAATATCGAAACCAATGAAAAAGTAGAACTGGAAT |
| | PpARG1: | CGTCAATCGAAATTCGCAACCAAGTGGAACCCAAAGA |
| | | CTTGAATCTTTCTAAAGTCTATTCTAGTGACACTAATG |
| | | GCAACAGAAGATTTGAGCTGACTTTTCAAATGAATCT |
| | | CAATAATGCAATATCAACATCAGACAATCAATGGGCT |
| | | TTGTCTAGTGACACAGGATCAATTATAGTAGTGTCTTC |
| | | TGCAGGAAGAATAACTTCCCCGATCCTAGAAGTCGGG |
| | | GCATCCGTCTGTGTCTTAAGATCGTACAACGAACACCT |
| | | TTTGGCAATAACTTGTGAAGGAACATGCTTTTCATGGA |
| | | ATTTAAAGAAGCAAGAATGTGTTCTAAACAGCATTTC |
| | | ATTAGCACCTATAGTCAATTCACACATGCTAGTTAAG |
| | | AAAGTTGGAGATGCAAGGAACTATTCTATTGTATCTG |
| | | CCGAAGGAGACAACAATCCGTTACCCCAGATTCTAGA |
| | | CTGCGAACTTTCCAAAAATGGCGCTCCAATTGTGGCTC |
| | | TTAGCACGAAAGACATCTACTCTTATTCAAAGAAAAT |
| | | GAAATGCTGGATCCATTTGATTGATTCGAAATACTTTG |
| | | AATTGTTGGGTGCTGACAATGCACTGTTTGAGTGTGTG |
| | | GAAGCGCTAGAAGGTCCAATTGGAATGCTAATTCATA |
| | | GATTGGTAGATGAGTTCTTCCATGAAAACACTGCCGG |
| | | TAAAAAACTCAAACTTTACAACAAGCGAGTACTGGAG |
| | | GACCTTTCAAATTCACTTGAAGAACTAGGTGAAAATG |
| | | CGTCTCAATTAAGAGAGAAACTTGACAAACTCTATGG |
| | | TGATGAGGTTGAGGCTTCTTGACCTCTTCTCTCTATCT |
| | | GCGTTTCTTTTTTTTTTTTTTTTTTTTTTTTTTTCAGTTG |
| | | AGCCAGACCGCGCTAAACGCATACCAATTGCCAAATC |
| | | AGGCAATTGTGAGACAGTGGTAAAAAAGATGCCTGCA |
| | | AAGTTAGATTCACACAGTAAGAGAGATCCTACTCATA |
| | | AATGAGGCGCTTATTTAGTAGCTAGTGATAGCCACTG |
| | | CGGTTCTGCTTTATGCTATTTGTTGTATGCCTTACTATC |
| | | TTTGTTTGGCTCCTTTTTCTTGACGTTTTCCGTTGGAGG |
| | | GACTCCCTATTCTGAGTCATGAGCCGCACAGATTATCG |
| | | CCCAAAATTGACAAAATCTTCTGGCGAAAAAAGTATA |
| | | AAAGGAGAAAAAAGCTCACCCTTTTCCAGCGTAGAAA |
| | | GTATATATCAGTCATTGAAGAC |
| 45 | Pichia pastoris | GGGACTTTAACTCAAGTAAAAGGATAGTTGTACAATT |
| | Sequence of the | ATATATACGAAGAATAAATCATTACAAAAAGTATTCG |
| | 3′-Region used | TTTCTTTGATTCTTAACAGGATTCATTTTCTGGGTGTCA |
| | for knock out of | TCAGGTACAGCGCTGAATATCTTGAAGTTAACATCGA |
| | PpARG1: | GCTCATCATCGACGTTCATCACACTAGCCACGTTTCCG |
| | | CAACGGTAGCAATAATTAGGAGCGGACCACACAGTGA |
| | | CGACATCTTTCTCTTTGAAATGGTATCTGAAGCCTTCC |
| | | ATGACCAATTGATGGGCTCTAGCGATGAGTTGCAAGT |
| | | TATTAATGTGGTTGAACTCACGTGCTACTCGAGCACCG |
| | | AATAACCAGCCAGCTCCACGAGGAGAAACAGCCCAA |
| | | CTGTCGACTTCATCTGGGTCAGACCAAACCAAGTCAC |
| | | AAAATCCTCCTTCATGAGGGACCTCTTGCGCTCGGCTG |
| | | AGAACTCTGATTTGATCTAACATGCGAATATCGGGAG |
| | | AGAGACCACCATGGATACATAATATTTTACCATCAAT |
| | | GATGGCACTAAGGGTTAAAAAGTCGAACACCTGGCAA |
| | | CAGTACTTCCAGACAGTGGTGGAACCATATTTATTGA |
| | | GACATTCCTCATAAAATCCATAAACCTGAGTGATCTGT |
| | | CTGGATTCATGATTTCCCCTTACCAATGTGATATGTTG |
| | | AGGAAACTTAATTTTTAAAATCATGAGTAACGTGAAC |
| | | GTCTCCAACGAGAAATAGCCTCTATCCACATAGTCTCC |
| | | TAGGAAGATATAGTTCTGTTTTATTCCATTAGAGGAGG |
| | | ATCCGGGAAACCCACCACTAATCTTGAAAAGTTCCAG |
| | | TAGATCGTGAAATTGGCCGTGAATATCTCCGCATACT |
| | | GTCACTGGACTCTGCACTGGCTGTATATTGGATTCCTC |
| | | CATCAGCAAATCCTTCACCCGTTCGCAAAGATGCTTCA |
| | | TATCATTTTCACTTAAAGCCTTGCAGCTTTTGACTTCTT |
| | | CAAACCACTGATCTGGTCCTCTTTCTGGCATGATTAAG |
| | | GTCTATAATATTTCTGAGCTGAGATGTAAAAAAAAAT |
| | | AATAAAAATGGGGAGTGAAAAAGTGTGTAGCTTTTAG |
| | | GAGTTTGGGATTGATACCCCAAAATGATCTTTATGAG |
| | | AATTAAAAGGTAGATACGCTTTTAATAAGAACACCTA |
| | | TCTATAGTACTTTGTGGTCTTGAGTAATTGAGATGTTC |
| | | AGCTTCTGAGGTTTGCCGTTATTCTGGGATAGTAGTGC |
| | | GCGACCAAACAACCCGCCAGGCAAAGTGTGTTGTGCT |
| | | CGAAGACGATTGCCAGAAGAGTAAGTCCGTCCTGCCT |
| | | CAGATGTTACACACTTTCTTCCCTAGACAGTCGATGCA |
| | | TCATCGGATTTAAACCTGAAACTTTGATGCCATGATAC |
| | | GCCTAGTCACGTCGACTGAGATTTTAGATAAGCCCCG |
| | | ATCCCTTTAGTACATTCCTGTTATCCATGGATGGAATG |
| | | GCCTGATA |
| 46 | Pichia pastoris | AAGCTTGTTCACCGTTGGGACTTTTCCGTGGACAATGT |
| | Sequence of the | TGACTACTCCAGGAGGGATTCCAGCTTTCTCTACTAGC |
| | 5′-Region used | TCAGCAATAATCAATGCAGCCCCAGGCGCCCGTTCTG |
| | for knock out of | ATGGCTTGATGACCGTTGTATTGCCTGTCACTATAGCC |
| | BMT4 | AGGGGTAGGGTCCATAAAGGAATCATAGCAGGGAAA |
| | | TTAAAAGGGCATATTGATGCAATCACTCCCAATGGCT |
| | | CTCTTGCCATTGAAGTCTCCATATCAGCACTAACTTCC |
| | | AAGAAGGACCCCTTCAAGTCTGACGTGATAGAGCACG |
| | | CTTGCTCTGCCACCTGTAGTCCTCTCAAAACGTCACCT |
| | | TGTGCATCAGCAAAGACTTTACCTTGCTCCAATACTAT |
| | | GACGGAGGCAATTCTGTCAAAATTCTCTCTCAGCAATT |
| | | CAACCAACTTGAAAGCAAATTGCTGTCTCTTGATGAT |
| | | GGAGACTTTTTTCCAAGATTGAAATGCAATGTGGGAC |
| | | GACTCAATTGCTTCTTCCAGCTCCTCTTCGGTTGATTG |
| | | AGGAACTTTTGAAACCACAAAATTGGTCGTTGGGTCA |
| | | TGTACATCAAACCATTCTGTAGATTTAGATTCGACGAA |
| | | AGCGTTGTTGATGAAGGAAAAGGTTGGATACGGTTTG |
| | | TCGGTCTCTTTGGTATGGCCGGTGGGGTATGCAATTGC |
| | | AGTAGAAGATAATTGGACAGCCATTGTTGAAGGTAGA |
| | | GAAAAGGTCAGGGAACTTGGGGGTTATTTATACCATT |
| | | TTACCCCACAAATAACAACTGAAAAGTACCCATTCCA |
| | | TAGTGAGAGGTAACCGACGGAAAAAGACGGGCCCAT |
| | | GTTCTGGGACCAATAGAACTGTGTAATCCATTGGGAC |
| | | TAATCAACAGACGATTGGCAATATAATGAAATAGTTC |
| | | GTTGAAAAGCCACGTCAGCTGTCTTTTCATTAACTTTG |
| | | GTCGGACACAACATTTTCTACTGTTGTATCTGTCCTAC |
| | | TTTGCTTATCATCTGCCACAGGGCAAGTGGATTTCCTT |
| | | CTCGCGCGGCTGGGTGAAAACGGTTAACGTGAA |
| 47 | Pichia pastoris | GCCTTGGGGGACTTCAAGTCTTTGCTAGAAACTAGAT |
| | Sequence of the | GAGGTCAGGCCCTCTTATGGTTGTGTCCCAATTGGGCA |
| | 3′-Region used | ATTTCACTCACCTAAAAAGCATGACAATTATTTAGCG |
| | for knock out of | AAATAGGTAGTATATTTTCCCTCATCTCCCAAGCAGTT |
| | BMT4 | TCGTTTTTGCATCCATATCTCTCAAATGAGCAGCTACG |
| | | ACTCATTAGAACCAGAGTCAAGTAGGGGTGAGCTCAG |
| | | TCATCAGCCTTCGTTTCTAAAACGATTGAGTTCTTTTG |
| | | TTGCTACAGGAAGCGCCCTAGGGAACTTTCGCACTTT |
| | | GGAAATAGATTTTGATGACCAAGAGCGGGAGTTGATA |
| | | TTAGAGAGGCTGTCCAAAGTACATGGGATCAGGCCGG |
| | | CCAAATTGATTGGTGTGACTAAACCATTGTGTACTTGG |
| | | ACACTCTATTACAAAAGCGAAGATGATTTGAAGTATT |
| | | ACAAGTCCCGAAGTGTTAGAGGATTCTATCGAGCCCA |
| | | GAATGAAATCATCAACCGTTATCAGCAGATTGATAAA |
| | | CTCTTGGAAAGCGGTATCCCATTTTCATTATTGAAGAA |
| | | CTACGATAATGAAGATGTGAGAGACGGCGACCCTCTG |
| | | AACGTAGACGAAGAAACAAATCTACTTTTGGGGTACA |
| | | ATAGAGAAAGTGAATCAAGGGAGGTATTTGTGGCCAT |
| | | AATACTCAACTCTATCATTAATG |
| 48 | Pichia pastoris | CATATGGTGAGAGCCGTTCTGCACAACTAGATGTTTTC |
| | Sequence of the | GAGCTTCGCATTGTTTCCTGCAGCTCGACTATTGAATT |
| | 5′-Region used | AAGATTTCCGGATATCTCCAATCTCACAAAAACTTATG |
| | for knock out of | TTGACCACGTGCTTTCCTGAGGCGAGGTGTTTTATATG |
| | BMT1 | CAAGCTGCCAAAAATGGAAAACGAATGGCCATTTTTC |
| | | GCCCAGGCAAATTATTCGATTACTGCTGTCATAAAGA |
| | | CAGTGTTGCAAGGCTCACATTTTTTTTTAGGATCCGAG |
| | | ATAAAGTGAATACAGGACAGCTTATCTCTATATCTTGT |
| | | ACCATTCGTGAATCTTAAGAGTTCGGTTAGGGGGACT |
| | | CTAGTTGAGGGTTGGCACTCACGTATGGCTGGGCGCA |
| | | GAAATAAAATTCAGGCGCAGCAGCACTTATCGATG |
| 49 | Pichia pastoris | GAATTCACAGTTATAAATAAAAACAAAAACTCAAAAA |
| | Sequence of the | GTTTGGGCTCCACAAAATAACTTAATTTAAATTTTTGT |
| | 3′-Region used | CTAATAAATGAATGTAATTCCAAGATTATGTGATGCA |
| | for knock out of | AGCACAGTATGCTTCAGCCCTATGCAGCTACTAATGTC |
| | BMT1 | AATCTCGCCTGCGAGCGGGCCTAGATTTTCACTACAA |
| | | ATTTCAAAACTACGCGGATTTATTGTCTCAGAGAGCA |
| | | ATTTGGCATTTCTGAGCGTAGCAGGAGGCTTCATAAG |
| | | ATTGTATAGGACCGTACCAACAAATTGCCGAGGCACA |
| | | ACACGGTATGCTGTGCACTTATGTGGCTACTTCCCTAC |
| | | AACGGAATGAAACCTTCCTCTTTCCGCTTAAACGAGA |
| | | AAGTGTGTCGCAATTGAATGCAGGTGCCTGTGCGCCT |
| | | TGGTGTATTGTTTTTGAGGGCCCAATTTATCAGGCGCC |
| | | TTTTTTCTTGGTTGTTTTCCCTTAGCCTCAAGCAAGGTT |
| | | GGTCTATTTCATCTCCGCTTCTATACCGTGCCTGATAC |
| | | TGTTGGATGAGAACACGACTCAACTTCCTGCTGCTCTG |
| | | TATTGCCAGTGTTTTGTCTGTGATTTGGATCGGAGTCC |
| | | TCCTTACTTGGAATGATAATAATCTTGGCGGAATCTCC |
| | | CTAAACGGAGGCAAGGATTCTGCCTATGATGATCTGC |
| | | TATCATTGGGAAGCTT |
| 50 | Pichia pastoris | GATATCTCCCTGGGGACAATATGTGTTGCAACTGTTCG |
| | Sequence of the | TTGTTGGTGCCCCAGTCCCCCAACCGGTACTAATCGGT |
| | 5′-Region used | CTATGTTCCCGTAACTCATATTCGGTTAGAACTAGAAC |
| | for knock out of | AATAAGTGCATCATTGTTCAACATTGTGGTTCAATTGT |
| | BMT3 | CGAACATTGCTGGTGCTTATATCTACAGGGAAGACGA |
| | | TAAGCCTTTGTACAAGAGAGGTAACAGACAGTTAATT |
| | | GGTATTTCTTTGGGAGTCGTTGCCCTCTACGTTGTCTC |
| | | CAAGACATACTACATTCTGAGAAACAGATGGAAGACT |
| | | CAAAAATGGGAGAAGCTTAGTGAAGAAGAGAAAGTT |
| | | GCCTACTTGGACAGAGCTGAGAAGGAGAACCTGGGTT |
| | | CTAAGAGGCTGGACTTTTTGTTCGAGAGTTAAACTGC |
| | | ATAATTTTTTCTAAGTAAATTTCATAGTTATGAAATTT |
| | | CTGCAGCTTAGTGTTTACTGCATCGTTTACTGCATCAC |
| | | CCTGTAAATAATGTGAGCTTTTTTCCTTCCATTGCTTG |
| | | GTATCTTCCTTGCTGCTGTTT |
| 51 | Pichia pastoris | ACAAAACAGTCATGTACAGAACTAACGCCTTTAAGAT |
| | Sequence of the | GCAGACCACTGAAAAGAATTGGGTCCCATTTTTCTTG |
| | 3′-Region used | AAAGACGACCAGGAATCTGTCCATTTTGTTTACTCGTT |
| | for knock out of | CAATCCTCTGAGAGTACTCAACTGCAGTCTTGATAAC |
| | BMT3 | GGTGCATGTGATGTTCTATTTGAGTTACCACATGATTT |
| | | TGGCATGTCTTCCGAGCTACGTGGTGCCACTCCTATGC |
| | | TCAATCTTCCTCAGGCAATCCCGATGGCAGACGACAA |
| | | AGAAATTTGGGTTTCATTCCCAAGAACGAGAATATCA |
| | | GATTGCGGGTGTTCTGAAACAATGTACAGGCCAATGT |
| | | TAATGCTTTTTGTTAGAGAAGGAACAAACTTTTTTGCT |
| | | GAGC |
| 52 | Trichoderma | CGCGCCGGATCTCCCAACCCTACGAGGGCGGCAGCAG |
| | reesei | TCAAGGCCGCATTCCAGACGTCGTGGAACGCTTACCA |
| | DNA encodes Tr | CCATTTTGCCTTTCCCCATGACGACCTCCACCCGGTCA |
| | Mani catalytic | GCAACAGCTTTGATGATGAGAGAAACGGCTGGGGCTC |
| | domain | GTCGGCAATCGATGGCTTGGACACGGCTATCCTCATG |
| | | GGGGATGCCGACATTGTGAACACGATCCTTCAGTATG |
| | | TACCGCAGATCAACTTCACCACGACTGCGGTTGCCAA |
| | | CCAAGGCATCTCCGTGTTCGAGACCAACATTCGGTAC |
| | | CTCGGTGGCCTGCTTTCTGCCTATGACCTGTTGCGAGG |
| | | TCCTTTCAGCTCCTTGGCGACAAACCAGACCCTGGTAA |
| | | ACAGCCTTCTGAGGCAGGCTCAAACACTGGCCAACGG |
| | | CCTCAAGGTTGCGTTCACCACTCCCAGCGGTGTCCCGG |
| | | ACCCTACCGTCTTCTTCAACCCTACTGTCCGGAGAAGT |
| | | GGTGCATCTAGCAACAACGTCGCTGAAATTGGAAGCC |
| | | TGGTGCTCGAGTGGACACGGTTGAGCGACCTGACGGG |
| | | AAACCCGCAGTATGCCCAGCTTGCGCAGAAGGGCGAG |
| | | TCGTATCTCCTGAATCCAAAGGGAAGCCCGGAGGCAT |
| | | GGCCTGGCCTGATTGGAACGTTTGTCAGCACGAGCAA |
| | | CGGTACCTTTCAGGATAGCAGCGGCAGCTGGTCCGGC |
| | | CTCATGGACAGCTTCTACGAGTACCTGATCAAGATGT |
| | | ACCTGTACGACCCGGTTGCGTTTGCACACTACAAGGA |
| | | TCGCTGGGTCCTTGCTGCCGACTCGACCATTGCGCATC |
| | | TCGCCTCTCACCCGTCGACGCGCAAGGACTTGACCTTT |
| | | TTGTCTTCGTACAACGGACAGTCTACGTCGCCAAACTC |
| | | AGGACATTTGGCCAGTTTTGCCGGTGGCAACTTCATCT |
| | | TGGGAGGCATTCTCCTGAACGAGCAAAAGTACATTGA |
| | | CTTTGGAATCAAGCTTGCCAGCTCGTACTTTGCCACGT |
| | | ACAACCAGACGGCTTCTGGAATCGGCCCCGAAGGCTT |
| | | CGCGTGGGTGGACAGCGTGACGGGCGCCGGCGGCTCG |
| | | CCGCCCTCGTCCCAGTCCGGGTTCTACTCGTCGGCAGG |
| | | ATTCTGGGTGACGGCACCGTATTACATCCTGCGGCCG |
| | | GAGACGCTGGAGAGCTTGTACTACGCATACCGCGTCA |
| | | CGGGCGACTCCAAGTGGCAGGACCTGGCGTGGGAAGC |
| | | GTTCAGTGCCATTGAGGACGCATGCCGCGCCGGCAGC |
| | | GCGTACTCGTCCATCAACGACGTGACGCAGGCCAACG |
| | | GCGGGGGTGCCTCTGACGATATGGAGAGCTTCTGGTT |
| | | TGCCGAGGCGCTCAAGTATGCGTACCTGATCTTTGCG |
| | | GAGGAGTCGGATGTGCAGGTGCAGGCCAACGGCGGG |
| | | AACAAATTTGTCTTTAACACGGAGGCGCACCCCTTTA |
| | | GCATCCGTTCATCATCACGACGGGGCGGCCACCTTGC |
| | | TTAA |
| 53 | Saccharomyces | ATGAGATTCCCATCCATCTTCACTGCTGTTTTGTTCGC |
| | cerevisiae | TGCTTCTTCTGCTTTGGCT |
| | mating factor | |
| | pre-signal | |
| | peptide (DNA) | |
| 54 | Saccharomyces | MRFPSIFTAVLFAASSALA |
| | cerevisiae | |
| | mating factor | |
| | pre-signal | |
| | peptide (protein) | |
| 55 | Pichia pastoris | AACATCCAAAGACGAAAGGTTGAATGAAACCTTTTTG |
| | Pp AOX1 | CCATCCGACATCCACAGGTCCATTCTCACACATAAGT |
| | promoter | GCCAAACGCAACAGGAGGGGATACACTAGCAGCAGA |
| | | CCGTTGCAAACGCAGGACCTCCACTCCTCTTCTCCTCA |
| | | ACACCCACTTTTGCCATCGAAAAACCAGCCCAGTTATT |
| | | GGGCTTGATTGGAGCTCGCTCATTCCAATTCCTTCTAT |
| | | TAGGCTACTAACACCATGACTTTATTAGCCTGTCTATC |
| | | CTGGCCCCCCTGGCGAGGTTCATGTTTGTTTATTTCCG |
| | | AATGCAACAAGCTCCGCATTACACCCGAACATCACTC |
| | | CAGATGAGGGCTTTCTGAGTGTGGGGTCAAATAGTTT |
| | | CATGTTCCCCAAATGGCCCAAAACTGACAGTTTAAAC |
| | | GCTGTCTTGGAACCTAATATGACAAAAGCGTGATCTC |
| | | ATCCAAGATGAACTAAGTTTGGTTCGTTGAAATGCTA |
| | | ACGGCCAGTTGGTCAAAAAGAAACTTCCAAAAGTCGG |
| | | CATACCGTTTGTCTTGTTTGGTATTGATTGACGAATGC |
| | | TCAAAAATAATCTCATTAATGCTTAGCGCAGTCTCTCT |
| | | ATCGCTTCTGAACCCCGGTGCACCTGTGCCGAAACGC |
| | | AAATGGGGAAACACCCGCTTTTTGGATGATTATGCAT |
| | | TGTCTCCACATTGTATGCTTCCAAGATTCTGGTGGGAA |
| | | TACTGCTGATAGCCTAACGTTCATGATCAAAATTTAAC |
| | | TGTTCTAACCCCTACTTGACAGCAATATATAAACAGA |
| | | AGGAAGCTGCCCTGTCTTAAACCTTTTTTTTTATCATC |
| | | ATTATTAGCTTACTTTCATAATTGCGACTGGTTCCAAT |
| | | TGACAAGCTTTTGATTTTAACGACTTTTAACGACAACT |
| | | TGAGAAGATCAAAAAACAACTAATTATTCGAAACG |
| 56 | Pichia pastoris | TACCAATTGCCAAATCAGGCAATTGTGAGACAGTGGT |
| | 5′ARG1 and | AAAAAAGATGCCTGCAAAGTTAGATTCACACAGTAAG |
| | ORF | AGAGATCCTACTCATAAATGAGGCGCTTATTTAGTAG |
| | | CTAGTGATAGCCACTGCGGTTCTGCTTTATGCTATTTG |
| | | TTGTATGCCTTACTATCTTTGTTTGGCTCCTTTTTCTTG |
| | | ACGTTTTCCGTTGGAGGGACTCCCTATTCTGAGTCATG |
| | | AGCCGCACAGATTATCGCCCAAAATTGACAAAATCTT |
| | | CTGGCGAAAAAAGTATAAAAGGAGAAAAAAGCTCAC |
| | | CCTTTTCCAGCGTAGAAAGTATATATCAGTCATTGAAG |
| | | ACTATTATTTAAATAACACAATGTCTAAAGGAAAAGT |
| | | TTGTTTGGCCTACTCCGGTGGTTTGGATACCTCCATCA |
| | | TCCTAGCTTGGTTGTTGGAGCAGGGATACGAAGTCGT |
| | | TGCCTTTTTAGCCAACATTGGTCAAGAGGAAGACTTTG |
| | | AGGCTGCTAGAGAGAAAGCTCTGAAGATCGGTGCTAC |
| | | CAAGTTTATCGTCAGTGACGTTAGGAAGGAATTTGTTG |
| | | AGGAAGTTTTGTTCCCAGCAGTCCAAGTTAACGCTATC |
| | | TACGAGAACGTCTACTTACTGGGTACCTCTTTGGCCAG |
| | | ACCAGTCATTGCCAAGGCCCAAATAGAGGTTGCTGAA |
| | | CAAGAAGGTTGTTTTGCTGTTGCCCACGGTTGTACCGG |
| | | AAAGGGTAACGATCAGGTTAGATTTGAGCTTTCCTTTT |
| | | ATGCTCTGAAGCCTGACGTTGTCTGTATCGCCCCATGG |
| | | AGAGACCCAGAATTCTTCGAAAGATTCGCTGGTAGAA |
| | | ATGACTTGCTGAATTACGCTGCTGAGAAGGATATTCC |
| | | AGTTGCTCAGACTAAAGCCAAGCCATGGTCTACTGAT |
| | | GAGAACATGGCTCACATCTCCTTCGAGGCTGGTATTCT |
| | | AGAAGATCCAAACACTACTCCTCCAAAGGACATGTGG |
| | | AAGCTCACTGTTGACCCAGAAGATGCACCAGACAAGC |
| | | CAGAGTTCTTTGACGTCCACTTTGAGAAGGGTAAGCC |
| | | AGTTAAATTAGTTCTCGAGAACAAAACTGAGGTCACC |
| | | GATCCGGTTGAGATCTTTTTGACTGCTAACGCCATTGC |
| | | TAGAAGAAACGGTGTTGGTAGAATTGACATTGTCGAG |
| | | AACAGATTCATCGGAATCAAGTCCAGAGGTTGTTATG |
| | | AAACTCCAGGTTTGACTCTACTGAGAACCACTCACAT |
| | | CGACTTGGAAGGTCTTACCGTTGACCGTGAAGTTAGA |
| | | TCGATCAGAGACACTTTTGTTACCCCAACCTACTCTAA |
| | | GTTGTTATACAACGGGTTGTACTTTACCCCAGAAGGTG |
| | | AGTACGTCAGAACTATGATTCAGCCTTCTCAAAACAC |
| | | CGTCAACGGTGTTGTTAGAGCCAAGGCCTACAAAGGT |
| | | AATGTGTATAACCTAGGAAGATACTCTGAAACCGAGA |
| | | AATTGTACGATGCTACCGAATCTTCCATGGATGAGTTG |
| | | ACCGGATTCCACCCTCAAGAAGCTGGAGGATTTATCA |
| | | CAACACAAGCCATCAGAATCAAGAAGTACGGAGAAA |
| | | GTGTCAGAGAGAAGGGAAAGTTTTTGGGACTTTAACT |
| | | CAAGTAAAAGGATAGTTGTACAATTATATATACGAAG |
| | | AATAAATCATTACAAAAAGTATTCGTTTCTTTGATTCT |
| | | TAACAGGATTCATTTTCTGGGTGTCATCAGGTACAGCG |
| | | CTGAATATCTTGAAGTTAACATCGAGCTCATCATCGAC |
| | | GTTCATCACACTAGCCACGTTTCCGCAACGGTAG |
| 57 | Pichia pastoris | GGGACTTTAACTCAAGTAAAAGGATAGTTGTACAATT |
| | Sequence of the | ATATATACGAAGAATAAATCATTACAAAAAGTATTCG |
| | 3′-Region used | TTTCTTTGATTCTTAACAGGATTCATTTTCTGGGTGTCA |
| | for knock out of | TCAGGTACAGCGCTGAATATCTTGAAGTTAACATCGA |
| | PpARG1: | GCTCATCATCGACGTTCATCACACTAGCCACGTTTCCG |
| | | CAACGGTAGCAATAATTAGGAGCGGACCACACAGTGA |
| | | CGACATCTTTCTCTTTGAAATGGTATCTGAAGCCTTCC |
| | | ATGACCAATTGATGGGCTCTAGCGATGAGTTGCAAGT |
| | | TATTAATGTGGTTGAACTCACGTGCTACTCGAGCACCG |
| | | AATAACCAGCCAGCTCCACGAGGAGAAACAGCCCAA |
| | | CTGTCGACTTCATCTGGGTCAGACCAAACCAAGTCAC |
| | | AAAATCCTCCTTCATGAGGGACCTCTTGCGCTCGGCTG |
| | | AGAACTCTGATTTGATCTAACATGCGAATATCGGGAG |
| | | AGAGACCACCATGGATACATAATATTTTACCATCAAT |
| | | GATGGCACTAAGGGTTAAAAAGTCGAACACCTGGCAA |
| | | CAGTACTTCCAGACAGTGGTGGAACCATATTTATTGA |
| | | GACATTCCTCATAAAATCCATAAACCTGAGTGATCTGT |
| | | CTGGATTCATGATTTCCCCTTACCAATGTGATATGTTG |
| | | AGGAAACTTAATTTTTAAAATCATGAGTAACGTGAAC |
| | | GTCTCCAACGAGAAATAGCCTCTATCCACATAGTCTCC |
| | | TAGGAAGATATAGTTCTGTTTTATTCCATTAGAGGAGG |
| | | ATCCGGGAAACCCACCACTAATCTTGAAAAGTTCCAG |
| | | TAGATCGTGAAATTGGCCGTGAATATCTCCGCATACT |
| | | GTCACTGGACTCTGCACTGGCTGTATATTGGATTCCTC |
| | | CATCAGCAAATCCTTCACCCGTTCGCAAAGATGCTTCA |
| | | TATCATTTTCACTTAAAGCCTTGCAGCTTTTGACTTCTT |
| | | CAAACCACTGATCTGGTCCTCTTTCTGGCATGATTAAG |
| | | GTCTATAATATTTCTGAGCTGAGATGTAAAAAAAAAT |
| | | AATAAAAATGGGGAGTGAAAAAGTGTGTAGCTTTTAG |
| | | GAGTTTGGGATTGATACCCCAAAATGATCTTTATGAG |
| | | AATTAAAAGGTAGATACGCTTTTAATAAGAACACCTA |
| | | TCTATAGTACTTTGTGGTCTTGAGTAATTGAGATGTTC |
| | | AGCTTCTGAGGTTTGCCGTTATTCTGGGATAGTAGTGC |
| | | GCGACCAAACAACCCGCCAGGCAAAGTGTGTTGTGCT |
| | | CGAAGACGATTGCCAGAAGAGTAAGTCCGTCCTGCCT |
| | | CAGATGTTACACACTTTCTTCCCTAGACAGTCGATGCA |
| | | TCATCGGATTTAAACCTGAAACTTTGATGCCATGATAC |
| | | GCCTAGTCACGTCGACTGAGATTTTAGATAAGCCCCG |
| | | ATCCCTTTAGTACATTCCTGTTATCCATGGATGGAATG |
| | | GCCTGATA |
| 58 | human | GAGGTTCAGTTGGTTGAATCTGGAGGAGGATTGGTTC |
| | Anti-Her2 | AACCTGGTGGTTCTTTGAGATTGTCCTGTGCTGCTTCC |
| | Heavy chain | GGTTTCAACATCAAGGACACTTACATCCACTGGGTTA |
| | (VH + IgG1 | GACAAGCTCCAGGAAAGGGATTGGAGTGGGTTGCTAG |
| | constant region) | AATCTACCCAACTAACGGTTACACAAGATACGCTGAC |
| | (DNA) | TCCGTTAAGGGAAGATTCACTATCTCTGCTGACACTTC |
| | | CAAGAACACTGCTTACTTGCAGATGAACTCCTTGAGA |
| | | GCTGAGGATACTGCTGTTTACTACTGTTCCAGATGGGG |
| | | TGGTGATGGTTTCTACGCTATGGACTACTGGGGTCAA |
| | | GGAACTTTGGTTACTGTTTCCTCCGCTTCTACTAAGGG |
| | | ACCATCTGTTTTCCCATTGGCTCCATCTTCTAAGTCTA |
| | | CTTCCGGTGGTACTGCTGCTTTGGGATGTTTGGTTAAA |
| | | GACTACTTCCCAGAGCCAGTTACTGTTTCTTGGAACTC |
| | | CGGTGCTTTGACTTCTGGTGTTCACACTTTCCCAGCTG |
| | | TTTTGCAATCTTCCGGTTTGTACTCTTTGTCCTCCGTTG |
| | | TTACTGTTCCATCCTCTTCCTTGGGTACTCAGACTTAC |
| | | ATCTGTAACGTTAACCACAAGCCATCCAACACTAAGG |
| | | TTGACAAGAAGGTTGAGCCAAAGTCCTGTGACAAGAC |
| | | ACATACTTGTCCACCATGTCCAGCTCCAGAATTGTTGG |
| | | GTGGTCCATCCGTTTTCTTGTTCCCACCAAAGCCAAAG |
| | | GACACTTTGATGATCTCCAGAACTCCAGAGGTTACAT |
| | | GTGTTGTTGTTGACGTTTCTCACGAGGACCCAGAGGTT |
| | | AAGTTCAACTGGTACGTTGACGGTGTTGAAGTTCACA |
| | | ACGCTAAGACTAAGCCAAGAGAAGAGCAGTACAACT |
| | | CCACTTACAGAGTTGTTTCCGTTTTGACTGTTTTGCAC |
| | | CAGGACTGGTTGAACGGTAAAGAATACAAGTGTAAGG |
| | | TTTCCAACAAGGCTTTGCCAGCTCCAATCGAAAAGAC |
| | | TATCTCCAAGGCTAAGGGTCAACCAAGAGAGCCACAG |
| | | GTTTACACTTTGCCACCATCCAGAGAAGAGATGACTA |
| | | AGAACCAGGTTTCCTTGACTTGTTTGGTTAAAGGATTC |
| | | TACCCATCCGACATTGCTGTTGAGTGGGAATCTAACG |
| | | GTCAACCAGAGAACAACTACAAGACTACTCCACCAGT |
| | | TTTGGATTCTGATGGTTCCTTCTTCTTGTACTCCAAGTT |
| | | GACTGTTGACAAGTCCAGATGGCAACAGGGTAACGTT |
| | | TTCTCCTGTTCCGTTATGCATGAGGCTTTGCACAACCA |
| | | CTACACTCAAAAGTCCTTGTCTTTGTCCCCTGGTTAA |
| 59 | human | GACATCCAAATGACTCAATCCCCATCTTCTTTGTCTGC |
| | Anti-Her2 light | TTCCGTTGGTGACAGAGTTACTATCACTTGTAGAGCTT |
| | chain (VL + | CCCAGGACGTTAATACTGCTGTTGCTTGGTATCAACAG |
| | Kappa constant | AAGCCAGGAAAGGCTCCAAAGTTGTTGATCTACTCCG |
| | region) (DNA) | CTTCCTTCTTGTACTCTGGTGTTCCATCCAGATTCTCTG |
| | | GTTCCAGATCCGGTACTGACTTCACTTTGACTATCTCC |
| | | TCCTTGCAACCAGAAGATTTCGCTACTTACTACTGTCA |
| | | GCAGCACTACACTACTCCACCAACTTTCGGACAGGGT |
| | | ACTAAGGTTGAGATCAAGAGAACTGTTGCTGCTCCAT |
| | | CCGTTTTCATTTTCCCACCATCCGACGAACAGTTGAAG |
| | | TCTGGTACAGCTTCCGTTGTTTGTTTGTTGAACAACTT |
| | | CTACCCAAGAGAGGCTAAGGTTCAGTGGAAGGTTGAC |
| | | AACGCTTTGCAATCCGGTAACTCCCAAGAATCCGTTA |
| | | CTGAGCAAGACTCTAAGGACTCCACTTACTCCTTGTCC |
| | | TCCACTTTGACTTTGTCCAAGGCTGATTACGAGAAGCA |
| | | CAAGGTTTACGCTTGTGAGGTTACACATCAGGGTTTGT |
| | | CCTCCCCAGTTACTAAGTCCTTCAACAGAGGAGAGTG |
| | | TTAA |
| 60 | Streptoalloteichus | ATGGCCAAGTTGACCAGTGCCGTTCCGGTGCTCACCG |
| | hindustanus | CGCGCGACGTCGCCGGAGCGGTCGAGTTCTGGACCGA |
| | Sequence of the | CCGGCTCGGGTTCTCCCGGGACTTCGTGGAGGACGAC |
| | Shble ORF | TTCGCCGGTGTGGTCCGGGACGACGTGACCCTGTTCAT |
| | (Zeocin | CAGCGCGGTCCAGGACCAGGTGGTGCCGGACAACACC |
| | resistance | CTGGCCTGGGTGTGGGTGCGCGGCCTGGACGAGCTGT |
| | marker): | ACGCCGAGTGGTCGGAGGTCGTGTCCACGAACTTCCG |
| | | GGACGCCTCCGGGCCGGCCATGACCGAGATCGGCGAG |
| | | CAGCCGTGGGGGCGGGAGTTCGCCCTGCGCGACCCGG |
| | | CCGGCAACTGCGTGCACTTCGTGGCCGAGGAGCAGGA |
| | | CTGA |
| 61 | Saccharomyces | GATCCCCCACACACCATAGCTTCAAAATGTTTCTACTC |
| | cerevisiae | CTTTTTTACTCTTCCAGATTTTCTCGGACTCCGCGCATC |
| | ScTEF 1 | GCCGTACCACTTCAAAACACCCAAGCACAGCATACTA |
| | promoter | AATTTCCCCTCTTTCTTCCTCTAGGGTGTCGTTAATTAC |
| | | CCGTACTAAAGGTTTGGAAAAGAAAAAAGAGACCGC |
| | | CTCGTTTCTTTTTCTTCGTCGAAAAAGGCAATAAAAAT |
| | | TTTTATCACGTTTCTTTTTCTTGAAAATTTTTTTTTTTG |
| | | ATTTTTTTCTCTTTCGATGACCTCCCATTGATATTTAAG |
| | | TTAATAAACGGTCTTCAATTTCTCAAGTTTCAGTTTCA |
| | | TTTTTCTTGTTCTATTACAACTTTTTTTACTTCTTGCTC |
| | | ATTAGAAAGAAAGCATAGCAATCTAATCTAAGTTTTA |
| | | ATTACAAA |
| 62 | Pichia pastoris | ATGAGTGTAAGTGATAGTCATCTTGCAACAGATTATTT |
| | PpTRP2 Region | TGGAACGCAACTAACAAAGCAGATACACCCTTCAGCA |
| | | GAATCCTTTCTGGATATTGTGAAGAATGATCGCCAAA |
| | | GTCACAGTCCTGAGACAGTTCCTAATCTTTACCCCATT |
| | | TACAAGTTCATCCAATCAGACTTCTTAACGCCTCATCT |
| | | GGCTTATATCAAGCTTACCAACAGTTCAGAAACTCCC |
| | | AGTCCAAGTTTCTTGCTTGAAAGTGCGAAGAATGGTG |
| | | ACACCGTTGACAGGTACACCTTTATGGGACATTCCCCC |
| | | AGAAAAATAATCAAGACTGGGCCTTTAGAGGGTGCTG |
| | | AAGTTGACCCCTTGGTGCTTCTGGAAAAAGAACTGAA |
| | | GGGCACCAGACAAGCGCAACTTCCTGGTATTCCTCGT |
| | | CTAAGTGGTGGTGCCATAGGATACATCTCGTACGATT |
| | | GTATTAAGTACTTTGAACCAAAAACTGAAAGAAAACT |
| | | GAAAGATGTTTTGCAACTTCCGGAAGCAGCTTTGATG |
| | | TTGTTCGACACGATCGTGGCTTTTGACAATGTTTATCA |
| | | AAGATTCCAGGTAATTGGAAACGTTTCTCTATCCGTTG |
| | | ATGACTCGGACGAAGCTATTCTTGAGAAATATTATAA |
| | | GACAAGAGAAGAAGTGGAAAAGATCAGTAAAGTGGT |
| | | ATTTGACAATAAAACTGTTCCCTACTATGAACAGAAA |
| | | GATATTATTCAAGGCCAAACGTTCACCTCTAATATTGG |
| | | TCAGGAAGGGTATGAAAACCATGTTCGCAAGCTGAAA |
| | | GAACATATTCTGAAAGGAGACATCTTCCAAGCTGTTC |
| | | CCTCTCAAAGGGTAGCCAGGCCGACCTCATTGCACCC |
| | | TTTCAACATCTATCGTCATTTGAGAACTGTCAATCCTT |
| | | CTCCATACATGTTCTATATTGACTATCTAGACTTCCAA |
| | | GTTGTTGGTGCTTCACCTGAATTACTAGTTAAATCCGA |
| | | CAACAACAACAAAATCATCACACATCCTATTGCTGGA |
| | | ACTCTTCCCAGAGGTAAAACTATCGAAGAGGACGACA |
| | | ATTATGCTAAGCAATTGAAGTCGTCTTTGAAAGACAG |
| | | GGCCGAGCACGTCATGCTGGTAGATTTGGCCAGAAAT |
| | | GATATTAACCGTGTGTGTGAGCCCACCAGTACCACGG |
| | | TTGATCGTTTATTGACTGTGGAGAGATTTTCTCATGTG |
| | | ATGCATCTTGTGTCAGAAGTCAGTGGAACATTGAGAC |
| | | CAAACAAGACTCGCTTCGATGCTTTCAGATCCATTTTC |
| | | CCAGCAGGAACCGTCTCCGGTGCTCCGAAGGTAAGAG |
| | | CAATGCAACTCATAGGAGAATTGGAAGGAGAAAAGA |
| | | GAGGTGTTTATGCGGGGGCCGTAGGACACTGGTCGTA |
| | | CGATGGAAAATCGATGGACACATGTATTGCCTTAAGA |
| | | ACAATGGTCGTCAAGGACGGTGTCGCTTACCTTCAAG |
| | | CCGGAGGTGGAATTGTCTACGATTCTGACCCCTATGA |
| | | CGAGTACATCGAAACCATGAACAAAATGAGATCCAAC |
| | | AATAACACCATCTTGGAGGCTGAGAAAATCTGGACCG |
| | | ATAGGTTGGCCAGAGACGAGAATCAAAGTGAATCCGA |
| | | AGAAAACGATCAATGA |
| 63 | Pichia pastoris | CCGGCCATTTAAATATGTGACGACTGGGTGATCCGGG |
| | PpCITI TT | TTAGTGAGTTGTTCTCCCATCTGTATATTTTTCATTTAC |
| | | GATGAATACGAAATGAGTATTAAGAAATCAGGCGTAG |
| | | CAATATGGGCAGTGTTCAGTCCTGTCATAGATGGCAA |
| | | GCACTGGCACATCCTTAATAGGTTAGAGAAAATCATT |
| | | GAATCATTTGGGTGGTGAAAAAAAATTGATGTAAACA |
| | | AGCCACCCACGCTGGGAGTCGAACCCAGAATCTTTTG |
| | | ATTAGAAGTCAAACGCGTTAACCATTACGCTACGCAG |
| | | GCATGTTTCACGTCCATTTTTGATTGCTTTCTATCATAA |
| | | TCTAAAGATGTGAACTCAATTAGTTGCAATTTGACCA |
| | | ATTCTTCCATTACAAGTCGTGCTTCCTCCGTTGATGCA |
| | | AC |
| 64 | Streptomyces | ATGGGTACCACTCTTGACGACACGGCTTACCGGTACC |
| | noursei | GCACCAGTGTCCCGGGGGACGCCGAGGCCATCGAGGC |
| | NatR ORF | ACTGGATGGGTCCTTCACCACCGACACCGTCTTCCGCG |
| | | TCACCGCCACCGGGGACGGCTTCACCCTGCGGGAGGT |
| | | GCCGGTGGACCCGCCCCTGACCAAGGTGTTCCCCGAC |
| | | GACGAATCGGACGACGAATCGGACGACGGGGAGGAC |
| | | GGCGACCCGGACTCCCGGACGTTCGTCGCGTACGGGG |
| | | ACGACGGCGACCTGGCGGGCTTCGTGGTCGTCTCGTA |
| | | CTCCGGCTGGAACCGCCGGCTGACCGTCGAGGACATC |
| | | GAGGTCGCCCCGGAGCACCGGGGGCACGGGGTCGGG |
| | | CGCGCGTTGATGGGGCTCGCGACGGAGTTCGCCCGCG |
| | | AGCGGGGCGCCGGGCACCTCTGGCTGGAGGTCACCAA |
| | | CGTCAACGCACCGGCGATCCACGCGTACCGGCGGATG |
| | | GGGTTCACCCTCTGCGGCCTGGACACCGCCCTGTACG |
| | | ACGGCACCGCCTCGGACGGCGAGCAGGCGCTCTACAT |
| | | GAGCATGCCCTGCCCCTAATCAGTACTG |
| 65 | Ashbya gossypii | GATCTGTTTAGCTTGCCTCGTCCCCGCCGGGTCACCCG |
| | TEF1 promoter | GCCAGCGACATGGAGGCCCAGAATACCCTCCTTGACA |
| | | GTCTTGACGTGCGCAGCTCAGGGGCATGATGTGACTG |
| | | TCGCCCGTACATTTAGCCCATACATCCCCATGTATAAT |
| | | CATTTGCATCCATACATTTTGATGGCCGCACGGCGCGA |
| | | AGCAAAAATTACGGCTCCTCGCTGCAGACCTGCGAGC |
| | | AGGGAAACGCTCCCCTCACAGACGCGTTGAATTGTCC |
| | | CCACGCCGCGCCCCTGTAGAGAAATATAAAAGGTTAG |
| | | GATTTGCCACTGAGGTTCTTCTTTCATATACTTCCTTTT |
| | | AAAATCTTGCTAGGATACAGTTCTCACATCACATCCG |
| | | AACATAAACAACC |
| 66 | Ashbya gossypii | TAATCAGTACTGACAATAAAAAGATTCTTGTTTTCAAG |
| | TEF1 | AACTTGTCATTTGTATAGTTTTTTTATATTGTAGTTGTT |
| | termination | CTATTTTAATCAAATGTTAGCGTGATTTATATTTTTTTT |
| | sequence | CGCCTCGACATCATCTGCCCAGATGCGAAGTTAAGTG |
| | | CGCAGAAAGTAATATCATGCGTCAATCGTATGTGAAT |
| | | GCTGGTCGCTATACTGCTGTCGATTCGATACTAACGCC |
| | | GCCATCCAGTGTCGAAAAC |
| 67 | Pichia pastoris | GCGGAAACGGCAGTAAACAATGGAGCTTCATTAGTGG |
| | PpTRP1 5′ | GTGTTATTATGGTCCCTGGCCGGGAACGAACGGTGAA |
| | region and ORF | ACAAGAGGTTGCGAGGGAAATTTCGCAGATGGTGCGG |
| | | GAAAAGAGAATTTCAAAGGGCTCAAAATACTTGGATT |
| | | CCAGACAACTGAGGAAAGAGTGGGACGACTGTCCTCT |
| | | GGAAGACTGGTTTGAGTACAACGTGAAAGAAATAAAC |
| | | AGCAGTGGTCCATTTTTAGTTGGAGTTTTTCGTAATCA |
| | | AAGTATAGATGAAATCCAGCAAGCTATCCACACTCAT |
| | | GGTTTGGATTTCGTCCAACTACATGGGTCTGAGGATTT |
| | | TGATTCGTATATACGCAATATCCCAGTTCCTGTGATTA |
| | | CCAGATACACAGATAATGCCGTCGATGGTCTTACCGG |
| | | AGAAGACCTCGCTATAAATAGGGCCCTGGTGCTACTG |
| | | GACAGCGAGCAAGGAGGTGAAGGAAAAACCATCGAT |
| | | TGGGCTCGTGCACAAAAATTTGGAGAACGTAGAGGAA |
| | | AATATTTACTAGCCGGAGGTTTGACACCTGATAATGTT |
| | | GCTCATGCTCGATCTCATACTGGCTGTATTGGTGTTGA |
| | | CGTCTCTGGTGGGGTAGAAACAAATGCCTCAAAAGAT |
| | | ATGGACAAGATCACACAATTTATCAGAAACGCTACAT |
| | | AA |
| 68 | Pichia pastoris | AAGTCAATTAAATACACGCTTGAAAGGACATTACATA |
| | PpTRP1 3′ | GCTTTCGATTTAAGCAGAACCAGAAATGTAGAACCAC |
| | region | TTGTCAATAGATTGGTCAATCTTAGCAGGAGCGGCTG |
| | | GGCTAGCAGTTGGAACAGCAGAGGTTGCTGAAGGTGA |
| | | GAAGGATGGAGTGGATTGCAAAGTGGTGTTGGTTAAG |
| | | TCAATCTCACCAGGGCTGGTTTTGCCAAAAATCAACTT |
| | | CTCCCAGGCTTCACGGCATTCTTGAATGACCTCTTCTG |
| | | CATACTTCTTGTTCTTGCATTCACCAGAGAAAGCAAAC |
| | | TGGTTCTCAGGTTTTCCATCAGGGATCTTGTAAATTCT |
| | | GAACCATTCGTTGGTAGCTCTCAACAAGCCCGGCATG |
| | | TGCTTTTCAACATCCTCGATGTCATTGAGCTTAGGAGC |
| | | CAATGGGTCGTTGATGTCGATGACGATGACCTTCCAG |
| | | TCAGTCTCTCCCTCATCCAACAAAGCCATAACACCGA |
| | | GGACCTTGACTTGCTTGACCTGTCCAGTGTAACCTACG |
| | | GCTTCACCAATTTCGCAAACGTCCAATGGATCATTGTC |
| | | ACCCTTGGCCTTGGTCTCTGGATGAGTGACGTTAGGGT |
| | | CTTCCCATGTCTGAGGGAAGGCACCGTAGTTGTGAAT |
| | | GTATCCGTGGTGAGGGAAACAGTTACGAACGAAACGA |
| | | AGTTTTCCCTTCTTTGTGTCCTGAAGAATTGGGTTCAG |
| | | TTTCTCCTCCTTGGAAATCTCCAACTTGGCGTTGGTCC |
| | | AACGGGGGACTTCAACAACCATGTTGAGAACCTTCTT |
| | | GGATTCGTCAGCATAAAGTGGGATGTCGTGGAAAGGA |
| | | GATACGACTT |
| 69 | Pichia pastoris | GTTAAATGACTCTAACACCTTGCACTTGA |
| | PpXRN1- | |
| | 5′out/UP | |
| 70 | Pichia pastoris | CCTCCCACTGGAACCGATGATATGGAA |
| | PpALG3TT/LP | |
| 71 | Pichia pastoris | GATGCGAAGTTAAGTGCGCAGAAAGTAATATCA |
| | PpTEFTT/UP | |
| 72 | Pichia pastoris | TTGCAAAAACCAGTGAGGAATAGC |
| | PpXRN1- | |
| | 3′out/LP | |
| 73 | Pichia pastoris | GAATGCTGAAGAACGTCAAAGAAACT |
| | PpXRN1/iUP | |
| 74 | Pichia pastoris | TGAGACTTCAGAGCTTTCCATACGA |
| | PpXRN1/iLP | |
| 75 | Pichia pastoris | ATGGGTATTCCAAAGTTTTTTCGGTACATCTCTGAGAG |
| | Sequence of the | ATGGCCGATGATATCTGAGCCAATAGAAGACAGCCAA |
| | PpXRN1 (DNA) | ATTGCCGAGTTTGATAACCTGTATCTGGACATGAACTC |
| | | AATTCTTCATAATTGTACACATAGTAACGATGGATCA |
| | | GTTGATTTAATGAAAGAAGAAGAGATGTTCAGTGCTA |
| | | TTTTTGCTTACATTGAACATCTTTTCACTTTGATCAAAC |
| | | CTGGAAAGACGTTTTTCATGGCCATTGATGGTGTTGCT |
| | | CCTAGAGCAAAGATGAACCAGCAACGATCTAGAAGAT |
| | | TCAGGACGGCCATTGAGGCAGAAAAAAGTGTAGAGAT |
| | | AGCCCAAAAGAATGGACTCATTACTAGCAAAGACGAG |
| | | AATTTTGACAGTAACTGTATCACTCCTGGAACGGAGTT |
| | | TATGGCCAAGGTTACCACTAACTTGAAGTTTTTCATCC |
| | | ACCAGAAAATCTCTTCAGATGCAAAATGGCAGAAAGT |
| | | CCAGGTTATTCTCAGTGGACATGAAGTCCCCGGAGAA |
| | | GGAGAGCACAAAATTATGGACTATATTCGTTTTTTGAA |
| | | GGCTCAAGAGGGGTATGATCCGAATACGAGACATTGT |
| | | ATTTACGGGCTGGATGCAGACTTGATCATGTTGGGATT |
| | | GGTCATTCACGATCCTCATTTTGCCATCTTGAGAGAAG |
| | | AGGTTGTGTTCGGAAGAGGATCCAAGGCCTCGTCAAC |
| | | AGATGTGTCAGAACAGCAATTCTACCTTCTACATCTCT |
| | | CCTTGTTACGTGAATACCTTGCTCTTGAGTTCAAAGAT |
| | | TTAGAAGACCAGATAAAGTTTGATTATGACTTAGAGA |
| | | GAATCTTGGACGATTTTATTTTTATCATGTATGTTATTG |
| | | GAAATGATTTCTTACCCAACTTACCTGACTTGCATATC |
| | | AACAAAGGTGCCTTCCCCAGACTCTTAGCAACGATCA |
| | | AAGAAACCATGATAGATTCGGATGGATATCTCCAAGA |
| | | AGGAGGTGTCATCAATATGGAACGATTCGGTCTGTGG |
| | | CTTGACCACTTGTCACAATTTGAGCTTCAAAATTTTGA |
| | | GCAGGTCGACGTGGATGTAGAATGGTTTAACAAGCAG |
| | | CTAGAGAACATATCCCTAGATGGTGAGCGAAAGAGGG |
| | | AAAGAGCTGGAAGAAAGTTGTTGCTTAGACGTCAACA |
| | | GGAATTAATTTCAAAACTTAGACCATGGATTCTCGAG |
| | | TTTTATTCATCAAGAGACAATATATATTCTGCTCATGA |
| | | TGATGACTCCTTAATTCCAACTTTGCAATTGGACACCG |
| | | AATTGATGGAAGAAGAAATCAAGTTACCCTTCATAAA |
| | | GCAGTTTGCACTTGATGTTGGTTTCTTTATTGT |
| | | GCACAGCAAATCTCAGAATACGTACCATGCTAAGATA |
| | | GACATTGATGGTATAAATGTCAATGAATCTGATGAAG |
| | | AATTTGAAGCTCGTGTCTTGTATATCAGGAAGAAGAT |
| | | CAAAGAATATGAAAACTCAATTTTTGTTGAAGATGAA |
| | | AACACTTTAGAGGAACAAAAGAACATTTATGATACCA |
| | | AGTTCGTCAACTGGAAAAACAAATATTACAAAGAAAA |
| | | GTTTGGATTTACTCTTTCTGACACAGAGGACATTTTGA |
| | | AGTTGACGGAGTCGTATATTCAGGGTCTTCAATGGGTT |
| | | TTGTTCTACTATTATCGAGGAGTTCCTTCTTGGCAATG |
| | | GTACTATCCTTATTACTATGCTCCTAGAATCTCTGACA |
| | | TCAAATTAGGGATTAAGGCTTGCCTGGAGTTTGACAA |
| | | AGGTACACCGTTCAAGCCATTTGAACAATTAATGGCT |
| | | GTCCTTCCTGCAAGATCAAAACAGTTAGTTCCTGCTTG |
| | | TTATAGACCGTTGATGACCGATCCAAACTCTCCCATTA |
| | | TTGAATTTTACCCTGATGAGGTTGAAATTGACAAGAAT |
| | | GGAAAGACTGCCTCTTGGGAGGCTGTTGTTAAAATCA |
| | | ACTTTGTCGATGAAAAACGACTATTAGAGGCACTGGA |
| | | ACCGTATAATAGTAAGTTGAATGCTGAAGAACGTCAA |
| | | AGAAACTCTTTGGGCACGAATATCGAGTTTAGCTATC |
| | | ATCCTCAAGTGAATCAAGTTCATTCTTCCCCAATTCCA |
| | | ACAATATTCCCAGATATCACTGAGGATCACTGCTACG |
| | | AAATAGTGGTCGAATTTGACAAGCTGCACTCTTCAAC |
| | | TTATGCTAAACCTATGAAAGGTGCCAAACAAGGTATA |
| | | AATCTATTGGCTGGATTCCCAACTTTGAAGACCATTCC |
| | | CTTCACTTCGCAGCTCATGTTAGCGGAATGCCATATTT |
| | | TTAACCAGCCAACAAAATCGGAATCGATGATTCTAAG |
| | | TACACAGAATCCGTTTGAAGGACTCACTGTAGAGCAG |
| | | TTTGCTGCTCAGAATTTGGGCCAAACAATCTATGTCAA |
| | | CTGGCCTTATTTGAAAGAGGCCAAAGTTGTTGCAGTTT |
| | | CTGACGGTTTGAACGTTTTTGAGCCTGGAAACAAGTCT |
| | | ATCCGAACGACTCGTATGGAAAGCTCTGAAGTCTCAG |
| | | AATTTGCCAGTGACGTTCGCTATATCAGTGAGACGCT |
| | | ATTCAAGAGAAAAGGTGTTTTACTGGTGAACTACACT |
| | | GAGGAAGAGTTGCAAGGAACACCTGAGCCTCGTAGAT |
| | | CCCCTCACAATGACGATGCGATCCAAGGAATTGTGTT |
| | | TGTAAAGAAAGTGAATGGTGTTATTCGCACTAGATCG |
| | | GGTGCCTATGTGAAGACATACAGTGACAAGATTGAGA |
| | | AATACCCTATTAATTTGCTTGTTGACGACGTGGTTAAC |
| | | AAGGATCGTCGATTATTAGAAAAACCTCCTGTGCCAT |
| | | TAGAAGAAGAATTCCCAAAGGGTACTACTCTCATAAG |
| | | TTTGGGAGCTTTTGCTTACGGAACTCCTGCTACTGTTG |
| | | TTGACCACCAAAATGACTTAATGACCGTCAGATTCGT |
| | | AAACCAGCCAATTAAGCATGAATTTAACTATGGTGAG |
| | | ATTCAAGCACAAAGGGAGGCACACACCAATGTGTATT |
| | | ATCCCTCCTTTAAAGTTGCCAAAATCGTTGGAATTACG |
| | | GCGCTAGCCTTGTCCAGAATAACCTCTTCCTATAGAAT |
| | | AGTTAATGGAGCTAAGAAAACTGTTAACATCGGATTA |
| | | GATTTGAAGTTTGAAGGAAGAAAGTTAAAGGTTCTGG |
| | | GATACACTAAAAGAAACGAGAAACATTGGTCATACAG |
| | | CGCTTTGGCTGTAAACTTGCTGCAACAATATCAGAAA |
| | | CGTTTCCCATCTGTTTTTAAGATAATTTCTCTCCGA |
| | | AATGATTCTTCAATTCCTGAAGCCAAAGAACTGTTTCC |
| | | TCAAGTCCCCGCTAGCCAAGTGGATGAAAAAGTCAAT |
| | | GAACTTGTGGCTTGGGTTAAAGAACAAAAGAAAAGCT |
| | | TTGTTGTTGCAACATTGGAGTCTGAGTCTTTGACCAAG |
| | | GTTAGTATCGGAAAGATTGAACAGGAAGTGATTAAGT |
| | | TTGTTTCAAAACCACATGAACATATTCCCCAAAAGGG |
| | | TTTGAAAGGAGTTCCTCGTGAGGCTACTCTGGATATCA |
| | | GCAACTCTTCTCAATTTCTTTCCAAGCAGACTTTTAAT |
| | | CTAGGAGACAGAGTGATCTACGTAGAAGATTCTGGTA |
| | | AGGTACCCAACTTCAGTAAAGGTACAGTTATTGGGGT |
| | | TCGAAGTGTCGGTACGAAAGTGACTTTGAACGTATTG |
| | | TTTGACTTGCCATTGCTCTCTGGTAATACCTTTGATGG |
| | | AAGACTTGCAACCCCCAGGGGTGTCACTGTTGATAGT |
| | | TCATTGGTATTGAACTTAACAAAGCGTCAATTGATTTA |
| | | CCACGATAGGAAACCCGCTAAGAAAGAAGGTAAGCC |
| | | TGGAGTTAAGAAGACATCTCAGGACTCCAAAAAGCAA |
| | | ACAGTGAAGTTGCAGAACGGTTCAGGTAAGGCCAAAC |
| | | AGGCACAAGATGCTGAACTGTCAGTCACTACGACACC |
| | | TGTTCCGGTTCCTGCCGCTAACACTGCACCAGTTACTG |
| | | CATCTGTACAGGCTACCTCGGTGGTTACTGCTACCGTT |
| | | CCAACTTCTAAACCTTCAAACAACTCTGAGGACGAAC |
| | | ATGAATTACTTCGCTTACTGAAAGGTAACAAGGACAG |
| | | TAGTGAATCGCAAGGCTCTCAAGAGCCTCTTGCCCGG |
| | | ACTTCTATCCAGCAAATTTACGGAACGGTGTTCAATCA |
| | | AGTGTTGAGCGCTCAACCTCAGTTGCAGCCTGTCAGA |
| | | GGCTTCTCAAATCCGGTACCTGAAACCCCTGTAAATG |
| | | GAGTCCAAGCGAATGAACAACACTCTGATTCTACCCC |
| | | TCAAAATCATTCTAGGGATGAAAACCAAGGAAGAGGT |
| | | CGTGGTAGAGGCAGAGGAAATAGAAGAGGAAGAGGT |
| | | CGAGGTAGAGGCAAAGGAGGACAGTAA |
| 76 | Pichia pastoris | MGIPKFFRYISERWPMISEPIEDSQIAEFDNLYLDMNSILH |
| | Sequence of the | NCTHSNDGSVDLMKEEEMFSAIFAYIEHLFTLIKPGKTFF |
| | PpXRN1 | MAIDGVAPRAKMNQQRSRRFRTAIEAEKSVEIAQKNGLI |
| | (protein) | TSKDENFDSNCITPGTEFMAKVTTNLKFFIHQKISSDAKW |
| | | QKVQVILSGHEVPGEGEHKIMDYIRFLKAQEGYDPNTRH |
| | | CIYGLDADLIMLGLVIHDPHFAILREEVVFGRGSKASSTD |
| | | VSEQQFYLLHLSLLREYLALEFKDLEDQIKFDYDLERILD |
| | | DFIFIMYVIGNDFLPNLPDLHINKGAFPRLLATIKETMIDS |
| | | DGYLQEGGVINMERFGLWLDHLSQFELQNFEQVDVDVE |
| | | WFNKQLENISLDGERKRERAGRKLLLRRQQELISKLRPWI |
| | | LEFYSSRDNIYSAHDDDSLIPTLQLDTELMEEEIKLPFIKQF |
| | | ALDVGFFIVHSKSQNTYHAKIDIDGINVNESDEEFEARVL |
| | | YIRKKIKEYENSIFVEDENTLEEQKNIYDTKFVNWKNKY |
| | | YKEKFGFTLSDTEDILKLTESYIQGLQWVLFYYYRGVPS |
| | | WQWYYPYYYAPRISDIKLGIKACLEFDKGTPFKPFEQLM |
| | | AVLPARSKQLVPACYRPLMTDPNSPIIEFYPDEVEIDKNG |
| | | KTASWEAVVKINFVDEKRLLEALEPYNSKLNAEERQRNS |
| | | LGTNIEFSYHPQVNQVHSSPIPTIFPDITEDHCYEIVVEFDK |
| | | LHSSTYAKPMKGAKQGINLLAGFPTLKTIPFTSQLMLAEC |
| | | HIFNQPTKSESMILSTQNPFEGLTVEQFAAQNLGQTIYVN |
| | | WPYLKEAKVVAVSDGLNVFEPGNKSIRTTRMESSEVSEF |
| | | ASDVRYISETLFKRKGVLLVNYTEEELQGTPEPRRSPHND |
| | | DAIQGIVFVKKVNGVIRTRSGAYVKTYSDKIEKYPINLLV |
| | | DDVVNKDRRLLEKPPVPLEEEFPKGTTLISLGAFAYGTPA |
| | | TVVDHQNDLMTVRFVNQPIKHEFNYGEIQAQREAHTNV |
| | | YYPSFKVAKIVGITALALSRITSSYRIVNGAKKTVNIGLDL |
| | | KFEGRKLKVLGYTKRNEKHWSYSALAVNLLQQYQKRFP |
| | | SVFKIISLRNDSSIPEAKELFPQVPASQVDEKVNELVAWV |
| | | KEQKKSFVVATLESESLTKVSIGKIEQEVIKFVSKPHEHIP |
| | | QKGLKGVPREATLDISNSSQFLSKQTFNLGDRVIYVEDSG |
| | | KVPNFSKGTVIGVRSVGTKVTLNVLFDLPLLSGNTFDGR |
| | | LATPRGVTVDSSLVLNLTKRQLIYHDRKPAKKEGKPGVK |
| | | KTSQDSKKQTVKLQNGSGKAKQAQDAELSVTTTPVPVP |
| | | AANTAPVTASVQATSVVTATVPTSKPSNNSEDEHELLRL |
| | | LKGNKDSSESQGSQEPLARTSIQQIYGTVFNQVLSAQPQL |
| | | QPVRGFSNPVPETPVNGVQANEQHSDSTPQNHSRDENQ |
| | | GRGRGRGRGNRRGRGRGRGKGGQ |
| 77 | Mouse CMP- | ATGGCTCCAGCTAGAGAAAACGTTTCCTTGTTCTTCAA |
| | sialic acid | GTTGTACTGTTTGGCTGTTATGACTTTGGTTGCTGCTG |
| | transporter | CTTACACTGTTGCTTTGAGATACACTAGAACTACTGCT |
| | (MmCST) | GAGGAGTTGTACTTCTCCACTACTGCTGTTTGTATCAC |
| | Codon | TGAGGTTATCAAGTTGTTGATCTCCGTTGGTTTGTTGG |
| | optimized | CTAAGGAGACTGGTTCTTTGGGAAGATTCAAGGCTTC |
| | | CTTGTCCGAAAACGTTTTGGGTTCCCCAAAGGAGTTG |
| | | GCTAAGTTGTCTGTTCCATCCTTGGTTTACGCTGTTCA |
| | | GAACAACATGGCTTTCTTGGCTTTGTCTAACTTGGACG |
| | | CTGCTGTTTACCAAGTTACTTACCAGTTGAAGATCCCA |
| | | TGTACTGCTTTGTGTACTGTTTTGATGTTGAACAGAAC |
| | | ATTGTCCAAGTTGCAGTGGATCTCCGTTTTCATGTTGT |
| | | GTGGTGGTGTTACTTTGGTTCAGTGGAAGCCAGCTCA |
| | | AGCTTCCAAAGTTGTTGTTGCTCAGAACCCATTGTTGG |
| | | GTTTCGGTGCTATTGCTATCGCTGTTTTGTGTTCCGGTT |
| | | TCGCTGGTGTTTACTTCGAGAAGGTTTTGAAGTCCTCC |
| | | GACACTTCTTTGTGGGTTAGAAACATCCAGATGTACTT |
| | | GTCCGGTATCGTTGTTACTTTGGCTGGTACTTACTTGT |
| | | CTGACGGTGCTGAGATTCAAGAGAAGGGATTCTTCTA |
| | | CGGTTACACTTACTATGTTTGGTTCGTTATCTTCTTGGC |
| | | TTCCGTTGGTGGTTTGTACACTTCCGTTGTTGTTAAGT |
| | | ACACTGACAACATCATGAAGGGATTCTCTGCTGCTGC |
| | | TGCTATTGTTTTGTCCACTATCGCTTCCGTTTTGTTGTT |
| | | CGGATTGCAGATCACATTGTCCTTTGCTTTGGGAGCTT |
| | | TGTTGGTTTGTGTTTCCATCTACTTGTACGGATTGCCA |
| | | AGACAAGACACTACTTCCATTCAGCAAGAGGCTACTT |
| | | CCAAGGAGAGAATCATCGGTGTTTAGTAG |
| 78 | Human UDP- | ATGGAAAAGAACGGTAACAACAGAAAGTTGAGAGTTT |
| | GlcNAc 2- | GTGTTGCTACTTGTAACAGAGCTGACTACTCCAAGTTG |
| | epimerase/N- | GCTCCAATCATGTTCGGTATCAAGACTGAGCCAGAGT |
| | acetylmannosamine | TCTTCGAGTTGGACGTTGTTGTTTTGGGTTCCCACTTG |
| | kinase | ATTGATGACTACGGTAACACTTACAGAATGATCGAGC |
| | (HsGNE) | AGGACGACTTCGACATCAACACTAGATTGCACACTAT |
| | codon | TGTTAGAGGAGAGGACGAAGCTGCTATGGTTGAATCT |
| | opitimized | GTTGGATTGGCTTTGGTTAAGTTGCCAGACGTTTTGAA |
| | | CAGATTGAAGCCAGACATCATGATTGTTCACGGTGAC |
| | | AGATTCGATGCTTTGGCTTTGGCTACTTCCGCTGCTTT |
| | | GATGAACATTAGAATCTTGCACATCGAGGGTGGTGAA |
| | | GTTTCTGGTACTATCGACGACTCCATCAGACACGCTAT |
| | | CACTAAGTTGGCTCACTACCATGTTTGTTGTACTAGAT |
| | | CCGCTGAGCAACACTTGATTTCCATGTGTGAGGACCA |
| | | CGACAGAATTTTGTTGGCTGGTTGTCCATCTTACGACA |
| | | AGTTGTTGTCCGCTAAGAACAAGGACTACATGTCCAT |
| | | CATCAGAATGTGGTTGGGTGACGACGTTAAGTCTAAG |
| | | GACTACATCGTTGCTTTGCAGCACCCAGTTACTACTGA |
| | | CATCAAGCACTCCATCAAGATGTTCGAGTTGACTTTGG |
| | | ACGCTTTGATCTCCTTCAACAAGAGAACTTTGGTTTTG |
| | | TTCCCAAACATTGACGCTGGTTCCAAAGAGATGGTTA |
| | | GAGTTATGAGAAAGAAGGGTATCGAACACCACCCAA |
| | | ACTTCAGAGCTGTTAAGCACGTTCCATTCGACCAATTC |
| | | ATCCAGTTGGTTGCTCATGCTGGTTGTATGATCGGTAA |
| | | CTCCTCCTGTGGTGTTAGAGAAGTTGGTGCTTTCGGTA |
| | | CTCCAGTTATCAACTTGGGTACTAGACAGATCGGTAG |
| | | AGAGACTGGAGAAAACGTTTTGCATGTTAGAGATGCT |
| | | GACACTCAGGACAAGATTTTGCAGGCTTTGCACTTGC |
| | | AATTCGGAAAGCAGTACCCATGTTCCAAAATCTACGG |
| | | TGACGGTAACGCTGTTCCAAGAATCTTGAAGTTTTTGA |
| | | AGTCCATCGACTTGCAAGAGCCATTGCAGAAGAAGTT |
| | | CTGTTTCCCACCAGTTAAGGAGAACATCTCCCAGGAC |
| | | ATTGACCACATCTTGGAGACATTGTCCGCTTTGGCTGT |
| | | TGATTTGGGTGGAACTAACTTGAGAGTTGCTATCGTTT |
| | | CCATGAAGGGAGAGATCGTTAAGAAGTACACTCAGTT |
| | | CAACCCAAAGACTTACGAGGAGAGAATCAACTTGATC |
| | | TTGCAGATGTGTGTTGAAGCTGCTGCTGAGGCTGTTAA |
| | | GTTGAACTGTAGAATCTTGGGTGTTGGTATCTCTACTG |
| | | GTGGTAGAGTTAATCCAAGAGAGGGTATCGTTTTGCA |
| | | CTCCACTAAGTTGATTCAGGAGTGGAACTCCGTTGATT |
| | | TGAGAACTCCATTGTCCGACACATTGCACTTGCCAGTT |
| | | TGGGTTGACAACGACGGTAATTGTGCTGCTTTGGCTG |
| | | AGAGAAAGTTCGGTCAAGGAAAGGGATTGGAGAACTT |
| | | CGTTACTTTGATCACTGGTACTGGTATTGGTGGTGGTA |
| | | TCATTCACCAGCACGAGTTGATTCACGGTTCTTCCTTC |
| | | TGTGCTGCTGAATTGGGACACTTGGTTGTTTCTTTGGA |
| | | CGGTCCAGACTGTTCTTGTGGTTCCCACGGTTGTATTG |
| | | AAGCTTACGCATCAGGAATGGCATTGCAGAGAGAGGC |
| | | TAAGAAGTTGCACGACGAGGACTTGTTGTTGGTTGAG |
| | | GGAATGTCTGTTCCAAAGGACGAGGCTGTTGGTGCTT |
| | | TGCATTTGATCCAGGCTGCTAAGTTGGGTAATGCTAA |
| | | GGCTCAGTCCATCTTGAGAACTGCTGGTACTGCTTTGG |
| | | GATTGGGTGTTGTTAATATCTTGCACACTATGAACCCA |
| | | TCCTTGGTTATCTTGTCCGGTGTTTTGGCTTCTCACTAC |
| | | ATCCACATCGTTAAGGACGTTATCAGACAGCAAGCTT |
| | | TGTCCTCCGTTCAAGACGTTGATGTTGTTGTTTCCGAC |
| | | TTGGTTGACCCAGCTTTGTTGGGTGCTGCTTCCATGGT |
| | | TTTGGACTACACTACTAGAAGAATCTACTAATAG |
| 79 | Pichia pastoris | CAGTTGAGCCAGACCGCGCTAAACGCATACCAATTGC |
| | Sequence of the | CAAATCAGGCAATTGTGAGACAGTGGTAAAAAAGATG |
| | PpARG1 | CCTGCAAAGTTAGATTCACACAGTAAGAGAGATCCTA |
| | auxotrophic | CTCATAAATGAGGCGCTTATTTAGTAGCTAGTGATAG |
| | marker: | CCACTGCGGTTCTGCTTTATGCTATTTGTTGTATGCCTT |
| | | ACTATCTTTGTTTGGCTCCTTTTTCTTGACGTTTTCCGT |
| | | TGGAGGGACTCCCTATTCTGAGTCATGAGCCGCACAG |
| | | ATTATCGCCCAAAATTGACAAAATCTTCTGGCGAAAA |
| | | AAGTATAAAAGGAGAAAAAAGCTCACCCTTTTCCAGC |
| | | GTAGAAAGTATATATCAGTCATTGAAGACTATTATTTA |
| | | AATAACACAATGTCTAAAGGAAAAGTTTGTTTGGCCT |
| | | ACTCCGGTGGTTTGGATACCTCCATCATCCTAGCTTGG |
| | | TTGTTGGAGCAGGGATACGAAGTCGTTGCCTTTTTAGC |
| | | CAACATTGGTCAAGAGGAAGACTTTGAGGCTGCTAGA |
| | | GAGAAAGCTCTGAAGATCGGTGCTACCAAGTTTATCG |
| | | TCAGTGACGTTAGGAAGGAATTTGTTGAGGAAGTTTT |
| | | GTTCCCAGCAGTCCAAGTTAACGCTATCTACGAGAAC |
| | | GTCTACTTACTGGGTACCTCTTTGGCCAGACCAGTCAT |
| | | TGCCAAGGCCCAAATAGAGGTTGCTGAACAAGAAGGT |
| | | TGTTTTGCTGTTGCCCACGGTTGTACCGGAAAGGGTAA |
| | | CGATCAGGTTAGATTTGAGCTTTCCTTTTATGCTCTGA |
| | | AGCCTGACGTTGTCTGTATCGCCCCATGGAGAGACCC |
| | | AGAATTCTTCGAAAGATTCGCTGGTAGAAATGACTTG |
| | | CTGAATTACGCTGCTGAGAAGGATATTCCAGTTGCTC |
| | | AGACTAAAGCCAAGCCATGGTCTACTGATGAGAACAT |
| | | GGCTCACATCTCCTTCGAGGCTGGTATTCTAGAAGATC |
| | | CAAACACTACTCCTCCAAAGGACATGTGGAAGCTCAC |
| | | TGTTGACCCAGAAGATGCACCAGACAAGCCAGAGTTC |
| | | TTTGACGTCCACTTTGAGAAGGGTAAGCCAGTTAAAT |
| | | TAGTTCTCGAGAACAAAACTGAGGTCACCGATCCGGT |
| | | TGAGATCTTTTTGACTGCTAACGCCATTGCTAGAAGAA |
| | | ACGGTGTTGGTAGAATTGACATTGTCGAGAACAGATT |
| | | CATCGGAATCAAGTCCAGAGGTTGTTATGAAACTCCA |
| | | GGTTTGACTCTACTGAGAACCACTCACATCGACTTGG |
| | | AAGGTCTTACCGTTGACCGTGAAGTTAGATCGATCAG |
| | | AGACACTTTTGTTACCCCAACCTACTCTAAGTTGTTAT |
| | | ACAACGGGTTGTACTTTACCCCAGAAGGTGAGTACGT |
| | | CAGAACTATGATTCAGCCTTCTCAAAACACCGTCAAC |
| | | GGTGTTGTTAGAGCCAAGGCCTACAAAGGTAATGTGT |
| | | ATAACCTAGGAAGATACTCTGAAACCGAGAAATTGTA |
| | | CGATGCTACCGAATCTTCCATGGATGAGTTGACCGGA |
| | | TTCCACCCTCAAGAAGCTGGAGGATTTATCACAACAC |
| | | AAGCCATCAGAATCAAGAAGTACGGAGAAAGTGTCA |
| | | GAGAGAAGGGAAAGTTTTTGGGACTTTAACTCAAGTA |
| | | AAAGGATAGTTGTACAATTATATATACGAAGAATAAA |
| | | TCATTACAAAAAGTATTCGTTTCTTTGATTCTTAACAG |
| | | GATTCATTTTCTGGGTGTCATCAGGTACAGCGCTGAAT |
| | | ATCTTGAAGTTAACATCGAGCTCATCATCGACGTTCAT |
| | | CACACTAGCCACGTTTCCGCAACGGTAGCAATAATTA |
| | | GGAGCGGACCACACAGTGACGACATC |
| 80 | Human CMP- | ATGGACTCTGTTGAAAAGGGTGCTGCTACTTCTGTTTC |
| | sialic acid | CAACCCAAGAGGTAGACCATCCAGAGGTAGACCTCCT |
| | synthase | AAGTTGCAGAGAAACTCCAGAGGTGGTCAAGGTAGAG |
| | (HsCSS) codon | GTGTTGAAAAGCCACCACACTTGGCTGCTTTGATCTTG |
| | optimized | GCTAGAGGAGGTTCTAAGGGTATCCCATTGAAGAACA |
| | | TCAAGCACTTGGCTGGTGTTCCATTGATTGGATGGGTT |
| | | TTGAGAGCTGCTTTGGACTCTGGTGCTTTCCAATCTGT |
| | | TTGGGTTTCCACTGACCACGACGAGATTGAGAACGTT |
| | | GCTAAGCAATTCGGTGCTCAGGTTCACAGAAGATCCT |
| | | CTGAGGTTTCCAAGGACTCTTCTACTTCCTTGGACGCT |
| | | ATCATCGAGTTCTTGAACTACCACAACGAGGTTGACA |
| | | TCGTTGGTAACATCCAAGCTACTTCCCCATGTTTGCAC |
| | | CCAACTGACTTGCAAAAAGTTGCTGAGATGATCAGAG |
| | | AAGAGGGTTACGACTCCGTTTTCTCCGTTGTTAGAAGG |
| | | CACCAGTTCAGATGGTCCGAGATTCAGAAGGGTGTTA |
| | | GAGAGGTTACAGAGCCATTGAACTTGAACCCAGCTAA |
| | | AAGACCAAGAAGGCAGGATTGGGACGGTGAATTGTAC |
| | | GAAAACGGTTCCTTCTACTTCGCTAAGAGACACTTGAT |
| | | CGAGATGGGATACTTGCAAGGTGGAAAGATGGCTTAC |
| | | TACGAGATGAGAGCTGAACACTCCGTTGACATCGACG |
| | | TTGATATCGACTGGCCAATTGCTGAGCAGAGAGTTTT |
| | | GAGATACGGTTACTTCGGAAAGGAGAAGTTGAAGGAG |
| | | ATCAAGTTGTTGGTTTGTAACATCGACGGTTGTTTGAC |
| | | TAACGGTCACATCTACGTTTCTGGTGACCAGAAGGAG |
| | | ATTATCTCCTACGACGTTAAGGACGCTATTGGTATCTC |
| | | CTTGTTGAAGAAGTCCGGTATCGAAGTTAGATTGATCT |
| | | CCGAGAGAGCTTGTTCCAAGCAAACATTGTCCTCTTTG |
| | | AAGTTGGACTGTAAGATGGAGGTTTCCGTTTCTGACA |
| | | AGTTGGCTGTTGTTGACGAATGGAGAAAGGAGATGGG |
| | | TTTGTGTTGGAAGGAAGTTGCTTACTTGGGTAACGAA |
| | | GTTTCTGACGAGGAGTGTTTGAAGAGAGTTGGTTTGTC |
| | | TGGTGCTCCAGCTGATGCTTGTTCCACTGCTCAAAAGG |
| | | CTGTTGGTTACATCTGTAAGTGTAACGGTGGTAGAGGT |
| | | GCTATTAGAGAGTTCGCTGAGCACATCTGTTTGTTGAT |
| | | GGAGAAAGTTAATAACTCCTGTCAGAAGTAGTAG |
| 81 | Human N- | ATGCCATTGGAATTGGAGTTGTGTCCTGGTAGATGGGT |
| | acetylneuraminate- | TGGTGGTCAACACCCATGTTTCATCATCGCTGAGATCG |
| | 9-phosphate | GTCAAAACCACCAAGGAGACTTGGACGTTGCTAAGAG |
| | synthase | AATGATCAGAATGGCTAAGGAATGTGGTGCTGACTGT |
| | (HsSPS) codon | GCTAAGTTCCAGAAGTCCGAGTTGGAGTTCAAGTTCA |
| | optimized | ACAGAAAGGCTTTGGAAAGACCATACACTTCCAAGCA |
| | | CTCTTGGGGAAAGACTTACGGAGAACACAAGAGACAC |
| | | TTGGAGTTCTCTCACGACCAATACAGAGAGTTGCAGA |
| | | GATACGCTGAGGAAGTTGGTATCTTCTTCACTGCTTCT |
| | | GGAATGGACGAAATGGCTGTTGAGTTCTTGCACGAGT |
| | | TGAACGTTCCATTCTTCAAAGTTGGTTCCGGTGACACT |
| | | AACAACTTCCCATACTTGGAAAAGACTGCTAAGAAAG |
| | | GTAGACCAATGGTTATCTCCTCTGGAATGCAGTCTATG |
| | | GACACTATGAAGCAGGTTTACCAGATCGTTAAGCCAT |
| | | TGAACCCAAACTTTTGTTTCTTGCAGTGTACTTCCGCT |
| | | TACCCATTGCAACCAGAGGACGTTAATTTGAGAGTTA |
| | | TCTCCGAGTACCAGAAGTTGTTCCCAGACATCCCAATT |
| | | GGTTACTCTGGTCACGAGACTGGTATTGCTATTTCCGT |
| | | TGCTGCTGTTGCTTTGGGTGCTAAGGTTTTGGAGAGAC |
| | | ACATCACTTTGGACAAGACTTGGAAGGGTTCTGATCA |
| | | CTCTGCTTCTTTGGAACCTGGTGAGTTGGCTGAACTTG |
| | | TTAGATCAGTTAGATTGGTTGAGAGAGCTTTGGGTTCC |
| | | CCAACTAAGCAATTGTTGCCATGTGAGATGGCTTGTA |
| | | ACGAGAAGTTGGGAAAGTCCGTTGTTGCTAAGGTTAA |
| | | GATCCCAGAGGGTACTATCTTGACTATGGACATGTTG |
| | | ACTGTTAAAGTTGGAGAGCCAAAGGGTTACCCACCAG |
| | | AGGACATCTTTAACTTGGTTGGTAAAAAGGTTTTGGTT |
| | | ACTGTTGAGGAGGACGACACTATTATGGAGGAGTTGG |
| | | TTGACAACCACGGAAAGAAGATCAAGTCCTAG |
| 82 | Mouse alpha- | GTTTTTCAAATGCCAAAGTCCCAGGAGAAAGTTGCTG |
| | 2,6-sialyl | TTGGTCCAGCTCCACAAGCTGTTTTCTCCAACTCCAAG |
| | transferase | CAAGATCCAAAGGAGGGTGTTCAAATCTTGTCCTACC |
| | catalytic domain | CAAGAGTTACTGCTAAGGTTAAGCCACAACCATCCTT |
| | (MmmST6) | GCAAGTTTGGGACAAGGACTCCACTTACTCCAAGTTG |
| | codon optimized | AACCCAAGATTGTTGAAGATTTGGAGAAACTACTTGA |
| | | ACATGAACAAGTACAAGGTTTCCTACAAGGGTCCAGG |
| | | TCCAGGTGTTAAGTTCTCCGTTGAGGCTTTGAGATGTC |
| | | ACTTGAGAGACCACGTTAACGTTTCCATGATCGAGGC |
| | | TACTGACTTCCCATTCAACACTACTGAATGGGAGGGA |
| | | TACTTGCCAAAGGAGAACTTCAGAACTAAGGCTGGTC |
| | | CATGGCATAAGTGTGCTGTTGTTTCTTCTGCTGGTTCC |
| | | TTGAAGAACTCCCAGTTGGGTAGAGAAATTGACAACC |
| | | ACGACGCTGTTTTGAGATTCAACGGTGCTCCAACTGA |
| | | CAACTTCCAGCAGGATGTTGGTACTAAGACTACTATC |
| | | AGATTGGTTAACTCCCAATTGGTTACTACTGAGAAGA |
| | | GATTCTTGAAGGACTCCTTGTACACTGAGGGAATCTTG |
| | | ATTTTGTGGGACCCATCTGTTTACCACGCTGACATTCC |
| | | ACAATGGTATCAGAAGCCAGACTACAACTTCTTCGAG |
| | | ACTTACAAGTCCTACAGAAGATTGCACCCATCCCAGC |
| | | CATTCTACATCTTGAAGCCACAAATGCCATGGGAATT |
| | | GTGGGACATCATCCAGGAAATTTCCCCAGACTTGATC |
| | | CAACCAAACCCACCATCTTCTGGAATGTTGGGTATCAT |
| | | CATCATGATGACTTTGTGTGACCAGGTTGACATCTACG |
| | | AGTTCTTGCCATCCAAGAGAAAGACTGATGTTTGTTAC |
| | | TACCACCAGAAGTTCTTCGACTCCGCTTGTACTATGGG |
| | | AGCTTACCACCCATTGTTGTTCGAGAAGAACATGGTT |
| | | AAGCACTTGAACGAAGGTACTGACGAGGACATCTACT |
| | | TGTTCGGAAAGGCTACTTTGTCCGGTTTCAGAAACAA |
| | | CAGATGTTAG |
| 83 | Pichia pastoris | ACTGGGCCTTTAGAGGGTGCTGAAGTTGACCCCTTGG |
| | Pp TRP2: 5′ and | TGCTTCTGGAAAAAGAACTGAAGGGCACCAGACAAGC |
| | ORF | GCAACTTCCTGGTATTCCTCGTCTAAGTGGTGGTGCCA |
| | | TAGGATACATCTCGTACGATTGTATTAAGTACTTTGAA |
| | | CCAAAAACTGAAAGAAAACTGAAAGATGTTTTGCAAC |
| | | TTCCGGAAGCAGCTTTGATGTTGTTCGACACGATCGTG |
| | | GCTTTTGACAATGTTTATCAAAGATTCCAGGTAATTGG |
| | | AAACGTTTCTCTATCCGTTGATGACTCGGACGAAGCTA |
| | | TTCTTGAGAAATATTATAAGACAAGAGAAGAAGTGGA |
| | | AAAGATCAGTAAAGTGGTATTTGACAATAAAACTGTT |
| | | CCCTACTATGAACAGAAAGATATTATTCAAGGCCAAA |
| | | CGTTCACCTCTAATATTGGTCAGGAAGGGTATGAAAA |
| | | CCATGTTCGCAAGCTGAAAGAACATATTCTGAAAGGA |
| | | GACATCTTCCAAGCTGTTCCCTCTCAAAGGGTAGCCA |
| | | GGCCGACCTCATTGCACCCTTTCAACATCTATCGTCAT |
| | | TTGAGAACTGTCAATCCTTCTCCATACATGTTCTATAT |
| | | TGACTATCTAGACTTCCAAGTTGTTGGTGCTTCACCTG |
| | | AATTACTAGTTAAATCCGACAACAACAACAAAATCAT |
| | | CACACATCCTATTGCTGGAACTCTTCCCAGAGGTAAA |
| | | ACTATCGAAGAGGACGACAATTATGCTAAGCAATTGA |
| | | AGTCGTCTTTGAAAGACAGGGCCGAGCACGTCATGCT |
| | | GGTAGATTTGGCCAGAAATGATATTAACCGTGTGTGT |
| | | GAGCCCACCAGTACCACGGTTGATCGTTTATTGACTGT |
| | | GGAGAGATTTTCTCATGTGATGCATCTTGTGTCAGAAG |
| | | TCAGTGGAACATTGAGACCAAACAAGACTCGCTTCGA |
| | | TGCTTTCAGATCCATTTTCCCAGCAGGTACCGTCTCCG |
| | | GTGCTCCGAAGGTAAGAGCAATGCAACTCATAGGAGA |
| | | ATTGGAAGGAGAAAAGAGAGGTGTTTATGCGGGGGCC |
| | | GTAGGACACTGGTCGTACGATGGAAAATCGATGGACA |
| | | CATGTATTGCCTTAAGAACAATGGTCGTCAAGGACGG |
| | | TGTCGCTTACCTTCAAGCCGGAGGTGGAATTGTCTACG |
| | | ATTCTGACCCCTATGACGAGTACATCGAAACCATGAA |
| | | CAAAATGAGATCCAACAATAACACCATCTTGGAGGCT |
| | | GAGAAAATCTGGACCGATAGGTTGGCCAGAGACGAG |
| | | AATCAAAGTGAATCCGAAGAAAACGATCAATGA |
| 84 | Pichia pastoris | ACGGAGGACGTAAGTAGGAATTTATGTAATCATGCCA |
| | PpTRP2 3′ | ATACATCTTTAGATTTCTTCCTCTTCTTTTTAACGAAAG |
| | region | ACCTCCAGTTTTGCACTCTCGACTCTCTAGTATCTTCC |
| | | CATTTCTGTTGCTGCAACCTCTTGCCTTCTGTTTCCTTC |
| | | AATTGTTCTTCTTTCTTCTGTTGCACTTGGCCTTCTTCC |
| | | TCCATCTTTCGTTTTTTTTCAAGCCTTTTCAGCAGTTCT |
| | | TCTTCCAAGAGCAGTTCTTTGATTTTCTCTCTCCAATCC |
| | | ACCAAAAAACTGGATGAATTCAACCGGGCATCATCAA |
| | | TGTTCCACTTTCTTTCTCTTATCAATAATCTACGTGCTT |
| | | CGGCATACGAGGAATCCAGTTGCTCCCTAATCGAGTC |
| | | ATCCACAAGGTTAGCATGGGCCTTTTTCAGGGTGTCA |
| | | AAAGCATCTGGAGCTCGTTTATTCGGAGTCTTGTCTGG |
| | | ATGGATCAGCAAAGACTTTTTGCGGAAAGTCTTTCTTA |
| | | TATCTTCCGGAGAACAACCTGGTTTCAAATCCAAGAT |
| | | GGCATAGCTGTCCAATTTGAAAGTGGAAAGAATCCTG |
| | | CCAATTTCCTTCTCTCGTGTCAGCTCGTTCTCCTCCTTT |
| | | TGCAACAGGTCCACTTCATCTGGCATTTTTCTTTATGT |
| | | TAACTTTAATTATTATTAATTATAAAGTTGATTATCGT |
| | | TATCAAAATAATCATATTCGAGAAATAATCCGTCCAT |
| | | GCAATATATAAATAAGAATTCATAATAATGTAATGAT |
| | | AACAGTACCTCTGATGACCTTTGATGAACCGCAATTTT |
| | | CTTTCCAATGACAAGACATCCCTATAATACAATTATAC |
| | | AGTTTATATATCACAAATAATCACCTTTTTATAAGAAA |
| | | ACCGTCCTCTCCGTAACAGAACTTATTATCCGCACGTT |
| | | ATGGTTAACACACTACTAATACCGATATAGTGTATGA |
| | | AGTCGCTACGAGATAGCCATCCAGGAAACTTACCAAT |
| | | TCATCAGCACTTTCATGATCCGATTGTTGGCTTTATTC |
| | | TTTGCGAGACAGATACTTGCCAATGAAATAACTGATC |
| | | CCACAGATGAGAATCCGGTGCTCGT |
| 85 | Pichia pastoris | TTGGGGGCCTCCAGGACTTGCTGAAATTTGCTGACTCA |
| | Sequence of the | TCTTCGCCATCCAAGGATAATGAGTTAGCTAATGTGA |
| | 5′-Region used | CAGTTAATGAGTCGTCTTGACTAACGGGGAACATTTC |
| | for knock out of | ATTATTTATATCCAGAGTCAATTTGATAGCAGAGTTTG |
| | STE13 | TGGTTGAAATACCTATGATTCGGGAGACTTTGTTGTAA |
| | | CGACCATTATCCACAGTTTGGACCGTGAAAATGTCAT |
| | | CGAAGAGAGCAGACGACATATTATCTATTGTGGTAAG |
| | | TGATAGTTGGAAGTCCGACTAAGGCATGAAAATGAGA |
| | | AGACTGAAAATTTAAAGTTTTTGAAAACACTAATCGG |
| | | GTAATAACTTGGAAATTACGTTTACGTGCCTTTAGCTC |
| | | TTGTCCTTACCCCTGATAATCTATCCATTTCCCGAGAG |
| | | ACAATGACATCTCGGACAGCTGAGAACCCGTTCGATA |
| | | TAGAGCTTCAAGAGAATCTAAGTCCACGTTCTTCCAAT |
| | | TCGTCCATATTGGAAAACATTAATGAGTATGCTAGAA |
| | | GACATCGCAATGATTCGCTTTCCCAAGAATGTGATAA |
| | | TGAAGATGAGAACGAAAATCTCAATTATACTGATAAC |
| | | TTGGCCAAGTTTTCAAAGTCTGGAGTATCAAGAAAGA |
| | | GCTGTATGCTAATATTTGGTATTTGCTTTGTTATCTGG |
| | | CTGTTTCTCTTTGCCTTGTATGCGAGGGACAATCGATT |
| | | TTCCAATTTGAACGAGTACGTTCCAGATTCAAACAG |
| 86 | Pichia pastoris | CTACTGGGAACCACGAGACATCACTGCAGTAGTTTCC |
| | Sequence of the | AAGTGGATTTCAGATCACTCATTTGTGAATCCTGACAA |
| | 3′-Region used | AACTGCGATATGGGGGTGGTCTTACGGTGGGTTCACT |
| | for knock out of | ACGCTTAAGACATTGGAATATGATTCTGGAGAGGTTTT |
| | STE13 | CAAATATGGTATGGCTGTTGCTCCAGTAACTAATTGGC |
| | | TTTTGTATGACTCCATCTACACTGAAAGATACATGAAC |
| | | CTTCCAAAGGACAATGTTGAAGGCTACAGTGAACACA |
| | | GCGTCATTAAGAAGGTTTCCAATTTTAAGAATGTAAA |
| | | CCGATTCTTGGTTTGTCACGGGACTACTGATGATAACG |
| | | TGCATTTTCAGAACACACTAACCTTACTGGACCAGTTC |
| | | AATATTAATGGTGTTGTGAATTACGATCTTCAGGTGTA |
| | | TCCCGACAGTGAACATAGCATTGCCCATCACAACGCA |
| | | AATAAAGTGATCTACGAGAGGTTATTCAAGTGGTTAG |
| | | AGCGGGCATTTAACGATAGATTTTTGTAACATTCCGTA |
| | | CTTCATGCCATACTATATATCCTGCAAGGTTTCCCTTT |
| | | CAGACACAATAATTGCTTTGCAATTTTACATACCACCA |
| | | ATTGGCAAAAATAATCTCTTCAGTAAGTTGAATGCTTT |
| | | TCAAGCCAGCACCGTGAGAAATTGCTACAGCGCGCAT |
| | | TCTAACATCACTTTAAAATTCCCTCGCCGGTGCTCACT |
| | | GGAGTTTCCAACCCTTAGCTTATCAAAATCGGGTGAT |
| | | AACTCTGAGTTTTTTTTTTCACTTCTATTCCTAAACCTT |
| | | CGCCCAATGCTACCACCTCCAATCAACATCCCGAAAT |
| | | GGATAGAAGAGAATGGACATCTCTTGCAACCTCCGGT |
| | | TAATAATTACTGTCTCCACAGAGGAGGATTTACGGTA |
| | | ATGATTGTAGGTGGGCCTAATG |
| 87 | Pichia pastoris | CACCTGGGCCTGTTGCTGCTGGTACTGCTGTTGGAACT |
| | Sequence of the | GTTGGTATTGTTGCTGATCTAAGGCCGCCTGTTCCACA |
| | 5′-Region used | CCGTGTGTATCGAATGCTTGGGCAAAATCATCGCCTG |
| | for knock out of | CCGGAGGCCCCACTACCGCTTGTTCCTCCTGCTCTTGT |
| | DAP2 | TTGTTTTGCTCATTGATGATATCGGCGTCAATGAATTG |
| | | ATCCTCAATCGTGTGGTGGTGGTGTCGTGATTCCTCTT |
| | | CTTTCTTGAGTGCCTTATCCATATTCCTATCTTAGTGTA |
| | | CCAATAATTTTGTTAAACACACGCTGTTGTTTATGAAA |
| | | AGTCGTCAAAAGGTTAAAAATTCTACTTGGTGTGTGTC |
| | | AGAGAAAGTAGTGCAGACCCCCAGTTTGTTGACTAGT |
| | | TGAGAAGGCGGCTCACTATTGCGCGAATAGCATGAGA |
| | | AATTTGCAAACATCTGGCAAAGTGGTCAATACCTGCC |
| | | AACCTGCCAATCTTCGCGACGGAGGCTGTTAAGCGGG |
| | | TTGGGTTCCCAAAGTGAATGGATATTACGGGCAGGAA |
| | | AAACAGCCCCTTCCACACTAGTCTTTGCTACTGACATC |
| | | TTCCCTCTCATGTATCCCGAACACAAGTATCGGGAGTA |
| | | TCAACGGAGGGTGCCCTTATGGCAGTACTCCCTGTTG |
| | | GTGATTGTACTGCTATACGGGTCTCATTTGCTTATCAG |
| | | CACCATCAACTTGATACACTATAACCACAAAAATTAT |
| | | CATGCACACCCAGTCAATAGTGGTATCGTTCTTAATGA |
| | | GTTTGCTGATGACGATTCATTCTCTTTGAATGGCACTC |
| | | TGAACTTGGAGAACTGGAGAAATGGTACCTTTTCCCC |
| | | TAAATTTCATTCCATTCAGTGGACCGAAATAGGTCAG |
| | | GAAGATGACCAGGGATATTACATTCTCTCTTCCAATTC |
| | | CTCTTACATAGTAAAGTCTTTATCCGACCCAGACTTTG |
| | | AATCTGTTCTATTCAACGAGTCTACAATCACTTACAACG |
| 88 | Pichia pastoris | GGCAGCAAAGCCTTACGTTGATGAGAATAGACTGGCC |
| | Sequence of the | ATTTGGGGTTGGTCTTATGGAGGTTACATGACGCTAAA |
| | 3′-Region used | GGTTTTAGAACAGGATAAAGGTGAAACATTCAAATAT |
| | for knock out of | GGAATGTCTGTTGCCCCTGTGACGAATTGGAAATTCTA |
| | DAP2 | TGATTCTATCTACACAGAAAGATACATGCACACTCCTC |
| | | AGGACAATCCAAACTATTATAATTCGTCAATCCATGA |
| | | GATTGATAATTTGAAGGGAGTGAAGAGGTTCTTGCTA |
| | | ATGCACGGAACTGGTGACGACAATGTTCACTTCCAAA |
| | | ATACACTCAAAGTTCTAGATTTATTTGATTTACATGGT |
| | | CTTGAAAACTATGATATCCACGTGTTCCCTGATAGTGA |
| | | TCACAGTATTAGATATCACAACGGTAATGTTATAGTGT |
| | | ATGATAAGCTATTCCATTGGATTAGGCGTGCATTCAA |
| | | GGCTGGCAAATAAATAGGTGCAAAAATATTATTAGAC |
| | | TTTTTTTTTCGTTCGCAAGTTATTACTGTGTACCATACC |
| | | GATCCAATCCGTATTGTAATTCATGTTCTAGATCCAAA |
| | | ATTTGGGACTCTAATTCATGAGGTCTAGGAAGATGAT |
| | | CATCTCTATAGTTTTCAGCGGGGGGCTCGATTTGCGGT |
| | | TGGTCAAAGCTAACATCAAAATGTTTGTCAGGTTCAG |
| | | TGAATGGTAACTGCTGCTCTTGAATTGGTCGTCTGACA |
| | | AATTCTCTAAGTGATAGCACTTCATCTACAATCATTTG |
| | | CTTCATCGTTTCTATATCGTCCACGACCTCAAACGAGA |
| | | AATCGAATTTGGAAGAACAGACGGGCTCATCGTTAGG |
| | | ATCATGCCAAACCTTGAGATATGGATGCTCTAAAGCC |
| | | TCAGTAACTGTAATTCTGTGAGTGGGATCTACCGTGA |
| | | GCATTCGATCCAGTAAGTCTATCGCTTCAGGGTTGGCA |
| | | CCGGGAAATAACTGGCTGAATGGGATCTTGGGCATGA |
| | | ATGGCAGGGAGCGAACATAATCCTGGGCACGCTCTGA |
| | | TCTGATAGACTGAAGTGTCTCTTCCGAAACAGTACCC |
| | | AGCGTACTCAAAATCAAGTTCAATTGATCCACATAGT |
| | | CTCTTCCTCTAAAAATGGGTCGGCCACCTA |
| 89 | Escherichia coli | GATCTGTTTAGCTTGCCTCGTCCCCGCCGGGTCACCCG |
| | HYG^{R} resistance | GCCAGCGACATGGAGGCCCAGAATACCCTCCTTGACA |
| | cassette | GTCTTGACGTGCGCAGCTCAGGGGCATGATGTGACTG |
| | | TCGCCCGTACATTTAGCCCATACATCCCCATGTATAAT |
| | | CATTTGCATCCATACATTTTGATGGCCGCACGGCGCGA |
| | | AGCAAAAATTACGGCTCCTCGCTGCGGACCTGCGAGC |
| | | AGGGAAACGCTCCCCTCACAGACGCGTTGAATTGTCC |
| | | CCACGCCGCGCCCCTGTAGAGAAATATAAAAGGTTAG |
| | | GATTTGCCACTGAGGTTCTTCTTTCATATACTTCCTTTT |
| | | AAAATCTTGCTAGGATACAGTTCTCACATCACATCCG |
| | | AACATAAACAACCATGGGTAAAAAGCCTGAACTCACC |
| | | GCGACGTCTGTCGAGAAGTTTCTGATCGAAAAGTTCG |
| | | ACAGCGTCTCCGACCTGATGCAGCTCTCGGAGGGCGA |
| | | AGAATCTCGTGCTTTCAGCTTCGATGTAGGAGGGCGT |
| | | GGATATGTCCTGCGGGTAAATAGCTGCGCCGATGGTT |
| | | TCTACAAAGATCGTTATGTTTATCGGCACTTTGCATCG |
| | | GCCGCGCTCCCGATTCCGGAAGTGCTTGACATTGGGG |
| | | AATTCAGCGAGAGCCTGACCTATTGCATCTCCCGCCGT |
| | | GCACAGGGTGTCACGTTGCAAGACCTGCCTGAAACCG |
| | | AACTGCCCGCTGTTCTGCAGCCGGTCGCGGAGGCCAT |
| | | GGATGCGATCGCTGCGGCCGATCTTAGCCAGACGAGC |
| | | GGGTTCGGCCCATTCGGACCGCAAGGAATCGGTCAAT |
| | | ACACTACATGGCGTGATTTCATATGCGCGATTGCTGAT |
| | | CCCCATGTGTATCACTGGCAAACTGTGATGGACGACA |
| | | CCGTCAGTGCGTCCGTCGCGCAGGCTCTCGATGAGCT |
| | | GATGCTTTGGGCCGAGGACTGCCCCGAAGTCCGGCAC |
| | | CTCGTGCACGCGGATTTCGGCTCCAACAATGTCCTGAC |
| | | GGACAATGGCCGCATAACAGCGGTCATTGACTGGAGC |
| | | GAGGCGATGTTCGGGGATTCCCAATACGAGGTCGCCA |
| | | ACATCTTCTTCTGGAGGCCGTGGTTGGCTTGTATGGAG |
| | | CAGCAGACGCGCTACTTCGAGCGGAGGCATCCGGAGC |
| | | TTGCAGGATCGCCGCGGCTCCGGGCGTATATGCTCCG |
| | | CATTGGTCTTGACCAACTCTATCAGAGCTTGGTTGACG |
| | | GCAATTTCGATGATGCAGCTTGGGCGCAGGGTCGATG |
| | | CGACGCAATCGTCCGATCCGGAGCCGGGACTGTCGGG |
| | | CGTACACAAATCGCCCGCAGAAGCGCGGCCGTCTGGA |
| | | CCGATGGCTGTGTAGAAGTACTCGCCGATAGTGGAAA |
| | | CCGACGCCCCAGCACTCGTCCGAGGGCAAAGGAATAA |
| | | TCAGTACTGACAATAAAAAGATTCTTGTTTTCAAGAA |
| | | CTTGTCATTTGTATAGTTTTTTTATATTGTAGTTGTTCT |
| | | ATTTTAATCAAATGTTAGCGTGATTTATATTTTTTTTCG |
| | | CCTCGACATCATCTGCCCAGATGCGAAGTTAAGTGCG |
| | | CAGAAAGTAATATCATGCGTCAATCGTATGTGAATGC |
| | | TGGTCGCTATACTGCTGTCGATTCGATACTAACGCCGC |
| | | CATCCAGTGTCGAAAACGAGCT |
| 90 | Pichia pastoris | ACGACGGCCAAATTCATGATACACACTCTGTTTCAGCT |
| | Sequence of | GGTTTGGACTACCCTGGAGTTGGTCCTGAATTGGCTGC |
| | PpTRP5 5′ | CTGGAAAGCAAATGGTAGAGCCCAATTTTCCGCTGTA |
| | integration | ACTGATGCCCAAGCATTAGAGGGATTCAAAATCCTGT |
| | fragment | CTCAATTGGAAGGGATCATTCCAGCACTAGAGTCTAG |
| | | TCATGCAATCTACGGCGCATTGCAAATTGCAAAGACT |
| | | ATGTCTTCGGACCAGTCCTTAGTTATTAATGTATCTGG |
| | | AAGGGGTGATAAGGACGTCCAGAGTGTAGCTGAGATT |
| | | TTACCTAAATTGGGACCTCAAATTGGATGGGATTTGC |
| | | GTTTCAGCGAAGACATTACTAAAGAGTGA |
| 91 | Pichia pastoris | TCGATAGCACAATATTCAACTTGACTGGGTGTTAAGA |
| | Sequence of | ACTAAGAGCTCTGGGAAACTTTGTATTTATTACTACCA |
| | PpTRP5 3′ | ACACAGTCAAATTATTGGATGTGTTTTTTTTTCCAGTA |
| | integration | CATTTCACTGAGCAGTTTGTTATACTCGGTCTTTAATC |
| | fragment | TCCATATACATGCAGATTGTAATACAGATCTGAACAG |
| | | TTTGATTCTGATTGATCTTGCCACCAATATTCTATTTTT |
| | | GTATCAAGTAACAGAGTCAATGATCATTGGTAACGTA |
| | | ACGGTTTTCGTGTATAGTAGTTAGAGCCCATCTTGTAA |
| | | CCTCATTTCCTCCCATATTAAAGTATCAGTGATTCGCT |
| | | GGAACGATTAACTAAGAAAAAAAAAATATCTGCACAT |
| | | ACTCATCAGTCTGTAAATCTAAGTCAAAACTGCTGTAT |
| | | CCAATAGAAATCGGGATATACCTGGATGTTTTTTCCAC |
| | | ATAAACAAACGGGAGTTCAGCTTACTTATGGTGTTGA |
| | | TGCAATTCAGTATGATCCTACCAATAAAACGAAACTT |
| | | TGGGATTTTGGCTGTTTGAGGGATCAAAAGCTGCACC |
| | | TTTACAAGATTGACGGATCGACCATTAGACCAAAGCA |
| | | AATGGCCACCAA |
| 92 | Saccharomyces | MKLKTVRSAVLSSLFASQVLG |
| | cerecisiae | |
| | Yps1ss | |
| 93 | Synthetic | QPIDDTESQTTSVNLMADDTESAFATQTNSGGLDVVGLI |
| | construct | SMAKR |
| | TA57 pro | |
| 94 | Synthetic | EEGEPK |
| | construct | |
| | N-terminal | |
| | spacer | |
| 95 | Synthetic | FVNQHLCGSHLVEALYLVCGERGFFYTNKT |
| | construct | |
| | Glycosylated | |
| | insulin B chain | |
| | P28N | |
| 96 | Human insulin A | GIVEQCCTSICSLYQLENYCN |
| | chain | |
| 97 | Synthetic | MKLKTVRSAVLSSLFASQVLGQPIDDTESQTTSVNLMAD |
| | construct | DTESAFATQTNSGGLDVVGLISMAKREEGEPKFVNQHLC |
| | Pre-proinsulin | GSHLVEALYLVCGERGFFYTNKTAAKGIVEQCCTSICSLY |
| | analogue: | QLENYCN |
| | Yps1ss + TA57 | |
| | propeptide + N- | |
| | terminal | |
| | spacer + B chain | |
| | P28N + C-peptide | |
| | “AAK” + insulin | |
| | A chain | |
| 98 | Synthetic | ATGAAGTTGAAGACTGTTAGATCCGCTGTTTTGTCTTC |
| | construct | TTTGTTTGCTTCTCAAGTTTTGGGTCAACCAATTGATG |
| | DNA encoding | ATACTGAATCTCAAACTACTTCTGTTAACTTGATGGCT |
| | pre-proinsulin | GATGATACTGAATCTGCTTTTGCTACTCAAACTAACTC |
| | analogue: | TGGTGGTTTGGATGTTGTTGGTTTGATTTCTATGGCTA |
| | Yps1ss + TA57 | AGAGAGAAGAAGGTGAACCAAAGTTTGTTAACCAACA |
| | propeptide + N- | TTTGTGTGGTTCTCATTTGGTTGAAGCTTTGTACTTGGT |
| | terminal | TTGTGGTGAAAGAGGTTTTTTTTACACTAACAAGACTG |
| | spacer + B chain | CTGCTAAGGGTATTGTTGAACAATGTTGTACTTCTATT |
| | P28N + C-peptide | TGTTCTTTGTACCAATTGGAAAACTACTGTAACTAA |
| | “AAK” + insulin | |
| | A chain | |

Patents, patent applications, publications, product descriptions, and protocols are cited throughout this application, the disclosures of which are incorporated herein by reference in their entireties for all purposes. All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. §1.57(b)(1), to relate to each and every individual publication, database entry (e.g. Genbank sequences or GeneID entries), patent application, or patent, each of which is clearly identified in compliance with 37 C.F.R. §1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein; the embodiments specifically set forth herein are not necessarily intended to be exhaustive. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

What is claimed is:

1. An isolated modified Pichia sp. host cell wherein the host cell has been modified to reduce or eliminate expression of a functional gene product of a nucleic acid sequence encoding a polypeptide having the amino acid sequence set forth in SEQ ID NO:76.

2. The host cell of claim 1, wherein said modified host cell comprises a disruption or deletion in the nucleic acid sequence encoding a polypeptide having the amino acid sequence set forth in SEQ ID NO:76.

3. The host cell of claim 1, which further comprises disruption or deletion of one or more of a functional gene products encoding an alpha-1,6-mannosyltransferase activity, mannosylphosphate transferase activity, β-mannosyltransferase activity, or a dolichol-P-Man dependent alpha(1-3) mannosyltransferaseactivity.

4. The host cell of claim 1, further comprising one or more nucleic acid sequences of interest.

5. The host cell of claim 4, wherein the nucleic acid sequences of interest encode one or more glycosylation enzymes or oligosaccharyltransferases.

6. The host cell of claim 5, wherein the glycosylation enzymes are selected from the group consisting of glycosidases, mannosidases, phosphomannosidases, phosphatases, nucleotide sugar transporters, mannosyltransferases, the N-acetylglucosaminyltransferases, the UDP-N-acetylglucosamine transporters, the galactosyltransferases, the sialyltransferases, the protein mannosyltransferases, and the oligosaccharyltransferases STT3A, STT3B, STT3C and STT3D.

7. The host cell of claim 6, wherein the nucleic acid sequences of interest encode one or more therapeutic proteins.

8. The host cell of claim 7, wherein the therapeutic proteins are selected from the group consisting of kringle domains of the human plasminogen, erythropoietin, cytokines, coagulation factors, soluble IgE receptor α-chain, IgG, IgG fragments, IgM, urokinase, chymase, urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1 antitrypsin, DNase II, α-feto proteins, insulin, Fc-fusions, and HSA-fusions.

9. The host cell of claim 7, wherein the cell is capable of expressing an increased amount of the therapeutic protein and wherein protein glycosylation quality of the therapeutic protein is improved compared with the XRN1 naïve parental host cell under similar culture conditions.

10. A Pichia sp. host cell comprising a disruption or deletion of the XRN1 gene in the genomic DNA of the host cell that encodes a protein having of a nucleic acid sequence encoding a polypeptide having the amino acid sequence set forth in SEQ ID NO:76.

11. The host cell of claim 10, wherein the host cell further comprises disruption or deletion of one or more of a functional gene product encoding an alpha-1,6-mannosyltransferase activity, mannosylphosphate transferase activity, β-mannosyltransferase activity, or a dolichol-P-Man dependent alpha(1-3) mannosyltransferaseactivity.

12. The host cell of claim 10, further comprising one or more nucleic acid sequences of interest.

13. The host cell of claim 12, wherein the nucleic acid sequences of interest encode one or more glycosylation enzymes or oligosaccharyltransferases.

14. The host cell of claim 13, wherein the glycosylation enzymes or oligosaccharyltransferases are selected from the group consisting of glycosidases, mannosidases, phosphomannosidases, phosphatases, nucleotide sugar transporters, mannosyltransferases, the N-acetylglucosaminyltransferases, the UDP-N-acetylglucosamine transporters, the galactosyltransferases, the sialyltransferases, the protein mannosyltransferases, and the oligosaccharyltransferases STT3A, STT3B, STT3C and STT3D.

15. The host cell of claim 12, wherein the nucleic acid sequences of interest encode one or more therapeutic proteins.

16. The host cell of claim 15, wherein the therapeutic proteins are selected from the group consisting of kringle domains of the human plasminogen, erythropoietin, cytokines, coagulation factors, soluble IgE receptor α-chain, IgG, IgG fragments, IgM, urokinase, chymase, urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1 antitrypsin, DNase II, α-feto proteins, insulin, Fc-fusions, an immunoglobulin heavy chain, an immunoglobulin light chain, and HSA-fusions.

17. The host cell of claim 15, which is capable of expressing an increased amount of the therapeutic protein and wherein protein glycosylation quality of the therapeutic protein is improved compared with the XRN1 naïve parental host cell under similar culture conditions.

18. A method for producing a glycoprotein composition in an isolated Pichia sp. host cell, said method comprising growing said host cell of claim 1 under inducing conditions.

19. The method of claim 18, wherein said host cell is capable of expressing an increased amount of the therapeutic protein and wherein protein glycosylation quality of the therapeutic protein is improved compared with the XRN1 naïve parental host cell under similar culture conditions.

20. A method for producing glycoprotein compositions in Pichia sp. host cells, said method comprising growing said host cell of claim 10 under inducing conditions.

21. The method of claim 20, wherein said host cell is capable of expressing an increased amount of the therapeutic protein and wherein protein glycosylation quality of the therapeutic protein is improved compared with the XRN1 naïve parental host cell under similar culture conditions.
